(19) United States
(12) Patent Application Publication
(10) Pub. No.: US 2024/0287158 A1
(43) Pub. Date: Aug. 29, 2024

(54) FORMULATIONS OF FACTOR VIII CHIMERIC PROTEINS AND USES THEREOF

(71) Applicant: BIOVERATIV THERAPEUTICS INC., Waltham, MA (US)

(72) Inventors: Tyler CARLAGE, Cambridge, MA (US); Randall MAULDIN, Cambridge, MA (US); Loubna Mzaalak TAZI, Cambridge, MA (US); Timothy R. MCCOY, Cambridge, MA (US)

(21) Appl. No.: 18/572,006

(22) PCT Filed: Jun. 23, 2022
(86) PCT No.: PCT/US2022/034747

§ 371 Date: Dec. 19, 2023

Related U.S. Application Data
(60) Provisional application No. 63/231,909, filed on Aug. 11, 2021.
(60) Provisional application No. 63/214,752, filed on Jun. 24, 2021.
(60) Provisional application No. 63/214,246, filed on Jun. 23, 2021.
(60) Provisional application No. 63/214,245, filed on Jun. 23, 2021.

(51) Int. Cl. C07K 14/755; A61K 38/00; A61K 47/02; A61K 47/18; A61K 47/22; A61K 47/26

(52) U.S. Cl. CPC C07K 14/755; A61K 47/02; A61K 47/183; A61K 47/22; A61K 47/26; A61K 38/00; C07K 2319/30

(57) ABSTRACT

The present disclosure provides pharmaceutical compositions of a chimeric protein comprising a factor VIII (FVIII) polypeptide and a von Willebrand factor (VWF) polypeptide. Also disclosed are pharmaceutical kits and methods of using the disclosed pharmaceutical compositions to treat hemophilia A.

### CROSS-REFERENCE TO RELATED APPLICATIONS

This application is a 35 U.S.C. § 371 filing of International Patent Application No. PCT/US2022/034747, filed Jun. 23, 2022, which claims the benefit of U.S. Provisional Application Ser. No. 63/214,245, filed Jun. 23, 2021, 63/214,246, filed Jun. 23, 2021, 63/214,752, filed Jun. 24, 2021, and 63/231,909, filed Aug. 11, 2021, each of which are incorporated herein by reference in their entirety.

### REFERENCE TO SEQUENCE LISTING SUBMITTED ELECTRONICALLY

The content of the electronically submitted sequence listing in ASCII text file (Name: 743583_SA9-480US_ST25.txt; Size: 161,604 bytes; Created: Dec. 18, 2023) is incorporated herein by reference in its entirety.

### BACKGROUND OF THE DISCLOSURE

Hemophilia A is a bleeding disorder caused by defects in the gene encoding coagulation factor VIII (FVIII) and affects 1-2 in 10,000 male births. Graw et al., Nat. Rev. Genet. 6(6): 488-501 (2005). Patients affected with hemophilia A can be treated with infusions of purified plasma FVIII or recombinantly produced FVIII. Many commercially available FVIII products are known to have a half-life of about 8-12 hours, requiring frequent intravenous administration to the patients. See Weiner M. A. and Cairo, M. S., Pediatric Hematology Secrets, Lee, M. T., 12. Disorders of Coagulation, Elsevier Health Sciences, 2001; Lillicrap, D. Thromb. Res. 122 Suppl 4:S2-8 (2008). In addition, a number of approaches have been tried in order to extend the FVIII half-life. For example, the approaches in development to extend the half-life of clotting factors include pegylation, glycopegylation, and conjugation with albumin. See Dumont et al., Blood. 119(13): 3024-3030 (2012). Consistent results have been demonstrated in humans, for example, an rFVIII-Fc fusion protein was reported to improve half-life up to ˜1.7-fold compared with ADVATER in hemophilia A patients. See Powell et al., Blood. 119(13): 3031-3037 (2012). Therefore, the half-life increases, despite minor improvements, indicate the presence of other half-life limiting factors, such as clearance by VWF. Pipe et al., Blood. 128(16):2007-2016 (2016)).

Efanesoctocog alfa (also known as Efa and BIVV001) is a fusion protein that is designed to uncouple recombinant clotting factor VIII from VWF in circulation. The chimeric protein comprises a single recombinant factor VIII protein fused to dimeric Fc, a D′D3 domain of VWF, and two ELNN Polypeptides. Chhabra et al. Blood 135(17): 1484-1496 (2020). In one early study involving patients with severe hemophilia A, a single intravenous injection of efanesoctocog alfa resulted in high sustained factor VIII activity levels, with a half-life that was up to four times the half-life associated with recombinant factor VIII. See Konkle et al., NEJM. 383:1018-27 (2020). However, there remains a need for improved pharmaceutical compositions.

### BRIEF SUMMARY OF THE DISCLOSURE

The present disclosure is directed to, inter alia, pharmaceutical compositions comprising a Factor VIII (“FVIII”) protein, kits comprising such pharmaceutical compositions, and therapeutic methods and uses of the pharmaceutical compositions.

In some embodiments, the pharmaceutical composition comprises a chimeric protein that comprises a first polypeptide chain which comprises a FVIII protein or a portion thereof and a first immunoglobulin (“Ig”) constant region or a portion thereof, and a second polypeptide chain which comprises a von Willebrand Factor (“VWF”) protein and a second Ig constant region or a portion thereof. In some embodiments, the chimeric protein comprises (i) a FVIII protein comprising a FVIII polypeptide, an ELNN Polypeptide inserted within the B domain (e.g., replacing at least a portion of the B domain) of the FVIII polypeptide, and a first Fc region; and (ii) a VWF protein comprising a VWF fragment (e.g., a fragment comprising the D′D3 domains of VWF, which fragment may comprise mutations), a second ELNN Polypeptide, a thrombin-cleavable linker (such as an a2 linker), and a second Fc region. In some embodiments, the chimeric protein disclosed herein is a FVIII-ELNN-Fc/D′D3-ELNN-Fc heterodimer.

In some embodiments, the pharmaceutical composition comprises: (a) a FVIII protein; (b) sucrose; (c) histidine; (d) arginine; (e) calcium chloride; and (f) a polysorbate. In some embodiments, the pharmaceutical composition comprises: (a) a FVIII protein; (b) sucrose; (c) L-histidine; (d) L-arginine; (e) calcium chloride; and (f) a polysorbate. In some embodiments, the pharmaceutical composition comprising: (a) a Factor VIII (“FVIII”) protein; (b) sucrose; (c) L-histidine; (d) L-arginine-HCl; (e) calcium chloride dihydrate; and (f) a polysorbate.

In some embodiments, the pharmaceutical composition comprises: (a) a FVIII protein; (b) about 1% (w/v) to about 4% (w/v) sucrose; (c) about 5 mM to about 15 mM histidine; (d) about 150 mM to about 300 mM arginine; (e) about 2.5 mM to about 10 mM calcium chloride; and (f) about 0.008% (w/v) to about 0.1% (w/v) of a polysorbate. In some embodiments, the pharmaceutical composition comprises: (a) a FVIII protein; (b) about 1% (w/v) to about 4% (w/V) sucrose; (c) about 5 mM to about 15 mM histidine; (d) about 200 mM to about 300 mM arginine; (e) about 2.5 mM to about 10 mM calcium chloride; and (f) about 0.008% (w/v) to about 0.1% (w/V) of a polysorbate. In some embodiments, the pharmaceutical composition comprises: (a) a FVIII protein; (b) about 1% (w/v) to about 4% (w/V) sucrose; (c) about 5 mM to about 15 mM L-histidine; (d) about 200 mM to about 300 mM L-arginine; (e) about 2.5 mM to about 10 mM calcium chloride; and (f) about 0.008% (w/v) to about 0.1% (w/v) of a polysorbate. In some embodiments, the pharmaceutical composition comprises: (a) a Factor VIII (“FVIII”) protein; (b) about 1% (w/v) to about 4% (w/v) sucrose; (c) about 5 mM to about 15 mM L-histidine; (d) about 200 mM to about 300 mM L-arginine-HCl; (e) about 2.5 mM to about 10 mM calcium chloride dihydrate; and (f) about 0.008% (w/v) to about 0.1% (w/v) of a polysorbate.

In some embodiments, the pharmaceutical composition comprises: (a) a FVIII protein; (b) about 5% (w/v) to about 7.5% (w/v) sucrose; (c) about 5 mM to about 15 mM histidine; (d) about 150 mM to about 300 mM arginine; (e) about 2.5 mM to about 10 mM calcium chloride; and (f) about 0.008% (w/v) to about 0.1% (w/v) of a polysorbate. In some embodiments, the pharmaceutical composition comprises: (a) a FVIII protein; (b) about 5% (w/v) to about 7.5% (w/v) sucrose; (c) about 5 mM to about 15 mM histidine; (d) about 200 mM to about 300 mM arginine; (e) about 2.5 mM to about 10 mM calcium chloride; and (f) about 0.008% (w/v) to about 0.1% (w/v) of a polysorbate. In some embodiments, the pharmaceutical composition comprises: (a) a FVIII protein; (b) about 5% (w/v) to about 7.5% (w/v) sucrose; (c) about 5 mM to about 15 mM L-histidine; (d) about 200 mM to about 300 mM L-arginine; (e) about 2.5 mM to about 10 mM calcium chloride; and (f) about 0.008% (w/v) to about 0.1% (w/v) of a polysorbate. In some embodiments, the pharmaceutical composition comprising: (a) a Factor VIII (“FVIII”) protein; (b) about 5% (w/V) to about 7.5% (w/v) sucrose; (c) about 5 mM to about 15 mM L-histidine; (d) about 200 mM to about 300 mM L-arginine-HCl; (e) about 2.5 mM to about 10 mM calcium chloride dihydrate; and (f) about 0.008% (w/v) to about 0.1% (w/v) of a polysorbate.

In some embodiments, the pharmaceutical composition comprises about 1% (w/v) to about 4% (w/v) sucrose. In some embodiments, the pharmaceutical composition comprises about 5% (w/v) to about 7.5% (w/v) sucrose. In some embodiments, the pharmaceutical composition comprises about 5 mM to about 15 mM histidine. In some embodiments, the pharmaceutical composition comprises at least 150 mM arginine. In some embodiments, the pharmaceutical composition comprises about 150 mM to about 300 mM arginine. In some embodiments, the pharmaceutical composition comprises at least 250 mM arginine. In some embodiments, the pharmaceutical composition comprises about 200 mM to about 300 mM arginine. In some embodiments, the pharmaceutical composition comprises about 2.5 mM to about 10 mM calcium chloride. In some embodiments, the pharmaceutical composition comprises polysorbate 20. In some embodiments, the pharmaceutical composition comprises polysorbate 80. In some embodiments, the pharmaceutical composition comprises about 0.008% (w/v) to about 0.1% (w/v) polysorbate 20 or polysorbate 80.

In some embodiments, the composition comprises at least about 250 mM L-arginine. In some embodiments, the composition comprises at least about 250 mM L-arginine-HCl. In some embodiments, the composition comprises about 250 mM L-arginine. In some embodiments, the composition comprises about 250 mM L-arginine-HCl. In some embodiments, the polysorbate is polysorbate 80. In some embodiments, the polysorbate is polysorbate 20.

In some embodiments, disclosed herein is a pharmaceutical composition comprising:
(a) a chimeric protein comprising a first polypeptide chain which comprises a Factor VIII (“FVIII”) protein and a first immunoglobulin (“Ig”) constant region or a portion thereof, and a second polypeptide chain which comprises a von Willebrand Factor (“VWF”) protein and a second Ig constant region or a portion thereof;
(b) sucrose;
(c) histidine;
(d) arginine;
(e) calcium chloride; and
(f) a polysorbate.

In some embodiments, disclosed herein is a pharmaceutical composition comprising:
(a) a chimeric protein comprising a first polypeptide chain which comprises a Factor VIII (“FVIII”) protein and a first immunoglobulin (“Ig”) constant region or a portion thereof, and a second polypeptide chain which comprises a von Willebrand Factor (“VWF”) protein and a second Ig constant region or a portion thereof;
(b) about 1% (w/v) to about 4% (w/v) sucrose;
(c) about 5 mM to about 15 mM histidine;
(d) about 150 mM to about 300 mM arginine;
(e) about 2.5 mM to about 10 mM calcium chloride; and
(f) about 0.008% (w/v) to about 0.1% (w/v) of a polysorbate.

In some embodiments, disclosed herein is a pharmaceutical composition comprising:
(a) about 5% (w/v) to about 7.5% (w/v) sucrose;
(b) about 5 mM to about 15 mM histidine;
(c) about 150 mM to about 300 mM arginine;
(d) about 2.5 mM to about 10 mM calcium chloride; and
(e) about 0.008% (w/v) to about 0.1% (w/v) polysorbate 20 or polysorbate 80.

In some embodiments, disclosed herein is a pharmaceutical composition comprising:
(a) a chimeric protein comprising a first polypeptide chain which comprises a Factor VIII (“FVIII”) protein and a first immunoglobulin (“Ig”) constant region or a portion thereof, and a second polypeptide chain which comprises a von Willebrand Factor (“VWF”) protein and a second Ig constant region or a portion thereof;
(b) about 1% (w/v) to about 4% (w/V) sucrose;
(c) about 5 mM to about 15 mM histidine;
(d) about 200 mM to about 300 mM arginine;
(e) about 2.5 mM to about 10 mM calcium chloride; and
(f) about 0.008% (w/v) to about 0.1% (w/v) of a polysorbate.

In some embodiments, disclosed herein is a pharmaceutical composition comprising:
(a) about 5% (w/v) to about 7.5% (w/v) sucrose;
(b) about 5 mM to about 15 mM histidine;
(c) about 200 mM to about 300 mM arginine;
(d) about 2.5 mM to about 10 mM calcium chloride; and
(e) about 0.008% (w/v) to about 0.1% (w/V) polysorbate 20 or polysorbate 80.

In some embodiments, the pharmaceutical composition comprises about 250 IU, 500 IU, 1000 IU, 2000 IU, 3000 IU, or 4,000 IU of the chimeric protein. In some embodiments, the first polypeptide chain comprises the amino acid sequence set forth as SEQ ID NO: 1 and the second polypeptide chain comprises the amino acid sequence set forth as SEQ ID NO: 2, wherein the first polypeptide chain and the second polypeptide chain are covalently linked by two disulfide bonds between Fc domains in the first and second polypeptide chains. In some embodiments, the chimeric protein is efanesoctocog alfa.

In some embodiments, the pharmaceutical composition comprises about 1% (w/v) to about 4% (w/v) sucrose. In some embodiments, the pharmaceutical composition comprises about 5 mM to about 15 mM histidine. In some embodiments, the pharmaceutical composition comprises at least 150 mM arginine. In some embodiments, the pharmaceutical composition comprises about 150 mM to about 300 mM arginine. In some embodiments, the pharmaceutical composition comprises at least 250 mM arginine. In some embodiments, the pharmaceutical composition comprises about 200 mM to about 300 mM arginine. In some embodiments, the pharmaceutical composition comprises about 2.5 mM to about 10 mM calcium chloride. In some embodiments, the pharmaceutical composition comprises polysorbate 20. In some embodiments, the pharmaceutical composition comprises polysorbate 80. In some embodiments, the pharmaceutical composition comprises about 0.008% (w/v) to about 0.1% (w/v) polysorbate 20 or polysorbate 80.

In some embodiments, disclosed herein is a pharmaceutical composition comprising:
(a) about 1% (w/v) to about 4% (w/v) sucrose;
(b) about 5 mM to about 15 mM histidine;
(c) about 150 mM to about 300 mM arginine;
(d) about 2.5 mM to about 10 mM calcium chloride; and
(e) about 0.008% (w/v) to about 0.1% (w/v) polysorbate 20 or polysorbate 80.

In some embodiments, disclosed herein is a pharmaceutical composition comprising:
(a) about 1% (w/v) to about 4% (w/v) sucrose;
(b) about 5 mM to about 15 mM histidine;
(c) about 200 mM to about 300 mM arginine;
(d) about 2.5 mM to about 10 mM calcium chloride; and
(e) about 0.008% (w/v) to about 0.1% (w/v) polysorbate 20 or polysorbate 80.

In some embodiments, disclosed herein is a pharmaceutical composition comprising:
(a) about 2% (w/v) sucrose;
(b) about 10 mM histidine;
(c) about 250 mM arginine;
(d) about 5 mM calcium chloride; and
(e) about 0.05% polysorbate 20 or polysorbate 80.

In some embodiments, disclosed herein is a pharmaceutical composition comprising:
(a) about 2% (w/v) sucrose;
(b) about 10 mM L-histidine;
(c) about 250 mM L-arginine-HCl;
(d) about 5 mM calcium chloride; and
(e) about 0.05% polysorbate 80.

In some embodiments, disclosed herein is a pharmaceutical composition comprising:
(a) 10 mg/mL to 40 mg/mL sucrose;
(b) 1.5 mg/mL to 2.0 mg/mL L-histidine;
(c) 40 mg/mL to 70 mg/mL L-arginine-HCl;
(d) 0.4 mg/mL to 0.9 mg/mL calcium chloride; and
(e) 0.4 mg/mL to 0.7 mg/mL polysorbate 80.

In some embodiments, disclosed herein is a pharmaceutical composition comprising:
(a) 22.45 mg/ml sucrose;
(b) 1.74 mg/ml L-histidine;
(c) 59.11 mg/ml L-arginine-HCl;
(d) 0.82 mg/ml calcium chloride dihydrate; and
(e) 0.56 mg/ml polysorbate 80.

In some embodiments, disclosed herein is a pharmaceutical composition comprising:
(a) 22.45 mg/ml sucrose;
(b) 1.74 mg/ml L-histidine;
(c) 59.11 mg/ml L-arginine-HCl;
(d) 0.62 mg/ml calcium chloride; and
(e) 0.56 mg/ml polysorbate 80.

In some embodiments, disclosed herein is a pharmaceutical composition comprising:
(a) 10 mg/mL to 40 mg/mL sucrose;
(b) 1.5 mg/mL to 2.0 mg/mL L-histidine;
(c) 40 mg/mL to 70 mg/mL L-arginine-HCl;
(d) 0.5 mg/mL to 0.9 mg/mL calcium chloride dihydrate; and
(e) 0.4 mg/mL to 0.7 mg/mL polysorbate 80.

In some embodiments, disclosed herein is a pharmaceutical composition comprising:
(a) about 20 mg/mL sucrose;
(b) about 1.6 mg/mL L-histidine;
(c) about 52.7 mg/mL L-arginine-HCl;
(d) about 0.7 mg/mL calcium chloride dihydrate; and
(e) about 0.5 mg/mL polysorbate 80.

In some embodiments, disclosed herein is a pharmaceutical composition comprising:
(a) about 20 mg/mL sucrose;
(b) about 1.6 mg/mL L-histidine;
(c) about 52.7 mg/mL L-arginine-HCl;
(d) about 0.6 mg/mL calcium chloride; and
(e) about 0.5 mg/mL polysorbate 80.

In some embodiments, disclosed herein is a pharmaceutical composition comprising:
(a) about 20 mg/mL sucrose;
(b) about 1.6 mg/mL L-histidine;
(c) about 43.6 mg/mL L-arginine;
(d) about 0.7 mg/mL calcium chloride dihydrate; and
(e) about 0.5 mg/mL polysorbate 80.

In some embodiments, disclosed herein is a pharmaceutical composition comprising:
(a) about 20 mg/mL sucrose;
(b) about 1.6 mg/mL L-histidine;
(c) about 43.6 mg/mL L-arginine;
(d) about 0.6 mg/mL calcium chloride; and
(e) about 0.5 mg/mL polysorbate 80.

In some embodiments, disclosed herein is a pharmaceutical composition comprising:
(a) 10 mg/mL to 40 mg/mL sucrose;
(b) 1.5 mg/mL to 2.0 mg/mL L-histidine;
(c) 50 mg/mL to 70 mg/mL L-arginine-HCl;
(d) 0.7 mg/mL to 0.9 mg/mL calcium chloride dihydrate; and
(e) 0.4 mg/mL to 0.7 mg/mL polysorbate 80.

In some embodiments, disclosed herein is a pharmaceutical composition comprising:
(a) 10 mg/mL to 40 mg/mL sucrose;
(b) 1.5 mg/mL to 2.0 mg/mL L-histidine;
(c) 50 mg/mL to 70 mg/mL L-arginine-HCl;
(d) 0.4 mg/mL to 0.8 mg/mL calcium chloride; and
(e) 0.4 mg/mL to 0.7 mg/mL polysorbate 80.

In some embodiments, disclosed herein is a pharmaceutical composition comprising:
(a) 10 mg/mL to 40 mg/mL sucrose;
(b) 1.5 mg/mL to 2.0 mg/mL L-histidine;
(c) 40 mg/mL to 60 mg/mL L-arginine;
(d) 0.7 mg/mL to 0.9 mg/mL calcium chloride; and
(e) 0.4 mg/mL to 0.7 mg/mL polysorbate 80.

In some embodiments, disclosed herein is a pharmaceutical composition comprising:
(a) 10 mg/mL to 40 mg/mL sucrose;
(b) 1.5 mg/mL to 2.0 mg/mL L-histidine;
(c) 40 mg/mL to 60 mg/mL L-arginine;
(d) 0.4 mg/mL to 0.7 mg/mL calcium chloride dihydrate; and
(e) 0.4 mg/mL to 0.7 mg/mL polysorbate 80.

In some embodiments, disclosed herein is a pharmaceutical composition comprising:
(a) 22.45 mg/ml sucrose;
(b) 1.74 mg/ml L-histidine;
(c) 59.11 mg/ml L-arginine-HCl;
(d) 0.82 mg/ml calcium chloride dihydrate; and
(e) 0.56 mg/ml polysorbate 80.

In some embodiments, disclosed herein is a pharmaceutical composition comprising:
(a) 22.45 mg/ml sucrose;
(b) 1.74 mg/ml L-histidine;
(c) 48.88 mg/ml L-arginine;
(d) 0.82 mg/ml calcium chloride dihydrate; and
(e) 0.56 mg/ml polysorbate 80.

In some embodiments, disclosed herein is a pharmaceutical composition comprising:
(a) 22.45 mg/ml sucrose;
(b) 1.74 mg/ml L-histidine;
(c) 59.11 mg/ml L-arginine-HCl;
(d) 0.62 mg/ml calcium chloride; and
(e) 0.56 mg/ml polysorbate 80.

In some embodiments, disclosed herein is a pharmaceutical composition comprising:
(a) 22.45 mg/ml sucrose;
(b) 1.74 mg/ml L-histidine;
(c) 48.88 mg/ml L-arginine;
(d) 0.62 mg/ml calcium chloride; and
(e) 0.56 mg/ml polysorbate 80.

In some embodiments, the pharmaceutical composition comprises about 5% (w/v) to about 7.5% (w/v) sucrose. In some embodiments, the pharmaceutical composition comprises about 5 mM to about 15 mM histidine. In some embodiments, the pharmaceutical composition comprises at least 150 mM arginine. In some embodiments, the pharmaceutical composition comprises about 150 mM to about 300 mM arginine. In some embodiments, the pharmaceutical composition comprises at least 250 mM arginine. In some embodiments, the pharmaceutical composition comprises about 200 mM to about 300 mM arginine. In some embodiments, the pharmaceutical composition comprises about 2.5 mM to about 10 mM calcium chloride. In some embodiments, the pharmaceutical composition comprises polysorbate 20. In some embodiments, the pharmaceutical composition comprises polysorbate 80. In some embodiments, the pharmaceutical composition comprises about 0.008% (w/v) to about 0.1% (w/v) polysorbate 20 or polysorbate 80.

In some embodiments, disclosed herein is a pharmaceutical composition comprising:
(a) about 5% (w/v) to about 7.5% (w/v) sucrose;
(b) about 5 mM to about 15 mM histidine;
(c) about 150 mM to about 300 mM arginine;
(d) about 2.5 mM to about 10 mM calcium chloride; and
(e) about 0.008% (w/v) to about 0.1% (w/v) polysorbate 20 or polysorbate 80.

In some embodiments, disclosed herein is a pharmaceutical composition comprising:
(a) about 5% (w/v) to about 7.5% (w/V) sucrose;
(b) about 5 mM to about 15 mM histidine;
(c) about 200 mM to about 300 mM arginine;
(d) about 2.5 mM to about 10 mM calcium chloride; and
(e) about 0.008% (w/v) to about 0.1% (w/v) polysorbate 20 or polysorbate 80.

In some embodiments, disclosed herein is a pharmaceutical composition comprising:
(a) about 5% (w/v) sucrose;
(b) about 10 mM histidine;
(c) about 250 mM arginine;
(d) about 5 mM calcium chloride; and
(e) about 0.05% polysorbate 20 or polysorbate 80.

In some embodiments, disclosed herein is a pharmaceutical composition comprising:
(a) about 5% (w/v) sucrose;
(b) about 10 mM L-histidine;
(c) about 250 mM L-arginine-HCl;
(d) about 5 mM calcium chloride; and
(e) about 0.05% polysorbate 80.

In some embodiments, disclosed herein is a pharmaceutical composition comprising:
(a) 45 mg/mL to 60 mg/mL sucrose;
(b) 1.5 mg/mL to 2.0 mg/mL L-histidine;
(c) 40 mg/mL to 70 mg/mL L-arginine-HCl;
(d) 0.4 mg/mL to 0.9 mg/mL calcium chloride; and
(e) 0.4 mg/mL to 0.7 mg/mL polysorbate 80.

In some embodiments, disclosed herein is a pharmaceutical composition comprising:
(a) 56.12 mg/ml sucrose;
(b) 1.74 mg/ml L-histidine;
(c) 59.11 mg/ml L-arginine-HCl;
(d) 0.82 mg/ml calcium chloride dihydrate; and
(e) 0.56 mg/ml polysorbate 80.

In some embodiments, disclosed herein is a pharmaceutical composition comprising:
(a) 56.12 mg/ml sucrose;
(b) 1.74 mg/ml L-histidine;
(c) 59.11 mg/ml L-arginine-HCl;
(d) 0.62 mg/ml calcium chloride; and
(e) 0.56 mg/ml polysorbate 80.

In some embodiments, disclosed herein is a pharmaceutical composition comprising:
(a) 45 mg/mL to 60 mg/mL sucrose;
(b) 1.5 mg/mL to 2.0 mg/mL L-histidine;
(c) 40 mg/mL to 70 mg/mL L-arginine-HCl;
(d) 0.5 mg/mL to 0.9 mg/mL calcium chloride dihydrate; and
(e) 0.4 mg/mL to 0.7 mg/mL polysorbate 80.

In some embodiments, disclosed herein is a pharmaceutical composition comprising:
(a) about 50 mg/mL sucrose;
(b) about 1.6 mg/mL L-histidine;
(c) about 52.7 mg/mL L-arginine-HCl;
(d) about 0.7 mg/mL calcium chloride dihydrate; and
(e) about 0.5 mg/mL polysorbate 80.

In some embodiments, disclosed herein is a pharmaceutical composition comprising:
(a) about 50 mg/ml sucrose;
(b) about 1.6 mg/mL L-histidine;
(c) about 52.7 mg/mL L-arginine-HCl;
(d) about 0.6 mg/mL calcium chloride; and
(e) about 0.5 mg/mL polysorbate 80.

In some embodiments, disclosed herein is a pharmaceutical composition comprising:
(a) about 50 mg/ml sucrose;
(b) about 1.6 mg/mL L-histidine;
(c) about 43.6 mg/mL L-arginine;
(d) about 0.7 mg/mL calcium chloride dihydrate; and
(e) about 0.5 mg/mL polysorbate 80.

In some embodiments, disclosed herein is a pharmaceutical composition comprising:
(a) about 50 mg/mL sucrose;
(b) about 1.6 mg/mL L-histidine;
(c) about 43.6 mg/mL L-arginine;
(d) about 0.6 mg/mL calcium chloride; and
(e) about 0.5 mg/mL polysorbate 80.

In some embodiments, disclosed herein is a pharmaceutical composition comprising:
(a) 45 mg/mL to 60 mg/mL sucrose;
(b) 1.5 mg/mL to 2.0 mg/mL L-histidine;
(c) 50 mg/mL to 70 mg/mL L-arginine-HCl;
(d) 0.7 mg/mL to 0.9 mg/mL calcium chloride dihydrate; and
(e) 0.4 mg/mL to 0.7 mg/mL polysorbate 80.

In some embodiments, disclosed herein is a pharmaceutical composition comprising:
(a) 45 mg/mL to 60 mg/mL sucrose;
(b) 1.5 mg/mL to 2.0 mg/mL L-histidine;
(c) 50 mg/mL to 70 mg/mL L-arginine-HCl;
(d) 0.4 mg/mL to 0.8 mg/mL calcium chloride; and
(e) 0.4 mg/mL to 0.7 mg/mL polysorbate 80.

In some embodiments, disclosed herein is a pharmaceutical composition comprising:
(a) 45 mg/mL to 60 mg/mL sucrose;
(b) 1.5 mg/mL to 2.0 mg/mL L-histidine;
(c) 40 mg/mL to 60 mg/mL L-arginine;
(d) 0.7 mg/mL to 0.9 mg/mL calcium chloride; and
(e) 0.4 mg/mL to 0.7 mg/mL polysorbate 80.

In some embodiments, disclosed herein is a pharmaceutical composition comprising:
(a) 45 mg/mL to 60 mg/mL sucrose;
(b) 1.5 mg/mL to 2.0 mg/mL L-histidine;
(c) 40 mg/mL to 60 mg/mL L-arginine;
(d) 0.4 mg/mL to 0.7 mg/mL calcium chloride dihydrate; and
(e) 0.4 mg/mL to 0.7 mg/mL polysorbate 80.

In some embodiments, disclosed herein is a pharmaceutical composition comprising:
(a) 56.12 mg/ml sucrose;
(b) 1.74 mg/ml L-histidine;
(c) 59.11 mg/ml L-arginine-HCl;
(d) 0.82 mg/ml calcium chloride dihydrate; and
(e) 0.56 mg/ml polysorbate 80.

In some embodiments, disclosed herein is a pharmaceutical composition comprising:
(a) 56.12 mg/ml sucrose;
(b) 1.74 mg/ml L-histidine;
(c) 48.88 mg/ml L-arginine;
(d) 0.82 mg/ml calcium chloride dihydrate; and
(e) 0.56 mg/ml polysorbate 80.

In some embodiments, disclosed herein is a pharmaceutical composition comprising:
(a) 56.12 mg/ml sucrose;
(b) 1.74 mg/ml L-histidine;
(c) 59.11 mg/ml L-arginine-HCl;
(d) 0.62 mg/ml calcium chloride; and
(e) 0.56 mg/ml polysorbate 80.

In some embodiments, disclosed herein is a pharmaceutical composition comprising:
(a) 56.12 mg/ml sucrose;
(b) 1.74 mg/ml L-histidine;
(c) 48.88 mg/ml L-arginine;
(d) 0.62 mg/ml calcium chloride; and
(e) 0.56 mg/ml polysorbate 80.

In some embodiments, disclosed herein is a pharmaceutical composition according to any of the disclosed embodiments, which further comprises a pH of about 6.5 to about 7.5. In some embodiments, the pharmaceutical composition disclosed herein has a pH of about 6.8 to about 7.3. In some embodiments, the pharmaceutical composition disclosed herein has a pH of about 7.0. In some embodiments, the pharmaceutical composition disclosed herein has a pH of about 6.8. In some embodiments the pharmaceutical composition disclosed herein does not comprise NaCl. In some embodiments, the pharmaceutical composition does not comprise NaOH. In some embodiments, the pharmaceutical composition does not comprise sodium ions. In some embodiments the pharmaceutical composition disclosed herein comprises less than 8.8 mg/ml sodium chloride (NaCl). In some embodiments, the pharmaceutical composition disclosed herein comprises L-histidine. In some embodiments, the pharmaceutical composition disclosed herein comprises L-arginine. In some embodiments, the pharmaceutical composition disclosed herein comprises arginine-HCl. In some embodiments, the pharmaceutical composition disclosed herein comprises L-arginine-HCl. In some embodiments, the pharmaceutical composition disclosed herein comprises calcium chloride dihydrate.

In some embodiments, disclosed herein is a pharmaceutical composition according to any of the disclosed embodiments, wherein the pharmaceutical composition has a chimeric protein concentration of about 0.8 to about 1.2 mg/mL. In some embodiments, the pharmaceutical composition disclosed herein comprises 75 IU/mL to 2,000 IU/mL of the chimeric protein. In some embodiments, the pharmaceutical composition disclosed herein has an osmolality about 525 to about 725 mOsm/kg. In some embodiments, the pharmaceutical composition disclosed herein has an osmolality about 600 to about 650 mOsm/kg. In some embodiments, the pharmaceutical composition disclosed herein has a turbidity of less than about 7 Nephelometric Turbidity Units (NTU).

Also disclosed herein is a method of treating hemophilia A in a subject in need thereof, comprising administering to the subject an effective amount of the pharmaceutical composition according to any of the embodiments disclosed herein. In some embodiments, the pharmaceutical composition is self-administered. In some embodiments, the pharmaceutical composition is administered intravenously. In some embodiments, the pharmaceutical composition is administered intravenously at a dose of about 20 IU/kg to about 70 IU/kg. In some embodiments, the pharmaceutical composition is administered intravenously at a dose of about 50 IU/kg. In some embodiments, the pharmaceutical composition is administered intravenously once every 7-10 days. In some embodiments, the pharmaceutical composition is administered intravenously once weekly.

Also disclosed herein is a pharmaceutical kit comprising (i) a first container comprising a lyophilized pharmaceutical composition comprising
(a) a chimeric protein comprising a first polypeptide chain which comprises a Factor VIII (“FVIII”) protein or a portion thereof and a first immunoglobulin (“Ig”) constant region or a portion thereof, and a second polypeptide chain which comprises a von Willebrand Factor (“VWF”) protein and a second Ig constant region or a portion thereof;
(b) sucrose;
(c) histidine;
(d) arginine;
(e) calcium chloride; and
(f) polysorbate 20 or polysorbate 80, and
(ii) a second container comprising sterile water.

In some embodiments, the chimeric protein comprises a first polypeptide chain comprising the amino acid sequence set forth as SEQ ID NO: 1 and a second polypeptide chain comprising the amino acid sequence set forth as SEQ ID NO: 2, wherein the first polypeptide chain and the second polypeptide chain are covalently linked by two disulfide bonds between Fc domains in the first and second polypeptide chains.

Also disclosed herein is a pharmaceutical kit comprising (i) a first container comprising a lyophilized pharmaceutical composition comprising
(a) a chimeric protein comprising a first polypeptide chain which comprises a Factor VIII (“FVIII”) protein or a portion thereof and a first immunoglobulin (“Ig”) constant region or a portion thereof, and a second polypeptide chain which comprises a von Willebrand Factor (“VWF”) protein and a second Ig constant region or a portion thereof;
(b) about 30 mg to about 135 mg sucrose;
(c) about 2.5 mg to about 7.5 mg histidine;
(d) about 140 mg to about 200 mg arginine;
(e) about 1.5 mg to about 5 mg calcium chloride; and
(f) about 1 mg to about 5 mg polysorbate 20 or polysorbate 80, and
(ii) a second container comprising sterile water.

In some embodiments, the chimeric protein comprises a first polypeptide chain comprising the amino acid sequence set forth as SEQ ID NO: 1 and a second polypeptide chain comprising the amino acid sequence set forth as SEQ ID NO: 2, wherein the first polypeptide chain and the second polypeptide chain are covalently linked by two disulfide bonds between Fc domains in the first and second polypeptide chains.

In some embodiments, the pharmaceutical kit disclosed herein comprises a lyophilized pharmaceutical composition comprising:
(a) about 30 mg to about 135 mg sucrose;
(b) about 2.5 mg to about 7.5 mg histidine;
(c) about 140 mg to about 200 mg arginine;
(d) about 1.5 mg to about 5 mg calcium chloride; and
(e) about 1 mg to about 5 mg polysorbate 20 or polysorbate 80.

In some embodiments the pharmaceutical kit comprises a lyophilized pharmaceutical composition that does not comprise NaCl. In some embodiments the lyophilized pharmaceutical composition comprises less than 8.8 mg/mL sodium chloride (NaCl). In some embodiments, the pharmaceutical composition does not comprise NaOH. In some embodiments, the pharmaceutical composition does not comprise sodium ions. In some embodiments, the lyophilized pharmaceutical composition comprises L-histidine. In some embodiments, the lyophilized pharmaceutical composition comprises L-arginine. In some embodiments, the lyophilized pharmaceutical composition comprises arginine-HCl. In some embodiments, the lyophilized pharmaceutical composition comprises L-arginine-HCl. In some embodiments, the lyophilized pharmaceutical composition disclosed herein comprises calcium chloride dihydrate.

In some embodiments, the pharmaceutical kit comprises a lyophilized pharmaceutical composition comprising:
(a) about 67.3 mg sucrose;
(b) about 5.2 mg L-histidine;
(c) about 177.3 mg L-arginine-HCl;
(d) about 2.5 mg calcium chloride; and
(e) about 1.7 mg polysorbate 20 or polysorbate 80.

In some embodiments, the pharmaceutical kit comprises a lyophilized pharmaceutical composition comprising:
(a) about 67.3 mg sucrose;
(b) about 5.2 mg L-histidine;
(c) about 146.6 mg L-arginine-HCl;
(d) about 2.5 mg calcium chloride; and
(e) about 1.7 mg polysorbate 20 or polysorbate 80.

In some embodiments, the pharmaceutical kit disclosed herein comprises a lyophilized pharmaceutical composition comprising:
(a) about 160 mg to about 200 mg sucrose;
(b) about 2.5 mg to about 7.5 mg histidine;
(c) about 140 mg to about 200 mg arginine;
(d) about 1.5 mg to about 5 mg calcium chloride; and
(e) about 1 mg to about 5 mg polysorbate 20 or polysorbate 80.

In some embodiments the pharmaceutical kit comprises a lyophilized pharmaceutical composition that does not comprise NaCl. In some embodiments the lyophilized pharmaceutical composition comprises less than 8.8 mg/mL sodium chloride (NaCl). In some embodiments, the pharmaceutical composition does not comprise NaOH. In some embodiments, the pharmaceutical composition does not comprise sodium ions. In some embodiments, the lyophilized pharmaceutical composition comprises L-histidine. In some embodiments, the lyophilized pharmaceutical composition comprises L-arginine. In some embodiments, the lyophilized pharmaceutical composition comprises arginine-HCl. In some embodiments, the lyophilized pharmaceutical composition comprises L-arginine-HCl. In some embodiments, the lyophilized pharmaceutical composition disclosed herein comprises calcium chloride dihydrate.

In some embodiments, the pharmaceutical kit comprises a lyophilized pharmaceutical composition comprising:
(a) about 168.3 mg sucrose;
(b) about 5.2 mg L-histidine;
(c) about 177.3 mg L-arginine-HCl;
(d) about 2.5 mg calcium chloride; and
(e) about 1.7 mg polysorbate 20 or polysorbate 80.

In some embodiments, the pharmaceutical kit comprises a lyophilized pharmaceutical composition comprising:
(a) about 168.3 mg sucrose;
(b) about 5.2 mg L-histidine;
(c) about 146.6 mg L-arginine-HCl;
(d) about 2.5 mg calcium chloride; and
(e) about 1.7 mg polysorbate 20 or polysorbate 80.

In some embodiments, the pharmaceutical kit comprises a lyophilized pharmaceutical composition having a moisture content of less than 2%. In some embodiments, the lyophilized pharmaceutical composition has a moisture content of less than 1.8%. In some embodiments, the lyophilized pharmaceutical composition has a moisture content of less than 1.6%. In some embodiments, the lyophilized pharmaceutical composition is in a lyophilized cake. In some embodiments, the lyophilized cake is white. In some embodiments, the lyophilized cake is less than Y4 in the European Pharmacopoeia color scale. In some embodiments, the lyophilized pharmaceutical composition is a powder.

In some embodiments, the pharmaceutical kit comprises a first container comprising 100 IU to 10,000 IU of the chimeric protein. In some embodiments, the first container comprises 250 IU, 500 IU, 1000 IU, 2000 IU, 3000 IU, or 4,000 IU of the chimeric protein.

In some embodiments, the pharmaceutical kit further comprises instructions for combining the lyophilized pharmaceutical composition and sterile water. In some embodiments, when the lyophilized pharmaceutical composition and the sterile water are combined, then the lyophilized pharmaceutical composition is reconstituted within 7 to 12 seconds.

In some embodiments, the pharmaceutical composition comprises a second container comprising sterilized water at a volume sufficient to produce, when combined with the lyophilized powder of the first container, a solution for injection disclosed herein is obtained.

In some embodiments, when the lyophilized pharmaceutical composition and the sterile water are combined, then the osmolality of the resulting solution is about 525 to about 725 mOsm/kg. In some embodiments, the osmolality of the resulting solution is about 600 to about 650 mOsm/kg. In some embodiments, the pH of the resulting solution is about 6.5 to about 7.5. In some embodiments, the pH of the resulting solution is about 7.0. In some embodiments, the pH of the resulting solution is about 6.8. In some embodiments, the turbidity of the resulting solution is less than about 7 Nephelometric Turbidity Units (NTU). In some embodiments, the protein concentration of the resulting solution is about 0.8 to about 1.2 mg/mL. In some embodiments, less than 3% of the protein is aggregated.

In some embodiments, the pharmaceutical kit comprises a second container comprising sterilized water at a volume sufficient to produce, when combined with the lyophilized pharmaceutical composition of the first container, a solution comprising:
(a) 10 mg/mL to 40 mg/mL sucrose;
(b) 1.5 mg/mL to 2.0 mg/mL L-histidine;
(c) 40 mg/mL to 70 mg/mL L-arginine-HCl;
(d) 0.5 mg/mL to 0.9 mg/mL calcium chloride; and
(e) 0.4 mg/mL to 0.7 mg/mL polysorbate 80.

In some embodiments of the pharmaceutical kit disclosed herein, when the lyophilized pharmaceutical composition and the sterile water are combined, then the resulting solution comprises:
(a) 10 mg/mL to 40 mg/mL sucrose;
(b) 1.5 mg/mL to 2.0 mg/mL L-histidine;
(c) 40 mg/mL to 70 mg/mL L-arginine-HCl;
(d) 0.5 mg/mL to 0.9 mg/mL calcium chloride; and
(e) 0.4 mg/mL to 0.7 mg/mL polysorbate 80.

In some embodiments of the pharmaceutical kit disclosed herein, when the lyophilized pharmaceutical composition and the sterile water are combined, then the resulting solution comprises:
(a) 22.45 mg/ml sucrose;
(b) 1.74 mg/ml L-histidine;
(c) 59.11 mg/ml L-arginine-HCl;
(d) 0.62 mg/ml calcium chloride; and
(e) 0.56 mg/ml polysorbate 80.

In some embodiments of the pharmaceutical kit disclosed herein, when the lyophilized pharmaceutical composition and the sterile water are combined, then the resulting solution comprises:
(a) 22.45 mg/ml sucrose;
(b) 1.74 mg/ml L-histidine;
(c) 48.88 mg/ml L-arginine;
(d) 0.62 mg/ml calcium chloride; and
(e) 0.56 mg/ml polysorbate 80.

In some embodiments, the pharmaceutical kit comprises a second container comprising sterilized water at a volume sufficient to produce, when combined with the lyophilized pharmaceutical composition of the first container, a solution comprising:
(a) 45 mg/mL to 60 mg/mL sucrose;
(b) 1.5 mg/mL to 2.0 mg/mL L-histidine;
(c) 40 mg/mL to 70 mg/mL L-arginine-HCl;
(d) 0.5 mg/mL to 0.9 mg/mL calcium chloride; and
(e) 0.4 mg/mL to 0.7 mg/mL polysorbate 80.

In some embodiments of the pharmaceutical kit disclosed herein, when the lyophilized pharmaceutical composition and the sterile water are combined, then the resulting solution comprises:
(a) 45 mg/mL to 60 mg/mL sucrose;
(b) 1.5 mg/ml to 2.0 mg/mL L-histidine;
(c) 40 mg/mL to 70 mg/mL L-arginine-HCl;
(d) 0.5 mg/mL to 0.9 mg/mL calcium chloride; and
(e) 0.4 mg/mL to 0.7 mg/mL polysorbate 80.

In some embodiments of the pharmaceutical kit disclosed herein, when the lyophilized pharmaceutical composition and the sterile water are combined, then the resulting solution comprises:
(a) 56.12 mg/ml sucrose;
(b) 1.74 mg/ml L-histidine;
(c) 59.11 mg/ml L-arginine-HCl;
(d) 0.62 mg/ml calcium chloride; and
(e) 0.56 mg/ml polysorbate 80.

In some embodiments of the pharmaceutical kit disclosed herein, when the lyophilized pharmaceutical composition and the sterile water are combined, then the resulting solution comprises:
(a) 56.12 mg/ml sucrose;
(b) 1.74 mg/ml L-histidine;
(c) 48.88 mg/ml L-arginine;
(d) 0.62 mg/ml calcium chloride; and
(e) 0.56 mg/ml polysorbate 80.

In some embodiments, the pharmaceutical kit comprises a second container comprising about 2 mL to about 5 mL of sterile water. In some embodiments, the pharmaceutical kit comprises a second container comprising about 3 mL of sterile water. In some embodiments, the pharmaceutical kit comprises a second container comprising about 3.3 mL of sterile water.

In some embodiments, the pharmaceutical kit comprises a first container which is a glass vial comprising a rubber stopper. In some embodiments, the pharmaceutical kit comprises a second container which is a syringe body. In some embodiments, the sterile water is in the syringe body. In some embodiments, the syringe body is associated with a plunger. In some embodiments, the pharmaceutical kit further comprises an adaptor to connect the glass vial to the syringe body. In some embodiments, the pharmaceutical kit further comprises infusion tubing associated with a needle to be connected to the syringe body, suitable for intravenous infusion.

Also disclosed herein is a method of treating hemophilia A in a subject in need thereof, comprising combining the lyophilized pharmaceutical composition and the sterile water of the pharmaceutical kit according to any of the embodiments disclosed herein, and administering to the subject an effective amount of the resulting combination (i.e. solution). In some embodiments, the subject combines the lyophilized pharmaceutical composition and the sterile water of the kit. In some embodiments, the combination is self-administered by the subject.

### BRIEF DESCRIPTION OF THE DRAWINGS/FIGURES

FIG. 1 is a schematic representation of efanesoctocog alfa, an exemplary FVIII-ELNN-Fc/D′D3-ELNN-Fc heterodimer. FVIII: factor VIII; VWF: von Willebrand Factor; A1, A2, A3, C1, C2: domains of FVIII; D′D3: domains of VWF; Fc: Fc region of immunoglobulin constant region.

FIG. 2 shows the pH of pharmaceutical compositions containing various concentrations of L-histidine (10 mM, 20 mM, 50 mM) at efanesoctocog alfa concentrations of 1 mg/ml, 0.67 mg/mL (4000 IU/mL), and 0.045 mg/mL (250 IU/mL). Controls are provided with compositions containing no DS, no excipients or compositions containing only excipients. Abbreviations: DP: drug product. BDP: bulk drug product. DS: drug substance. IU: International Units.

FIG. 3 shows the measured turbidity (NTU) for pharmaceutical compositions containing various concentrations of L-histidine (10 mM, 20 mM, 50 mM) at efanesoctocog alfa concentrations of 1 mg/mL, 0.67 mg/ml (4000 IU/mL), and 0.045 mg/mL (250 IU/mL). Results of compositions at 5, 25, and 40° C. are depicted. Time points were TO and 1 week.

FIG. 4 shows the aggregation levels (% HMWS) for pharmaceutical compositions containing various concentrations of L-histidine (10 mM, 20 mM, 50 mM) at efanesoctocog alfa concentrations of 1 mg/mL, 0.67 mg/mL (4000 IU/mL), and 0.045 mg/ml (250 IU/mL). Results of compositions at 5 and 25° C. are depicted. Time points were TO and 1 week.

FIG. 5 shows the efanesoctocog alfa protein concentration (μg/mL) measured for each of the 13 buffer compositions tested. Compositions were tested at both 2-8° C. and 30° C. Time points measured are start of experiment (T0), one month (T1), and 3 months (T3), and 6 months (T6). Abbreviations: DP: drug product. DDS: diluted drug substance. DoE: design of experiment.

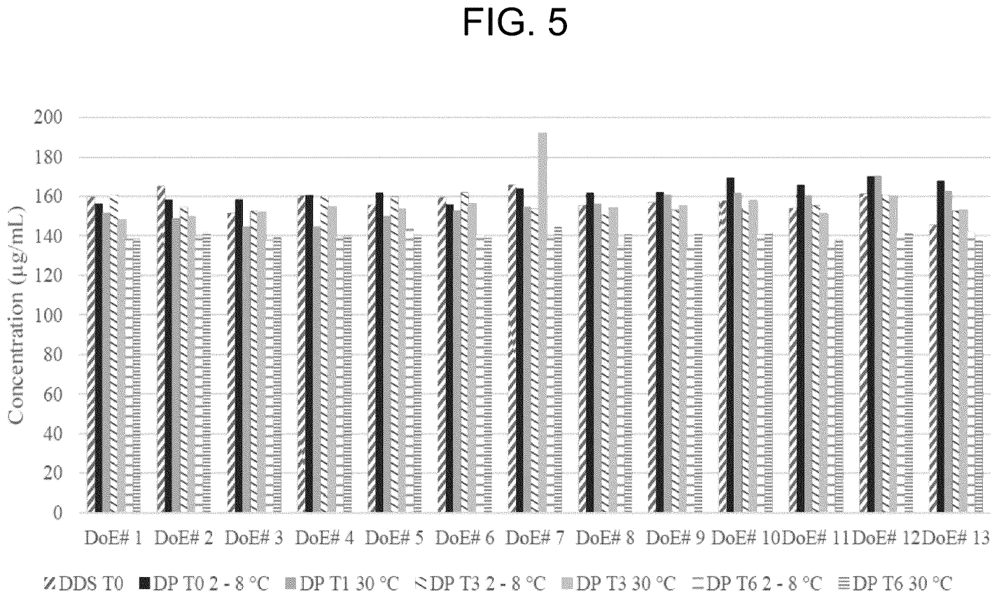

FIG. 6 shows the efanesoctocog alfa specific activity (IU/mg) measured for each of the 13 buffer compositions tested. Compositions were tested at both 2-8° C. and 30° C. Time points measured are start of experiment (T0), one month (T1), and 3 months (T3). Abbreviations: DP: drug product. DDS: diluted drug substance. DoE: design of experiment.

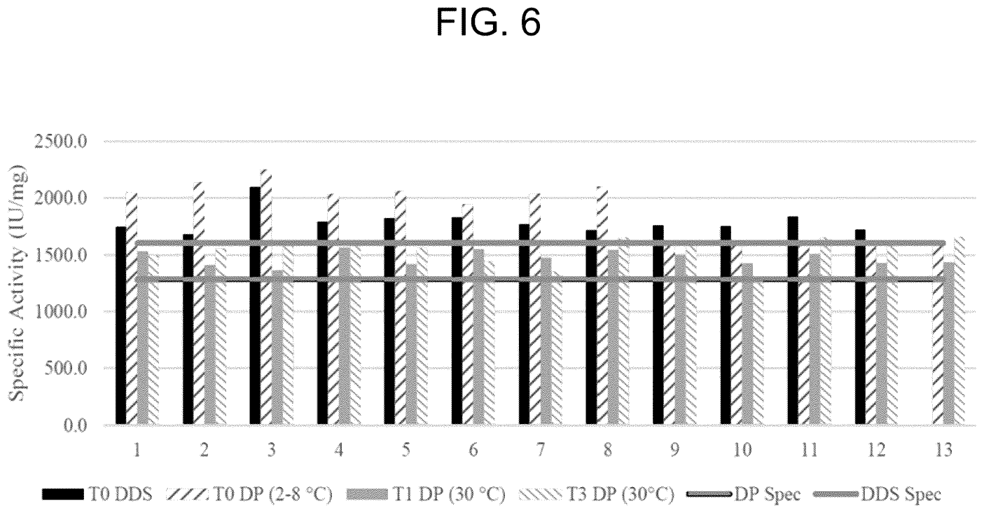

FIG. 7 shows the efanesoctocog alfa aggregation levels (% HMWS) measured for each of the 13 buffer compositions tested. Compositions were tested at both 2-8° C. and 30° C. Time points measured are start of experiment (T0), one month (T1), and 3 months (T3), and 6 months (T6). The 5% specification limit is noted on the graph. Abbreviations: DP: drug product. DDS: diluted drug substance. DoE: design of experiment.

FIG. 8 shows the glass transition temperature (Tg) measured using DSC for each of the 13 buffer compositions tested. Compositions were tested at both 2-8° C. and 30° C. Time points measured are start of experiment (T0), one month (T1), 3 months (T3), 6 months (T6), and 12 months (T12). Abbreviations: DP: drug product. DDS: diluted drug substance. DoE: design of experiment.

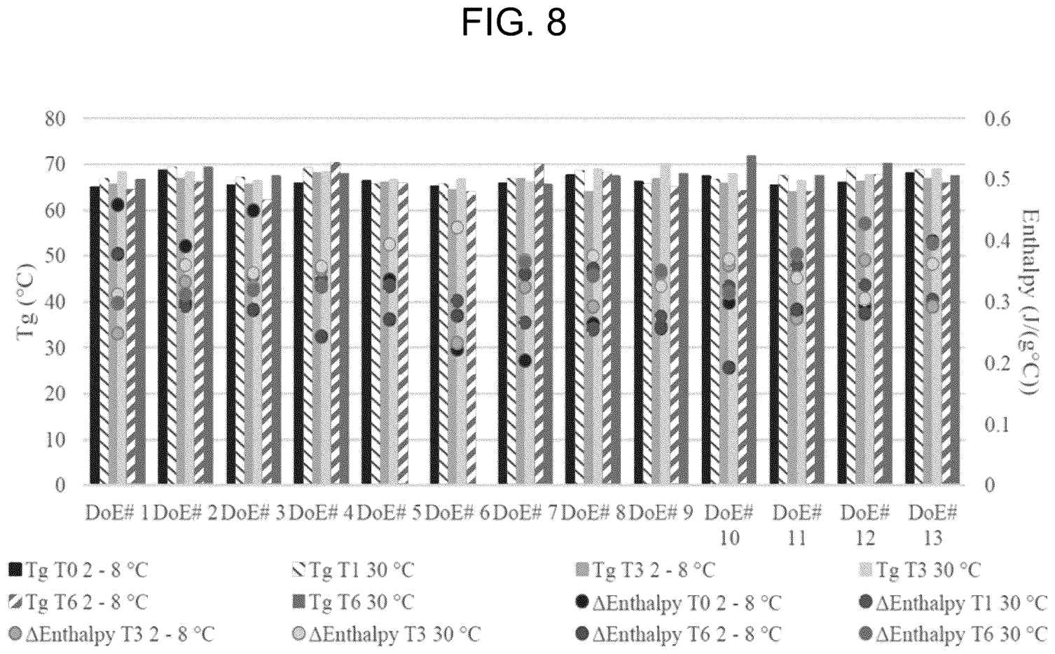

FIG. 9 shows the aggregation levels (% HMWS) over time (hours) of efanesoctocog alfa drug substance (DS) compositions (1 mg/mL) containing 5% sucrose (dashed line) or 1% sucrose (solid line) at room temperature (RT)/room light (RL) conditions.

FIG. 10 shows the aggregation levels (% HMWS) over time (hours) of efanesoctocog alfa drug substance (DS) compositions (1 mg/mL) containing 5% sucrose (dashed line) or 1% sucrose (solid line) at 2-8° C.

FIG. 11 shows the aggregation levels (% HMWS) over time (hours) of 250 IU efanesoctocog alfa bulk drug product (BDP) compositions containing 0, 1, 2, and 5% w/v sucrose concentrations at room temperature (RT)/room light (RL) conditions.

FIG. 12 shows the aggregation levels (% HMWS) over time (hours) of 250 IU efanesoctocog alfa bulk drug product (BDP) compositions containing 0, 1, 2, and 5% w/v sucrose concentrations at 2-8° C.

FIG. 13 shows the aggregation levels (% HMWS) of compositions of efanesoctocog alfa lyophilized drug product (Lyo DP) at 250 IU at 5° C. Lyo DP compositions containing 0, 1, 2, and 5% w/v sucrose were tested. Samples were tested at TO, 1 month, 2 months, 3 months, and 6 months.

FIG. 14 shows the aggregation levels (% HMWS) of compositions of efanesoctocog alfa lyophilized drug product (Lyo DP) at 250 IU at 30° C. Lyo DP compositions containing 0, 1, 2, and 5% w/v sucrose were tested. Samples were tested at TO, 1 month, 2 months, 3 months, and 6 months.

FIG. 15 shows the aggregation levels (% HMWS) of compositions of efanesoctocog alfa lyophilized drug product (Lyo DP) at 250 IU at 40° C. Lyo DP compositions containing 0, 1, 2, and 5% w/v sucrose were tested. Samples were tested at TO, 1 month, 2 months, 3 months, and 6 months.

FIG. 16 shows the aggregation levels (% HMWS) of compositions of efanesoctocog alfa lyophilized drug product (Lyo DP) at 4000 IU at 5° C. Lyo DP compositions containing 0, 1, 2, and 5% w/v sucrose were tested. Samples were tested at TO, 1 month, 2 months, 3 months, and 6 months.

FIG. 17 shows the aggregation levels (% HMWS) of compositions of efanesoctocog alfa lyophilized drug product (Lyo DP) at 4000 IU at 30° C. Lyo DP compositions containing 0, 1, 2, and 5% w/v sucrose were tested. Samples were tested at TO, 1 month, 2 months, 3 months, and 6 months.

FIG. 18 shows the aggregation levels (% HMWS) of compositions of efanesoctocog alfa lyophilized drug product (Lyo DP) at 4000 IU at 40° C. Lyo DP compositions containing 0, 1, 2, and 5% w/v sucrose were tested. Samples were tested at TO, 1 month, 2 months, 3 months, and 6 months.

FIG. 19 shows aggregation levels (% HMWS) over time (hours) of efanesoctocog alfa DS and 4000 IU bulk drug product (BDP) liquid compositions containing either 2% w/v sucrose (solid line) or 5% w/v sucrose (dashed line). Samples were held at room temperature or 5° C. and tested after 0, 5, 7, 25, 43, and 55 hours.

FIG. 20 shows the glass transition temperature (Tg) measured using DSC for compositions of efanesoctocog alfa DP at 250 IU or 4000 IU at 5° C. Compositions containing 0, 1, 2, and 5% w/v sucrose were tested at both strengths. Time points measured are start of experiment (T0), one month (T1), 3 months (T3), and 6 months (T6).

FIG. 21 shows the glass transition temperature (Tg) measured using DSC for compositions of efanesoctocog alfa DP at 250 IU or 4000 IU at 30° C. Compositions containing 0, 1, 2, and 5% w/v sucrose were tested at both strengths. Time points measured are start of experiment (T0), one month (T1), 3 months (T3), and 6 months (T6).

FIG. 22 shows the glass transition temperature (Tg) measured using DSC for compositions of efanesoctocog alfa DP at 250 IU or 4000 IU at 40° C. Compositions containing 0, 1, 2, and 5% w/v sucrose were tested at both strengths. Time points measured are start of experiment (T0), one month (T1), 3 months (T3), and 6 months (T6).

FIG. 23 shows the glass transition temperature (Tg) using DSC of efanesoctocog alfa liquid BDP at 250 IU or 4000 IU Compositions containing 0, 1, 2, and 5% w/v sucrose were tested at both strengths.

FIG. 24 shows the residual moisture content of compositions of efanesoctocog alfa lyophilized drug product (Lyo DP) at 250 IU or 4000 IU at 5° C. Lyo DP compositions containing 0, 1, 2, and 5% w/v sucrose were tested. Time points measured are start of experiment (T0), one month (T1), 3 months (T3), and 6 months (T6).

FIG. 25 shows the residual moisture content of compositions of efanesoctocog alfa lyophilized drug product (Lyo DP) at 250 IU or 4000 IU at 30° C. Lyo DP compositions containing 0, 1, 2, and 5% w/v sucrose were tested. Time points measured are start of experiment (T0), one month (T1), 3 months (T3), and 6 months (T6).

FIG. 26 shows the residual moisture content of compositions of efanesoctocog alfa lyophilized drug product (Lyo DP) at 250 IU or 4000 IU at 40° C. Lyo DP compositions containing 0, 1, 2, and 5% w/v sucrose were tested. Time points measured are start of experiment (T0), one month (T1), 3 months (T3), and 6 months (T6).

FIG. 27 is a graphical depiction of the percentage of high molecular weight (HMW) aggregates of efanesoctocog alfa (BIVV001) as analyzed by size exclusion chromatography (SEC) with buffers of varying arginine concentrations (Experiment 1). Data was collected at 0 minutes, 40 minutes, 80 minutes, 120 minutes, and 160 minutes post-thaw.

FIG. 28 is a graphical depiction of the percentage of high molecular weight (HMW) aggregates of efanesoctocog alfa (BIVV001) as analyzed by size exclusion chromatography (SEC) with buffers of varying arginine concentrations (Experiment 2). Data was collected at 0 minutes, 40 minutes, 80 minutes, 120 minutes, and 160 minutes post-thaw.

### DETAILED DESCRIPTION OF THE DISCLOSURE

The present disclosure is directed to, inter alia, formulations (including aqueous and lyophilized formulations, as well as related kits) comprising FVIII proteins. In some embodiments, the FVIII protein is a chimeric FVIII protein such as efanesoctocog alfa comprising two polypeptides, i.e., a first polypeptide comprising a FVIII protein comprising a first ELNN Polypeptide sequence insert fused to a first Fc region, and a second polypeptide comprising a VWF protein fused to a second Ig constant region by a second ELNN Polypeptide sequence, wherein the first ELNN Polypeptide sequence contains about 288 amino acids and the second ELNN Polypeptide sequence contains about 144 amino acids, and the first Ig constant region and the second Ig constant region are covalently linked together by disulfide bonds.

The present disclosure provides formulations (including aqueous and lyophilized formulations, as well as related kits) for a chimeric protein comprising (i) a factor VIII (FVIII) polypeptide and (ii) a von Willebrand factor (VWF) fragment comprising a D′ domain of VWF and a D3 domain of VWF. Included herein are compositions that may be lyophilized, as well as compositions formed upon reconstitution of lyophilized formulations with a diluent. Therapeutic methods and uses are also provided.

### I. Definitions

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, may provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

Depending on context, the term “polynucleotide” or “nucleotide” may encompass a singular nucleic acid as well as plural nucleic acids. In some embodiments, a polynucleotide is an isolated nucleic acid molecule or construct, e.g., messenger RNA (mRNA) or plasmid DNA (pDNA). In some embodiments, a polynucleotide comprises a conventional phosphodiester bond. In some embodiments, a polynucleotide comprises a non-conventional bond (e.g., an amide bond, such as found in peptide nucleic acids (PNA)). The term “nucleic acid” may refer to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a Factor VIII polypeptide contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals.

Certain proteins secreted by mammalian cells are associated with a secretory signal peptide which is cleaved from the mature protein once export of the growing protein chain across the rough endoplasmic reticulum has been initiated. Those of ordinary skill in the art are aware that signal peptides are generally fused to the N-terminus of the polypeptide, and are cleaved from the complete or “full-length” polypeptide to produce a secreted or “mature” form of the polypeptide. In some embodiments, a native signal peptide or a functional derivative of that sequence that retains the ability to direct the secretion of the polypeptide that is operably associated with it. Alternatively, a heterologous mammalian signal peptide, e.g., a human tissue plasminogen activator (TPA) or mouse β-glucuronidase signal peptide, or a functional derivative thereof, can be used.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced recombinant technology, but is not necessarily translated from a designated nucleic acid sequence. It can be generated in any manner, including by chemical synthesis.

An “isolated” polypeptide or a fragment, variant, or derivative thereof refers to a polypeptide that is not in its natural milieu. No particular level of purification is required. For example, an isolated polypeptide can simply be removed from its native or natural environment. Recombinantly produced polypeptides and proteins expressed in host cells are considered isolated for the purpose of the disclosure, as are native or recombinant polypeptides which have been separated, fractionated, or partially or substantially purified by any suitable technique.

Also included in the present disclosure are fragments or variants of polypeptides, and any combination thereof. The term “fragment” or “variant” when referring to polypeptide binding domains or binding molecules of the present disclosure include any polypeptides which retain at least some of the properties (e.g., FcRn binding affinity for an FcRn binding domain or Fc variant, coagulation activity for an FVIII variant, or FVIII binding activity for the VWF fragment) of the reference polypeptide. Fragments of polypeptides include proteolytic fragments, as well as deletion fragments, in addition to specific antibody fragments discussed elsewhere herein, but do not include the naturally occurring full-length polypeptide (or mature polypeptide). Variants of polypeptide binding domains or binding molecules of the present disclosure include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions.

The term “VWF protein” as used herein means any VWF fragment that interacts with FVIII and retains at least one or more properties that are normally provided to FVIII by full-length VWF, e.g., preventing premature activation to FVIIIa, preventing premature proteolysis, preventing clearance, preventing association with phospholipid membranes that could lead to premature clearance, preventing binding to FVIII clearance receptors that can bind naked FVIII but not VWF-bound FVIII, and/or stabilizing the FVIII heavy chain and light chain interactions. A VWF fragment referred to herein is a VWF polypeptide that is less than the full-length VWF protein, wherein the VWF fragment retains the ability to interact with and/or bind to FVIII. In some embodiments, a VWF protein is a fragment (which may be mutated) of full-length VWF that binds to a FVIII protein such that the FVIII protein has reduced binding to, or does not bind, full length VWF (e.g., endogenous VWF in a subject).

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In some embodiments, a string of amino acids can be conservatively replaced with a structurally similar string that differs in order and/or composition of side chain family members.

As known in the art, “sequence identity” between two polypeptides is determined by comparing the amino acid sequence of one polypeptide to the sequence of a second polypeptide. Similarly, “sequence identity” between two polynucleotides is determined by comparing the nucleotide sequence of one polynucleotide to the sequence of a second polynucleotide. The terms “% identical”, “% identity” or similar terms are intended to refer, in particular, to the percentage of nucleotides or amino acids (as applicable) which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. For example, the optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g, at blast.ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, −2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment. When discussed herein, whether any particular polypeptide is at least about 50%, 60%, 70%, 75%, 80%, 85%, 90%, 95%, 99%, or 100% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, WI 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present disclosure, the parameters are set, of course, such that the percentage of identity is calculated over the full-length of the reference polypeptide sequence and that gaps in homology of up to 5% of the total number of amino acids in the reference sequence are allowed.

As used herein, an “amino acid corresponding to” or an “equivalent amino acid” in a VWF sequence or a FVIII protein sequence is identified by alignment to maximize the identity or similarity between a first VWF or FVIII sequence and a second VWF or FVIII sequence. The number used to identify an equivalent amino acid in a second VWF or FVIII sequence is based on the number used to identify the corresponding amino acid in the first VWF or FVIII sequence.

As used herein, the term “insertion site” refers to a position in a FVIII polypeptide, or fragment, variant, or derivative thereof, which is immediately downstream of the position at which a half-life extending moiety or heterologous moiety can be inserted. An “insertion site” is specified as a number, the number being the number of the amino acid in mature native FVIII (SEQ ID NO: 8) to which the insertion site corresponds, which is immediately C-terminal to the position of the insertion. For example, the phrase “comprises an ELNN Polypeptide at an insertion site which corresponds to amino acid 1656 of SEQ ID NO: 8” indicates that the heterologous moiety is located between two amino acids corresponding to amino acid 1656 and amino acid 1657 of SEQ ID NO: 8.

The phrase “immediately downstream of an amino acid” as used herein refers to position right next to the terminal carboxyl group of the amino acid. For example, an insertion site immediately downstream of amino acid 745 corresponding to the mature wild type FVIII protein (SEQ ID NO: 8) means that the insertion site is between amino acid 745 and amino acid 746 corresponding to the mature wild type FVIII protein. Similarly, the phrase “immediately upstream of an amino acid” refers to the position right next to the terminal amine group of the amino acid.

The phrase “between two amino acids of an insertion site” as used herein refers to a position in which an ELNN Polypeptide or any other polypeptide is inserted between two adjacent amino acids. Thus, the phrases “inserted immediately downstream of an amino acid” and “inserted between two amino acids of an insertion site” are used synonymously with “inserted at an insertion site.”

The terms “inserted,” “is inserted,” “inserted into” or grammatically related terms, as used herein with respect to insertions of ELNN Polypeptide into FVIII refers to the position of an ELNN Polypeptide in a chimeric protein relative to the analogous position in native mature human FVIII. As used herein the terms refer to the characteristics of the recombinant FVIII polypeptide relative to native mature human FVIII, and do not indicate, imply or infer any methods or process by which the chimeric protein was made. For example, in reference to a chimeric protein provided herein, the phrase “an ELNN Polypeptide is inserted immediately downstream of residue 745 of the FVIII polypeptide” means that the chimeric protein comprises an ELNN Polypeptide immediately downstream of an amino acid which corresponds to amino acid 745 in native mature human FVIII, e.g., bounded by amino acids corresponding to amino acids 745 and 746 of native mature human FVIII (without requiring the presence of an amino acid corresponding to 746 of native mature human FVIII), and does not connote an order or method of production for which the chimeric protein was constructed.

As used herein, the terms “ELNN Polypeptide” and “ELNN” are synonymous, and refer to extended length polypeptides with non-naturally occurring, substantially non-repetitive sequences that are composed mainly of small hydrophilic amino acids, with the sequence having a low degree or no secondary or tertiary structure under physiologic conditions. ELNNs can confer certain desirable pharmacokinetic, physicochemical and pharmaceutical properties when linked to a VWF protein or a FVIII sequence of the disclosure to create a chimeric polypeptide. Such desirable properties include but are not limited to enhanced pharmacokinetic parameters and solubility characteristics. As used herein, the terms “ELNN Polypeptide” and “ELNN” specifically exclude antibodies or antibody fragments such as single-chain antibodies or Fc fragments of a light chain or a heavy chain. ELNN polypeptides are known in the art, and non-limiting descriptions relating to and examples of ELNN polypeptides known as XTEN® polypeptides are available in Schellenberger et al., (2009) Nat Biotechnol 27(12):1186-90; Brandl et al., (2020) Journal of Controlled Release 327:186-197; and Radon et al., (2021) Advanced Functional Materials 31, 2101633 (pages 1-33), the entire contents of each of which are incorporated herein by reference.

A “fusion” or “chimeric” protein comprises a first amino acid sequence linked to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of a Factor VIII domain of the disclosure with an Ig Fc domain. A fusion protein is created, for example, by chemical synthesis, or by creating and translating a polynucleotide in which the peptide regions are encoded in the desired relationship. A chimeric protein can further comprise a second amino acid sequence associated with the first amino acid sequence by a covalent, non-peptide bond or a non-covalent bond.

With respect to sequences, the term “linked” as used herein refers to a first amino acid sequence or nucleotide sequence covalently or non-covalently joined to a second amino acid sequence or nucleotide sequence, respectively. The first amino acid or nucleotide sequence can be directly joined or juxtaposed to the second amino acid or nucleotide sequence or alternatively an intervening sequence can covalently join the first sequence to the second sequence. Depending on context, the term “linked” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In some embodiments, the first amino acid sequence can be linked to a second amino acid sequence by a peptide bond or a linker. The first nucleotide sequence can be linked to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The term “linked” may also be indicated by a hyphen (-).

With respect to two polypeptides, the term “associated with” refers to one or more covalent or non-covalent bonds formed between a first polypeptide and a second polypeptide. In some embodiments, the term “associated with” means a covalent, non-peptide bond or a non-covalent bond. This association can be indicated by a colon, i.e., (:). In some embodiments, it means a covalent bond except a peptide bond. For example, the amino acid cysteine comprises a thiol group that can form a disulfide bond or bridge with a thiol group on a second cysteine residue. In most naturally occurring IgG molecules, the CH1 and CL regions are associated by a disulfide bond and the two heavy chains are associated by two disulfide bonds at positions corresponding to 239 and 242 using the Kabat numbering system (position 226 or 229, EU numbering system). Examples of covalent bonds include, but are not limited to, a peptide bond, a metal bond, a hydrogen bond, a disulfide bond, a sigma bond, a pi bond, a delta bond, a glycosidic bond, an agnostic bond, a bent bond, a dipolar bond, a Pi backbond, a double bond, a triple bond, a quadruple bond, a quintuple bond, a sextuple bond, conjugation, hyperconjugation, aromaticity, hapticity, or antibonding. Non-limiting examples of non-covalent bond include an ionic bond (e.g., cation-pi bond or salt bond), a metal bond, a hydrogen bond (e.g., dihydrogen bond, dihydrogen complex, low-barrier hydrogen bond, or symmetric hydrogen bond), van der Walls force, London dispersion force, a mechanical bond, a halogen bond, aurophilicity, intercalation, stacking, entropic force, or chemical polarity. In some embodiments, the one or more covalent bonds between the first amino acid chain and the second amino acid chain is two disulfide bonds. In some embodiments, the one or more covalent bonds between the first amino acid chain and the second amino acid chain is two disulfide bonds between a first Fc portion on the first amino acid chain and a second Fc portion on the second amino acid chain, wherein the two disulfide bonds occur in the hinge region of the two Fc portions.

In some embodiments, a polypeptide has an enzymatic cleavage site cleaved by an enzyme that is activated during the clotting cascade, such that cleavage of such sites occurs at the site of clot formation. Exemplary such sites include, e.g., those recognized by thrombin, Factor XIa or Factor Xa. Other enzymatic cleavage sites are known in the art and described in elsewhere herein. In constructs that include more than one processing or cleavage site, it will be understood that such sites can be the same or different. As used herein, the term “half-life” refers to a biological half-life of a particular polypeptide in vivo. Half-life can be represented by the time required for half the quantity administered to a subject to be cleared from the circulation and/or other tissues in the animal. In some embodiments, when a clearance curve of a given polypeptide is constructed as a function of time, the curve is usually biphasic with a rapid α-phase and longer β-phase. The α-phase typically represents an equilibration of the administered Fc polypeptide between the intra- and extra-vascular space and is, in part, determined by the size of the polypeptide. The β-phase typically represents the catabolism of the polypeptide in the intravascular space. In some embodiments, FVIII and chimeric proteins comprising FVIII are monophasic, and thus do not have an alpha phase, but just the single beta phase. Therefore, in some embodiments, the term half-life as used herein refers to the half-life of the polypeptide in the β-phase. The typical beta phase half-life of a human antibody in humans is 21 days. In some embodiments, the half-life is expressed as the half-life of the terminal phase.

In some embodiments, a subject has hemophilia A. In some embodiments, the hemophilia A is severe hemophilia A.

“Administer” or “administering,” as used herein refers to delivering to a subject a composition described herein, e.g., a chimeric protein. The composition, e.g., the chimeric protein, can be administered to a subject using methods known in the art. In particular, the composition can be administered intravenously, subcutaneously, intramuscularly, intradermally, or via any mucosal surface, e.g., orally, sublingually, buccally, nasally, rectally, vaginally or via pulmonary route. In some embodiments, the administration is intravenous. In some embodiments, the administration is subcutaneous. In some embodiments, the administration is self-administration. In some embodiments, a parent administers the chimeric protein to a child. In some embodiments, the chimeric protein is administered to a subject by a healthcare practitioner such as a medical doctor, a medic, or a nurse.

As used herein, the term “dose” refers to a single administration of a composition to a subject. A single dose can be administered all at once, e.g., as a bullous, or over a period of time, e.g., via an intravenous infusion. The term “multiple doses” means more than one dose, e.g., more than one administration.

When referring to co-administration of more than one composition, a dose of composition A can be administered concurrently with a dose of composition B. Alternatively, a dose of composition A can be administered before or after a dose of composition B. In some embodiments, composition A and composition B are combined into a single formulation.

As used herein, the term “interval” or “dosing interval” refers to the amount of time that elapses between a first dose of composition A and a subsequent dose of the same composition administered to a subject. A dosing interval can refer to the time that elapses between a first dose and a second dose, or a dosing interval can refer to the amount of time that elapses between multiple doses.

The term “dosing frequency” as used herein refers to the number of doses administered per a specific dosing interval. For example, a dosing frequency can be written as once a week, once every two weeks, etc. Therefore, a dosing interval of 7 days can be also written as a dosing interval of once in 7 days or once every week, or once a week.

As used herein the term “prophylactic treatment” refers to the administration of a therapy for the treatment of hemophilia A, where such treatment is intended to prevent or reduce the severity of one or more symptoms of hemophilia A, e.g., bleeding episodes, e.g., one or more spontaneous bleeding episodes, and/or joint damage. See Jimenez-Yuste et al., Blood Transfus. 12(3):314-19 (2014). To prevent or reduce the severity of such symptoms, e.g., bleeding episodes and the progression of joint disease, hemophilia A patients may receive regular infusions of clotting factor as part of a prophylactic treatment regimen. The basis of such prophylactic treatment is the observation that hemophilia patients with a clotting factor, e.g., FVIII, level of 1% or more rarely experience spontaneous bleeding episodes and have fewer hemophilia-related comorbidities as compared to patients with severe hemophilia. See, e.g., Coppola A. et al, Semin. Thromb. Hemost. 38(1): 79-94 (2012). Health care practitioners treating these hemophilia patients surmised that maintaining factor levels at around 1% with regular infusions could potentially reduce the risk of hemophilia symptoms, including bleeding episodes and joint damage. See id. Subsequent research has confirmed these benefits in pediatric hemophilia patients receiving prophylactic treatment with clotting factor, rendering prophylactic treatment the goal for people with severe hemophilia. See id.

A “prophylactic” treatment can also refer to the preemptive administration of the composition described herein, e.g., a protein (such as a chimeric protein), to a subject in order to control, manage, prevent, or reduce the occurrence or severity of one or more symptoms of hemophilia A, e.g., bleeding episodes. In some embodiments, prophylactic treatment with a clotting factor, e.g., FVIII, is used to treat subjects with severe hemophilia A. In some embodiments, prophylactic treatment refers to administering a composition disclosed herein to a subject in need thereof to reduce the occurrence of one or more symptom of hemophilia A. A prophylactic treatment can include administration of multiple doses. The multiple doses used in prophylactic treatment are typically administered at particular dosing intervals. In some embodiments, the annualized bleeding rate can be reduced to less than 10, less than 9, less than 8, less than 7, less than 6, less than 5, less than 4, less than 3, less than 2, or less than 1.

The term “on-demand treatment” or “episodic treatment” refers to the “as needed” administration of a chimeric molecule in response to symptoms of hemophilia A, e.g., a bleeding episode, or before an activity that can cause bleeding. In some aspects, the on-demand treatment can be given to a subject when bleeding starts, such as after an injury, or when bleeding is expected, such as before surgery. In some aspects, the on-demand treatment can be given prior to activities that increase the risk of bleeding, such as contact sports. In some embodiments, the on-demand treatment is given as a single dose. In some embodiments, the on-demand treatment is given as a first dose, followed by one or more additional doses. When the chimeric protein is administered on-demand, the one or more additional doses can be administered at least about 12 hours, at least about 24 hours, at least about 36 hours, at least about 48 hours, at least about 60 hours, at least about 72 hours, at least about 84 hours, at least about 96 hours, at least about 108 hours, or at least about 120 hours after the first dose. It should be noted, however, that the dosing interval associated with on-demand treatment is not the same as the dosing interval used for prophylactic treatment.

In some embodiments, the subject in need of a general hemostatic agent is undergoing, or is about to undergo, surgery. The chimeric protein of the disclosure can be administered prior to or after surgery. The chimeric protein of the disclosure can also be administered during or after surgery to control an acute bleeding episode. When the chimeric protein is administered prior to surgery, the administration can be at least about 1 hour, at least about 2 hours, at least about 4 hours, at least about 8 hours, at least about 12 hours, at least about 24 hours, at least about 36 hours, at least about 48 hours, or at least about 72 hours prior to surgery. When the chimeric protein is administered to after surgery, the administration can be at least about 1 hour, at least about 2 hours, at least about 4 hours, at least about 8 hours, at least about 12 hours, at least about 24 hours, at least about 36 hours, at least about 48 hours, or at least about 72 hours after surgery. The surgery can include, but is not limited to, liver transplantation, liver resection, dental procedures, or stem cell transplantation.

“Treat”, “treatment”, “treating”, as used herein refers to, e.g., the reduction in severity of a disease or condition; the reduction in the duration of a disease course; the amelioration of one or more symptoms associated with a disease or condition; the provision of beneficial effects to a subject with a disease or condition, without necessarily curing the disease or condition, or the prophylaxis of one or more symptoms associated with a disease or condition. In some embodiments, treating or treatment comprises maintaining a FVIII trough level at least about 1 IU/dL, 2 IU/dL, 3 IU/dL, 4 IU/dL, 5 IU/dL, 6 IU/dL, 7 IU/dL, 8 IU/dL, 9 IU/dL, 10 IU/dL, 11 IU/dL, 12 IU/dL, 13 IU/dL, 14 IU/dL, 15 IU/dL, 16 IU/dL, 17 IU/dL, 18 IU/dL, 19 IU/dL, or 20 IU/dL in a subject by administering a chimeric protein of the disclosure. As used herein, a “trough level” in a hemophilia A patient is the measurement of the lowest concentration reached by a factor therapy, e.g., a FVIII therapy, before the next dose is administered. In some embodiments, treating or treatment means maintaining a FVIII trough level of at least about 1 IU/dL between the dosing interval. In some embodiments, treating or treatment means maintaining a FVIII trough level of at least about 3 IU/dL between the dosing interval. In some embodiments, treating or treatment means maintaining a FVIII trough level of at least about 5 IU/dL between the dosing interval. In some embodiments, treating or treatment means maintaining a FVIII trough level between about 1 and about 20 IU/dL, about 2 and about 20 IU/dL, about 3 and about 20 IU/dL, about 4 and about 20 IU/dL, about 5 and about 20 IU/dL, about 6 and about 20 IU/dL, about 7 and about 20 IU/dL, about 8 and about 20 IU/dL, about 9 and about 20 IU/dL, or about 10 and about 20 IU/dL during the dosing interval.

In some embodiments, treatment or treating of a disease or condition comprises maintaining FVIII activity in a subject at a level comparable to at least about 1%, 2%, 3%, 4%, 5%, 6%, 7%, 8%, 9%, 10%, 11%, 12%, 13%, 14%, 15%, 16%, 17%, 18%, 19%, or 20% of the FVIII activity in a non-hemophiliac subject between the doses. In some embodiments, treating or treatment means maintaining a FVIII activity level of at least about 1% between the dosing interval. In some embodiments, treating or treatment means maintaining a FVIII activity level of at least about 2% between the dosing interval. In some embodiments, treating or treatment means maintaining a FVIII activity level of at least about 3% between the dosing interval. In some embodiments, treating or treatment means maintaining a FVIII activity level of at least about 4% between the dosing interval. In some embodiments, treating or treatment means maintaining a FVIII activity level of at least about 5%. between the dosing interval. In some embodiments, treating or treatment means maintaining a FVIII activity level of at least about 6% between the dosing interval. In some embodiments, treating or treatment means maintaining a FVIII activity level of at least about 7% between the dosing interval. In some embodiments, treating or treatment means maintaining a FVIII activity level of at least about 8% between the dosing interval. In some embodiments, treating or treatment means maintaining a FVIII activity level of at least about 9% between the dosing interval. In some embodiments, treating or treatment means maintaining a FVIII activity level of at least about 10% between the dosing interval. In some embodiments, the minimum trough level required for treatment can be measured by one or more known methods (for example, the activated partial thromboplastin time (aPTT) assays or chromogenic assays, which are well known in the art) and can be adjusted (increased or decreased) for each person. Non-limiting examples of assays for measuring trough level are disclosed in U.S. Application Publication No. 20190375822, which is hereby incorporated by reference in its entirety.

### II. Chimeric Proteins

In an aspect, the present disclosure is directed to pharmaceutical compositions comprising a chimeric protein or protein which comprises a first polypeptide chain which comprises a Factor VIII (“FVIII”) protein or a portion thereof and a first immunoglobulin (“Ig”) constant region or a portion thereof, and a second polypeptide chain which comprises a von Willebrand Factor (“VWF”) protein and a second Ig constant region or a portion thereof. In some embodiments, the chimeric protein comprises (i) a FVIII protein comprising a FVIII polypeptide, an ELNN Polypeptide inserted within the B domain of the FVIII polypeptide, and a first Fc region; and (ii) a VWF protein comprising a VWF fragment, a second ELNN Polypeptide sequence, an a2 linker, and a second Fc region. In some embodiments, the chimeric protein disclosed herein is a FVIII-ELNN-Fc/D′D3-ELNN-Fc heterodimer. Non-limiting examples of chimeric proteins that may be used in various embodiments are described in U.S. Pat. Nos. 10,138,291 and 11,192,936 B2, the entire contents of each of which are incorporated herein by reference.

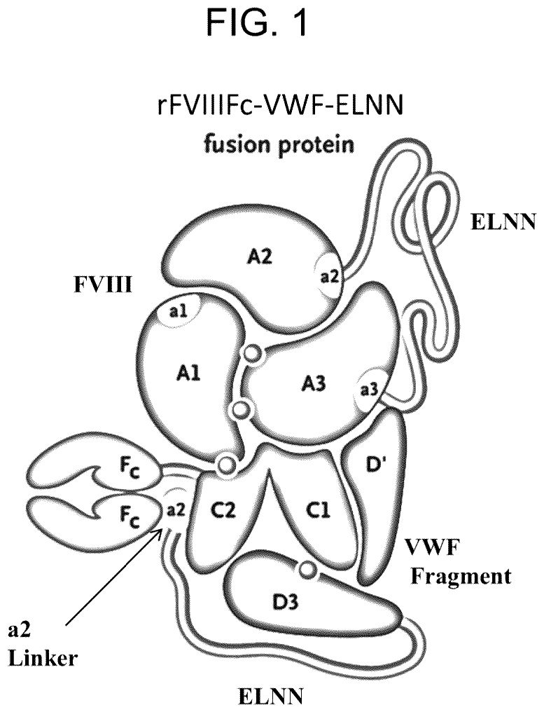

In some embodiments, the chimeric protein is efanesoctocog alfa. Efanesoctocog alfa, also known as “BIVV001”, “efanesoctocogum alfa” and “rFVIIIFc-VWF-XTEN”, is described in Chhabra et al. Blood 135(17): 1484-1496 (2020), the entire contents of which are hereby incorporated by reference in its entirety. A schematic representation of efanesoctocog alfa, as an exemplary FVIII-ELNN-Fc/D′D3-ELNN-Fc heterodimer, is presented in FIG. 1.

Efanesoctocog alfa is a large protein (over 300 kDa) comprising multiple parts on each of two polypeptide chains that are associated by covalent bonds and non-covalent interactions. The protein has a tendency to aggregate under certain conditions, which can reduce the stability of formulations unless an excipient such as L-arginine is selected and present in an amount that is sufficient to reduce the aggregation. For example, the aggregation may be reduced by adding high levels of L-arginine (e.g., about 250 mM).

Additional information regarding efanesoctocog alfa can be found in the International Nonproprietary Names for Pharmaceutical Substances (INN) WHO Drug Information, 2019, Vol. 33, No. 4, p. 828-30. In some embodiments, the chimeric protein is a FVIII-ELNN-Fc/D′D3-ELNN-Fc heterodimer comprising (i) a first polypeptide comprising the amino acid sequence of SEQ ID NO: 1 and (ii) a second polypeptide comprising the amino acid sequence of SEQ ID NO: 2. In some embodiments, the chimeric protein comprises (i) a first polypeptide and (ii) a second polypeptide that are covalently linked via one or more disulfide bonds (e.g., two disulfide bonds). In some embodiments, the chimeric protein comprises a FVIII protein encoded by the nucleic acid sequence of SEQ ID NO: 4. In some embodiments, the chimeric protein comprises a VWF protein encoded by the nucleic acid sequence of SEQ ID NO: 6. In some embodiments, the efanesoctocog alfa has an activity of at least 1600 IU/mg. In some embodiments, the efanesoctocog alfa has an activity of at least 1700 IU/mg. In some embodiments, the efanesoctocog alfa has an activity of at least 1800 IU/mg. In some embodiments, the efanesoctocog alfa has an activity of at least 1900 IU/mg. In some embodiments, the efanesoctocog alfa has an activity of 1600 IU/mg to 2000 IU/mg.

In some embodiments, the chimeric protein comprises a FVIII protein comprising the amino acid sequence of SEQ ID NO: 1. In some embodiments, the chimeric protein comprises a FVIII protein comprising one or more disulfide bridges at one or more of the following locations: residues 153-179, 248-329, 528-554, 630-711, 1220-1246, 1287-1291, 1409-1557, 1562-1714, 1761-1821, and/or 1867-1925 of SEQ ID NO: 1. In some embodiments, the chimeric protein comprises a FVIII protein comprising one or more disulfide bridges at each of the following locations: residues 153-179, 248-329, 528-554, 630-711, 1220-1246, 1287-1291, 1409-1557, 1562-1714, 1761-1821, and 1867-1925 of SEQ ID NO: 1. In some embodiments, the chimeric protein comprises a FVIII protein comprising one or more Cys-SH residues at residues 310, 692, and/or 1388 of SEQ ID NO: 1. In some embodiments, the chimeric protein comprises a FVIII protein comprising a Cys-SH residues at each of residues 310, 692, and/or 1388 of SEQ ID NO: 1.

In some embodiments, the chimeric protein comprises a FVIII protein comprises one or more N-glycosylation sites at residues N41, N239, N1198, N1506, and/or N1797 of SEQ ID NO: 1. In some embodiments, the chimeric protein comprises a FVIII protein comprises one or more O-glycosylation sites at residues 746-1036 of SEQ ID NO: 1 and/or the Ser and Thr residues in the linker peptides. In some embodiments, the chimeric protein comprises a FVIII protein comprises one or more Tyr-sulfation sites at residues 346, 718, 719, 723, 729, 1052, and/or 1068 of SEQ ID NO: 1.

In some embodiments, the chimeric protein comprises a VWF protein comprising the amino acid sequence of SEQ ID NO: 2. In some embodiments, the chimeric protein comprises a VWF protein comprising one or more disulfide bridges at one or more of the following locations: residues 4-45, 13-41, 25-36, 29-64, 47-58, 66-88, 83-100, 86-95, 104-233, 126-268, 135-230, 151-158, 283-326, 297-321, 308-348, 328-334, 338-363, 367-410, 386-406, 390-402, 394-433, 414-427, 436-464, 459-474, 462-471, 698-758, and/or 804-862 of SEQ ID NO: 2. In some embodiments, the chimeric protein comprises a VWF protein comprising one or more disulfide bridges at each of the following locations: residues 4-45, 13-41, 25-36, 29-64, 47-58, 66-88, 83-100, 86-95, 104-233, 126-268, 135-230, 151-158, 283-326, 297-321, 308-348, 328-334, 338-363, 367-410, 386-406, 390-402, 394-433, 414-427, 436-464, 459-474, 462-471, 698-758, and/or 804-862 of SEQ ID NO: 2.

In some embodiments, the chimeric protein comprises a VWF protein comprising one or more N-glycosylation sites at residues N94, N384, N734 of SEQ ID NO: 2. In some embodiments, the chimeric protein comprises a VWF protein comprising one or more O-glycosylation sites at residues 478-625 of SEQ ID NO: 2 and/or the Ser and Thr residues in the linker peptides. In some embodiments, the chimeric protein comprises a VWF protein comprising one or more Tyr-sulfation sites at residues 632, 633, 637, and/or 643 of SEQ ID NO: 2. In some embodiments, the VWF protein comprises a VWF fragment comprising a D1, D2, D′, and/or D3 domain of VWF. In one embodiment, the VWF fragment comprises a D1D2 region of VWF comprising the amino acid sequence of SEQ ID NO: 20. In some embodiments, the VWF protein further comprises a VWF signal peptide sequence. In one embodiment, the VWF signal peptide comprises the amino acid sequence of SEQ ID NO: 19. In one specific embodiment, the VWF protein comprises a VWF signal peptide comprising the amino acid sequence of SEQ ID NO: 19, a D1D2 region of VWF comprising the amino acid sequence of SEQ ID NO: 20, a D′ domain of VWF comprising the amino acid sequence of SEQ ID NO: 21, a D3 domain of VWF comprising the amino acid sequence of SEQ ID NO: 22, an ELNN Polypeptide sequence comprising the amino acid sequence of SEQ ID NO: 14 (AE144_5A), an a2 linker comprising the amino acid sequence of SEQ ID NO: 15, and/or a Fc region comprising the amino acid sequence of SEQ ID NO: 23.

In some embodiments, the chimeric protein of the present disclosure comprises: (i) a FVIII protein comprising a FVIII polypeptide, a first ELNN Polypeptide sequence, and a first Fc region; and (ii) a VWF fragment comprising a D′ domain of VWF and a D3 domain of VWF, a second ELNN Polypeptide sequence, an a2 linker of FVIII, and a second Fc region; wherein: the FVIII protein has a deletion of amino acids 746 to 1648 corresponding to mature FVIII; the first ELNN Polypeptide sequence is inserted within the FVIII polypeptide immediately downstream of amino acid 745 corresponding to mature FVIII; the first ELNN Polypeptide sequence comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the amino acid sequence of AE288 (SEQ ID NO: 9); the first Fc region is fused to the C-terminus of the FVIII polypeptide; the second ELNN Polypeptide sequence is fused to the C-terminus of the VWF fragment; the second ELNN Polypeptide sequence comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the amino acid sequence of AE144_5A (SEQ ID NO: 14); the a2 linker is fused to the C-terminus of the ELNN Polypeptide; the a2 linker comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the amino acid sequence of SEQ ID NO: 15; the second Fc region is fused to the C-terminus of the a2 linker; and the first Fc region is covalently linked to the second Fc region by a disulfide bond (e.g., two disulfide bonds).

In some embodiments, the chimeric protein of the disclosure comprises two polypeptide sequences, a first polypeptide sequence comprising an amino acid sequence at least about 80%, 90%, 95%, or 100% identical to the amino acid sequence set forth in SEQ ID NO: 1; and a second polypeptide sequence comprising a VWF fragment comprising a D′ domain of VWF and a D3 domain of VWF and an Fc region. In some embodiments, the chimeric protein of the disclosure comprises two polypeptide sequences, a first polypeptide sequence comprising FVIII polypeptide and an Fc region; and a second polypeptide sequence comprising an amino acid sequence at least about 80%, 90%, 95%, or 100% identical to the amino acid sequence set forth in SEQ ID NO: 2. In some embodiments, the chimeric protein of the disclosure comprises two polypeptide sequences, a first polypeptide sequence comprising an amino acid sequence at least about 80%, 90%, 95%, or 100% identical to the amino acid sequence set forth in SEQ ID NO: 1 and a second polypeptide sequence comprising an amino acid sequence at least about 80%, 90%, 95%, or 100% identical to the amino acid sequence set forth in SEQ ID NO: 2. In some embodiments, the chimeric protein of the disclosure comprises two polypeptide sequences, a first polypeptide sequence comprising the amino acid sequence set forth in SEQ ID NO: 7 and a second polypeptide sequence comprising the amino acid sequence set forth in SEQ ID NO: 2. In some embodiments, the chimeric protein of the disclosure comprises two polypeptide sequences, a first polypeptide sequence comprising the amino acid sequence set forth in SEQ ID NO: 1 and a second polypeptide sequence comprising the amino acid sequence set forth in SEQ ID NO: 2, wherein the first polypeptide sequence and the second polypeptide sequence are linked to each other by a disulfide bond. In some embodiments, the chimeric protein of the disclosure comprises two polypeptide sequences, a first polypeptide sequence comprising the amino acid sequence set forth in SEQ ID NO: 1 and a second polypeptide sequence comprising the amino acid sequence set forth in SEQ ID NO: 2, wherein the first polypeptide sequence and the second polypeptide sequence are linked to each other by two disulfide bonds. In some embodiments, the chimeric protein of the disclosure comprises two polypeptide sequences, a first polypeptide sequence comprising the amino acid sequence set forth in SEQ ID NO: 1 and a second polypeptide sequence comprising the amino acid sequence set forth in SEQ ID NO: 2, wherein the first polypeptide sequence comprises a first Fc portion, wherein the second polypeptide sequence comprises a second Fc portion, wherein the first Fc portion and the second Fc portion are linked to each other by two disulfide bonds in the hinge region.

In some embodiments, the chimeric protein of the disclosure comprises a FVIII protein comprising an amino acid sequence at least about 80%, 90%, 95%, or 100% identical to SEQ ID NO: 7, SEQ ID NO: 3, or SEQ ID NO: 1; and a VWF protein comprising an amino acid sequence at least about 80%, 90%, 95%, or 100% identical to SEQ ID NO: 2 or SEQ ID NO: 5.

In some embodiments, the chimeric protein of the disclosure comprises: (i) a FVIII protein comprising a first FVIII polypeptide fragment comprising the amino acid sequence of SEQ ID NO: 17; a first ELNN Polypeptide sequence comprising the amino acid sequence of SEQ ID NO: 9 (AE288); a second FVIII polypeptide fragment comprising the amino acid sequence of SEQ ID NO: 18; and a first Fc region comprising the amino acid sequence of SEQ ID NO: 23; and (ii) a VWF protein comprising: a D′ domain of VWF comprising the amino acid sequence of SEQ ID NO: 21; a D3 domain of VWF comprising the amino acid sequence of SEQ ID NO: 22; a second ELNN Polypeptide sequence comprising the amino acid sequence of SEQ ID NO: 14 (AE144_5A); an a2 linker comprising the amino acid sequence of SEQ ID NO: 15; and a second Fc region comprising the amino acid sequence of SEQ ID NO: 23, and wherein the first Fc region is covalently linked to the second Fc region by a disulfide bond (e.g., two disulfide bonds).

In some embodiments, the chimeric protein of the disclosure comprises a FVIII protein comprising a FVIII polypeptide, a first ELNN Polypeptide sequence, a first Fc region, and a VWF protein comprising a D′ domain of VWF, a D3 domain of VWF, a second ELNN Polypeptide sequence, an a2 linker of FVIII and a second Fc region, wherein the FVIII polypeptide comprises the amino acid sequence of SEQ ID NO: 17, the first ELNN Polypeptide sequence comprises the amino acid sequence of AE288 (SEQ ID NO: 9) and is fused to the C-terminus of SEQ ID NO: 17, the FVIII polypeptide further comprises the amino acid sequence of SEQ ID NO: 18, the first Fc region comprises the amino acid sequence of SEQ ID NO: 23 and is fused to the C-terminus of SEQ ID NO: 18; the D′ domain of VWF comprises the amino acid sequence of SEQ ID NO: 21; the D3 domain of VWF comprises the amino acid sequence of SEQ ID NO: 21, the second ELNN Polypeptide sequence comprises the amino acid sequence of AE144_5A (SEQ ID NO: 14) and is fused to the C-terminus of the D3 domain of VWF; the a2 linker comprises the amino acid sequence of SEQ ID NO: 15 and is fused to the C-terminus of the second ELNN Polypeptide sequence; the second Fc region comprises the amino acid sequence of SEQ ID NO: 23 and is fused to the C-terminus of the a2 linker; and wherein the first Fc region is covalently linked to the second Fc region by a disulfide bond.

In some embodiments, the chimeric protein of the disclosure comprises a FVIII protein comprising a FVIII signal peptide comprising the amino acid sequence of SEQ ID NO: 16. In some embodiments, the chimeric protein comprises a VWF protein comprising a VWF signal peptide comprising the amino acid sequence of SEQ ID NO: 19. In some embodiments, the chimeric protein comprises a VWF protein comprising a D1D2 domain of VWF comprising the amino acid sequence of SEQ ID NO: 20.

In some embodiments, the chimeric protein comprises a first polypeptide comprising the amino acid sequence of SEQ ID NO: 3 and a second polypeptide comprising the amino acid sequence of SEQ ID NO: 5. In some embodiments, the chimeric protein comprises a first polypeptide comprising the amino acid sequence of SEQ ID NO: 7 and a second polypeptide comprising the amino acid sequence of SEQ ID NO: 2. In some embodiments, the chimeric protein comprises a first polypeptide comprising the amino acid sequence of SEQ ID NO: 1 and a second polypeptide comprising the amino acid sequence of SEQ ID NO: 2.

In some embodiments, the chimeric protein comprises one or more disulfide bridges between the first polypeptide and the second polypeptide. In some embodiments, the chimeric protein comprises two disulfide bridges between the first polypeptide and the second polypeptide. In some embodiments, the chimeric protein comprises a first polypeptide comprising the amino acid sequence of SEQ ID NO: 1 and a second polypeptide comprising the amino acid sequence of SEQ ID NO: 2, wherein the chimeric protein comprises a disulfide bridge between residue 1726 of SEQ ID NO: 1 and residue 663 of SEQ ID NO: 2, and a disulfide bridge between residue 1729 of SEQ ID NO: 1 and residue 666 of SEQ ID NO: 2.

### IV. Pharmaceutical Compositions

In an aspect, the present disclosure is directed to pharmaceutical compositions of a chimeric protein which are formulated to improve protein stability. In some embodiments, the disclosed pharmaceutical compositions demonstrate increased stability based on analysis by visual inspection, protein concentration, pH stability, formation of high molecular weight species (HMWS), and/or change in turbidity. Analysis of these properties of stability can be made using conventional techniques, including size exclusion chromatography (SEC), reversed-phase high-performance liquid chromatography (RP-HPLC), and many others.

The pharmaceutical compositions disclosed herein comprise a specified amount of the chimeric protein. In some embodiments, the pharmaceutical composition has a chimeric protein concentration of about 0.8 to about 1.2 mg/mL. In some embodiments, the pharmaceutical composition has a chimeric protein concentration of about 0.8 mg/mL. In some embodiments, the pharmaceutical composition has a chimeric protein concentration of about 0.9 mg/mL. In some embodiments, the pharmaceutical composition has a chimeric protein concentration of about 1.0 mg/mL. In some embodiments, the pharmaceutical composition has a chimeric protein concentration of about 1.1 mg/mL. In some embodiments, the pharmaceutical composition has a chimeric protein concentration of about 1.2 mg/mL.

In some embodiments, the pharmaceutical composition comprises about 75 IU/mL to about 2,000 IU/mL of the chimeric protein. In some embodiments, the pharmaceutical composition comprises about 75 IU/mL of the chimeric protein. In some embodiments, the pharmaceutical composition comprises about 100 IU/mL of the chimeric protein. In some embodiments, the pharmaceutical composition comprises about 150 IU/mL of the chimeric protein. In some embodiments, the pharmaceutical composition comprises about 200 IU/mL of the chimeric protein. In some embodiments, the pharmaceutical composition comprises about 250 IU/mL of the chimeric protein. In some embodiments, the pharmaceutical composition comprises about 300 IU/mL of the chimeric protein. In some embodiments, the pharmaceutical composition comprises about 350 IU/mL of the chimeric protein. In some embodiments, the pharmaceutical composition comprises about 400 IU/mL of the chimeric protein. In some embodiments, the pharmaceutical composition comprises about 450 IU/mL of the chimeric protein. In some embodiments, the pharmaceutical composition comprises about 500 IU/mL of the chimeric protein. In some embodiments, the pharmaceutical composition comprises about 550 IU/ml of the chimeric protein. In some embodiments, the pharmaceutical composition comprises about 600 IU/ml of the chimeric protein. In some embodiments, the pharmaceutical composition comprises about 650 IU/mL of the chimeric protein. In some embodiments, the pharmaceutical composition comprises about 700 IU/mL of the chimeric protein. In some embodiments, the pharmaceutical composition comprises about 750 IU/mL of the chimeric protein. In some embodiments, the pharmaceutical composition comprises about 800 IU/mL of the chimeric protein. In some embodiments, the pharmaceutical composition comprises about 850 IU/mL of the chimeric protein. In some embodiments, the pharmaceutical composition comprises about 900 IU/ml of the chimeric protein. In some embodiments, the pharmaceutical composition comprises about 950 IU/ml of the chimeric protein. In some embodiments, the pharmaceutical composition comprises about 1000 IU/mL of the chimeric protein.

In some embodiments, the pharmaceutical composition comprises about 1100 IU/mL of the chimeric protein. In some embodiments, the pharmaceutical composition comprises about 1150 IU/mL of the chimeric protein. In some embodiments, the pharmaceutical composition comprises about 1200 IU/mL of the chimeric protein. In some embodiments, the pharmaceutical composition comprises about 1250 IU/mL of the chimeric protein. In some embodiments, the pharmaceutical composition comprises about 1300 IU/mL of the chimeric protein. In some embodiments, the pharmaceutical composition comprises about 1350 IU/mL of the chimeric protein. In some embodiments, the pharmaceutical composition comprises about 1400 IU/ml of the chimeric protein. In some embodiments, the pharmaceutical composition comprises about 1450 IU/mL of the chimeric protein. In some embodiments, the pharmaceutical composition comprises about 1500 IU/mL of the chimeric protein. In some embodiments, the pharmaceutical composition comprises about 1550 IU/mL of the chimeric protein. In some embodiments, the pharmaceutical composition comprises about 1600 IU/mL of the chimeric protein. In some embodiments, the pharmaceutical composition comprises about 1650 IU/mL of the chimeric protein. In some embodiments, the pharmaceutical composition comprises about 1700 IU/mL of the chimeric protein. In some embodiments, the pharmaceutical composition comprises about 1750 IU/mL of the chimeric protein. In some embodiments, the pharmaceutical composition comprises about 1800 IU/mL of the chimeric protein. In some embodiments, the pharmaceutical composition comprises about 1850 IU/mL of the chimeric protein. In some embodiments, the pharmaceutical composition comprises about 1900 IU/mL of the chimeric protein. In some embodiments, the pharmaceutical composition comprises about 1950 IU/mL of the chimeric protein. In some embodiments, the pharmaceutical composition comprises about 2000 IU/ml of the chimeric protein.

Pharmaceutical compositions containing the chimeric protein of the present disclosure also contain a suitable pharmaceutically acceptable carrier. For example, they can contain excipients and/or auxiliaries that provide enhanced stability of the chimeric protein or facilitate processing of the active compounds into preparations designed for delivery to the site of action.

In an aspect, disclosed herein are pharmaceutical compositions comprising a specified amount of a chimeric protein along with excipients as disclosed. The pharmaceutical compositions disclosed herein comprise various concentrations of these excipients as disclosed, and the concentrations can be expressed in various ways. For example, the concentration of a given excipient can be expressed as a molar concentration (e.g., M or mM), as a weight/volume percent, (e.g., grams per 100 ml diluent), or as milligrams per milliliter (mg/ml). Pharmaceutical compositions provided herein can contain specified amounts of the various excipients at a level of precision ranging from approximate, e.g., concentrations expressed only to one significant figure (e.g., about 0.1% (w/V)), or with more precision, e.g., out to 2, 3, 4, 5, or 6 significant figures (e.g., about 3.88 mg/ml, with precision out to three significant figures). The necessary level of precision can vary depending on, e.g., the requirements of a given regulatory agency, or the manufacturing process.

Pharmaceutical compositions disclosed herein may include a stabilizing agent.

In some embodiments, the pharmaceutical compositions disclosed herein include specified amounts or concentrations of sucrose.

In some embodiments, the pharmaceutical composition comprises 4.5% (w/v) to 8% (w/v) sucrose. In some embodiments, the pharmaceutical composition comprises about 5% (w/V) to about 7.5% (w/v) sucrose.

In some embodiments, the pharmaceutical composition comprises about 4.5% (w/V) sucrose. In some embodiments, the pharmaceutical composition comprises about 4.6% (w/V) sucrose. In some embodiments, the pharmaceutical composition comprises about 4.7% (w/V) sucrose. In some embodiments, the pharmaceutical composition comprises about 4.8% (w/V) sucrose. In some embodiments, the pharmaceutical composition comprises about 4.9% (w/v) sucrose. In some embodiments, the pharmaceutical composition comprises about 5.0% (w/V) sucrose. In some embodiments, the pharmaceutical composition comprises about 5.1% (w/V) sucrose. In some embodiments, the pharmaceutical composition comprises about 5.2% (w/V) sucrose. In some embodiments, the pharmaceutical composition comprises about 5.3% (w/V) sucrose. In some embodiments, the pharmaceutical composition comprises about 5.4% (w/V) sucrose. In some embodiments, the pharmaceutical composition comprises about 5.5% (w/v) sucrose. In some embodiments, the pharmaceutical composition comprises about 5.6% (w/v) sucrose. In some embodiments, the pharmaceutical composition comprises about 5.7% (w/v) sucrose. In some embodiments, the pharmaceutical composition comprises about 5.8% (w/v) sucrose. In some embodiments, the pharmaceutical composition comprises about 5.9% (w/V) sucrose. In some embodiments, the pharmaceutical composition comprises about 6.0% (w/V) sucrose. In some embodiments, the pharmaceutical composition comprises about 6.1% (w/V) sucrose. In some embodiments, the pharmaceutical composition comprises about 6.2% (w/V) sucrose. In some embodiments, the pharmaceutical composition comprises about 6.3% (w/v) sucrose. In some embodiments, the pharmaceutical composition comprises about 6.4% (w/V) sucrose. In some embodiments, the pharmaceutical composition comprises about 6.5% (w/v) sucrose. In some embodiments, the pharmaceutical composition comprises about 6.6% (w/V) sucrose. In some embodiments, the pharmaceutical composition comprises about 6.7% (w/v) sucrose. In some embodiments, the pharmaceutical composition comprises about 6.8% (w/V) sucrose. In some embodiments, the pharmaceutical composition comprises about 6.9% (w/v) sucrose. In some embodiments, the pharmaceutical composition comprises about 7.0% (w/V) sucrose. In some embodiments, the pharmaceutical composition comprises about 7.1% (w/v) sucrose. In some embodiments, the pharmaceutical composition comprises about 7.2% (w/V) sucrose. In some embodiments, the pharmaceutical composition comprises about 7.3% (w/v) sucrose. In some embodiments, the pharmaceutical composition comprises about 7.4% (w/V) sucrose. In some embodiments, the pharmaceutical composition comprises about 7.5% (w/v) sucrose. In some embodiments, the pharmaceutical composition comprises about 7.6% (w/V) sucrose. In some embodiments, the pharmaceutical composition comprises about 7.7% (w/v) sucrose. In some embodiments, the pharmaceutical composition comprises about 7.8% (w/V) sucrose. In some embodiments, the pharmaceutical composition comprises about 7.9% (w/V) sucrose. In some embodiments, the pharmaceutical composition comprises about 8% (w/V) sucrose.

In some embodiments, the pharmaceutical composition comprises 168.3 mg sucrose. In some embodiments, the pharmaceutical composition comprises about 168.3 mg sucrose. In some embodiments, the amount of sucrose can vary up to 10% of a specific amount. In some embodiments, the specific amount of sucrose is 168.3 mg. In some embodiments, the amount of sucrose can vary up to 5% of a specific amount. In some embodiments, the specific amount of sucrose is 168.3 mg. In some embodiments, the amount of sucrose can vary up to 1% of a specific amount. In some embodiments, the specific amount of sucrose is 168.3 mg.

In some embodiments, the pharmaceutical composition comprises 1% (w/v) to 4% (w/v) sucrose. In some embodiments, the pharmaceutical composition comprises about 1.5% (w/v) to about 2.5% (w/v) sucrose.

In some embodiments, the pharmaceutical composition comprises about 1.0% (w/v) sucrose. In some embodiments, the pharmaceutical composition comprises about 1.1% (w/v) sucrose. In some embodiments, the pharmaceutical composition comprises about 1.2% (w/V) sucrose. In some embodiments, the pharmaceutical composition comprises about 1.3% (w/V) sucrose. In some embodiments, the pharmaceutical composition comprises about 1.4% (w/v) sucrose. In some embodiments, the pharmaceutical composition comprises about 1.5% (w/V) sucrose. In some embodiments, the pharmaceutical composition comprises about 1.6% (w/V) sucrose. In some embodiments, the pharmaceutical composition comprises about 1.7% (w/V) sucrose. In some embodiments, the pharmaceutical composition comprises about 1.8% (w/V) sucrose. In some embodiments, the pharmaceutical composition comprises about 1.9% (w/v) sucrose. In some embodiments, the pharmaceutical composition comprises about 2.0% (w/V) sucrose. In some embodiments, the pharmaceutical composition comprises about 2.1% (w/V) sucrose. In some embodiments, the pharmaceutical composition comprises about 2.2% (w/v) sucrose. In some embodiments, the pharmaceutical composition comprises about 2.3% (w/v) sucrose. In some embodiments, the pharmaceutical composition comprises about 2.4% (w/V) sucrose. In some embodiments, the pharmaceutical composition comprises about 2.5% (w/V) sucrose. In some embodiments, the pharmaceutical composition comprises about 2.6% (w/V) sucrose. In some embodiments, the pharmaceutical composition comprises about 2.7% (w/V) sucrose. In some embodiments, the pharmaceutical composition comprises about 2.8% (w/v) sucrose. In some embodiments, the pharmaceutical composition comprises about 2.9% (w/V) sucrose. In some embodiments, the pharmaceutical composition comprises about 3.0% (w/v) sucrose. In some embodiments, the pharmaceutical composition comprises about 3.1% (w/V) sucrose. In some embodiments, the pharmaceutical composition comprises about 3.2% (w/v) sucrose. In some embodiments, the pharmaceutical composition comprises about 3.3% (w/V) sucrose. In some embodiments, the pharmaceutical composition comprises about 3.4% (w/v) sucrose. In some embodiments, the pharmaceutical composition comprises about 3.5% (w/V) sucrose. In some embodiments, the pharmaceutical composition comprises about 3.6% (w/v) sucrose. In some embodiments, the pharmaceutical composition comprises about 3.7% (w/v) sucrose. In some embodiments, the pharmaceutical composition comprises about 3.8% (w/v) sucrose. In some embodiments, the pharmaceutical composition comprises about 3.9% (w/V) sucrose. In some embodiments, the pharmaceutical composition comprises about 4.0% (w/v) sucrose.

In some embodiments, the pharmaceutical composition comprises 67.34 mg sucrose. In some embodiments, the pharmaceutical composition comprises about 67.34 mg sucrose. In some embodiments, the amount of sucrose can vary up to 10% of a specific amount. In some embodiments, the specific amount of sucrose is 67.34 mg. In some embodiments, the amount of sucrose can vary up to 5% of a specific amount. In some embodiments, the specific amount of sucrose is 67.34 mg. In some embodiments, the amount of sucrose can vary up to 1% of a specific amount. In some embodiments, the specific amount of sucrose is 67.34 mg.

Pharmaceutical compositions disclosed herein may include a buffer. In some embodiments, the pharmaceutical compositions disclosed herein include specified amounts or concentrations of histidine. In some embodiments, the histidine included in the pharmaceutical composition is L-histidine. In some embodiments, the pharmaceutical composition comprises about 5 mM to about 15 mM histidine.

In some embodiments, the pharmaceutical composition comprises about 5 mM histidine. In some embodiments, the pharmaceutical composition comprises about 5.5 mM histidine. In some embodiments, the pharmaceutical composition comprises about 6 mM histidine. In some embodiments, the pharmaceutical composition comprises about 6.5 mM histidine. In some embodiments, the pharmaceutical composition comprises about 7 mM histidine. In some embodiments, the pharmaceutical composition comprises about 7.5 mM histidine. In some embodiments, the pharmaceutical composition comprises about 8 mM histidine. In some embodiments, the pharmaceutical composition comprises about 8.5 mM histidine. In some embodiments, the pharmaceutical composition comprises about 9 mM histidine. In some embodiments, the pharmaceutical composition comprises about 9.5 mM histidine. In some embodiments, the pharmaceutical composition comprises about 10 mM histidine. In some embodiments, the pharmaceutical composition comprises about 10.5 mM histidine. In some embodiments, the pharmaceutical composition comprises about 11 mM histidine. In some embodiments, the pharmaceutical composition comprises about 11.5 mM histidine. In some embodiments, the pharmaceutical composition comprises about 12 mM histidine. In some embodiments, the pharmaceutical composition comprises about 12.5 mM histidine. In some embodiments, the pharmaceutical composition comprises about 13 mM histidine. In some embodiments, the pharmaceutical composition comprises about 13.5 mM histidine. In some embodiments, the pharmaceutical composition comprises about 14 mM histidine. In some embodiments, the pharmaceutical composition comprises about 14.5 mM histidine. In some embodiments, the pharmaceutical composition comprises about 15 mM histidine. In some embodiments, the histidine is L-histidine.

In some embodiments, the pharmaceutical compositions disclosed herein include specified amounts or concentrations of arginine. In some embodiments, the pharmaceutical composition comprises arginine hydrochloride (HCl). In some embodiments, the arginine is L-arginine. In some embodiments, the composition comprises L-arginine-HCl.

In some embodiments, the pharmaceutical composition comprises at least 150 mM arginine. In some embodiments, the pharmaceutical composition comprises at least 200 mM arginine. In some embodiments, the pharmaceutical composition comprises at least 250 mM arginine. In some embodiments, the pharmaceutical composition comprises about 150 mM to about 300 mM arginine.

In some embodiments, the pharmaceutical composition comprises about 200 mM to about 300 mM arginine.

In some embodiments, the pharmaceutical composition comprises about 150 mM arginine. In some embodiments, the pharmaceutical composition comprises about 160 mM arginine. In some embodiments, the pharmaceutical composition comprises about 170 mM arginine. In some embodiments, the pharmaceutical composition comprises about 180 mM arginine. In some embodiments, the pharmaceutical composition comprises about 190 mM arginine. In some embodiments, the pharmaceutical composition comprises about 200 mM arginine. In some embodiments, the pharmaceutical composition comprises about 210 mM arginine. In some embodiments, the pharmaceutical composition comprises about 220 mM arginine. In some embodiments, the pharmaceutical composition comprises about 230 mM arginine. In some embodiments, the pharmaceutical composition comprises about 240 mM arginine. In some embodiments, the pharmaceutical composition comprises about 250 mM arginine. In some embodiments, the pharmaceutical composition comprises about 260 mM arginine. In some embodiments, the pharmaceutical composition comprises about 270 mM arginine. In some embodiments, the pharmaceutical composition comprises about 280 mM arginine. In some embodiments, the pharmaceutical composition comprises about 290 mM arginine. In some embodiments, the pharmaceutical composition comprises about 300 mM arginine. In some embodiments, the arginine is L-arginine. In some embodiments, the composition comprises L-arginine-HCl.

Pharmaceutical compositions disclosed herein may include a bulking agent. In some embodiments, the pharmaceutical compositions disclosed herein include specified amounts or concentrations of calcium chloride (CaCl₂)). In some embodiments, the composition comprises CaCl₂·2H₂O, CaCl₂) (anhydrous), CaCl₂·4H₂O, or CaCl₂·6H₂O. In some embodiments, the composition comprises calcium chloride dihydrate. In some embodiments, the pharmaceutical composition comprises about 2.5 mM to about 10 mM calcium chloride. In some embodiments, the composition comprises calcium chloride dihydrate.

In some embodiments, the pharmaceutical composition comprises about 2.5 mM calcium chloride. In some embodiments, the pharmaceutical composition comprises about 3 mM calcium chloride. In some embodiments, the pharmaceutical composition comprises about 3.5 mM calcium chloride. In some embodiments, the pharmaceutical composition comprises about 4 mM calcium chloride. In some embodiments, the pharmaceutical composition comprises about 4.5 mM calcium chloride. In some embodiments, the pharmaceutical composition comprises about 5 mM calcium chloride. In some embodiments, the pharmaceutical composition comprises about 5.5 mM calcium chloride. In some embodiments, the pharmaceutical composition comprises about 6 mM calcium chloride. In some embodiments, the pharmaceutical composition comprises about 6.5 mM calcium chloride. In some embodiments, the pharmaceutical composition comprises about 7 mM calcium chloride. In some embodiments, the pharmaceutical composition comprises about 7.5 mM calcium chloride. In some embodiments, the pharmaceutical composition comprises about 8 mM calcium chloride. In some embodiments, the pharmaceutical composition comprises about 8.5 mM calcium chloride. In some embodiments, the pharmaceutical composition comprises about 9 mM calcium chloride. In some embodiments, the pharmaceutical composition comprises about 9.5 mM calcium chloride. In some embodiments, the pharmaceutical composition comprises about 10 mM calcium chloride. In some embodiments, the composition comprises calcium chloride dihydrate.

In some embodiments, the pharmaceutical compositions disclosed herein do not include bulking agents other than calcium chloride. In some embodiments, calcium chloride is the sole bulking agent. In some embodiments, the pharmaceutical composition comprises less than 8.8 mg/mL sodium chloride (NaCl). In some embodiments, the pharmaceutical composition is substantially free of sodium chloride. In some embodiments, the pharmaceutical composition is free of sodium chloride.

In some embodiments, the pharmaceutical compositions disclosed herein include specified amounts or concentrations of polysorbate 20 (PS20) or polysorbate 80 (PS80). In some embodiments, the pharmaceutical composition comprises about 0.008% (w/v) to about 0.1% (w/V) PS80 or PS20. In some embodiments, the pharmaceutical composition comprises at least about 0.03% PS20 or PS80. In some embodiments, the pharmaceutical composition comprises about 0.05% PS20 or PS80. In some embodiments, the pharmaceutical composition comprises PS20. In some embodiments, the pharmaceutical composition comprises PS80.

In some embodiments, the pharmaceutical composition comprises about 0.008% (w/v) polysorbate 20. In some embodiments, the pharmaceutical composition comprises about 0.01% (w/v) polysorbate 20. In some embodiments, the pharmaceutical composition comprises about 0.02% (w/v) polysorbate 20. In some embodiments, the pharmaceutical composition comprises about 0.03% (w/v) polysorbate 20. In some embodiments, the pharmaceutical composition comprises about 0.04% (w/v) polysorbate 20. In some embodiments, the pharmaceutical composition comprises about 0.05% (w/v) polysorbate 20. In some embodiments, the pharmaceutical composition comprises about 0.06% (w/v) polysorbate 20. In some embodiments, the pharmaceutical composition comprises about 0.07% (w/v) polysorbate 20. In some embodiments, the pharmaceutical composition comprises about 0.08% (w/v) polysorbate 20. In some embodiments, the pharmaceutical composition comprises about 0.09% (w/v) polysorbate 20. In some embodiments, the pharmaceutical composition comprises about 0.1% (w/V) polysorbate 20.

In some embodiments, the pharmaceutical composition comprises about 0.008% (w/v) polysorbate 80. In some embodiments, the pharmaceutical composition comprises about 0.01% (w/v) polysorbate 80. In some embodiments, the pharmaceutical composition comprises about 0.02% (w/v) polysorbate 80. In some embodiments, the pharmaceutical composition comprises about 0.03% (w/v) polysorbate 80. In some embodiments, the pharmaceutical composition comprises about 0.04% (w/v) polysorbate 80. In some embodiments, the pharmaceutical composition comprises about 0.05% (w/v) polysorbate 80. In some embodiments, the pharmaceutical composition comprises about 0.06% (w/v) polysorbate 80. In some embodiments, the pharmaceutical composition comprises about 0.07% (w/v) polysorbate 80. In some embodiments, the pharmaceutical composition comprises about 0.08% (w/v) polysorbate 80. In some embodiments, the pharmaceutical composition comprises about 0.09% (w/v) polysorbate 80. In some embodiments, the pharmaceutical composition comprises about 0.1% (w/V) polysorbate 80.

In some embodiments, the pharmaceutical composition is a pre-lyophilization solution. In some embodiments, pre-lyophilization solution does not comprise NaCl. In some embodiments, the pre-lyophilization solution does not comprise NaOH. In some embodiments, the pre-lyophilization solution does not comprise sodium ions.

In some embodiments, the pharmaceutical composition comprises
(a) about 1% (w/v) to about 4% (w/v) sucrose;
(b) about 5 mM to about 15 mM histidine;
(c) about 150 mM to about 300 mM arginine;
(d) about 2.5 mM to about 10 mM calcium chloride; and
(e) about 0.008% (w/v) to about 0.1% (w/v) polysorbate 20 or polysorbate 80.

In some embodiments, the pharmaceutical composition comprises
(a) about 1% (w/v) to about 4% (w/v) sucrose;
(b) about 5 mM to about 15 mM histidine;
(c) about 200 mM to about 300 mM arginine;
(d) about 2.5 mM to about 10 mM calcium chloride; and
(e) about 0.008% (w/v) to about 0.1% (w/v) polysorbate 20 or polysorbate 80. In some embodiments, the composition comprises polysorbate 80. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises
(a) 2% (w/v) to 3% (w/V) sucrose;
(b) 7.5 mM to 12.5 mM histidine;
(c) 225 mM to about 300 mM arginine;
(d) 5 mM to 6 mM calcium chloride; and
(e) 0.01% (w/v) to about 0.075% (w/v) polysorbate 20 or polysorbate 80. In some embodiments, the composition comprises polysorbate 80. In some embodiments, the histidine is L-histidine. In some embodiments, the arginine is L-arginine. In some embodiments the composition comprises 225 mM to about 300 mM L-arginine-HCl. In some embodiments, the composition comprises 5 mM to 6 mM calcium chloride dihydrate. In some embodiments, the pharmaceutical composition is a pre-lyophilization solution.

In some embodiments, the pharmaceutical composition comprises
(a) 2% (w/v) to 3% (w/v) sucrose;
(b) 7.5 mM to 12.5 mM L-histidine;
(c) 225 mM to about 300 mM L-arginine;
(d) 5 mM to 6 mM calcium chloride; and
(e) 0.01% (w/v) to about 0.075% (w/v) polysorbate 80.

In some embodiments, the pharmaceutical composition comprises
(a) 2% (w/v) to 3% (w/v) sucrose;
(b) 7.5 mM to 12.5 mM L-histidine;
(c) 225 mM to about 300 mM L-arginine-HCl;
(d) 5 mM to 6 mM calcium chloride; and
(e) 0.01% (w/v) to about 0.075% (w/v) polysorbate 80.

In some embodiments, the pharmaceutical composition comprises
(a) 2% (w/v) to 3% (w/v) sucrose;
(b) 7.5 mM to 12.5 mM L-histidine;
(c) 225 mM to about 300 mM L-arginine-HCl;
(d) 5 mM to 6 mM calcium chloride dihydrate; and
(e) 0.01% (w/v) to about 0.075% (w/v) polysorbate 80.

In some embodiments, the pharmaceutical composition comprises:
(a) about 2.25% (w/v) sucrose;
(b) about 11.2 mM L-histidine;
(c) about 280.58 mM L-arginine-HCl;
(d) about 5.61 mM calcium chloride dihydrate; and
(e) 0.056% (w/v) polysorbate 80.

In some embodiments, the pharmaceutical composition comprises:
(a) about 2.25% (w/v) sucrose;
(b) about 11.2 mM L-histidine;
(c) about 280.58 mM L-arginine;
(d) about 5.61 mM calcium chloride; and
(e) about 0.056% (w/v) polysorbate 80.

In some embodiments, the pharmaceutical composition comprises:
(a) 2.25% (w/v) sucrose;
(b) 11.2 mM L-histidine;
(c) 280.58 mM L-arginine-HCl;
(d) about 5.61 mM calcium chloride dihydrate; and
(e) 0.056% (w/v) polysorbate 80.

In some embodiments, the pharmaceutical composition comprises:
(a) 2.25% (w/v) sucrose;
(b) 11.2 mM L-histidine;
(c) 280.58 mM L-arginine;
(d) 5.61 mM calcium chloride; and
(e) 0.056% (w/v) polysorbate 80.

In some embodiments, when the lyophilized pharmaceutical composition and the sterile water are combined, then the resulting solution comprises:
(a) about 2.25% (w/v) sucrose;
(b) about 11.2 mM L-histidine;
(c) about 280.58 mM L-arginine-HCl;
(d) about 5.61 mM calcium chloride dihydrate; and
(e) 0.056% (w/v) polysorbate 80.

In some embodiments, when the lyophilized pharmaceutical composition and the sterile water are combined, then the resulting solution comprises:
(a) about 2.25% (w/v) sucrose;
(b) about 11.2 mM L-histidine;
(c) about 280.58 mM L-arginine;
(d) about 5.61 mM calcium chloride; and
(e) about 0.056% (w/v) polysorbate 80.

In some embodiments, when the lyophilized pharmaceutical composition and the sterile water are combined, then the resulting solution comprises:
(a) 2.25% (w/v) sucrose;
(b) 11.2 mM L-histidine;
(c) 280.58 mM L-arginine-HCl;
(d) about 5.61 mM calcium chloride dihydrate (5.61 mM calcium chloride); and
(e) 0.056% (w/v) polysorbate 80.

In some embodiments, when the lyophilized pharmaceutical composition and the sterile water are combined, then the resulting solution comprises:
(a) 2.25% (w/v) sucrose;
(b) 11.2 mM L-histidine;
(c) 280.58 mM L-arginine;
(d) 5.61 mM calcium chloride; and
(e) 0.056% (w/v) polysorbate 80.

In some embodiments, when the lyophilized pharmaceutical composition and the sterile water are combined, then the resulting solution comprises:
(a) 2% (w/v) to 3% (w/v) sucrose;
(b) 11.2 mM L-histidine;
(c) 280.58 mM L-arginine;
(d) 5.61 mM calcium chloride; and
(e) 0.056% (w/v) polysorbate 80.

In some embodiments, when the lyophilized pharmaceutical composition and the sterile water are combined, then the resulting solution comprises:
(a) 2.25% (w/v) sucrose;
(b) 7.5 mM to 12.5 mM L-histidine;
(c) 280.58 mM L-arginine;
(d) 5.61 mM calcium chloride; and
(e) 0.056% (w/v) polysorbate 80.

In some embodiments, when the lyophilized pharmaceutical composition and the sterile water are combined, then the resulting solution comprises:
(a) 2.25% (w/v) sucrose;
(b) 11.2 mM L-histidine;
(c) 225 mM to about 300 mM L-arginine;
(e) 0.056% (w/v) polysorbate 80.

In some embodiments, when the lyophilized pharmaceutical composition and the sterile water are combined, then the resulting solution comprises:
(a) 2.25% (w/v) sucrose;
(b) 11.2 mM L-histidine;
(c) 280.58 mM L-arginine;
(d) 5 mM to 6 mM calcium chloride; and
(e) 0.056% (w/v) polysorbate 80.

In some embodiments, when the lyophilized pharmaceutical composition and the sterile water are combined, then the resulting solution comprises:
(a) 2.25% (w/v) sucrose;
(b) 11.2 mM L-histidine;
(c) 280.58 mM L-arginine;
(d) 5.61 mM calcium chloride; and
(e) 0.01% (w/v) to about 0.075% (w/v) (w/v) polysorbate 80.

In some embodiments, when the lyophilized pharmaceutical composition and the sterile water are combined, then the resulting solution does not comprise NaCl. In some embodiments, when the lyophilized pharmaceutical composition and the sterile water are combined, then the resulting solution does not comprise NaOH. In some embodiments, when the lyophilized pharmaceutical composition and the sterile water are combined, then the resulting solution does not comprise sodium ions.

In some embodiments, the pharmaceutical composition comprises
(a) about 2% (w/v) sucrose;
(b) about 10 mM histidine;
(c) about 250 mM arginine;
(d) about 5 mM calcium chloride; and
(e) about 0.05% polysorbate 20 or polysorbate 80. In some embodiments, the composition comprises polysorbate 80. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises
(a) about 2% (w/v) to about 3% (w/v) sucrose;
(b) about 10 mM histidine;
(c) about 250 mM arginine;
(d) about 5 mM calcium chloride; and
(e) about 0.05% polysorbate 20 or polysorbate 80. In some embodiments, the composition comprises polysorbate 80. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises
(a) about 2% (w/v) sucrose;
(b) about 5 mM to about 15 mM histidine;
(c) about 250 mM arginine;
(d) about 5 mM calcium chloride; and
(e) about 0.05% polysorbate 80. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises
(a) about 2% (w/v) sucrose;
(b) about 10 mM histidine;
(c) about 200 mM to about 300 mM arginine;
(d) about 5 mM calcium chloride; and
(e) about 0.05% polysorbate 80. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises
(a) about 2% (w/v) sucrose;
(b) about 10 mM histidine;
(c) about 250 mM arginine;
(d) about 2.5 mM to about 10 mM calcium chloride; and
(e) about 0.05% polysorbate 80. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises
(a) about 2% (w/v) sucrose;
(b) about 10 mM histidine;
(c) about 250 mM arginine;
(d) about 5 mM calcium chloride; and
(e) about 0.008% (w/V) to about 0.1% (w/v) polysorbate 80. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises:
(a) about 20 mg/ml sucrose;
(b) about 1.552 mg/ml L-histidine;
(c) about 52.665 mg/ml L-arginine-HCl;
(d) about 0.735 mg/ml calcium chloride; and
(e) about 0.5 mg/ml polysorbate 80.

In some embodiments, the pharmaceutical composition comprises:
(a) about 20 mg/ml sucrose;
(b) about 1.552 mg/ml L-histidine;
(c) about 52.665 mg/ml L-arginine-HCl;
(d) about 0.735 mg/ml calcium chloride dihydrate; and
(e) about 0.5 mg/ml polysorbate 80.

In some embodiments, the pharmaceutical composition comprises:
(a) about 20 mg/ml sucrose;
(b) about 1.552 mg/ml L-histidine;
(c) about 52.665 mg/ml L-arginine-HCl;
(d) about 0.555 mg/ml calcium chloride; and
(e) about 0.5 mg/ml polysorbate 80. In some embodiments, the composition comprises calcium chloride dihydrate. In some embodiments, the pharmaceutical composition is a pre-lyophilization solution.

In some embodiments, the pharmaceutical composition comprises:
(a) about 20 mg/ml sucrose;
(b) about 1.552 mg/ml L-histidine;
(c) about 43.550 mg/ml L-arginine;
(d) about 0.735 mg/ml calcium chloride dihydrate; and
(e) about 0.5 mg/ml polysorbate 80. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises:
(a) about 20 mg/ml sucrose;
(b) about 1.552 mg/ml L-histidine;
(c) about 43.550 mg/ml . . . arginine;
(d) about 0.735 mg/ml calcium chloride; and
(e) about 0.5 mg/ml polysorbate 80. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises:
(a) about 20 mg/ml sucrose;
(b) about 1.552 mg/ml L-histidine;
(c) about 43.550 mg/ml L-arginine;
(d) about 0.555 mg/ml calcium chloride; and
(e) about 0.5 mg/ml polysorbate 80. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises:
(a) 22.45 mg/ml sucrose;
(b) 1.74 mg/ml L-histidine;
(c) 59.11 mg/ml L-arginine-HCl;
(d) 0.82 mg/ml calcium chloride dihydrate; and
(e) 0.56 mg/ml polysorbate 80.

In some embodiments, the pharmaceutical composition comprises:
(a) 22.45 mg/ml sucrose;
(b) 1.74 mg/ml L-histidine;
(c) 59.11 mg/ml L-arginine-HCl;
(d) 0.62 mg/ml calcium chloride; and
(e) 0.56 mg/ml polysorbate 80. In some embodiments, the composition comprises calcium chloride dihydrate.

In some embodiments, the pharmaceutical composition comprises:
(a) 22.45 mg/ml sucrose;
(b) 1.74 mg/ml L-histidine;
(c) 48.88 mg/ml L-arginine;
(d) 0.82 mg/ml calcium chloride dihydrate; and
(e) 0.56 mg/ml polysorbate 80.

In some embodiments, the pharmaceutical composition comprises:
(a) 22.45 mg/ml sucrose;
(b) 1.74 mg/ml L-histidine;
(c) 48.88 mg/ml L-arginine;
(d) 0.62 mg/ml calcium chloride; and
(e) 0.56 mg/ml polysorbate 80. In some embodiments the composition comprises L-arginine-HCl. In some embodiments, the composition comprises calcium chloride dihydrate.

In some embodiments, the pharmaceutical composition comprises
(a) about 5% (w/v) to about 7.5% (w/v) sucrose;
(b) about 5 mM to about 15 mM histidine;
(c) about 150 mM to about 300 mM arginine;
(d) about 2.5 mM to about 10 mM calcium chloride; and
(e) about 0.008% (w/v) to about 0.1% (w/v) polysorbate 20 or polysorbate 80.

In some embodiments, the pharmaceutical composition comprises
(a) about 5% (w/v) to about 7.5% (w/v) sucrose;
(b) about 5 mM to about 15 mM histidine;
(c) about 200 mM to about 300 mM arginine;
(d) about 2.5 mM to about 10 mM calcium chloride; and
(e) about 0.008% (w/v) to about 0.1% (w/v) polysorbate 20 or polysorbate 80. In some embodiments, the composition comprises polysorbate 80. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises
(a) 5% (w/v) to 6% (w/v) sucrose;
(b) 7.5 mM to 12.5 mM histidine;
(c) 225 mM to about 300 mM arginine;
(d) 5 mM to 6 mM calcium chloride; and
(e) 0.01% (w/v) to about 0.075% (w/v) polysorbate 20 or polysorbate 80. In some embodiments, the composition comprises polysorbate 80. In some embodiments, the histidine is L-histidine. In some embodiments, the arginine is L-arginine. In some embodiments the composition comprises 225 mM to about 300 mM L-arginine-HCl. In some embodiments, the composition comprises 5 mM to 6 mM calcium chloride dihydrate. In some embodiments, the pharmaceutical composition is a pre-lyophilization solution.

In some embodiments, the pharmaceutical composition comprises
(a) 5% (w/v) to 6% (w/v) sucrose;
(b) 7.5 mM to 12.5 mM L-histidine;
(c) 225 mM to about 300 mM L-arginine;
(d) 5 mM to 6 mM calcium chloride; and
(e) 0.01% (w/v) to about 0.075% (w/v) polysorbate 80.

In some embodiments, the pharmaceutical composition comprises
(a) 5% (w/v) to 6% (w/v) sucrose;
(b) 7.5 mM to 12.5 mM L-histidine;
(c) 225 mM to about 300 mM L-arginine-HCl;
(d) 5 mM to 6 mM calcium chloride; and
(e) 0.01% (w/v) to about 0.075% (w/v) polysorbate 80.

In some embodiments, the pharmaceutical composition comprises
(a) 5% (w/v) to 6% (w/v) sucrose;
(b) 7.5 mM to 12.5 mM L-histidine;
(c) 225 mM to about 300 mM L-arginine-HCl;
(d) 5 mM to 6 mM calcium chloride dihydrate; and
(e) 0.01% (w/v) to about 0.075% (w/v) polysorbate 80.

In some embodiments, the pharmaceutical composition comprises:
(a) about 5.61% (w/v) sucrose;
(b) about 11.2 mM L-histidine;
(c) about 280.58 mM L-arginine-HCl;
(d) about 5.61 mM calcium chloride dihydrate; and
(e) 0.056% (w/v) polysorbate 80.

In some embodiments, the pharmaceutical composition comprises:
(a) about 5.61% (w/v) sucrose;
(b) about 11.2 mM L-histidine;
(c) about 280.58 mM L-arginine;
(d) about 5.61 mM calcium chloride; and
(e) about 0.056% (w/v) polysorbate 80.

In some embodiments, the pharmaceutical composition comprises:
(a) 5.61% (w/v) sucrose;
(b) 11.2 mM L-histidine;
(c) 280.58 mM L-arginine-HCl;
(d) about 5.61 mM calcium chloride dihydrate; and
(e) 0.056% (w/v) polysorbate 80.

In some embodiments, the pharmaceutical composition comprises:
(a) 5.61% (w/v) sucrose;
(b) 11.2 mM L-histidine;
(c) 280.58 mM L-arginine;
(d) 5.61 mM calcium chloride; and
(e) 0.056% (w/v) polysorbate 80.

In some embodiments, when the lyophilized pharmaceutical composition and the sterile water are combined, then the resulting solution comprises:
(a) about 5.61% (w/v) sucrose;
(b) about 11.2 mM L-histidine;
(c) about 280.58 mM L-arginine-HCl;
(d) about 5.61 mM calcium chloride dihydrate; and
(e) 0.056% (w/v) polysorbate 80.

In some embodiments, when the lyophilized pharmaceutical composition and the sterile water are combined, then the resulting solution comprises:
(a) about 5.61% (w/v) sucrose;
(b) about 11.2 mM L-histidine;
(c) about 280.58 mM L-arginine;
(d) about 5.61 mM calcium chloride; and
(e) about 0.056% (w/v) polysorbate 80.

In some embodiments, when the lyophilized pharmaceutical composition and the sterile water are combined, then the resulting solution comprises:
(a) 5.61% (w/v) sucrose;
(b) 11.2 mM L-histidine;
(c) 280.58 mM L-arginine-HCl;
(d) about 5.61 mM calcium chloride dihydrate (5.61 mM calcium chloride); and
(e) 0.056% (w/v) polysorbate 80.

In some embodiments, when the lyophilized pharmaceutical composition and the sterile water are combined, then the resulting solution comprises:
(a) 5.61% (w/v) sucrose;
(b) 11.2 mM L-histidine;
(c) 280.58 mM L-arginine;
(d) 5.61 mM calcium chloride; and
(e) 0.056% (w/v) polysorbate 80.

In some embodiments, when the lyophilized pharmaceutical composition and the sterile water are combined, then the resulting solution comprises:
(a) 5% (w/v) to 6% (w/v) sucrose;
(b) 11.2 mM L-histidine;
(c) 280.58 mM L-arginine;
(d) 5.61 mM calcium chloride; and
(e) 0.056% (w/v) polysorbate 80.

In some embodiments, when the lyophilized pharmaceutical composition and the sterile water are combined, then the resulting solution comprises:
(a) 5.61% (w/v) sucrose;
(b) 7.5 mM to 12.5 mM L-histidine;
(c) 280.58 mM L-arginine;
(d) 5.61 mM calcium chloride; and
(e) 0.056% (w/v) polysorbate 80.

In some embodiments, when the lyophilized pharmaceutical composition and the sterile water are combined, then the resulting solution comprises:
(a) 5.61% (w/v) sucrose;
(b) 11.2 mM L-histidine;
(c) 225 mM to about 300 mM L-arginine;
(d) 5.61 mM calcium chloride; and
(e) 0.056% (w/v) polysorbate 80.

In some embodiments, when the lyophilized pharmaceutical composition and the sterile water are combined, then the resulting solution comprises:
(a) 5.61% (w/v) sucrose;
(b) 11.2 mM L-histidine;
(c) 280.58 mM L-arginine;
(d) 5 mM to 6 mM calcium chloride; and
(e) 0.056% (w/v) polysorbate 80.

In some embodiments, when the lyophilized pharmaceutical composition and the sterile water are combined, then the resulting solution comprises:
(a) 5.61% (w/v) sucrose;
(b) 11.2 mM L-histidine;
(c) 280.58 mM L-arginine;
(d) 5.61 mM calcium chloride; and
(e) 0.01% (w/v) to about 0.075% (w/v) (w/v) polysorbate 80.

In some embodiments, when the lyophilized pharmaceutical composition and the sterile water are combined, then the resulting solution does not comprise NaCl. In some embodiments, when the lyophilized pharmaceutical composition and the sterile water are combined, then the resulting solution does not comprise NaOH. In some embodiments, when the lyophilized pharmaceutical composition and the sterile water are combined, then the resulting solution does not comprise sodium ions.

In some embodiments, the pharmaceutical composition comprises
(a) about 5% (w/v) sucrose;
(b) about 10 mM histidine;
(c) about 250 mM arginine;
(d) about 5 mM calcium chloride; and
(e) about 0.05% polysorbate 20 or polysorbate 80. In some embodiments, the composition comprises polysorbate 80. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises
(a) about 5% (w/v) to about 7.5% (w/v) sucrose;
(b) about 10 mM histidine;
(c) about 250 mM arginine;
(d) about 5 mM calcium chloride; and
(e) about 0.05% polysorbate 20 or polysorbate 80. In some embodiments, the composition comprises polysorbate 80. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises
(a) about 5% (w/v) sucrose;
(b) about 5 mM to about 15 mM histidine;
(c) about 250 mM arginine;
(d) about 5 mM calcium chloride; and
(e) about 0.05% polysorbate 80. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises
(a) about 5% (w/v) sucrose;
(b) about 10 mM histidine;
(c) about 200 mM to about 300 mM arginine;
(d) about 5 mM calcium chloride; and
(e) about 0.05% polysorbate 80. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises
(a) about 5% (w/v) sucrose;
(b) about 10 mM histidine;
(c) about 250 mM arginine;
(d) about 2.5 mM to about 10 mM calcium chloride; and
(e) about 0.05% polysorbate 80. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises
(a) about 5% (w/v) sucrose;
(b) about 10 mM histidine;
(c) about 250 mM arginine;
(d) about 5 mM calcium chloride; and
(e) about 0.008% (w/v) to about 0.1% (w/v) polysorbate 80. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises:
(a) about 50 mg/ml sucrose;
(b) about 1.552 mg/ml L-histidine;
(c) about 52.665 mg/ml L-arginine-HCl;
(d) about 0.735 mg/ml calcium chloride; and
(e) about 0.5 mg/ml polysorbate 80.

In some embodiments, the pharmaceutical composition comprises:
(a) about 50 mg/ml sucrose;
(b) about 1.552 mg/ml L-histidine;
(c) about 52.665 mg/ml L-arginine-HCl;
(d) about 0.735 mg/ml calcium chloride dihydrate; and
(e) about 0.5 mg/ml polysorbate 80.

In some embodiments, the pharmaceutical composition comprises:
(a) about 50 mg/ml sucrose;
(b) about 1.552 mg/ml L-histidine;
(c) about 52.665 mg/ml L-arginine-HCl;
(d) about 0.555 mg/ml calcium chloride; and
(e) about 0.5 mg/ml polysorbate 80. In some embodiments, the composition comprises calcium chloride dihydrate. In some embodiments, the pharmaceutical composition is a pre-lyophilization solution.

In some embodiments, the pharmaceutical composition comprises:
(a) about 50 mg/ml sucrose;
(b) about 1.552 mg/ml L-histidine;
(c) about 43.550 mg/ml L-arginine;
(d) about 0.735 mg/ml calcium chloride dihydrate; and
(e) about 0.5 mg/ml polysorbate 80. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises:
(a) about 50 mg/ml sucrose;
(b) about 1.552 mg/ml L-histidine;
(c) about 43.550 mg/ml L-arginine;
(d) about 0.735 mg/ml calcium chloride; and
(e) about 0.5 mg/ml polysorbate 80. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises:
(a) about 50 mg/ml sucrose;
(b) about 1.552 mg/ml L-histidine;
(c) about 43.550 mg/ml L-arginine;
(d) about 0.555 mg/ml calcium chloride; and
(e) about 0.5 mg/ml polysorbate 80. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises:
(a) 56.12 mg/ml sucrose;
(b) 1.74 mg/ml L-histidine;
(c) 59.11 mg/ml L-arginine-HCl;
(d) 0.82 mg/ml calcium chloride dihydrate; and
(e) 0.56 mg/ml polysorbate 80.

In some embodiments, the pharmaceutical composition comprises:
(a) 56.12 mg/ml sucrose;
(b) 1.74 mg/ml L-histidine;
(c) 59.11 mg/ml L-arginine-HCl;
(d) 0.62 mg/ml calcium chloride; and
(e) 0.56 mg/ml polysorbate 80. In some embodiments, the composition comprises calcium chloride dihydrate.

In some embodiments, the pharmaceutical composition comprises:
(a) 56.12 mg/ml sucrose;
(b) 1.74 mg/ml L-histidine;
(c) 48.88 mg/ml L-arginine;
(d) 0.82 mg/ml calcium chloride dihydrate; and
(e) 0.56 mg/ml polysorbate 80.

In some embodiments, the pharmaceutical composition comprises:
(a) 56.12 mg/ml sucrose;
(b) 1.74 mg/ml L-histidine;
(c) 48.88 mg/ml L-arginine;
(d) 0.62 mg/ml calcium chloride; and
(e) 0.56 mg/ml polysorbate 80. In some embodiments the composition comprises L-arginine-HCl. In some embodiments, the composition comprises calcium chloride dihydrate.

In some embodiments, the pharmaceutical composition has a pH of about 6.5 to about 7.5. In some embodiments, the pharmaceutical composition has a pH of about 7.0. In some embodiments, the pharmaceutical composition has a pH of about 6.8.

In some embodiments, the pharmaceutical composition has a pH of about 6.5. In some embodiments, the pharmaceutical composition has a pH of about 6.6. In some embodiments, the pharmaceutical composition has a pH of about 6.7. In some embodiments, the pharmaceutical composition has a pH of about 6.8. In some embodiments, the pharmaceutical composition has a pH of about 6.9. In some embodiments, the pharmaceutical composition has a pH of about 7.0. In some embodiments, the pharmaceutical composition has a pH of about 7.1. In some embodiments, the pharmaceutical composition has a pH of about 7.2. In some embodiments, the pharmaceutical composition has a pH of about 7.3. In some embodiments, the pharmaceutical composition has a pH of about 7.4. In some embodiments, the pharmaceutical composition has a pH of about 7.5.

In some embodiments, the pharmaceutical composition has a pH of 6.5. In some embodiments, the pharmaceutical composition has a pH of 6.6. In some embodiments, the pharmaceutical composition has a pH of 6.7. In some embodiments, the pharmaceutical composition has a pH of 6.8. In some embodiments, the pharmaceutical composition has a pH of 6.9. In some embodiments, the pharmaceutical composition has a pH of 7.0. In some embodiments, the pharmaceutical composition has a pH of 7.1. In some embodiments, the pharmaceutical composition has a pH of 7.2. In some embodiments, the pharmaceutical composition has a pH of 7.3. In some embodiments, the pharmaceutical composition has a pH of 7.4. In some embodiments, the pharmaceutical composition has a pH of 7.5.

In some embodiments, a volume of 3.367 mL of the pre-lyophilization solution is added to a container or vial. In some embodiments, the pre-lyophilized solution is subjected to lyophilization, resulting in a lyophilized pharmaceutical composition.

In some embodiments, the lyophilized pharmaceutical composition comprises:
(a) about 30 mg to about 135 mg sucrose;
(b) about 2.5 mg to about 7.5 mg histidine;
(c) about 140 mg to about 200 mg arginine;
(d) about 1.5 mg to about 5 mg calcium chloride; and
(e) about 1 mg to about 5 mg polysorbate 20 or polysorbate 80.

In some embodiments, the lyophilized pharmaceutical composition comprises:
(a) about 30 mg to about 135 mg sucrose;
(b) about 2.5 mg to about 7.5 mg L-histidine;
(c) about 140 mg to about 200 mg L-arginine;
(d) about 1.5 mg to about 5 mg calcium chloride; and
(e) about 1 mg to about 5 mg polysorbate 80. In some embodiments the composition comprises L-arginine-HCl. In some embodiments, the composition comprises calcium chloride dihydrate.

In some embodiments, the lyophilized pharmaceutical composition comprises:
(a) 30 mg to 135 mg sucrose;
(b) 2.5 mg to 7.5 mg histidine;
(c) 140 mg to 200 mg arginine;
(d) 1.5 mg to 5 mg calcium chloride; and
(e) 1 mg to 5 mg polysorbate 20 or polysorbate 80.

In some embodiments, the lyophilized pharmaceutical composition comprises:
(a) 30 mg to 135 mg sucrose;
(b) 2.5 mg to 7.5 mg L-histidine;
(c) 140 mg to 200 mg L-arginine-HCl;
(d) 1.5 mg to 5 mg calcium chloride; and
(e) 1 mg to 5 mg polysorbate 80. In some embodiments, the composition comprises calcium chloride dihydrate.

In some embodiments, the lyophilized pharmaceutical composition comprises:
(a) 30 mg to 135 mg sucrose;
(b) 2.5 mg to 7.5 mg L-histidine;
(c) 140 mg to 200 mg L-arginine;
(d) 1.5 mg to 5 mg calcium chloride; and
(e) 1 mg to 5 mg polysorbate 80. In some embodiments the composition comprises L-arginine-HCl. In some embodiments, the composition comprises calcium chloride dihydrate.

In some embodiments, the lyophilized pharmaceutical composition comprises:
(a) about 67.34 mg sucrose;
(b) about 5.2 mg L-histidine;
(c) about 177.3 mg L-arginine-HCl;
(d) about 2.5 mg calcium chloride; and
(e) about 1.7 mg polysorbate 20 or polysorbate 80.

In some embodiments, the lyophilized pharmaceutical composition comprises:
(a) about 67.34 mg sucrose;
(b) about 5.2 mg L-histidine;
(c) about 146.6 mg L-arginine;
(d) about 2.5 mg calcium chloride; and
(e) about 1.7 mg polysorbate 20 or polysorbate 80.

In some embodiments, the lyophilized pharmaceutical composition comprises:
(a) 67.34 mg sucrose;
(b) 5.2 mg L-histidine;
(c) 177.3 mg L-arginine-HCl;
(d) 2.5 mg calcium chloride; and
(e) 1.7 mg polysorbate 80.

In some embodiments, the lyophilized pharmaceutical composition comprises:
(a) 67.34 mg sucrose;
(b) 5.2 mg L-histidine;
(c) 146.6 mg L-arginine;
(d) 2.5 mg calcium chloride; and
(e) 1.7 mg polysorbate 80.

In some embodiments, the lyophilized pharmaceutical composition comprises:
(a) 67.34 mg sucrose;
(b) 5.23 mg L-histidine;
(c) 177.32 mg L-arginine-HCl;
(d) 2.47 mg calcium chloride dihydrate; and
(e) 1.68 mg polysorbate 80. In some embodiments, the lyophilized pharmaceutical composition comprises:
(a) 67.34 mg sucrose;
(b) 5.23 mg L-histidine;
(c) 177.32 mg L-arginine-HCl;
(d) 1.87 mg calcium chloride; and
(e) 1.68 mg polysorbate 80.

In some embodiments, the lyophilized pharmaceutical composition comprises:
(a) 67.34 mg sucrose;
(b) 5.23 mg L-histidine;
(c) 146.63 mg L-arginine;
(d) 2.47 mg calcium chloride dihydrate; and
(e) 1.68 mg polysorbate 80.

In some embodiments, the lyophilized pharmaceutical composition comprises:
(a) 67.34 mg sucrose;
(b) 5.23 mg L-histidine;
(c) 146.63 mg L-arginine;
(d) 1.87 mg calcium chloride; and
(e) 1.68 mg polysorbate 80.

In some embodiments, the lyophilized pharmaceutical composition comprises:
(a) 30 mg to 135 mg sucrose;
(b) 5.2 mg L-histidine;
(c) 177.3 mg L-arginine-HCl;
(d) 2.5 mg calcium chloride dihydrate; and
(e) 1.7 mg polysorbate 80.

In some embodiments, the lyophilized pharmaceutical composition comprises:
(a) 30 mg to 135 mg sucrose;
(b) 5.2 mg L-histidine;
(c) 177.3 mg L-arginine-HCl;
(d) 1.9 mg calcium chloride; and
(e) 1.7 mg polysorbate 80.

In some embodiments, the lyophilized pharmaceutical composition comprises:
(a) 30 mg to 135 mg sucrose;
(b) 5.2 mg L-histidine;
(c) 146.6 mg L-arginine;
(d) 2.5 mg calcium chloride dihydrate; and
(e) 1.7 mg polysorbate 80.

In some embodiments, the lyophilized pharmaceutical composition comprises:
(a) 30 mg to 135 mg sucrose;
(b) 5.2 mg L-histidine;
(c) 146.6 mg L-arginine;
(d) 1.9 mg calcium chloride; and
(e) 1.7 mg polysorbate 80.

In some embodiments, the lyophilized pharmaceutical composition comprises:
(a) 67.34 mg sucrose;
(b) 2.5 mg to 7.5 mg L-histidine;
(c) 177.3 mg L-arginine-HCl;
(d) 2.5 mg calcium chloride dihydrate; and
(e) 1.7 mg polysorbate 80.

In some embodiments, the lyophilized pharmaceutical composition comprises:
(a) 67.34 mg sucrose;
(b) 2.5 mg to 7.5 mg L-histidine;
(c) 177.3 mg L-arginine-HCl;
(d) 1.9 mg calcium chloride; and
(e) 1.7 mg polysorbate 80.

In some embodiments, the lyophilized pharmaceutical composition comprises:
(a) 67.34 mg sucrose;
(b) 2.5 mg to 7.5 mg L-histidine;
(c) 146.6 mg L-arginine;
(d) 2.5 mg calcium chloride dihydrate; and
(e) 1.7 mg polysorbate 80.

In some embodiments, the lyophilized pharmaceutical composition comprises:
(a) 67.34 mg sucrose;
(b) 2.5 mg to 7.5 mg L-histidine;
(c) 146.6 mg L-arginine;
(d) 1.9 mg calcium chloride; and
(e) 1.7 mg polysorbate 80.

In some embodiments, the lyophilized pharmaceutical composition comprises:
(a) 67.34 mg sucrose;
(b) 5.23 mg L-histidine;
(c) 140 mg to 200 mg L-arginine;
(d) 1.5 mg to 5 mg calcium chloride; and
(e) 1.7 mg polysorbate 80. In some embodiments, the composition comprises 140 mg to 200 mg L-arginine-HCl.

In some embodiments, the lyophilized pharmaceutical composition comprises:
(a) 67.34 mg sucrose;
(b) 5.23 mg L-histidine;
(c) 177.32 mg L-arginine-HCl;
(d) 1.5 mg to 5 mg calcium chloride; and
(e) 1.7 mg polysorbate 80.

In some embodiments, the lyophilized pharmaceutical composition comprises:
(a) 67.34 mg sucrose;
(b) 5.23 mg L-histidine;
(c) 146.63 mg L-arginine;
(d) 1.5 mg to 5 mg calcium chloride; and
(e) 1.7 mg polysorbate 80.

In some embodiments, the lyophilized pharmaceutical composition comprises:
(a) 67.34 mg sucrose;
(b) 5.23 mg L-histidine;
(c) 177.32 mg L-arginine-HCl;
(d) 2.47 mg calcium chloride dihydrate; and
(e) 1 mg to 5 mg polysorbate 80.

In some embodiments, the lyophilized pharmaceutical composition comprises:
(a) 67.34 mg sucrose;
(b) 5.23 mg L-histidine;
(c) 177.32 mg L-arginine-HCl;
(d) 1.87 mg calcium chloride; and
(e) 1 mg to 5 mg polysorbate 80.

In some embodiments, the lyophilized pharmaceutical composition comprises:
(a) 67.34 mg sucrose;
(b) 5.23 mg L-histidine;
(c) 146.63 mg L-arginine;
(d) 2.47 mg calcium chloride dihydrate; and
(e) 1 mg to 5 mg polysorbate 80.

In some embodiments, the lyophilized pharmaceutical composition comprises:
(a) 67.34 mg sucrose;
(b) 5.23 mg L-histidine;
(c) 146.63 mg L-arginine;
(d) 1.87 mg calcium chloride; and
(e) 1 mg to 5 mg polysorbate 80.

In some embodiments, the lyophilized pharmaceutical composition comprises:
(a) about 160 mg to about 200 mg sucrose;
(b) about 2.5 mg to about 7.5 mg histidine;
(c) about 140 mg to about 200 mg arginine;
(d) about 1.5 mg to about 5 mg calcium chloride; and
(e) about 1 mg to about 5 mg polysorbate 20 or polysorbate 80.

In some embodiments, the lyophilized pharmaceutical composition comprises:
(a) about 160 mg to about 200 mg sucrose;
(b) about 2.5 mg to about 7.5 mg L-histidine;
(c) about 140 mg to about 200 mg L-arginine;
(d) about 1.5 mg to about 5 mg calcium chloride; and
(e) about 1 mg to about 5 mg polysorbate 80. In some embodiments the composition comprises L-arginine-HCl. In some embodiments, the composition comprises calcium chloride dihydrate.

In some embodiments, the lyophilized pharmaceutical composition comprises:
(a) 160 mg to 200 mg sucrose;
(b) 2.5 mg to 7.5 mg histidine;
(c) 140 mg to 200 mg arginine;
(d) 1.5 mg to 5 mg calcium chloride; and
(e) 1 mg to 5 mg polysorbate 20 or polysorbate 80.

In some embodiments, the lyophilized pharmaceutical composition comprises:
(a) 160 mg to 200 mg sucrose;
(b) 2.5 mg to 7.5 mg L-histidine;
(c) 140 mg to 200 mg L-arginine-HCl;
(d) 1.5 mg to 5 mg calcium chloride; and
(e) 1 mg to 5 mg polysorbate 80. In some embodiments, the composition comprises calcium chloride dihydrate.

In some embodiments, the lyophilized pharmaceutical composition comprises:
(a) 160 mg to 200 mg sucrose;
(b) 2.5 mg to 7.5 mg L-histidine;
(c) 140 mg to 200 mg L-arginine;
(d) 1.5 mg to 5 mg calcium chloride; and
(e) 1 mg to 5 mg polysorbate 80. In some embodiments the composition comprises L-arginine-HCl. In some embodiments, the composition comprises calcium chloride dihydrate.

In some embodiments, the lyophilized pharmaceutical composition comprises:
(a) about 168.3 mg sucrose;
(b) about 5.2 mg L-histidine;
(c) about 177.3 mg L-arginine-HCl;
(d) about 2.5 mg calcium chloride; and
(e) about 1.7 mg polysorbate 20 or polysorbate 80.

In some embodiments, the lyophilized pharmaceutical composition comprises:
(a) about 168.3 mg sucrose;
(b) about 5.2 mg L-histidine;
(c) about 146.6 mg L-arginine;
(d) about 2.5 mg calcium chloride; and
(e) about 1.7 mg polysorbate 20 or polysorbate 80.

In some embodiments, the lyophilized pharmaceutical composition comprises:
(a) 168.3 mg sucrose;
(b) 5.2 mg L-histidine;
(c) 177.3 mg L-arginine-HCl;
(d) 2.5 mg calcium chloride; and
(e) 1.7 mg polysorbate 80.

In some embodiments, the lyophilized pharmaceutical composition comprises:
(a) 168.3 mg sucrose;
(b) 5.2 mg L-histidine;
(c) 146.6 mg L-arginine;
(d) 2.5 mg calcium chloride; and
(e) 1.7 mg polysorbate 80.

In some embodiments, the lyophilized pharmaceutical composition comprises:
(a) 168.35 mg sucrose;
(b) 5.23 mg L-histidine;
(c) 177.32 mg L-arginine-HCl;
(d) 2.47 mg calcium chloride dihydrate; and
(e) 1.68 mg polysorbate 80.

In some embodiments, the lyophilized pharmaceutical composition comprises:
(a) 168.35 mg sucrose;
(b) 5.23 mg L-histidine;
(c) 177.32 mg L-arginine-HCl;
(d) 1.87 mg calcium chloride; and
(e) 1.68 mg polysorbate 80.

In some embodiments, the lyophilized pharmaceutical composition comprises:
(a) 168.35 mg sucrose;
(b) 5.23 mg L-histidine;
(c) 146.63 mg L-arginine;
(d) 2.47 mg calcium chloride dihydrate; and
(e) 1.68 mg polysorbate 80.

In some embodiments, the lyophilized pharmaceutical composition comprises:
(a) 168.35 mg sucrose;
(b) 5.23 mg L-histidine;
(c) 146.63 mg L-arginine;
(d) 1.87 mg calcium chloride; and
(e) 1.68 mg polysorbate 80.

In some embodiments, the lyophilized pharmaceutical composition comprises:
(a) 160 mg to 200 mg sucrose;
(b) 5.2 mg L-histidine;
(c) 177.3 mg L-arginine-HCl;
(d) 2.5 mg calcium chloride dihydrate; and
(e) 1.7 mg polysorbate 80.

In some embodiments, the lyophilized pharmaceutical composition comprises:
(a) 160 mg to 200 mg sucrose;
(b) 5.2 mg L-histidine;
(c) 177.3 mg L-arginine-HCl;
(d) 1.9 mg calcium chloride; and
(e) 1.7 mg polysorbate 80.

In some embodiments, the lyophilized pharmaceutical composition comprises:
(a) 160 mg to 200 mg sucrose;
(b) 5.2 mg L-histidine;
(c) 146.6 mg L-arginine;
(d) 2.5 mg calcium chloride dihydrate; and
(e) 1.7 mg polysorbate 80.

In some embodiments, the lyophilized pharmaceutical composition comprises:
(a) 160 mg to 200 mg sucrose;
(b) 5.2 mg L-histidine;
(c) 146.6 mg L-arginine;
(d) 1.9 mg calcium chloride; and
(e) 1.7 mg polysorbate 80.

In some embodiments, the lyophilized pharmaceutical composition comprises:
(a) 168.3 mg sucrose;
(b) 2.5 mg to 7.5 mg L-histidine;
(c) 177.3 mg L-arginine-HCl;
(d) 2.5 mg calcium chloride dihydrate; and
(e) 1.7 mg polysorbate 80.

In some embodiments, the lyophilized pharmaceutical composition comprises:
(a) 168.3 mg sucrose;
(b) 2.5 mg to 7.5 mg L-histidine;
(c) 177.3 mg L-arginine-HCl;
(d) 1.9 mg calcium chloride; and
(e) 1.7 mg polysorbate 80.

In some embodiments, the lyophilized pharmaceutical composition comprises:
(a) 168.3 mg sucrose;
(b) 2.5 mg to 7.5 mg L-histidine;
(c) 146.6 mg L-arginine;
(d) 2.5 mg calcium chloride dihydrate; and
(e) 1.7 mg polysorbate 80.

In some embodiments, the lyophilized pharmaceutical composition comprises:
(a) 168.3 mg sucrose;
(b) 2.5 mg to 7.5 mg L-histidine;
(c) 146.6 mg L-arginine;
(d) 1.9 mg calcium chloride; and
(e) 1.7 mg polysorbate 80.

In some embodiments, the lyophilized pharmaceutical composition comprises:
(a) 168.35 mg sucrose;
(b) 5.23 mg L-histidine;
(c) 140 mg to 200 mg L-arginine;
(d) 1.5 mg to 5 mg calcium chloride; and
(e) 1.7 mg polysorbate 80. In some embodiments, the composition comprises 140 mg to 200 mg L-arginine-HCl.

In some embodiments, the lyophilized pharmaceutical composition comprises:
(a) 168.35 mg sucrose;
(b) 5.23 mg L-histidine;
(c) 177.32 mg L-arginine-HCl;
(d) 1.5 mg to 5 mg calcium chloride; and
(e) 1.7 mg polysorbate 80.

In some embodiments, the lyophilized pharmaceutical composition comprises:
(a) 168.35 mg sucrose;
(b) 5.23 mg L-histidine;
(c) 146.63 mg L-arginine;
(d) 1.5 mg to 5 mg calcium chloride; and
(e) 1.7 mg polysorbate 80.

In some embodiments, the lyophilized pharmaceutical composition comprises:
(a) 168.35 mg sucrose;
(b) 5.23 mg L-histidine;
(c) 177.32 mg L-arginine-HCl;
(d) 2.47 mg calcium chloride dihydrate; and
(e) 1 mg to 5 mg polysorbate 80.

In some embodiments, the lyophilized pharmaceutical composition comprises:
(a) 168.35 mg sucrose;
(b) 5.23 mg L-histidine;
(c) 177.32 mg L-arginine-HCl;
(d) 1.87 mg calcium chloride; and
(e) 1 mg to 5 mg polysorbate 80.

In some embodiments, the lyophilized pharmaceutical composition comprises:
(a) 168.35 mg sucrose;
(b) 5.23 mg L-histidine;
(c) 146.63 mg L-arginine;
(d) 2.47 mg calcium chloride dihydrate; and
(e) 1 mg to 5 mg polysorbate 80.

In some embodiments, the lyophilized pharmaceutical composition comprises:
(a) 168.35 mg sucrose;
(b) 5.23 mg L-histidine;
(c) 146.63 mg L-arginine;
(d) 1.87 mg calcium chloride; and
(e) 1 mg to 5 mg polysorbate 80.

In some embodiments, the lyophilized pharmaceutical composition has a moisture content of less than 2%. In some embodiments, the lyophilized pharmaceutical composition has a moisture content of less than 1.8%. In some embodiments, the lyophilized pharmaceutical composition has a moisture content of less than 1.6%.

In some embodiments, the lyophilized pharmaceutical composition is in a lyophilized cake. In some embodiments, the lyophilized cake is white. In some embodiments, the lyophilized cake is less than Y4 in the European Pharmacopoeia color scale. See Degree of Coloration of Liquids (Method 2.2.2), European Pharmacopoeia, 10^{th} Ed. (2021).

In some embodiments, the lyophilized pharmaceutical composition and sterile water are combined to produce an injectable solution. In some embodiments, the lyophilized pharmaceutical composition is combined with about 2 mL to about 5 mL of sterile water. In some embodiments, the lyophilized pharmaceutical composition is combined with about 3 mL of sterile water. In some embodiments, the lyophilized pharmaceutical composition is combined with 3 mL of sterile water. In some embodiments, the sterile water is USP grade sterile water. In some embodiments, the sterile water is USP grade sterile water for injection. In some embodiments, the sterile water is pyrogen-free or nonpyrogenic. In some embodiments, the sterile water does not contain a bacteriostatic or antimicrobial agent. In some embodiments, the sterile water contains a bacteriostatic or antimicrobial agent. In some embodiments, the sterile water is sterilized using a filter. In some embodiments, the sterile water is sterilized using a 0.1 μm filter. In some embodiments, the sterile water is distilled water. In some embodiments, the sterile water is sterile, nonpyrogenic, distilled water, hypotonic, with an osmolarity of zero mOsmol/L, and does not contain a bacteriostatic or antimicrobial agent.

In some embodiments, when the lyophilized pharmaceutical composition and the sterile water are combined, then the resulting solution comprises:
(a) 10 mg/mL to 40 mg/mL sucrose;
(b) 1.5 mg/mL to 2.0 mg/mL L-histidine;
(c) 50 mg/mL to 70 mg/mL L-arginine;
(d) 0.7 mg/mL to 0.9 mg/mL calcium chloride dihydrate; and
(e) 0.4 mg/mL to 0.7 mg/mL polysorbate 80. In some embodiments the composition comprises L-arginine-HCl. In some embodiments, when the lyophilized pharmaceutical composition and the sterile water are combined, then the resulting solution comprises:
(a) 10 mg/mL to 40 mg/mL sucrose;
(b) 1.5 mg/mL to 2.0 mg/mL L-histidine;
(c) 50 mg/mL to 70 mg/mL L-arginine;
(d) 0.5 mg/mL to 0.8 mg/mL calcium chloride; and
(e) 0.4 mg/mL to 0.7 mg/mL polysorbate 80. In some embodiments the composition comprises L-arginine-HCl. In some embodiments, the composition comprises calcium chloride dihydrate

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 59.11 mg/ml L-arginine-HCl;
(d) about 0.82 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 59.11 mg/ml L-arginine-HCl;
(d) about 0.62 mg/ml calcium chloride; and
(e) about 0.56 mg/ml polysorbate 80. In some embodiments, the composition comprises calcium chloride dihydrate.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 48.88 mg/ml L-arginine;
(d) about 0.82 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 48.88 mg/ml L-arginine;
(d) about 0.62 mg/ml calcium chloride; and
(e) about 0.56 mg/ml polysorbate 80. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 10 mg/mL to about 40 mg/ml sucrose;
(b) about 1.74 mg/ml L-histidine;
(c) about 59.11 mg/ml L-arginine-HCl;
(d) about 0.82 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 10 mg/mL to about 40 mg/ml sucrose;
(b) about 1.74 mg/ml L-histidine;
(c) about 59.11 mg/ml L-arginine-HCl;
(d) about 0.62 mg/ml calcium chloride; and
(e) about 0.56 mg/ml polysorbate 80. In some embodiments, the composition comprises calcium chloride dihydrate.

In some embodiments, when the lyophilized pharmaceutical composition and the sterile water are combined, then the resulting solution comprises:
(a) about 10 mg/mL to about 40 mg/ml sucrose;
(b) about 1.74 mg/ml L-histidine;
(c) about 44.88 mg/ml L-arginine;
(d) about 0.82 mg/ml calcium chloride dihydrate; and
(e) about 0.56 mg/ml polysorbate 80

In some embodiments, when the lyophilized pharmaceutical composition and the sterile water are combined, then the resulting solution comprises:
(a) about 10 mg/mL to about 40 mg/ml sucrose;
(b) about 1.74 mg/ml L-histidine;
(c) about 44.88 mg/ml L-arginine;
(d) about 0.62 mg/ml calcium chloride; and
(e) about 0.56 mg/ml polysorbate 80. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 22.45 mg/ml sucrose;
(b) about 1.5 mg/ml to about 2.0 mg/mL L-histidine;
(c) about 59.11 mg/ml L-arginine-HCl;
(d) about 0.82 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 22.45 mg/ml sucrose;
(b) about 1.5 mg/mL to about 2.0 mg/mL L-histidine;
(c) about 59.11 mg/ml L-arginine-HCl;
(d) about 0.62 mg/ml calcium chloride; and
(e) about 0.56 mg/ml polysorbate 80. In some embodiments, the composition comprises calcium chloride dihydrate.

In some embodiments, when the lyophilized pharmaceutical composition and the sterile water are combined, then the resulting solution comprises:
(a) about 22.45 mg/ml sucrose;
(b) about 1.5 mg/mL to about 2.0 mg/mL L-histidine;
(c) about 48.88 mg/ml L-arginine;
(d) about 0.82 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 22.45 mg/ml sucrose;
(b) about 1.5 mg/mL to about 2.0 mg/mL L-histidine;
(c) about 48.88 mg/ml L-arginine;
(d) about 0.62 mg/ml calcium chloride; and
(e) about 0.56 mg/ml polysorbate 80. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 50 mg/mL to about 70 mg/mL L-arginine-HCl;
(d) about 0.62 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 50 mg/mL to about 70 mg/mL L-arginine-HCl;
(d) about 0.62 mg/ml calcium chloride; and
(e) about 0.56 mg/ml polysorbate 80. In some embodiments, the composition comprises calcium chloride dihydrate.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 40 mg/mL to about 60 mg/mL L-arginine;
(d) about 0.82 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 40 mg/mL to about 60 mg/mL L-arginine;
(d) about 0.62 mg/ml calcium chloride; and
(e) about 0.56 mg/ml polysorbate 80. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 59.11 mg/ml L-arginine-HCl;
(d) about 0.7 mg/mL to about 0.9 mg/mL calcium chloride dihydrate; and
(e) about 0.56 mg/ml polysorbate 80.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 59.11 mg/ml L-arginine-HCl;
(d) about 0.5 mg/mL to about 0.9 mg/mL calcium chloride; and
(e) about 0.56 mg/ml polysorbate 80. In some embodiments, the composition comprises calcium chloride dihydrate.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 48.88 mg/ml L-arginine;
(d) about 0.7 mg/mL to about 0.9 mg/mL calcium chloride dihydrate; and
(e) about 0.56 mg/ml polysorbate 80. In some embodiments the composition comprises L-arginine-HCl.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 48.88 mg/ml L-arginine;
(d) about 0.5 mg/mL to about 0.7 mg/mL calcium chloride; and
(e) about 0.56 mg/ml polysorbate 80. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 59.11 mg/ml L-arginine-HCl;
(d) about 0.82 mg/ml calcium chloride dihydrate; and
(e) about 0.4 mg/mL to about 0.7 mg/mL polysorbate 80.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 59.11 mg/ml L-arginine-HCl;
(d) about 0.62 mg/ml calcium chloride; and
(e) about 0.4 mg/mL to about 0.7 mg/mL polysorbate 80. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 48.88 mg/ml L-arginine;
(d) about 0.82 mg/ml calcium chloride dihydrate; and
(e) about 0.4 mg/mL to about 0.7 mg/mL polysorbate 80.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 48.88 mg/ml L-arginine;
(d) about 0.62 mg/ml calcium chloride; and
(e) about 0.4 mg/mL to about 0.7 mg/mL polysorbate 80. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 59.11 mg/ml L-arginine-HCl;
(d) about 0.82 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 59.11 mg/ml L-arginine-HCl;
(d) about 0.62 mg/ml calcium chloride; and
(e) about 0.56 mg/ml polysorbate 80.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 48.88 mg/ml L-arginine;
(d) about 0.82 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 22.45 mg/ml sucrose;
(b) about 1.74 mg/ml L-histidine;
(c) about 48.88 mg/ml L-arginine;
(d) about 0.62 mg/ml calcium chloride; and
(e) about 0.56 mg/ml polysorbate 80.

In some embodiments, when the lyophilized pharmaceutical composition and the sterile water are combined, then the resulting solution comprises:
(a) about 67.34 mg sucrose;
(b) about 5.23 mg L-histidine;
(c) about 177.32 mg L-arginine-HCl;
(d) about 2.47 mg calcium chloride; and
(e) about 1.68 mg polysorbate 80 in 3 mL of sterile water.

In some embodiments, when the lyophilized pharmaceutical composition and the sterile water are combined, then the resulting solution comprises:
(a) about 67.34 mg sucrose;
(b) about 5.23 mg L-histidine;
(c) about 146.63 mg L-arginine;
(d) about 2.47 mg calcium chloride dihydrate; and
(e) about 1.68 mg polysorbate 80 in 3 mL of sterile water.

In some embodiments, when the lyophilized pharmaceutical composition and the sterile water are combined, then the resulting solution comprises:
(a) about 67.34 mg sucrose;
(b) about 5.23 mg L-histidine;
(c) about 146.63 mg L-arginine;
(d) about 1.87 mg calcium chloride; and
(e) about 1.68 mg polysorbate 80 in 3 mL of sterile water.

In some embodiments, when the lyophilized pharmaceutical composition and the sterile water are combined, then the resulting solution comprises:
(a) 67.34 mg sucrose;
(b) 5.23 mg L-histidine;
(c) 177.32 mg L-arginine-HCl;
(d) 2.47 mg calcium chloride dihydrate; and
(e) 1.68 mg polysorbate 80 in 3 mL of sterile water.

In some embodiments, when the lyophilized pharmaceutical composition and the sterile water are combined, then the resulting solution comprises:
(a) 67.34 mg sucrose;
(b) 5.23 mg L-histidine;
(c) 177.32 mg L-arginine-HCl;
(d) 1.87 mg calcium chloride; and
(e) 1.68 mg polysorbate 80 in 3 mL of sterile water.

In some embodiments, when the lyophilized pharmaceutical composition and the sterile water are combined, then the resulting solution comprises:
(a) 67.34 mg sucrose;
(b) 5.23 mg L-histidine;
(c) 146.63 mg L-arginine;
(d) 2.47 mg calcium chloride dihydrate; and
(e) 1.68 mg polysorbate 80 in 3 mL of sterile water.

In some embodiments, when the lyophilized pharmaceutical composition and the sterile water are combined, then the resulting solution comprises:
(a) 67.34 mg sucrose;
(b) 5.23 mg L-histidine;
(c) 146.63 mg L-arginine;
(d) 1.87 mg calcium chloride; and
(e) 1.68 mg polysorbate 80 in 3 mL of sterile water.

In some embodiments, when the lyophilized pharmaceutical composition and the sterile water are combined, then the resulting solution comprises:
(a) 45 mg/mL to 60 mg/mL sucrose;
(b) 1.5 mg/mL to 2.0 mg/mL L-histidine;
(c) 50 mg/mL to 70 mg/mL L-arginine;
(d) 0.7 mg/mL to 0.9 mg/mL calcium chloride dihydrate; and
(e) 0.4 mg/mL to 0.7 mg/mL polysorbate 80. In some embodiments the composition comprises L-arginine-HCl. In some embodiments, when the lyophilized pharmaceutical composition and the sterile water are combined, then the resulting solution comprises:
(a) 45 mg/mL to 60 mg/mL sucrose;
(b) 1.5 mg/mL to 2.0 mg/mL L-histidine;
(c) 50 mg/mL to 70 mg/mL L-arginine;
(d) 0.5 mg/mL to 0.8 mg/mL calcium chloride; and
(e) 0.4 mg/mL to 0.7 mg/mL polysorbate 80. In some embodiments the composition comprises L-arginine-HCl. In some embodiments, the composition comprises calcium chloride dihydrate

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 59.11 mg/ml L-arginine-HCl;
(d) about 0.82 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 59.11 mg/ml L-arginine-HCl;
(d) about 0.62 mg/ml calcium chloride; and
(e) about 0.56 mg/ml polysorbate 80. In some embodiments, the composition comprises calcium chloride dihydrate.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 48.88 mg/ml L-arginine;
(d) about 0.82 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 48.88 mg/ml L-arginine;
(d) about 0.62 mg/ml calcium chloride; and
(e) about 0.56 mg/ml polysorbate 80. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 45 mg/mL to about 60 mg/ml sucrose;
(b) about 1.74 mg/ml L-histidine;
(c) about 59.11 mg/ml L-arginine-HCl;
(d) about 0.82 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 45 mg/mL to about 60 mg/ml sucrose;
(b) about 1.74 mg/ml L-histidine;
(c) about 59.11 mg/ml L-arginine-HCl;
(d) about 0.62 mg/ml calcium chloride; and
(e) about 0.56 mg/ml polysorbate 80. In some embodiments, the composition comprises calcium chloride dihydrate.

In some embodiments, when the lyophilized pharmaceutical composition and the sterile water are combined, then the resulting solution comprises:
(a) about 45 mg/mL to about 60 mg/ml sucrose;
(b) about 1.74 mg/ml L-histidine;
(c) about 44.88 mg/ml L-arginine;
(d) about 0.82 mg/ml calcium chloride dihydrate; and
(e) about 0.56 mg/ml polysorbate 80

In some embodiments, when the lyophilized pharmaceutical composition and the sterile water are combined, then the resulting solution comprises:
(a) about 45 mg/mL to about 60 mg/ml sucrose;
(b) about 1.74 mg/ml L-histidine;
(c) about 44.88 mg/ml L-arginine;
(d) about 0.62 mg/ml calcium chloride; and
(e) about 0.56 mg/ml polysorbate 80. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 56.12 mg/ml sucrose;
(b) about 1.5 mg/mL to about 2.0 mg/mL L-histidine;
(c) about 59.11 mg/ml L-arginine-HCl;
(d) about 0.82 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 56.12 mg/ml sucrose;
(b) about 1.5 mg/mL to about 2.0 mg/mL L-histidine;
(c) about 59.11 mg/ml L-arginine-HCl;
(d) about 0.62 mg/ml calcium chloride; and
(e) about 0.56 mg/ml polysorbate 80. In some embodiments, the composition comprises calcium chloride dihydrate.

In some embodiments, when the lyophilized pharmaceutical composition and the sterile water are combined, then the resulting solution comprises:
(a) about 56.12 mg/ml sucrose;
(b) about 1.5 mg/mL to about 2.0 mg/mL L-histidine;
(c) about 48.88 mg/ml L-arginine;
(d) about 0.82 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 56.12 mg/ml sucrose;
(b) about 1.5 mg/mL to about 2.0 mg/mL L-histidine;
(c) about 48.88 mg/ml L-arginine;
(d) about 0.62 mg/ml calcium chloride; and
(e) about 0.56 mg/ml polysorbate 80. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 50 mg/mL to about 70 mg/mL L-arginine-HCl;
(d) about 0.62 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 50 mg/mL to about 70 mg/mL L-arginine-HCl;
(d) about 0.62 mg/ml calcium chloride; and
(e) about 0.56 mg/ml polysorbate 80. In some embodiments, the composition comprises calcium chloride dihydrate.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 40 mg/mL to about 60 mg/mL L-arginine;
(d) about 0.82 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 40 mg/mL to about 60 mg/mL L-arginine;
(d) about 0.62 mg/ml calcium chloride; and
(e) about 0.56 mg/ml polysorbate 80. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 59.11 mg/ml L-arginine-HCl;
(d) about 0.7 mg/mL to about 0.9 mg/mL calcium chloride dihydrate; and
(e) about 0.56 mg/ml polysorbate 80.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 59.11 mg/ml L-arginine-HCl;
(d) about 0.5 mg/mL to about 0.9 mg/mL calcium chloride; and
(e) about 0.56 mg/ml polysorbate 80. In some embodiments, the composition comprises calcium chloride dihydrate.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 48.88 mg/ml L-arginine;
(d) about 0.7 mg/mL to about 0.9 mg/mL calcium chloride dihydrate; and
(e) about 0.56 mg/ml polysorbate 80. In some embodiments the composition comprises L-arginine-HCl.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 48.88 mg/ml L-arginine;
(d) about 0.5 mg/mL to about 0.7 mg/mL calcium chloride; and
(e) about 0.56 mg/ml polysorbate 80. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 59.11 mg/ml L-arginine-HCl;
(d) about 0.82 mg/ml calcium chloride dihydrate; and
(e) about 0.4 mg/mL to about 0.7 mg/mL polysorbate 80.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 59.11 mg/ml L-arginine-HCl;
(d) about 0.62 mg/ml calcium chloride; and
(e) about 0.4 mg/mL to about 0.7 mg/mL polysorbate 80. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 48.88 mg/ml L-arginine;
(d) about 0.82 mg/ml calcium chloride dihydrate; and
(e) about 0.4 mg/mL to about 0.7 mg/mL polysorbate 80.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 48.88 mg/ml L-arginine;
(d) about 0.62 mg/ml calcium chloride; and
(e) about 0.4 mg/mL to about 0.7 mg/mL polysorbate 80. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 59.11 mg/ml L-arginine-HCl;
(d) about 0.82 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 59.11 mg/ml L-arginine-HCl;
(d) about 0.62 mg/ml calcium chloride; and
(e) about 0.56 mg/ml polysorbate 80.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 48.88 mg/ml L-arginine;
(d) about 0.82 mg/ml calcium chloride dihydrate; and
(e) about 0.56 mg/ml polysorbate 80.

In some embodiments, when the lyophilized pharmaceutical composition and the sterile water are combined, then the resulting solution comprises:
(a) about 56.12 mg/ml sucrose;
(b) about 1.74 mg/ml L-histidine;
(c) about 48.88 mg/ml L-arginine;
(d) about 0.62 mg/ml calcium chloride; and
(e) about 0.56 mg/ml polysorbate 80.

In some embodiments, when the lyophilized pharmaceutical composition and the sterile water are combined, then the resulting solution comprises:
(a) about 168.4 mg sucrose;
(b) about 5.23 mg L-histidine;
(c) about 177.32 mg L-arginine-HCl;
(d) about 2.47 mg calcium chloride; and
(e) about 1.68 mg polysorbate 80 in 3 mL of sterile water.

In some embodiments, when the lyophilized pharmaceutical composition and the sterile water are combined, then the resulting solution comprises:
(a) about 168.4 mg sucrose;
(b) about 5.23 mg L-histidine;
(c) about 146.63 mg L-arginine;
(d) about 2.47 mg calcium chloride dihydrate; and
(e) about 1.68 mg polysorbate 80 in 3 mL of sterile water.

In some embodiments, when the lyophilized pharmaceutical composition and the sterile water are combined, then the resulting solution comprises:
(a) about 168.4 mg sucrose;
(b) about 5.23 mg L-histidine;
(c) about 146.63 mg L-arginine;
(d) about 1.87 mg calcium chloride; and
(e) about 1.68 mg polysorbate 80 in 3 mL of sterile water.

In some embodiments, when the lyophilized pharmaceutical composition and the sterile water are combined, then the resulting solution comprises:
(a) 168.35 mg sucrose;
(b) 5.23 mg L-histidine;
(c) 177.32 mg L-arginine-HCl;
(d) 2.47 mg calcium chloride dihydrate; and
(e) 1.68 mg polysorbate 80 in 3 mL of sterile water.

In some embodiments, when the lyophilized pharmaceutical composition and the sterile water are combined, then the resulting solution comprises:
(a) 168.35 mg sucrose;
(b) 5.23 mg L-histidine;
(c) 177.32 mg L-arginine-HCl;
(d) 1.87 mg calcium chloride; and
(e) 1.68 mg polysorbate 80 in 3 mL of sterile water.

In some embodiments, when the lyophilized pharmaceutical composition and the sterile water are combined, then the resulting solution comprises:
(a) 168.35 mg sucrose;
(b) 5.23 mg L-histidine;
(c) 146.63 mg L-arginine;
(d) 2.47 mg calcium chloride dihydrate; and
(e) 1.68 mg polysorbate 80 in 3 mL of sterile water.

In some embodiments, when the lyophilized pharmaceutical composition and the sterile water are combined, then the resulting solution comprises:
(a) 168.35 mg sucrose;
(b) 5.23 mg L-histidine;
(c) 146.63 mg L-arginine;
(d) 1.87 mg calcium chloride; and
(e) 1.68 mg polysorbate 80 in 3 mL of sterile water.

In some embodiments, when the lyophilized pharmaceutical composition and the sterile water are combined, then the lyophilized pharmaceutical composition is reconstituted within 7 to 12 seconds.

In some embodiments, when the lyophilized pharmaceutical composition and the sterile water are combined, then the osmolality of the resulting solution is about 525 to about 725 mOsm/kg. In some embodiments, when the lyophilized pharmaceutical composition and the sterile water are combined, then the osmolality of the resulting solution is about 600 to about 650 mOsm/kg.

In some embodiments, the pharmaceutical composition has an osmolality of about 525 mOsm/kg. In some embodiments, the pharmaceutical composition has an osmolality of about 550 mOsm/kg. In some embodiments, the pharmaceutical composition has an osmolality of about 575 mOsm/kg. In some embodiments, the pharmaceutical composition has an osmolality of about 600 mOsm/kg. In some embodiments, the pharmaceutical composition has an osmolality of about 625 mOsm/kg. In some embodiments, the pharmaceutical composition has an osmolality of about 650 mOsm/kg. In some embodiments, the pharmaceutical composition has an osmolality of about 675 mOsm/kg. In some embodiments, the pharmaceutical composition has an osmolality of about 700 mOsm/kg. In some embodiments, the pharmaceutical composition has an osmolality of about 725 mOsm/kg.

In some embodiments, when the lyophilized pharmaceutical composition and the sterile water are combined, then the pH of the resulting solution is about 6.5 to about 7.5. In some embodiments, the pharmaceutical composition has a pH of about 7.0. In some embodiments, the pharmaceutical composition has a pH of about 6.8. In some embodiments, the pharmaceutical composition has a pH of 6.8.

In some embodiments, when the lyophilized pharmaceutical composition and the sterile water are combined, then the pH of the resulting solution is about 6.5. In some embodiments, when the lyophilized pharmaceutical composition and the sterile water are combined, then the pH of the resulting solution is about 6.6. In some embodiments, when the lyophilized pharmaceutical composition and the sterile water are combined, then the pH of the resulting solution is about 6.7. In some embodiments, when the lyophilized pharmaceutical composition and the sterile water are combined, then the pH of the resulting solution is about 6.8. In some embodiments, when the lyophilized pharmaceutical composition and the sterile water are combined, then the pH of the resulting solution is about 6.9. In some embodiments, when the lyophilized pharmaceutical composition and the sterile water are combined, then the pH of the resulting solution is about 7.0. In some embodiments, when the lyophilized pharmaceutical composition and the sterile water are combined, then the pH of the resulting solution is about 7.1. In some embodiments, when the lyophilized pharmaceutical composition and the sterile water are combined, then the pH of the resulting solution is about 7.2. In some embodiments, when the lyophilized pharmaceutical composition and the sterile water are combined, then the pH of the resulting solution is about 7.3. In some embodiments, when the lyophilized pharmaceutical composition and the sterile water are combined, then the pH of the resulting solution is about 7.4. In some embodiments, when the lyophilized pharmaceutical composition and the sterile water are combined, then the pH of the resulting solution is about 7.5.

In some embodiments, when the lyophilized pharmaceutical composition and the sterile water are combined, then the protein concentration of the resulting solution is about 0.8 to about 1.2 mg/mL.

In some embodiments, when the lyophilized pharmaceutical composition and the sterile water are combined, then the protein concentration of the resulting solution is about 0.8 mg/mL. In some embodiments, when the lyophilized pharmaceutical composition and the sterile water are combined, then the protein concentration of the resulting solution is about 0.9 mg/mL. In some embodiments, when the lyophilized pharmaceutical composition and the sterile water are combined, then the protein concentration of the resulting solution is about 1.0 mg/mL. In some embodiments, when the lyophilized pharmaceutical composition and the sterile water are combined, then the protein concentration of the resulting solution is about 1.1 mg/mL. In some embodiments, when the lyophilized pharmaceutical composition and the sterile water are combined, then the protein concentration of the resulting solution is about 1.2 mg/mL.

In some embodiments, when the lyophilized pharmaceutical composition and the sterile water are combined, then the protein concentration of the resulting solution is 0.8 mg/mL. In some embodiments, when the lyophilized pharmaceutical composition and the sterile water are combined, then the protein concentration of the resulting solution is 0.9 mg/ml. In some embodiments, when the lyophilized pharmaceutical composition and the sterile water are combined, then the protein concentration of the resulting solution is 1.0 mg/mL. In some embodiments, when the lyophilized pharmaceutical composition and the sterile water are combined, then the protein concentration of the resulting solution is 1.1 mg/mL. In some embodiments, when the lyophilized pharmaceutical composition and the sterile water are combined, then the protein concentration of the resulting solution is 1.2 mg/mL.

In some embodiments, when the lyophilized pharmaceutical composition and the sterile water are combined, then the turbidity of the resulting solution is less than about 7 Nephelometric Turbidity Units.

In some embodiments, when the lyophilized pharmaceutical composition and the sterile water are combined, less than 1% of the chimeric protein is aggregated. In some embodiments, when the lyophilized pharmaceutical composition and the sterile water are combined, less than 2% of the chimeric protein is aggregated. In some embodiments, when the lyophilized pharmaceutical composition and the sterile water are combined, less than 2.5% of the chimeric protein is aggregated. In some embodiments, when the lyophilized pharmaceutical composition and the sterile water are combined, less than 3% of the chimeric protein is aggregated. In some embodiments, when the lyophilized pharmaceutical composition and the sterile water are combined, less than 3.5% of the chimeric protein is aggregated. In some embodiments, when the lyophilized pharmaceutical composition and the sterile water are combined, less than 4% of the chimeric protein is aggregated.

### V. Pharmaceutical Kits

In some embodiments, the pharmaceutical composition is provided with a second container comprising sterile water. Disclosed herein are pharmaceutical kits which comprise a first container containing the pharmaceutical composition and a second container containing sterile water.

Disclosed herein is a pharmaceutical kit comprising:
(i) a first container comprising a lyophilized pharmaceutical composition comprising
   (a) a chimeric protein comprising a first polypeptide chain which comprises a Factor VIII (“FVIII”) protein and a first immunoglobulin (“Ig”) constant region or a portion thereof, and a second polypeptide chain which comprises a von Willebrand Factor (“VWF”) protein and a second Ig constant region or a portion thereof;
   (b) sucrose;
   (c) histidine;
   (d) arginine;
   (e) calcium chloride; and
   (f) a polysorbate (such as polysorbate 20 or polysorbate 80), and
(ii) a second container comprising sterile water.

Also disclosed herein is a pharmaceutical kit comprising:
(i) a first container comprising a lyophilized pharmaceutical composition comprising
   (a) a chimeric protein comprising a first polypeptide chain which comprises a Factor VIII (“FVIII”) protein and a first immunoglobulin (“Ig”) constant region or a portion thereof, and a second polypeptide chain which comprises a von Willebrand Factor (“VWF”) protein and a second Ig constant region or a portion thereof;
   (b) about 30 mg to about 135 mg sucrose;
   (c) about 2.5 mg to about 7.5 mg histidine;
   (d) about 140 mg to about 200 mg arginine;
   (e) about 1.5 mg to about 5 mg calcium chloride; and
   (f) about 1 mg to about 5 mg of a polysorbate (such as polysorbate 20 or polysorbate 80), and
(ii) a second container comprising sterile water.

Also disclosed herein is a pharmaceutical kit comprising:
(i) a first container comprising a lyophilized pharmaceutical composition comprising
   (a) a chimeric protein comprising a first polypeptide chain which comprises a Factor VIII (“FVIII”) protein and a first immunoglobulin (“Ig”) constant region or a portion thereof, and a second polypeptide chain which comprises a von Willebrand Factor (“VWF”) protein and a second Ig constant region or a portion thereof;
   (b) about 160 mg to about 200 mg sucrose;
   (c) about 2.5 mg to about 7.5 mg histidine;
   (d) about 140 mg to about 200 mg arginine;
   (e) about 1.5 mg to about 5 mg calcium chloride; and
   (f) about 1 mg to about 5 mg of a polysorbate (such as polysorbate 20 or polysorbate 80), and
(ii) a second container comprising sterile water.

In some embodiments, the first container comprises 100 IU to 10,000 IU of the chimeric protein.

In some embodiments, the first container comprises 250 IU, 500 IU, 1000 IU, 2000 IU, 3000 IU, or 4,000 IU of the chimeric protein. In some embodiments, the first container comprises 250 IU of the chimeric protein. In some embodiments, the first container comprises 500 IU of the chimeric protein. In some embodiments, the first container comprises 1000 IU of the chimeric protein. In some embodiments, the first container comprises 2000 IU of the chimeric protein. In some embodiments, the first container comprises 3000 IU of the chimeric protein. In some embodiments, the first container comprises 4,000 IU of the chimeric protein.

In some embodiments, the second container comprises about 2 mL to about 5 mL of sterile water. In some embodiments, the second container comprises 2 mL to 5 mL of sterile water. In some embodiments, the second container comprises about 3 mL of sterile water. In some embodiments, the second container comprises 3 mL of sterile water.

In some embodiments, the second container comprises about 2 mL of sterile water. In some embodiments, the second container comprises about 2.1 mL of sterile water. In some embodiments, the second container comprises about 2.2 mL of sterile water. In some embodiments, the second container comprises about 2.3 mL of sterile water. In some embodiments, the second container comprises about 2.4 mL of sterile water. In some embodiments, the second container comprises about 2.5 mL of sterile water. In some embodiments, the second container comprises about 2.6 mL of sterile water. In some embodiments, the second container comprises about 2.7 mL of sterile water. In some embodiments, the second container comprises about 2.8 mL of sterile water. In some embodiments, the second container comprises about 2.9 mL of sterile water. In some embodiments, the second container comprises about 3 mL of sterile water. In some embodiments, the second container comprises about 3.1 mL of sterile water. In some embodiments, the second container comprises about 3.2 mL of sterile water. In some embodiments, the second container comprises about 3.3 mL of sterile water. In some embodiments, the second container comprises about 3.4 mL of sterile water. In some embodiments, the second container comprises about 3.5 mL of sterile water. In some embodiments, the second container comprises about 3.6 mL of sterile water. In some embodiments, the second container comprises about 3.7 mL of sterile water. In some embodiments, the second container comprises about 3.8 mL of sterile water. In some embodiments, the second container comprises about 3.9 mL of sterile water. In some embodiments, the second container comprises about 4 mL of sterile water. In some embodiments, the second container comprises about 4.1 mL of sterile water. In some embodiments, the second container comprises about 4.2 mL of sterile water. In some embodiments, the second container comprises about 4.3 mL of sterile water. In some embodiments, the second container comprises about 4.4 mL of sterile water. In some embodiments, the second container comprises about 4.5 mL of sterile water. In some embodiments, the second container comprises about 4.6 mL of sterile water. In some embodiments, the second container comprises about 4.7 mL of sterile water. In some embodiments, the second container comprises about 4.8 mL of sterile water. In some embodiments, the second container comprises about 4.9 mL of sterile water. In some embodiments, the second container comprises about 5 mL of sterile water.

In some embodiments, the pharmaceutical kit further comprises instructions for combining the lyophilized pharmaceutical composition and the sterile water.

In some embodiments, the first container is a glass vial comprising a rubber stopper.

In some embodiments, the second container is a syringe body. In some embodiments, the syringe body is associated with a plunger. In some embodiments, the pharmaceutical kit further comprises an adaptor to connect the glass vial to the syringe body. In some embodiments, the pharmaceutical kit further comprises infusion tubing associated with a needle to be connected to the syringe body, suitable for intravenous infusion. In some embodiments, the second container is a pre-filled syringe.

### VI. Methods and Uses of the Pharmaceutical Composition

Also disclosed herein is a use of the pharmaceutical composition or a method for treating hemophilia A in a subject in need thereof, comprising administering to the subject an effective amount of the pharmaceutical composition of the disclosure. In some embodiments, the treatment of hemophilia A comprises preventing a bleeding episode in a human subject in need thereof. In some embodiments, the treatment of hemophilia A comprises treating a bleeding episode in a human subject in need thereof. In some embodiments, the treatment of hemophilia A comprises controlling the incidence or frequency of a bleeding episode in a human subject in need thereof. In some embodiments, the treatment of hemophilia A comprises decreasing the incidence or frequency of a bleeding episode in a human subject in need thereof.

In some embodiments, the composition is used to treat a bleeding disease or condition in a subject in need thereof. The bleeding disease or condition is selected from the group consisting of a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, bleeding in the illiopsoas sheath and any combinations thereof. In still other embodiments, the subject is scheduled to undergo a surgery. In some embodiments, the treatment is prophylactic or on-demand.

In some embodiments, the use or method comprises combining the lyophilized pharmaceutical composition and the sterile water of a kit of the disclosure, and administering to the subject an effective amount of the resulting combination. In some embodiments, the subject combines the lyophilized pharmaceutical composition and the sterile water of the kit. In some embodiments, the combination is self-administered by the subject.

In some embodiments, the multiple doses comprise at least two doses, at least three doses, at least four doses, at least five doses, at least six doses, at least seven doses, at least eight doses, at least nine doses, at least ten doses, at least eleven doses, at least twelve doses, at least thirteen doses, at least fourteen doses, at least fifteen doses, at least sixteen doses, at least seventeen doses, at least eighteen doses, at least nineteen doses, at least twenty doses, or more. In some embodiments, the multiple doses are administered for at least about 1 month, at least about 2 months, at least about 3 months, at least about 4 months, at least about 5 months, at least about 6 months, at least about 12 months, at least about 18 months, at least about 2 years, at least about 3 years, at least about 4 years, at least about 5 years, at least about 10 years, at least about 15 years, at least about 20 years, or for at least about 25 years.

A chimeric protein described herein can be administered by any means known in the art. In some embodiments, the chimeric protein is administered by a route selected from the group consisting of intravenous injection, intravenous infusion, subcutaneous administration, intramuscular administration, oral administration, nasal administration, and pulmonary administration. In some embodiments, the chimeric protein is administered intravenously. In some embodiments, the chimeric protein is administered subcutaneously.

Having now described the present disclosure in detail, the same will be more clearly understood by reference to the following examples, which are included herewith for purposes of illustration only and are not intended to be limiting of the disclosure. All patents, publications, and articles referred to herein are expressly and specifically incorporated herein by reference.

### EXAMPLES

### Example 1: Evaluation of Buffer Selection and Target pH on Stability of Chimeric Protein

The experiments of this example showed favorable pH and buffer species from various buffers like acetate, succinate, histidine, potassium phosphate, and Tris within a wide pH range from 4.0-8.0. Eleven buffers were studied in combination with efanesoctocog alfa at a target concentration of 1 mg/mL. The buffers studied are shown in Table 1. No additional excipients were included in the pharmaceutical compositions in this study. Each pharmaceutical composition comprising efanesoctocog alfa and the specific buffer was evaluated by measuring actual protein concentration, pH, visual inspection, high molecular weight species (HMWS), and turbidity. Studied samples were stored for up to 4 weeks at 5° C., 25° C., and 40° C.

TABLE 1
Buffers Studied

| Abbreviation | Full Name |
|---|---|
| A4.0 | 10 mM sodium acetate pH 4.0 |
| A5.0 | 10 mM sodium acetate pH 5.0 |
| S5.5 | 10 mM sodium succinate pH 5.5 |
| H5.5 | 10 mM histidine-HCl pH 5.5 |
| H6.0 | 10 mM histidine-HCl pH 6.0 |
| H6.5 | 10 mM histidine-HCl pH 6.5 |
| H7.0 | 10 mM histidine-HCl pH 7.0 |
| H7.5 | 10 mM histidine-HCl pH 7.5 |
| KP6.5 | 10 mM potassium phosphate pH 6.5 |
| KP7.0 | 10 mM potassium phosphate pH 7.0 |
| T8.0 | 10 mM Tris pH 8.0 |

Results of initial visual appearance, measured protein concentration, and pH of preparations of 1 mg/mL efanesoctocog alfa in the respective buffers are shown in Table 2. For H7.0, H7.5, and T8.0, the measured pH was 6.88, 7.05, and 7.84 respectively, which was lower than the target (expected) pH.

TABLE 2

| | | Measured | |
|---|---|---|---|
| | | Concen- | |
| Buff- | | tration | Measured |
| er | Visual Appearance | (mg/mL) | pH |
| A4.0 | Precipitated | Not | Not |
| | | measured | measured |
| A5.0 | Cloudy, White, No visible particles | Not | Not |
| | | measured | measured |
| S5.5 | Clear, White, No visible particles | 1.06 | 5.48 |
| H5.5 | Clear, White, No visible particles | 1.06 | 5.46 |
| H6.0 | Clear, White, No visible particles | 1.04 | 5.99 |
| H6.5 | Clear, White, No visible particles | 1.05 | 6.40 |
| H7.0 | Clear, White, No visible particles | 1.04 | 6.88 |
| H7.5 | Clear, White, No visible particles | 1.06 | 7.05 |
| KP6.5 | Clear, White, No visible particles | 1.03 | 6.49 |
| KP7.0 | Clear, White, No visible particles | 1.07 | 6.94 |
| T8.0 | Cloudy, White, No visible particles | 1.04 | 7.84 |

Pharmaceutical compositions A4.0 and A5.0 were eliminated from further study as they showed precipitation and poor visual appearance. The remaining pharmaceutical compositions were stored at 5° C. and percentage of HMWS (% HMWS) were measured by size exclusion chromatography (SEC) at 0, 7, and 30 days. Results are shown in Table 3.

TABLE 3
Aggregation (% HMWS) by SEC at 5° C.

| Sample | 0 Days | 7 Days | 30 Days |
|---|---|---|---|
| S5.5 | 88.14 | 86.50 | 95.15 |
| H5.5 | 58.21 | 60.79 | 87.86 |
| H6.0 | 68.38 | 68.50 | 69.73 |
| H6.5 | 28.39 | 27.64 | 28.64 |
| H7.0 | 7.01 | 6.31 | 4.32 |
| H7.5 | 6.63 | 7.07 | 4.29 |
| KP6.5 | 24.00 | 22.15 | 23.92 |
| KP7.0 | 18.48 | 17.34 | 16.86 |
| T8.0 | 27.25 | 49.11 | 65.52 |
| standard | 2.56 | 2.48 | 2.85 |

Pharmaceutical compositions containing histidine buffer at pH 6.88 (H7.0) and pH 7.05 (H7.5) showed the lowest % HMWS. Slight loss in HMWS was observed at 1 month (30 days). Pharmaceutical compositions containing histidine buffer at pH 6.5 (H6.5) or potassium phosphate buffer at pH 6.5 (KP6.5) and pH 7 (KP7.0) produced comparable % HMWS, but showed significantly higher % HMWS than pharmaceutical compositions containing histidine buffer at pH 6.88 (H7.0) and pH 7.05 (H7.5).

The pharmaceutical compositions were stored at 25° C. and % HMWS was measured by size exclusion chromatography (SEC) at 0, 0.25, 4, and 7 days. Results are shown in Table 4. Pharmaceutical compositions containing histidine buffer at pH 6.88 (H7.0) and pH 7.05 (H7.5) showed the lowest % HMWS at the start of the experiment (0 days).

TABLE 4
Aggregation (% HMWS) by SEC at 25° C.

| Sample | 0 Days | 0.25 Day | 4 Days | 7 Days |
|---|---|---|---|---|
| S5.5 | 88.14 | 87.53 | 94.28 | 95.30 |
| H5.5 | 58.21 | 87.23 | 93.53 | 93.63 |
| H6.0 | 68.38 | 78.07 | 91.17 | 93.00 |
| H6.5 | 28.39 | 28.39 | 39.27 | 65.32 |
| H7.0 | 7.01 | 7.47 | 14.07 | 16.72 |
| H7.5 | 6.63 | 5.23 | 39.41 | 46.55 |
| KP6.5 | 24.00 | 22.99 | 24.77 | 25.26 |
| KP7.0 | 18.48 | 19.43 | 19.61 | 20.22 |
| T8.0 | 27.25 | 27.45 | 25.17 | 46.22 |
| standard | 2.56 | 2.36 | 2.85 | 2.27 |

The pharmaceutical compositions were stored at 40° C. and % HMWS was measured by size exclusion chromatography (SEC) at 0, 0.25, 1, and 7 days. Results are shown in Table 5.

TABLE 5
Aggregation (% HMWS) by SEC at 40° C.

| Sample | 0 Days | 0.25 Day | 1 Day | 7 Days |
|---|---|---|---|---|
| S5.5 | 88.14 | 97.11 | 97.25 | 97.21 |
| H5.5 | 58.21 | 98.85 | 97.44 | 98.35 |
| H6.0 | 68.38 | 72.70 | 82.80 | 96.48 |
| H6.5 | 28.39 | 73.51 | 85.18 | 93.39 |
| H7.0 | 7.01 | 22.06 | 42.36 | 68.71 |
| H7.5 | 6.63 | 7.79 | 14.71 | 46.99 |
| KP6.5 | 24.00 | 23.14 | 25.82 | 35.79 |
| KP7.0 | 18.48 | 18.69 | 20.00 | 28.70 |
| T8.0 | 27.25 | 30.33 | 24.96 | 58.07 |
| standard | 2.56 | 2.28 | 2.79 | 2.79 |

Pharmaceutical compositions containing histidine buffer at pH 6.88 (H7.0) and pH 7.05 (H7.5) showed the lowest % HMWS at the start. Significant degradation of the efanesoctocog alfa was observed at 40° C. for all buffers tested.

Concentration stability was also assessed for the pharmaceutical compositions at 5° C., 25° C., and 40° C. at various time points. Results are shown in Table 6. For all but one sample, there was a slight increase in concentration (mg/mL) at 1 month at 5° C., demonstrating possible instability or aggregation; only potassium phosphate at pH 6.5 (KP6.5) did not show increase in concentration. The samples did not show any significant change in concentration after 1 week at all temperatures.

TABLE 6
Concentration Stability Results
pH BUFFER STUDY: Concentration (mg/mL), Time (days)

| 5° C. | | | | 25° C. | | | | | 40° C. | | | | |
|---|---|---|---|---|---|---|---|---|---|---|---|---|---|
| Sample | 0 | 7 | 30 | Sample | 0 | 0.25 | 4 | 7 | Sample | 0 | 0.25 | 1 | 7 |
| S5.5 | 1.01 | 1.01 | 1.25 | S5.5 | 1.01 | 1.06 | 0.95 | 1.04 | S5.5 | 1.01 | 1.02 | 0.97 | 1.04 |
| H5.5 | 1.05 | 1.04 | 1.68 | H5.5 | 1.05 | 0.99 | 1.04 | 1.07 | H5.5 | 1.05 | 1.06 | 0.96 | 1.06 |
| H6.0 | 1.05 | 1.05 | 1.50 | H6.0 | 1.05 | 1.07 | 1.04 | 1.09 | H6.0 | 1.05 | 1.06 | 1.01 | 1.12 |
| H6.5 | 1.06 | 1.06 | 1.62 | H6.5 | 1.06 | 0.97 | 1.07 | 1.04 | H6.5 | 1.06 | 1.11 | 1.08 | 1.11 |
| H6.8 | 1.06 | 1.04 | 1.38 | H6.8 | 1.06 | 0.96 | 1.01 | 1.04 | H6.8 | 1.06 | 1.10 | 1.07 | 1.07 |
| H7.3 | 1.07 | 1.06 | 1.41 | H7.3 | 1.07 | 1.01 | 1.05 | 1.06 | H7.3 | 1.07 | 1.03 | 1.05 | 1.03 |
| KP6.5 | 1.06 | 1.06 | 1.06 | KP6.5 | 1.06 | 1.08 | 0.99 | 1.00 | KP6.5 | 1.06 | 1.11 | 0.92 | 1.04 |
| KP7.0 | 1.07 | 1.01 | 1.36 | KP7.0 | 1.07 | 1.07 | 1.04 | 1.04 | KP7.0 | 1.07 | 1.08 | 1.08 | 1.02 |
| T 8.0 | 1.10 | 1.00 | 1.55 | T8.0 | 1.10 | 1.05 | 1.07 | 1.08 | T8.0 | 1.10 | 1.11 | 1.03 | 1.06 |

pH stability was also assessed for the pharmaceutical compositions at 5° C., 25° C., and 40° C. at various time points. pH values for the samples did not change significantly when stored for one month at 5° C., one week at 25° C., or 1 week at 40° C. (data not shown). Measured values were all within +/−0.5 units from time zero value over all time points.

Turbidity was also assessed for the pharmaceutical compositions at 5° C., 25° C., and 40° C. at various time points. There was a 2- to 3-fold increase in absorbance in compositions containing potassium phosphate 7.0 (KP7.0) and histidine 5.5 (H5.5) after 7 days at 25° C. and 40° C., respectively. Overall, the absorbance values did not change significantly when stored for one month at 5° C., one week at 25° C., or 1 week at 40° C. (data not shown).

### SUMMARY

Pharmaceutical compositions containing histidine and potassium phosphate within a pH range of 6.8 to 7.3 showed minimal change in concentration, pH, and turbidity, as well as low aggregation (% HMWS). Efanesoctocog alfa appears to be most stable in a pharmaceutical composition having a pH range of 6.8 to 7.3.

The initial aggregation was significantly lower in histidine buffer than in potassium phosphate buffer. The smallest increase in aggregation was seen in phosphate buffer pH 6.5 and pH 7.0 over 7 days at stress temperatures of 25° C. and 40° C.

### Example 2: Evaluation of Histidine Molarity and Protein Concentration on Stability of Efanesoctocog Alfa

These determined the effect of the molarity of histidine and the concentration of protein on achieving a target pH of 7.0. The studies were also conducted to determine the effect of buffer molarity on the stability of efanesoctocog alfa at protein concentrations of 1, 0.67 and 0.045 mg/mL.

Study Design: Pharmaceutical compositions were studied with each containing one of three different molarities of histidine (10 mM, 20 mM, and 50 mM) and one of three different concentrations of efanesoctocog alfa (1 mg/mL, 0.67 mg/mL (4000 IU/mL), and 0.045 mg/ml (250 IU/mL)) in the presence of other excipients at constant concentrations. In addition to histidine and efanesoctocog alfa, all pharmaceutical compositions were formulated to include the following excipients: 250 mM Arginine hydrochloride (ArgHCl), 5 mM CaCl₂), 5% sucrose, and 0.05% polysorbate 80 (PS80). Liquid stability was evaluated for one week at 5° C., 25° C. and 40° C. Stability of each pharmaceutical composition was evaluated by visual inspection, turbidity, protein concentration (Lunatic, Unchained Labs, Pleasanton CA), pH, osmolality, % HMWS by SEC, and protein concentration by RP-HPLC.

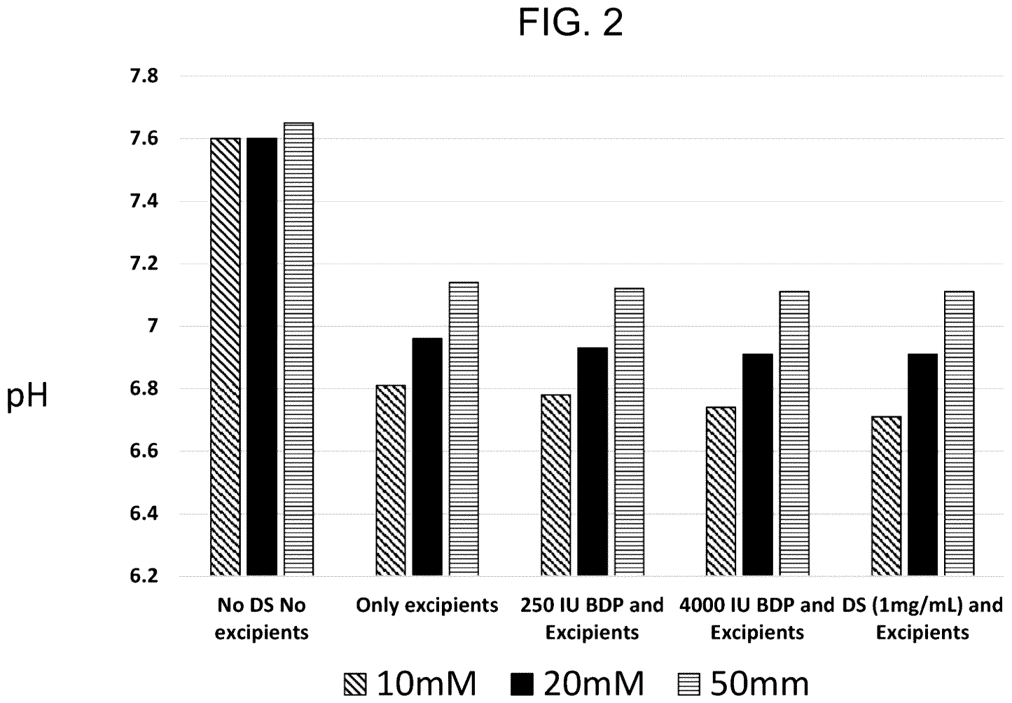

The maximum pH for pharmaceutical compositions comprising histidine at 10 mM, 20 mM, and 50 mM at any protein concentration (1 mg/mL, 4000 IU/mL, or 250 IU/mL) was 6.8, 6.95, and 7.1, respectively. Target pH 7.0 was achieved at 20 mM and 50 mM histidine. As the molarity of histidine increased, the pH of the formulation increased, potentially due to the increased buffering capacity. (see FIG. 2). There was no significant effect of protein concentration on the pH and there was no significant change in pH over time and temperature (data not shown).

At the start of the experiment (T0), higher osmolality was observed as the histidine molarity increased from 10 mM to 50 mM. Visual inspection showed the nine formulations studied at TO and 1 week at 5° C., 25° C. and 40° C. were clear and colorless. However, the protein aggregated significantly at 40° C. after 1 week, and there was a significant increase in % HMWS at 1 week at 40° C. There was no effect of molarity on concentration and osmolality over 1 week at the three different temperatures (data not shown).

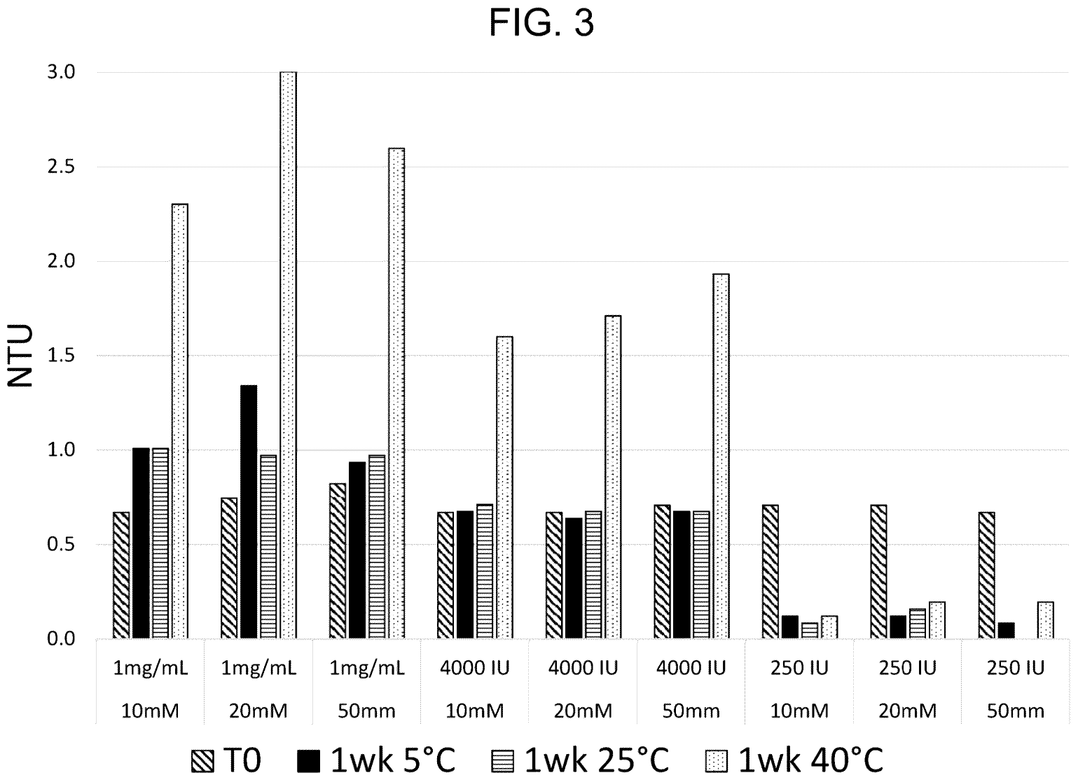

Turbidity results are shown in Table 7. Turbidity (NTU) showed a direct relation with protein concentration. At 5° C. and 25° C. the samples were clear at TO and remained clear after 1 week. Although turbidity increased significantly at 40° C. for 1 mg/mL and 4000 IU samples, 1 mg/mL samples were clear after 1 week. Further results of turbidity testing are shown in FIG. 3.

TABLE 7

NTU

| | T0 | 1 week | | |
|---|---|---|---|---|
| SAMPLE | T0 | 5° C. | 25° C. | 40° C. |
| 10 mM HIS (1 mg/mL) | 0.7 | 1.0 | 1.0 | 2.3 |
| 20 mM HIS (1 mg/mL) | 0.7 | 1.3 | 1.0 | 3.0 |
| 50 mM HIS (1 mg/mL) | 0.8 | 0.9 | 1.0 | 2.6 |
| 10 mM HIS 4000 IU | 0.7 | 0.7 | 0.7 | 1.6 |
| 20 mM HIS 4000 IU | 0.7 | 0.6 | 0.7 | 1.7 |
| 50 mM HIS 4000 IU | 0.7 | 0.7 | 0.7 | 1.9 |
| 10 mM HIS 250 IU | 0.7 | 0.1 | 0.1 | 0.1 |
| 20 mM HIS 250 IU | 0.7 | 0.1 | 0.2 | 0.2 |
| 50 mM HIS 250 IU | 0.7 | 0.1 | n/a | 0.2 |

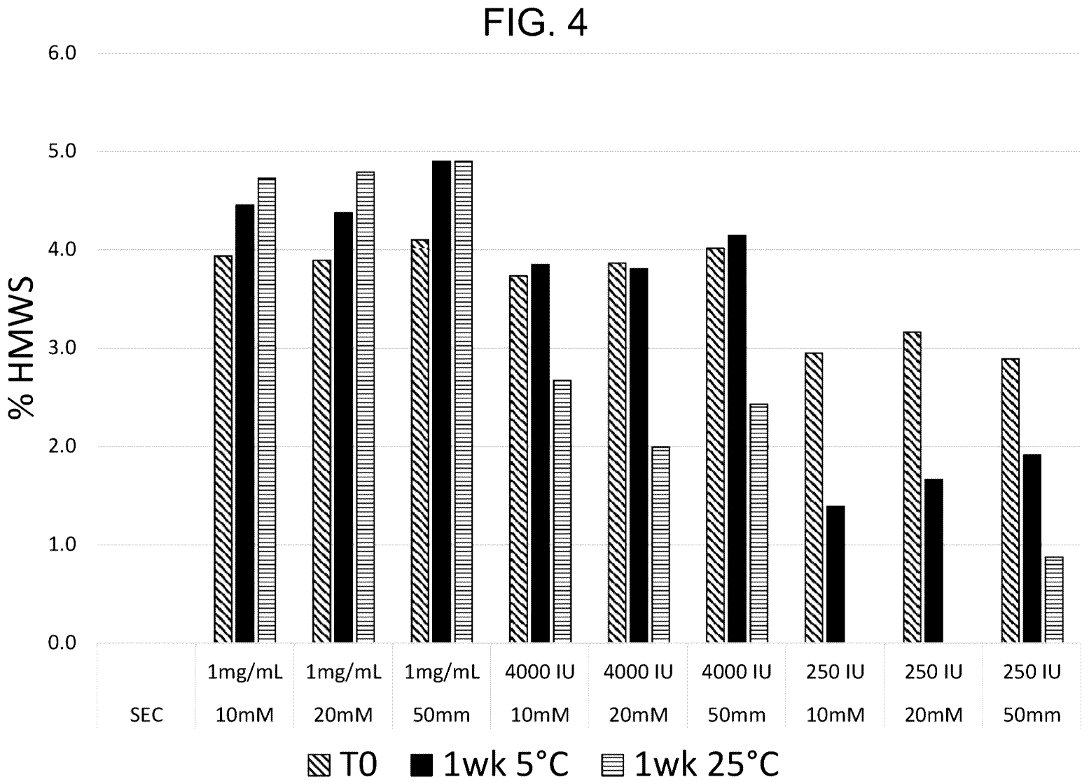

Results of the analysis of % HMWS by SEC for samples observed at 5° C. and 25° C. are shown in FIG. 4. Aggregation was significantly higher for all molarities and protein concentrations at 40° C. (data not shown). SEC testing revealed that the greater the protein concentration, the greater the % HMWS at TO. It was also observed that the % HMWS of efanesoctocog alfa was comparable at 5° C., 25° C., and 40° C. across all concentrations of histidine tested (10, 20 or 50 mM). Thus, changes in molarity of histidine did not appear to have an effect on aggregation levels. Samples containing 1 mg/ml of protein showed aggregation increases with temperature.

Samples containing 4000 IU/mL (0.67 mg/ml) of protein showed loss of aggregates after 1 week at 25° C. Samples containing 250 IU/mL (0.045 mg/mL) showed loss of aggregates after 1 week at at both 25° C. and 40° C. Generally, at 25° C., there was some HMWS aggregation, but over time the HMWS appeared to be breaking down into smaller molecules and presented as LMW peaks after a short duration (approximately 25 minutes). In contrast to 25° C., HMWS did not appear to break down into LMW species at 40° C. The 250 IU/mL (0.045 mg/mL) sample was not stable at 40° C.

### SUMMARY

A maximum pH of 6.8 was achieved for the pharmaceutical compositions containing efanesoctocog alfa at 1 mg/mL, 4000 IU/mL, or 250 IU/mL; 10 mM histidine; 250 mM ArgHCl; 5% sucrose; 5 mM CaCl₂); and 0.05% PS80. Increasing molarity of histidine buffer showed no significant effects on pH, osmolality, protein concentration, turbidity, % HMWS at TO, or % HMWS over 1 week stability at 5° C., 25° C., and 40° C. Efanesoctocog alfa showed significant aggregation at a stress temperature of 40° C. after 1 week for all protein concentrations studied. For pharmaceutical compositions containing 4000 IU/mL and 250 IU/mL protein, significant loss of aggregate peaks was observed at 25° C. and 40° C. over 1 week. There was a significant increase in turbidity at 1 week at 40° C. for pharmaceutical compositions containing 1 mg/ml and 4000 IU/mL of efanesoctocog alfa.

### Example 3: Evaluation of Polysorbate 80 (PS80) Concentration on Protein Stability

These studies were determined the effect of the concentration of PS80 on the stability of efanesoctocog alfa formulations.

Two concentrations of efanesoctocog alfa (0.67 mg/ml (4000 IU/mL) and 0.045 mg/mL (250 IU/mL)) in 10 mM Histidine, 250 mM Arginine hydrochloride (ArgHCl), 5 mM CaCl₂), 5% sucrose, and polysorbate 80 (PS80) were studied. The range of concentrations of PS80 for the 250 IU/mL samples was 0.002% to 0.07%. The range of concentrations of PS80 for the 4000 IU/mL samples was 0.03% to 0.1%. Samples were subjected to stress by orbital agitation (250 rpm for 0, 6, and 24 hours) or freeze/thaw (−80° C. not less than 24 hrs, thaw at room temperature) for 0, 1, 3, and 5 cycles. Analysis included aggregation by SEC, PS80 concentration by high performance liquid chromatography (HPLC) with charged aerosol detection (CAD), particle test by micro-flow imaging (MFI) and high accuracy liquid particle counter (HIAC), pH, turbidity, and protein concentration. Results of each analysis is shown in Table 8.

### SUMMARY

The results of this study indicated that a concentration of PS80≥0.03% stabilized both 250 IU and 4000 IU concentrations of efanesoctocog alfa. No changes in PS80 content were observed for samples after 24 hrs agitation or 5 freeze/thaw cycles. No trend in particulates was observed for the whole study of PS80 range. No trends or changes in pH or turbidity were observed over the PS80 range. Therefore, a pharmaceutical composition containing a concentration of PS80≥0.03% may increase stability of efanesoctocog alfa at a minimum concentration of 250 IU and a maximum concentration of 4000 IU with minimal impact on other composition properties.

TABLE 8
Sub-Visible Particles by MFI and HIAC

| | | PS80 | Agitation Study | | | | | | Freeze/Thaw study | | | | | |
|---|---|---|---|---|---|---|---|---|---|---|---|---|---|---|
| | | Con. | T0 | | T6 hrs | | T24 hrs | | F/T 1X | | F/T 3X | | F/T 5X | |
| | Strength | (%) | ≥10 μm | ≥25 μm | ≥10 μm | ≥25 μm | ≥10 μm | ≥25 μm | ≥10 μm | ≥25 μm | ≥10 μm | ≥25 μm | ≥10 μm | ≥25 μm |
| Particle | 250 IU | 0.002 | 44 | 11 | 14 | 5 | 23 | 2 | 14 | 7 | 7 | 2 | 28 | 5 |
| # by | | 0.005 | 11 | 5 | 11 | 7 | 9 | 5 | 9 | 0 | 11 | 0 | 9 | 2 |
| MFI | | 0.010 | 14 | 2 | 11 | 5 | 28 | 5 | 14 | 0 | 11 | 5 | 28 | 9 |
| | | 0.030 | 39 | 7 | 39 | 7 | 14 | 7 | 2 | 0 | 21 | 0 | 5 | 2 |
| | | 0.050 | 18 | 0 | 9 | 5 | 41 | 11 | 50 | 7 | 57 | 0 | 32 | 9 |
| | | 0.070 | 14 | 7 | 57 | 18 | 25 | 5 | 16 | 5 | 9 | 0 | 0 | 0 |
| | 4000 IU | 0.030 | 7 | 2 | 9 | 2 | 257 | 64 | 18 | 5 | 5 | 0 | 21 | 5 |
| | | 0.050 | 7 | 2 | 11 | 0 | 18 | 5 | 41 | 14 | 16 | 5 | 7 | 5 |
| | | 0.070 | 9 | 0 | 7 | 0 | 156 | 48 | 41 | 5 | 32 | 7 | 9 | 0 |
| | | 0.100 | 11 | 2 | 9 | 5 | 119 | 32 | 7 | 0 | 16 | 2 | 11 | 2 |
| Particle | 250 IU | 0.002 | 60 | 8 | 18 | 0 | 15 | 0 | 100 | 25 | 2 | 0 | 7 | 0 |
| # by | | 0.005 | 27 | 0 | 27 | 0 | 7 | 2 | 0 | 0 | 0 | 0 | 0 | 0 |
| HIAC | | 0.010 | 20 | 0 | 17 | 0 | 27 | 2 | 2 | 0 | 3 | 2 | 0 | 0 |
| | | 0.030 | 28 | 3 | 20 | 0 | 33 | 2 | 2 | 2 | 3 | 0 | 2 | 0 |
| | | 0.050 | 22 | 2 | 10 | 2 | 23 | 2 | 7 | 0 | 0 | 0 | 0 | 0 |
| | | 0.070 | 17 | 2 | 43 | 2 | 10 | 0 | 23 | 7 | 2 | 0 | 2 | 0 |
| | 4000 IU | 0.030 | 25 | 0 | 17 | 0 | 18 | 0 | 0 | 0 | 5 | 2 | 2 | 0 |
| | | 0.050 | 12 | 2 | 25 | 0 | 12 | 2 | 2 | 0 | 2 | 0 | 2 | 0 |
| | | 0.070 | 10 | 2 | 2 | 0 | 17 | 3 | 0 | 0 | 2 | 0 | 3 | 0 |
| | | 0.100 | 32 | 0 | 12 | 0 | 13 | 2 | 2 | 0 | 52 | 22 | 2 | 0 |

### Example 4: Evaluation of Excipient Concentrations on Protein Stability

The experiments described in this example assess the stability of pharmaceutical compositions comprising efanesoctocog alfa drug product (DP) at 1000 IU formulated with dilution buffers containing varied concentrations of excipients. Minimum and maximum excipient concentration ranges were intentionally designed to deviate from current manufacturing tolerances in order to challenge the formulation robustness. A series of 13 different dilution buffers were evaluated (see Table 9). The Design of Experiment (DoE) model was based on five parameters (i.e., five excipients) at two levels (i.e., +5% of target values). This included a control center point with the target formulation excipient quantities (i.e., Buffer #13).

Efanesoctocog alfa drug substance (DS) was pre-formulated at a concentration of 1 mg/ml in 10 mM L-Histidine, 250 mM L-Arginine Hydrochloride (HCl), 5 mM Calcium Chloride, 5% (w/v) Sucrose and 0.05% (w/v) Polysorbate 80 (PS80) at pH 7.01. Efanesoctocog alfa DS was diluted down to one of several strengths using one of the 13 dilution buffers to generate diluted drug substance (DDS) of the efanesoctocog alfa.

TABLE 9
Dilution Buffers Tested

| | | | | Calcium | |
|---|---|---|---|---|---|
| | L-Arginine | | | Chloride | Polysorbate- |
| Buffer | HCl | Sucrose | L-Histidine | Dihydrate | 80 |
| No. | (g/L) | (g/L) | (g/L) | (g/L) | (g/L) |
| 1 | 50.032 | 52.500 | 1.630 | 0.698 | 0.475 |
| 2 | 55.298 | 47.500 | 1.474 | 0.698 | 0.525 |
| 3 | 55.298 | 52.500 | 1.474 | 0.698 | 0.475 |
| 4 | 55.298 | 47.500 | 1.630 | 0.772 | 0.525 |
| 5 | 50.032 | 47.500 | 1.630 | 0.772 | 0.475 |
| 6 | 55.298 | 52.500 | 1.630 | 0.772 | 0.525 |
| 7 | 50.032 | 52.500 | 1.474 | 0.772 | 0.525 |
| 8 | 50.032 | 52.500 | 1.630 | 0.698 | 0.525 |
| 9 | 55.298 | 47.500 | 1.630 | 0.698 | 0.475 |
| 10 | 50.032 | 47.500 | 1.474 | 0.698 | 0.525 |
| 11 | 55.298 | 52.500 | 1.474 | 0.772 | 0.475 |
| 12 | 50.032 | 47.500 | 1.474 | 0.772 | 0.475 |
| 13 | 52.665 | 50.000 | 1.552 | 0.735 | 0.500 |

Efanesoctocog alfa DDS was then lyophilized to generate efanesoctocog alfa DP at the intended strength per vial of 1000 IU. The resulting pharmaceutical compositions containing efanesoctocog alfa DP at 1000 IU were assessed at various stability conditions (2-8° C. and 30° C.) and stability timepoints (T0, one month (T1), 3 months (T3), 6 months (T6), and 12 months (T12)). Pharmaceutical composition samples were tested for specific activity, protein concentration, aggregation, visual inspection, reconstitution time, pH, residual moisture, product/excipient crystallization, and glass transition temperature.

Protein concentration was measured using a RP-HPLC method with fluorescence detection. A stability specification range of 75-125% of initial value was targeted for protein concentration.

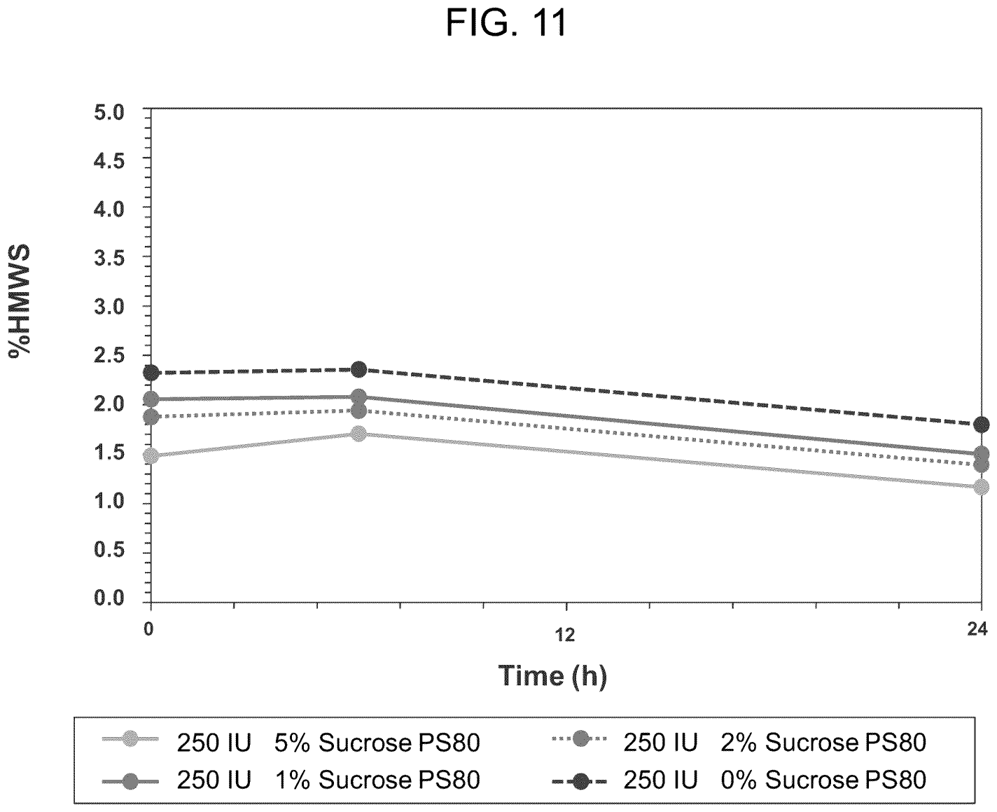

Table 10 and FIG. 11 display concentration stability data up to 6 months (T6). Concentration values were generally lower than the associated target value of 167 μg/mL but remained within the 75-125% of initial DP concentration value, thus falling within the acceptable range of the specification.

TABLE 10
Protein concentration results ( μg/mL)

| | | | | | | | | DP % |
|---|---|---|---|---|---|---|---|---|
| Buffer | T0 | T0 DP | T1 DP | T3 DP | T3 DP | T6 DP | T6 DP | Decrease |
| No. | DDS | 2-8° C. | 30° C. | 2-8° C. | 30° C. | 2-8° C. | 30° C. | T0 → T6 |
| 1 | 159.7 | 156.1 | 151.6 | 160.6 | 148.1 | 138.5 | 138.6 | 11.21 |
| 2 | 165.2 | 158.2 | 148.9 | 154.5 | 149.5 | 139.7 | 142 | 10.24 |
| 3 | 151.5 | 158.5 | 144.7 | 152.5 | 151.7 | 138 | 139.2 | 12.18 |
| 4 | 160.4 | 160.6 | 144.7 | 160 | 154.5 | 139.6 | 139.9 | 12.89 |
| 5 | 155.6 | 161.8 | 150.1 | 159.7 | 153.2 | 144 | 140.3 | 13.29 |
| 6 | 159.6 | 156.0 | 152.8 | 162 | 156 | 140.2 | 138.9 | 10.96 |
| 7 | 165.8 | 163.8 | 154.7 | 153.6 | NA | 138.9 | 144.5 | 11.78 |
| 8 | 155.4 | 161.8 | 156.3 | 150.7 | 154.2 | 134.9 | 140.7 | 13.04 |
| 9 | 157 | 162.2 | 160.8 | 153.1 | 155.2 | 134.1 | 141.8 | 12.58 |
| 10 | 157.7 | 169.5 | 161.9 | 154 | 157.6 | 140.6 | 141.8 | 16.34 |
| 11 | 154.1 | 165.8 | 160.6 | 155.4 | 151.1 | 135.4 | 138.2 | 16.65 |
| 12 | 161.2 | 170.2 | 170.4 | 160.6 | 159.9 | 141.8 | 141.2 | 17.04 |
| 13 | 145.4 | 167.9 | 162.7 | 152.6 | 153.1 | 142.5 | 137.6 | 18.05 |
| Average | 157.58 | 162.49 | 155.40 | 156.10 | 156.62 | 139.09 | 140.36 | N/A |
| Std. Dev | 5.49 | 4.76 | 7.63 | 3.88 | 11.10 | 2.96 | 1.91 | N/A |

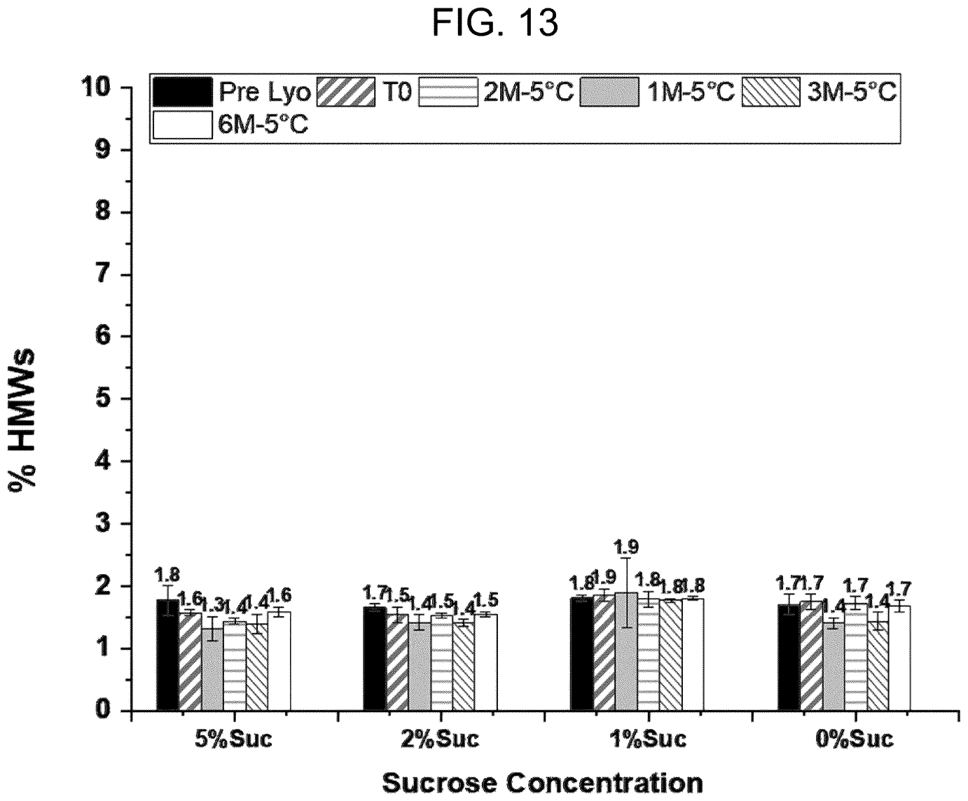

Specific activities of DP and DDS were calculated using concentration data from FIG. 11. Results are shown in Table 11 and FIG. 13. The specific activity specification is >1600 IU/mg for DDS and >1280 IU/mg for DP of all strengths. For DP, the specific activity of each tested sample exceeded the specification at each time point.

Testing of efanesoctocog alfa DDS found that pre-lyophilization activity values were within the 80-125% acceptable range of the specification.

TABLE 11
Specific activity results (IU/mg)

| Buffer | | DDS | T0 DP | T1 DP | T3 DP | T3 DP | DP |
|---|---|---|---|---|---|---|---|
| No. | T0 DDS | Spec | (2-8° C.) | (30° C.) | (2-8° C.) | (30° C.) | Spec |
| 1 | 1748.3 | 1600 | 2053.7 | 1528.5 | N/A | 1497.0 | 1280.0 |
| 2 | 1679.8 | 1600 | 2138.1 | 1409.6 | N/A | 1554.5 | 1280.0 |
| 3 | 2093.7 | 1600 | 2251.5 | 1361.5 | N/A | 1624.3 | 1280.0 |
| 4 | 1791.1 | 1600 | 2036.6 | 1563.5 | N/A | 1622.7 | 1280.0 |
| 5 | 1822.0 | 1600 | 2060.9 | 1416.6 | N/A | 1560.1 | 1280.0 |
| 6 | 1830.8 | 1600 | 1946.4 | 1550.0 | N/A | 1443.6 | 1280.0 |
| 7 | 1769.6 | 1600 | 2040.5 | 1473.7 | N/A | 1352.60 | 1280.0 |
| 8 | 1718.8 | 1600 | 2098.6 | 1542.0 | N/A | 1655.0 | 1280.0 |
| 9 | 1759.2 | 1600 | 1600.6 | 1496.3 | N/A | 1607.6 | 1280.0 |
| 10 | 1750.8 | 1600 | 1620.5 | 1423.6 | N/A | N/A | 1280.0 |
| 11 | 1835.8 | 1600 | 1591.8 | 1504.8 | N/A | 1653.9 | 1280.0 |
| 12 | 1719.6 | 1600 | 1608.5 | 1426.6 | N/A | 1627.9 | 1280.0 |
| 13 | N/A | 1600 | 1626.2 | 1432.4 | N/A | 1659.0 | 1280.0 |

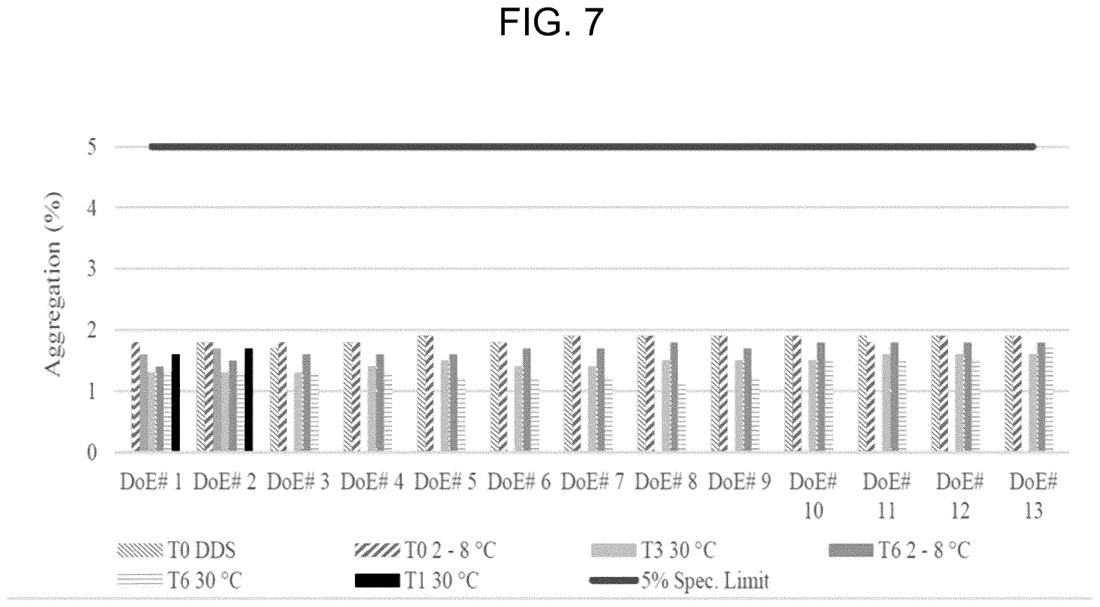

DP and DDS aggregation testing was performed by SEC-HPLC and results are shown in FIG. 7. At TO, DP aggregation ranged between 1.8-1.9%. At T3 and T6, 30° C. samples displayed reduced aggregation values as low as 1.1% which may be due to column performance or system variation. When comparing T0 to T6 2-8° C. results, no significant change in aggregation was observed. All aggregation values obtained are below 5% for efanesoctocog alfa DP. T1 30° C. and T3 2-8° C. sample aggregation analyses are yet to be completed.

Residual moisture values did not show significant variation on stability, with all results remaining below 1.2%, within the 3% stability specification limit.

Upon visual inspection, efanesoctocog alfa DP lyophilate cakes were observed to be white to off-white in color. These cakes fully dissolved upon reconstitution within ˜ 60 seconds. Reconstitution values at TO and T1 timepoints were slightly lower (within 25 seconds). All efanesoctocog alfa DP samples were reconstituted within 70 seconds (well below the proposed 3.5-minute specification), and no unexpected appearance observations were noted.

Samples were tested using powder X-ray diffraction. Samples appeared as amorphous content at T1 to T6 timepoints as confirmed by the amorphous halo pattern of the diffractogram. No evidence of product or excipient crystallisation such as crystallisation peaks was observed.

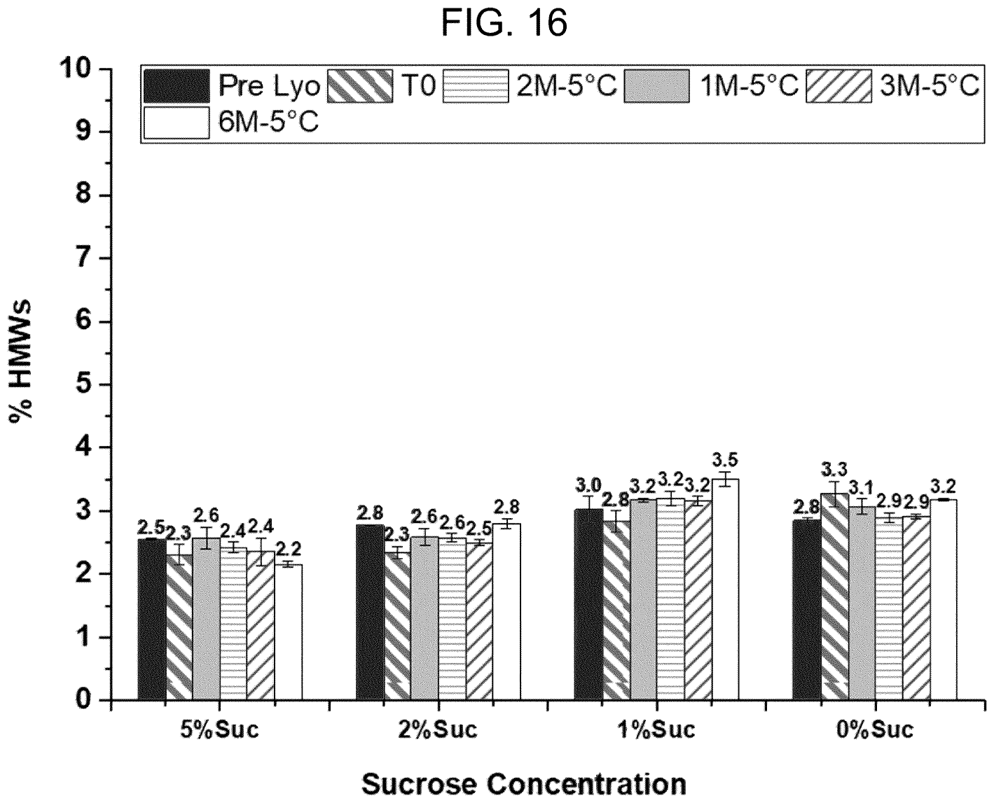

The glass transition temperature (Tg) was measured by differential scanning calorimetry (DSC) in duplicate for each sample; averaged results are shown in FIG. 16. The highest and lowest values for Tg were 71.75° C. (T6 30° C. Buffer #10) and 61.95° C. (Buffer #3 T6 2-8° C.) respectively. The highest Tg value (Buffer #10 T6 30° C.) coincided with the lowest moisture content (0.9%) observed across the samples at that timepoint. The lowest Tg value (Buffer #3 T6 2-8° C.) coincided with the higher relative moisture content (1.11%) observed for Buffer #3 at this timepoint. Samples did not show any obvious trend toward increasing or decreasing Tg values at different storage conditions on stability. Enthalpy values measured between 0.19 and 0.46 J/g° C.

All stability sample results shown in FIG. 16 displayed Tg values above 60° C. This is favorable toward stable long-term storage of the efanesoctocog alfa at a temperature of 2-8° C., with an inclusive six month storage at room temperature.

### Example 5: Evaluation of Arginine HCl Concentration on Protein Stability

The experiments described in this example are used to assess the stability of efanesoctocog alfa in pharmaceutical compositions formulated with different concentrations of Arginine HCl.

Two pharmaceutical compositions are evaluated where the only difference is concentration of L-Arginine-HCl. The first composition contains 1.0 mg/mL efanesoctocog alfa in 10 mM Histidine, pH 7.0, 250 mM L-Arginine-HCl, 5 mM CaCl₂), 5% sucrose, and 0.05% Polysorbate-80. The second composition contains 1.0 mg/mL efanesoctocog alfa in 10 mM Histidine, pH 7.0, 175 mM L-Arginine-HCl, 5 mM CaCl₂), 5% sucrose, and 0.05% Polysorbate-80. Each composition is diluted approximately 60, 120 and 240 minutes after thawing at room temperature in preparation for size exclusion chromatography (SEC) analysis. Duplicate SEC injections of each sample are made and the % HMWS is determined for each injection.

Lyophilized efanesoctocog alfa drug product is reconstituted in 3.0 ml of water for injection (WFI). An aliquot of the reconstituted solution is transferred to an HPLC vial for analysis.

The SEC assay uses an HPLC instrument equipped with a pump, a temperature controlled autosampler, a column heater, and Fluorescence detector. The sample solutions are analyzed using the following instrumentation and method parameters:
Mobile Phase: Dulbecco's Phosphate-Buffered Saline (D-PBS) containing Calcium and Magnesium adding 0.36 M Sodium Chloride (0.9 mM Calcium Chloride, 0.5 mM Magnesium Chloride, 2.7 mM Potassium Chloride, 1.5 mM Potassium Phosphate Mono basic, 496 mM Sodium Chloride, 8.1 mM Sodium Phosphate Dibasic, pH 7.0±0.1)
Column Heater: 26° C.
Run Time: 40 minutes
FL Detector: Ex/Em=280/350 nm, PMT Gain=5
Injection Mass Load: 2 μg
Autosampler Temperature: 5° C.
Flow Rate: 0.5 mL/min, Isocratic

Pharmaceutical compositions of efanesoctocog alfa containing 250 mM L-Arginine-HCl are found to be more stable than compositions containing 175 mM L-Arginine-HCl.

### Example 6: Further Evaluation of L-Arginine-HCl Concentration on Protein Stability

The effect of arginine concentration over a broader range was also examined. Eight concentrations of L-arginine-HCl (50, 100, 125, 150, 175, 200, 250, and 300 mM) were added to a base formulation of 10 mM histidine, 5 mM calcium chloride, 5% sucrose, and 0.05% polysorbate 80. A summary of the study design is provided in Table 12. Upon thawing at room temperature (25° C.), the formulations were prepared, placed in an HPLC vial and stored in the autosampler at 5° C.±3°. HPLC injections for each time point were taken from the same vial. The % aggregation for each formulation were obtained. Aggregation was measured by SEC (% HMWS) for each sample.

The SEC assay uses an HPLC instrument equipped with a pump, a temperature controlled autosampler, a column heater, and Fluorescence detector. The sample solutions are analyzed using the following instrumentation and method parameters:

Mobile Phase: Dulbecco's Phosphate-Buffered Saline (D-PBS) containing Calcium and Magnesium adding 0.36 M Sodium Chloride (0.9 mM Calcium Chloride, 0.5 mM Magnesium Chloride, 2.7 mM Potassium Chloride, 1.5 mM Potassium Phosphate Mono basic, 496 mM Sodium Chloride, 8.1 mM Sodium Phosphate Dibasic, pH 7.0±0.1)
Column Heater: 26° C.
Run Time: 40 minutes
FL Detector: Ex/Em=280/350 nm, PMT Gain=5
Injection Mass Load: 2 μg
Autosampler Temperature: 5° C.
Flow Rate: 0.5 mL/min, Isocratic

TABLE 12
Arginine Concentration Study Overview

| | efanesoctocog | | | | |
|---|---|---|---|---|---|
| | alfa | Arginine | Study | Study | Quality |
| Study | concentration | Levels | conditions | timepoints | Attributes |
| Selection of L- | 1 mg/mL | 50, 100, 125, | Freezing | 40-, 80-, 120-, | % Aggregates |
| Arginine HCl as | | 150, 175, 200, | at −80° C. and | and 160- | by SE-HPLC |
| a stabilizing | | 250, and 300 | ambient room | minutes post | |
| excipient | | mM | termperature | thaw | |
| | | | thaw | | |

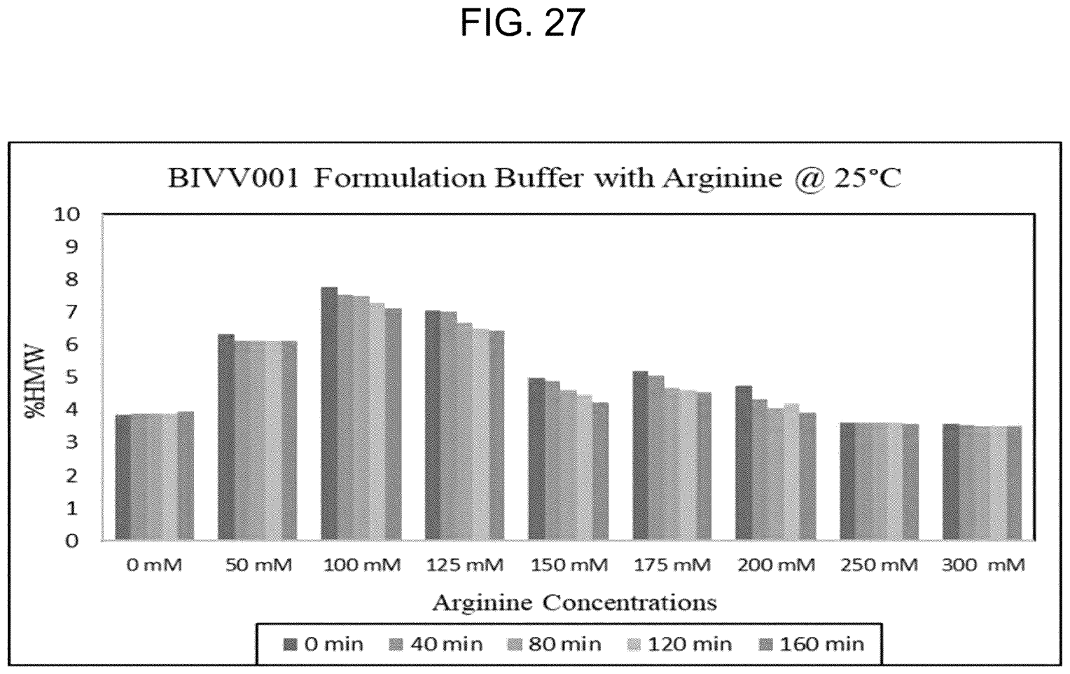

Two experiments (each run in duplicate) were run using the conditions outlined in Table 12. The results from Experiment 1 are shown in Table 13 and FIG. 27. In Experiment 1, High Molecular Weight (HMW) aggregates were found at a higher percentage in formulations with lower concentrations of arginine. Higher concentrations of arginine (200 mM to 300 mM) exhibited a consistently low percentage of HMW aggregates out to 160 minutes post-thaw.

TABLE 13
High Molecular Weight Aggregate Percentage - Experiment 1

| | | % HMW AREA | | | | |
|---|---|---|---|---|---|---|
| Arginine | | 0 min | 40 min | 80 min | 120 min | 160 min |
| 0 | mM | 4.24 | 4.22 | 4.25 | 4.24 | 4.26 |
| 50 | mM | 8.38 | 8.27 | 8.16 | 8.05 | 7.93 |
| 100 | mM | 7.36 | 7.13 | 6.94 | 6.83 | 6.60 |
| 125 | mM | 7.35 | 6.92 | 6.56 | 6.12 | 5.85 |
| 150 | mM | 4.79 | 4.42 | 4.32 | 4.19 | 4.01 |
| 175 | mM | 5.73 | 5.13 | 4.46 | 4.26 | 3.97 |
| 200 | mM | 4.10 | 3.77 | 3.78 | 3.54 | 3.63 |
| 250 | mM | 3.72 | 3.71 | 3.70 | 3.71 | 3.70 |
| 300 | mM | 3.60 | 3.60 | 3.60 | 3.59 | 3.61 |

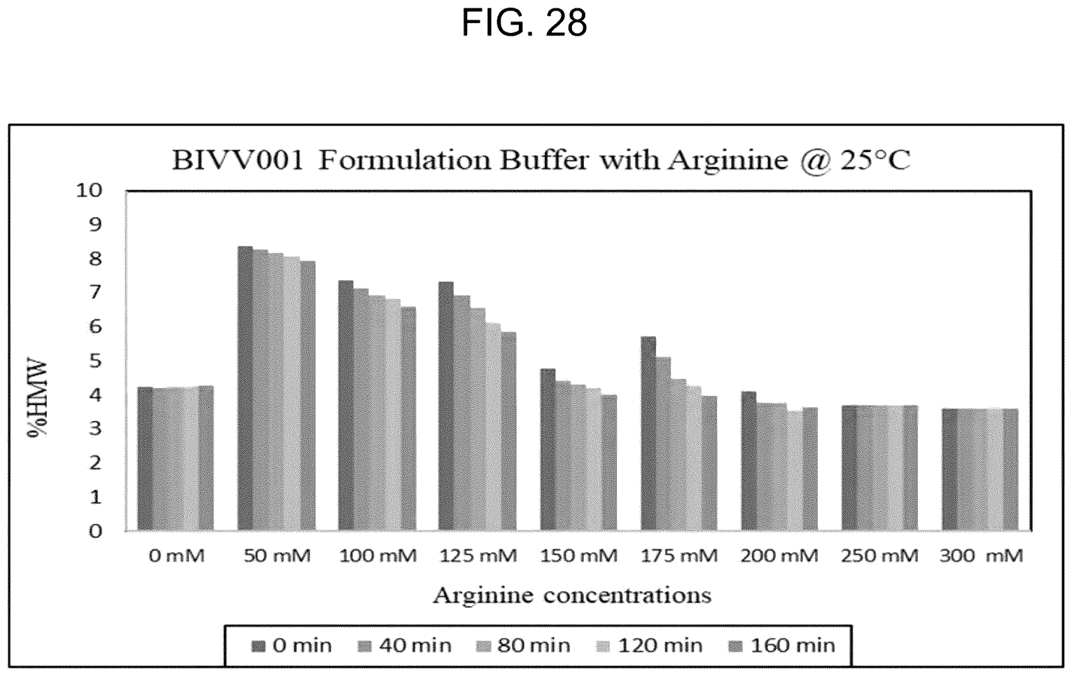

The results from Experiment 2 can be seen in Table 14 and FIG. 28. In Experiment 2, HMW aggregates were found at a higher percentage in formulations with lower concentrations of arginine. Consistent with the observations of Experiment 1, Experiment 2 also showed that, higher concentrations of arginine (200 mM to 300 mM) exhibited a consistently low percentage of HMW aggregates out to 160 minutes post-thaw.

TABLE 14
High Molecular Weight Aggregate Percentage - Experiment 2

| | | % HMW AREA | | | | |
|---|---|---|---|---|---|---|
| Arginine | | 0 min | 40 min | 80 min | 120 min | 160 min |
| 0 | mM | 4.24 | 4.22 | 4.25 | 4.24 | 4.26 |
| 50 | mM | 8.38 | 8.27 | 8.16 | 8.05 | 7.93 |
| 100 | mM | 7.36 | 7.13 | 6.94 | 6.83 | 6.60 |
| 125 | mM | 7.35 | 6.92 | 6.56 | 6.12 | 5.85 |
| 150 | mM | 4.79 | 4.42 | 4.32 | 4.19 | 4.01 |
| 175 | mM | 5.73 | 5.13 | 4.46 | 4.26 | 3.97 |
| 200 | mM | 4.10 | 3.77 | 3.78 | 3.54 | 3.63 |
| 250 | mM | 3.72 | 3.71 | 3.70 | 3.71 | 3.70 |
| 300 | mM | 3.60 | 3.60 | 3.60 | 3.59 | 3.61 |

The osmolality of samples across the range of arginine concentrations was also studied in two separate experiments (each run in duplicate). The osmolality (mOsm/kg) of the formulations was shown to increase with increased concentration of arginine. In Experiment 1, the first set of replicates was assayed on the osmometer after having been out at room temperature for 24 hours. The second set of replicates is from the retains that were stored at −80° C. The results from Experiment 1 are displayed in Table 15. In Experiment #2, the single set of replicate samples is from the retains that were stored at −80° C. The results from Experiment 2 are displayed in Table 16.

TABLE 15
Osmolality of formulations with increasing
arginine concentrations - Experiment 1

Experiment 1

| | Rep. 1 | Rep. 2 |
|---|---|---|
| | (RT) | (−80° C.) |
| Sample ID | mOsm/kg | mOsm/kg |
| 290 reference | 295 | 288 |
| 0 mM | 218 | n/a |
| 50 mM Arg | 327 | 331 |
| 100 mM Arg | 371 | 420 |
| 125 mM Arg | 408 | 425 |
| 150 mM Arg | 495 | 484 |
| 175 mM Arg | 530 | 524 |
| 200 mM Arg | 534 | 537 |
| 250 mM Arg | 655 | 667 |
| 300 mM Arg | 758 | 699 |

TABLE 16
Osmolality of formulations with increasing
arginine concentrations - Experiment 2

| | Experiment 2 |
|---|---|
| | (−80° C.) |
| Sample ID | mOsm/kg |
| 290 reference | 288 |
| 0 mM | 184 |
| 50 mM Arg | 302 |
| 100 mM Arg | 356 |
| 125 mM Arg | 398 |
| 150 mM Arg | 476 |
| 175 mM Arg | 513 |
| 200 mM Arg | 538 |
| 250 mM Arg | n/a |
| 300 mM Arg | 701 |

The pH was not adjusted for the samples tested, and the pH of formulations across the range of arginine concentrations was subsequently assessed. The results are shown in Table 17.

TABLE 17
pH of formulations with increasing arginine concentrations

| | Desired | replicate 1 | replicate 2 | replicate 3 |
|---|---|---|---|---|
| | Arg | pH as | pH as | pH as |
| | concen- | measured by | measured by | measured by |
| Sample | tration | pH meter 1 | pH meter 2 | pH meter 3 |
| Formulation 1 | 0 | 7.00 | 6.99 | 6.95 |
| Formulation 2 | 50 | 6.74 | 6.74 | 6.76 |
| Formulation 3 | 100 | 6.74 | 6.70 | 6.75 |
| Formulation 4 | 125 | 6.74 | 6.69 | 6.70 |
| Formulation 5 | 150 | 6.69 | 6.65 | 6.67 |
| Formulation 6 | 175 | 6.69 | 6.62 | 6.67 |
| Formulation 7 | 200 | 6.67 | 6.60 | 6.70 |
| Formulation 8 | 250 | 6.54 | 6.54 | 6.65 |
| Formulation 9 | 300 | 6.50 | 6.51 | 6.62 |

Conclusion: In these experiments, surprisingly the formulations comprising efanesoctocog alfa having at least 250 mM arginine showed the lowest % HMW at time 0 (T0) and subsequent timepoints tested (see FIG. 27 and FIG. 28), while lower levels of arginine increased aggregation at TO. At the beginning of time zero, the control, 0% arginine, had a TO of ˜ 4% HMW area, and the 50 mM, 100 mM, 125 mM and 150 mM started with a substantially greater % HMW, showing that the arginine at low concentrations from 50 mM-200 mM began at higher aggregation levels that were greater than the control, 0 mM arginine. There was a decrease in % HMW area over time at the 50 mM, 100 mM, 125 mM and 150 mM concentrations.

Thus, the use of at least 250 mM arginine in the formulation appears to stabilize efanesoctocog alfa over time with the least amount of aggregation.

### Example 7: Evaluation of Low Sucrose on Protein Stability

The experiments described in this example assess the stability of efanesoctocog alfa in pharmaceutical compositions formulated with different concentrations of sucrose. In addition to sucrose, each composition also contained 10 mM L-histidine, 250 mM L-arginine-HCl, 5 mM CaCl₂), and 0.05% (w/v) Polysorbate 80, at a pH of 6.8.

Efanesoctocog alfa drug substance (DS) comprises 1 mg/ml efanesoctocog alfa, 10 mM L-histidine, 250 mM L-arginine-HCl, 5 mM CaCl₂), and 0.05% (w/v) polysorbate 80, and sucrose, at a pH of 6.8. Efanesoctocog alfa drug product (DP) is DS diluted to a desired activity in international units (IU), such as IU per vial (e.g., IU per 3.367 mL when a vial is filled with 3.367 mL of drug product). DP may be lyophilized (e.g., in a vial) resulting in lyophilized DP that may then be reconstituted prior to injection.

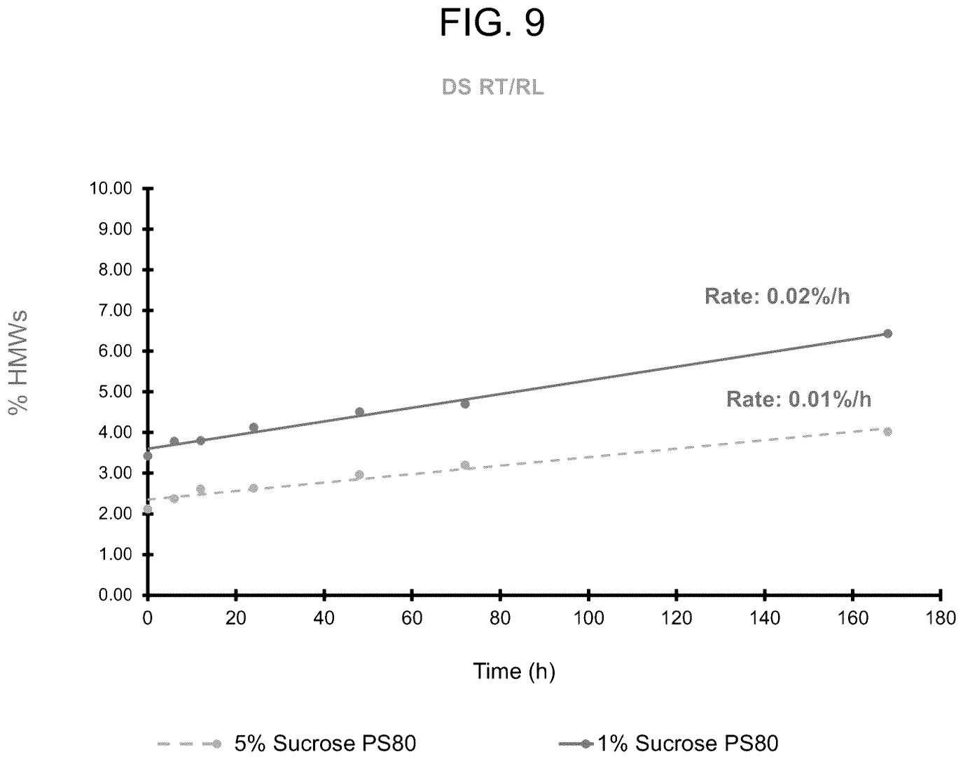

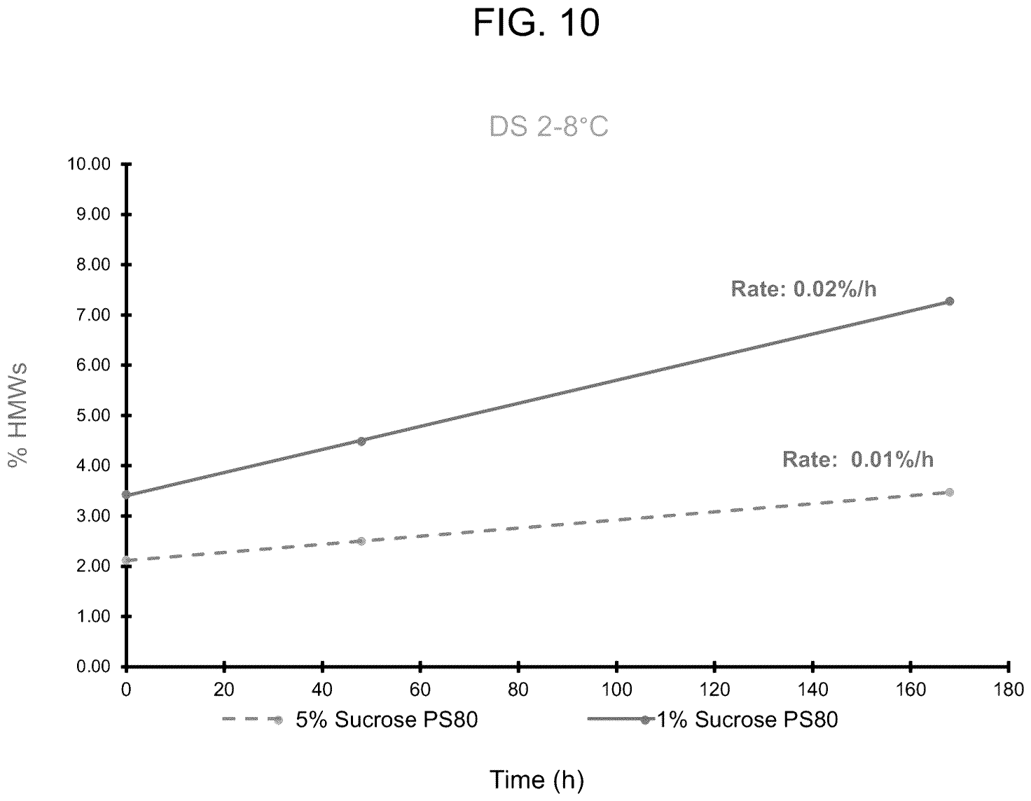

Aggregation of efanesoctocog alfa drug substance (DS) compositions (1 mg/mL) containing 5% or 1% sucrose were tested at either room temperature (RT)/room light (RL) conditions or at 2-8° C. As shown in FIG. 9 and FIG. 10, DS compositions containing 5% sucrose showed less aggregation (% HMWS) over time than DS compositions containing 1% sucrose.

To further analyze the impact on sucrose concentration on efanesoctocog alfa compositions, pharmaceutical compositions of efanesoctocog alfa at 0, 1, 2, and 5% w/v sucrose were subjected to different stability conditions and stability was assessed using several methods. Compositions were tested at the following product stages: drug substance (DS, which was had not been lyophilized or adjusted for any particular IU strength) frozen storage stability, DS freeze/thaw (FT) stability, liquid DS post-thaw stability, diluted DS and drug product (DP) stability, DP lyophilization stability, and DP-post reconstitution. Drug substance was tested at freezing temperatures (−80° C. or −30° C.). Drug product samples were tested at room temperature (RT), 5° C., 30° C., and 40° C. Lyophilized DP samples were tested at two different DP concentrations (4000 IU and 250 IU).

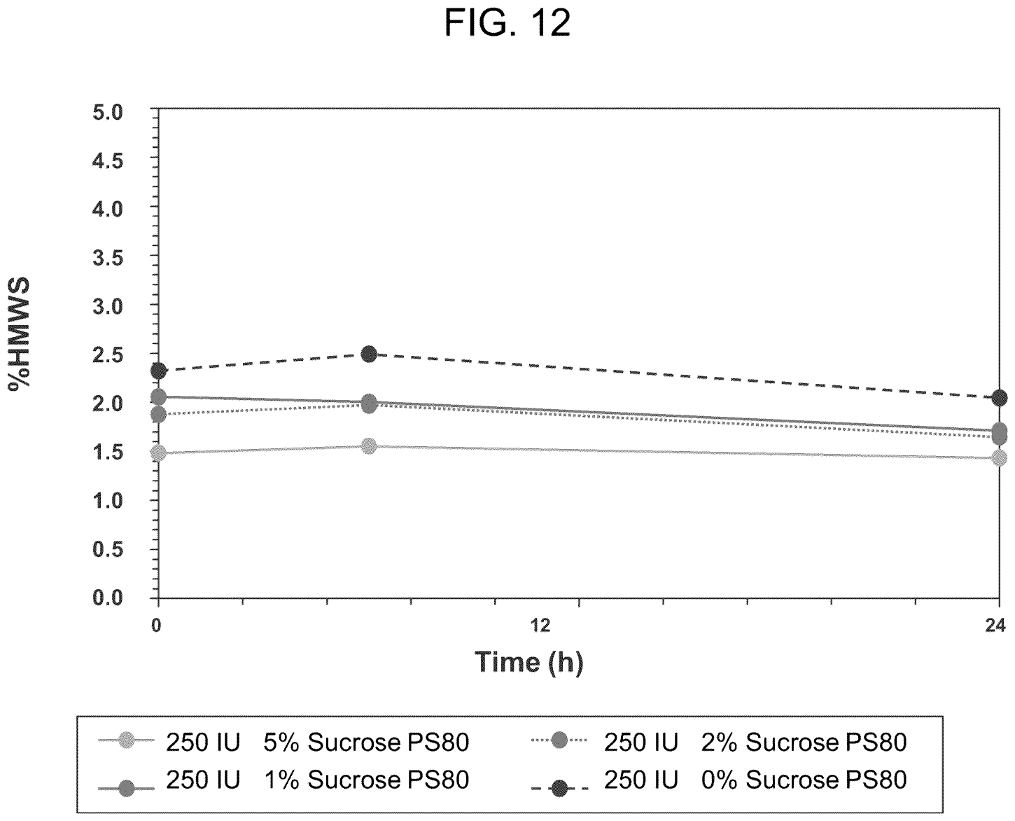

Aggregation of efanesoctocog alfa bulk drug product (BDP) compositions at 250 IU were tested at either room temperature (RT)/room light (RL) conditions or at 2-8° C. BDP compositions containing 0, 1, 2, and 5% w/v sucrose were tested. Results at RT/RL are shown in FIG. 11 and results at 2-8° C. are shown in FIG. 12. BDP compositions containing 0% sucrose showed the highest aggregation levels for both conditions. However, none of the BDP compositions at 250 IU showed significant increases in % HMWS over the 24 hour time period tested. For all compositions and both conditions, it was observed that aggregation levels began to decrease over time after about 7 days (data not shown).

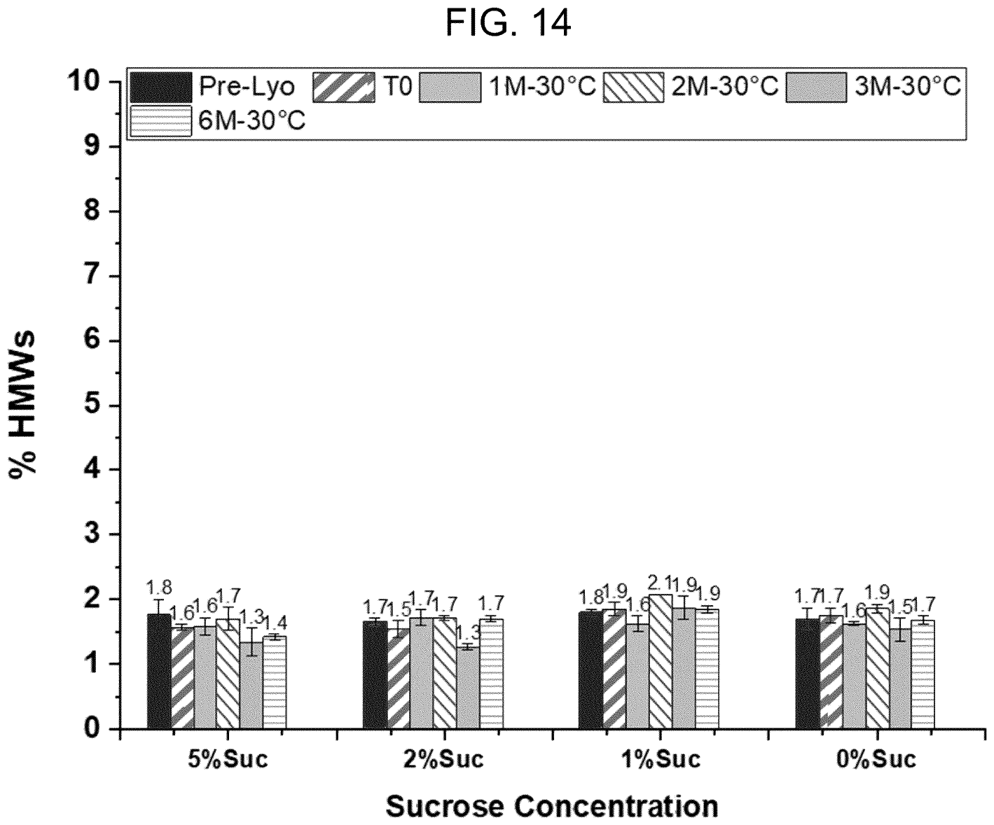

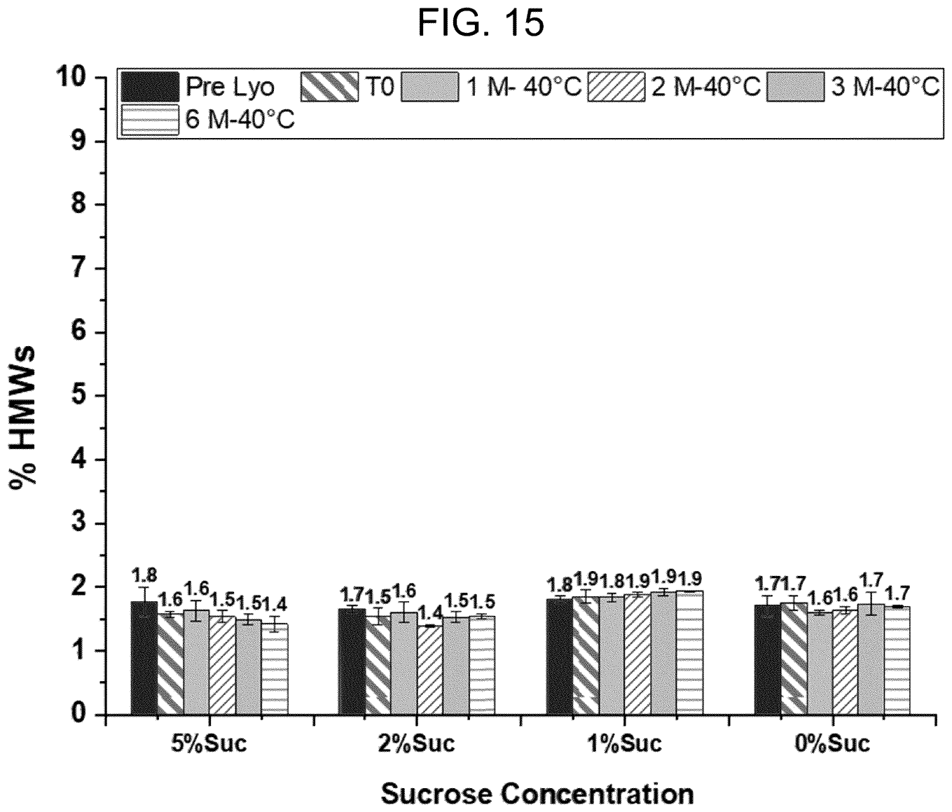

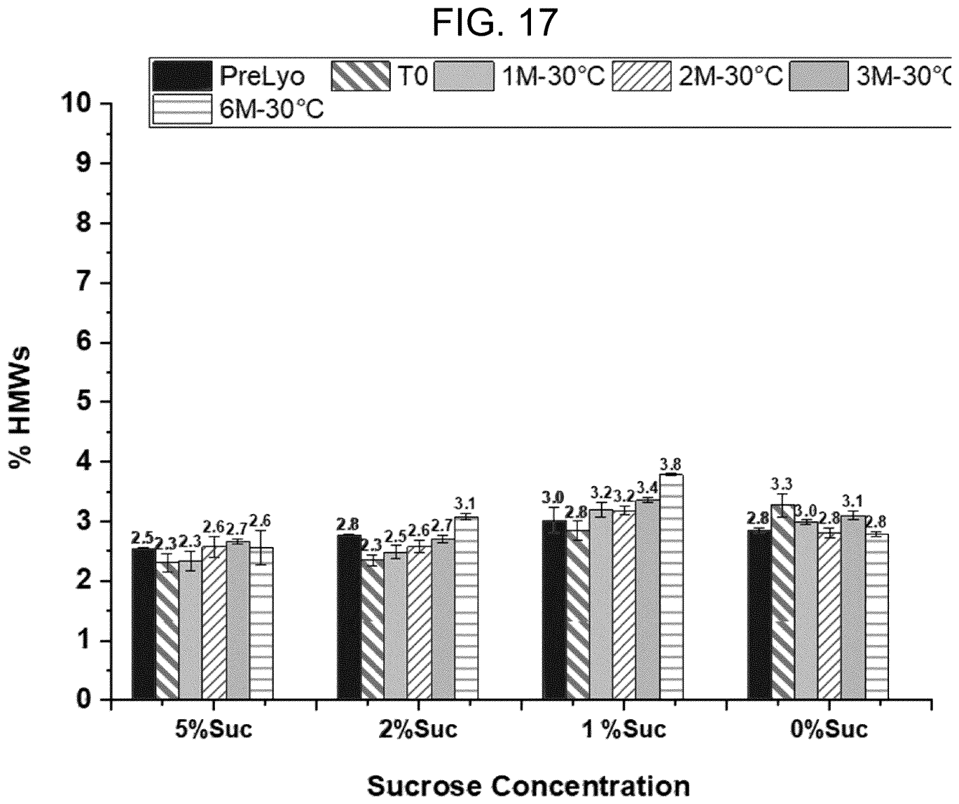

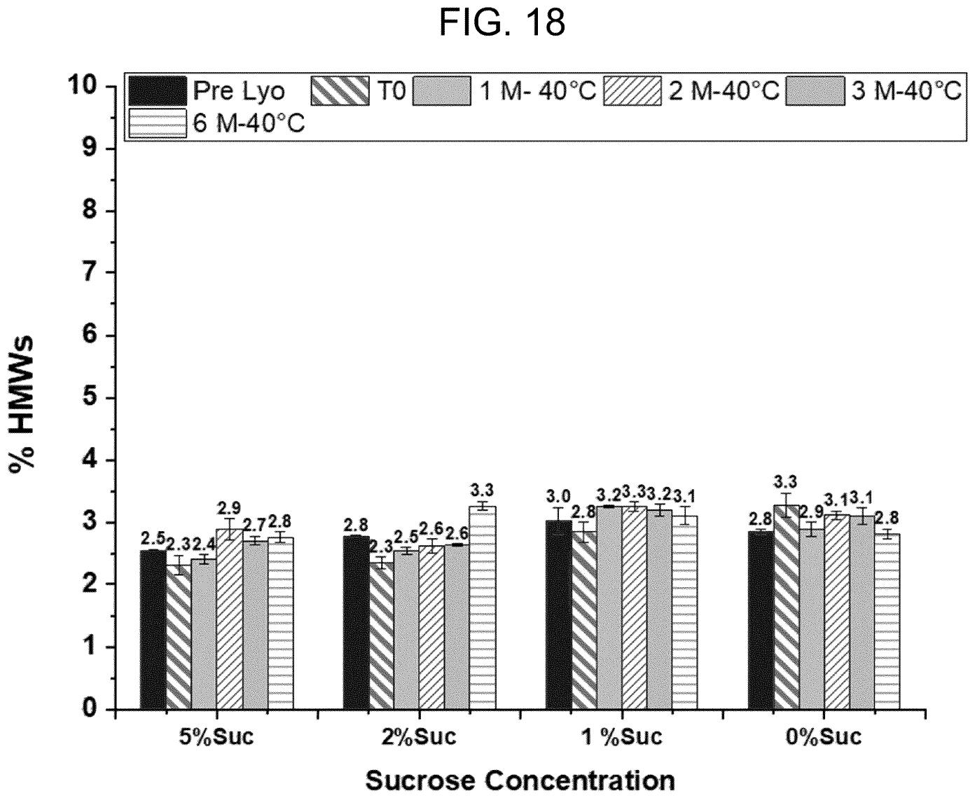

Aggregation of compositions of efanesoctocog alfa lyophilized drug product (Lyo DP) at 250 IU or 4000 IU were tested at 5° C., 30° C., and 40° C. Aggregation was measured by SEC (% HMWS) for each sample. Lyo DP compositions containing 0, 1, 2, and 5% w/v sucrose were tested. Samples were tested at TO, 1 month, 2 months, 3 months, and 6 months. Results of Lyo DP compositions at 250 IU are shown in FIG. 13 (5° C.), FIG. 14 (30° C.), and FIG. 15 (40° C.). Results of Lyo DP compositions at 4000 IU are shown in FIG. 16 (5° C.), FIG. 17 (30° C.), and FIG. 18 (40° C.). Generally, all sucrose concentrations showed no significant increase in % HMWs over 6 months at 5° C., 30° C. and 40° C. None of the compositions tested showed significant aggregation trends.

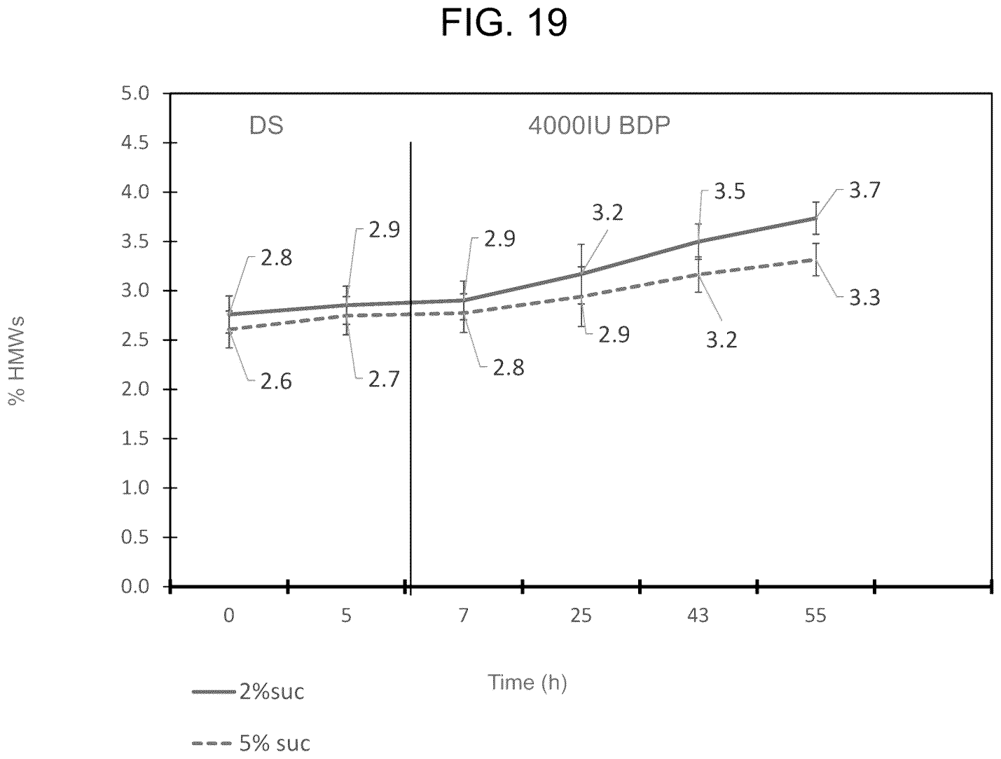

Aggregation of efanesoctocog alfa DS and 4000 IU bulk drug product (BDP) liquid compositions containing either 2% or 5% sucrose were tested by SEC. Samples were held at room temperature or 5° C. and tested after 0, 5, 7, 25, 43, and 55 hours. Samples were also tested after lyophilization. Results are shown in FIG. 19. Less aggregation over time was observed for compositions containing 5% sucrose. However, compositions containing 2% sucrose did not show significant increases in aggregation over the time period tested.

A summary of the results of the aggregation testing is provided in Table 18. An increase 1.0 or more % HMWS from a baseline value set forth as the corresponding value for 5% sucrose was deemed to be unacceptable. 0% sucrose was found to be unacceptable.

TABLE 18
Sucrose screening stability results (% HMWS).

| | Frozen | | | | | | | | | | | |
|---|---|---|---|---|---|---|---|---|---|---|---|---|
| | Storage, % | | Liquid DS | Liquid BDP | | | | | | | | |
| | (−80° C.) | | Rate %/h | Rate %/h | | Cum. %/h | Lyo DP (Δ6M) | | | Lyo DP (Δ6M) | | |
| Sucrose | (1 mg/mL) | | (1 mg/mL) | (4000 IU) | | (4000 IU) | (4000 IU) | | | (250 IU) | | |
| conc, % | Δ6M | Δ5XFT | RT | 5° C. | RT | 5° C./RT | 5° C. | 30° C. | 40° C. | 5° C. | 30° C. | 40° C. |
| 5 | 0.3 | 1.0 | 0.03 | 0.01 | 0.02 | 0.01 | 0.3 | 0.2 | 0.4 | 0.0 | −0.1 | 0.0 |
| 2 | 0.3 | 1.2 | 0.02 | 0.02 | 0.02 | 0.02 | 0.5 | 0.7 | 0.9 | 0.0 | 0.2 | 0.0 |
| 1 | 0.3 | 1.3 | 0.08 | 0.03 | 0.04 | 0.03 | 0.7 | 0.9 | 0.3 | 0.1 | 0.0 | 0.0 |
| 0 | 0.6 | 2.2 | ND | ND | ND | ND | 0.2 | 0.0 | −0.2 | 0.2 | 0.1 | 0.1 |

ND = Not Determined

As shown in Table 18, stability results indicated that freeze/thaw and frozen storage stability data was favorable for 1% to 5% w/v sucrose at −80° C. Freeze/thaw and frozen storage stability was unfavorable at all concentrations at −30° C. (data not shown). DP samples showed a minor but acceptable increase in aggregation through 6 months at 40° C. for the 1% and 2% formulations.

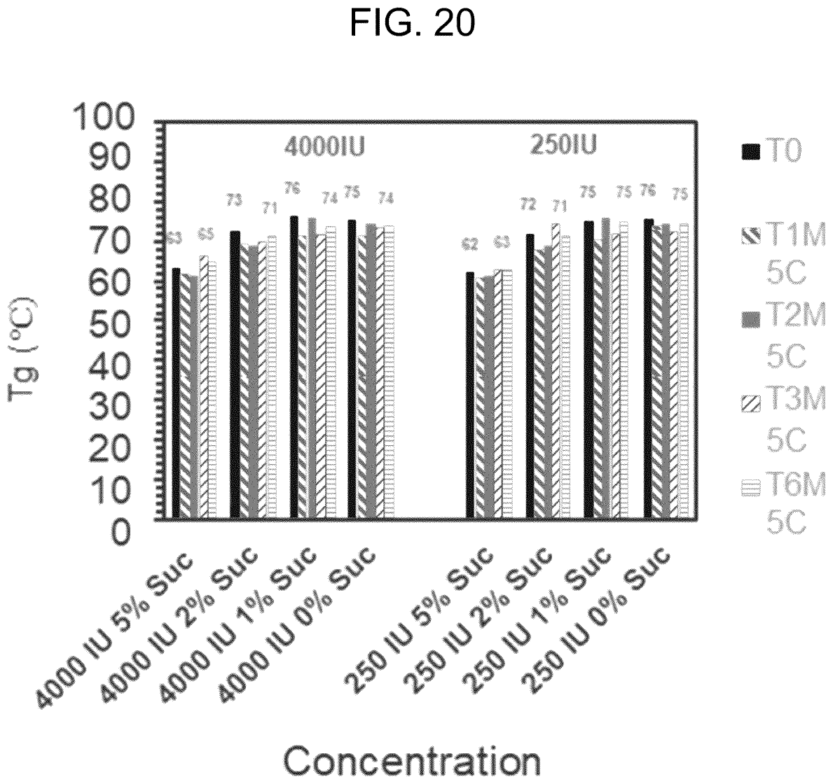

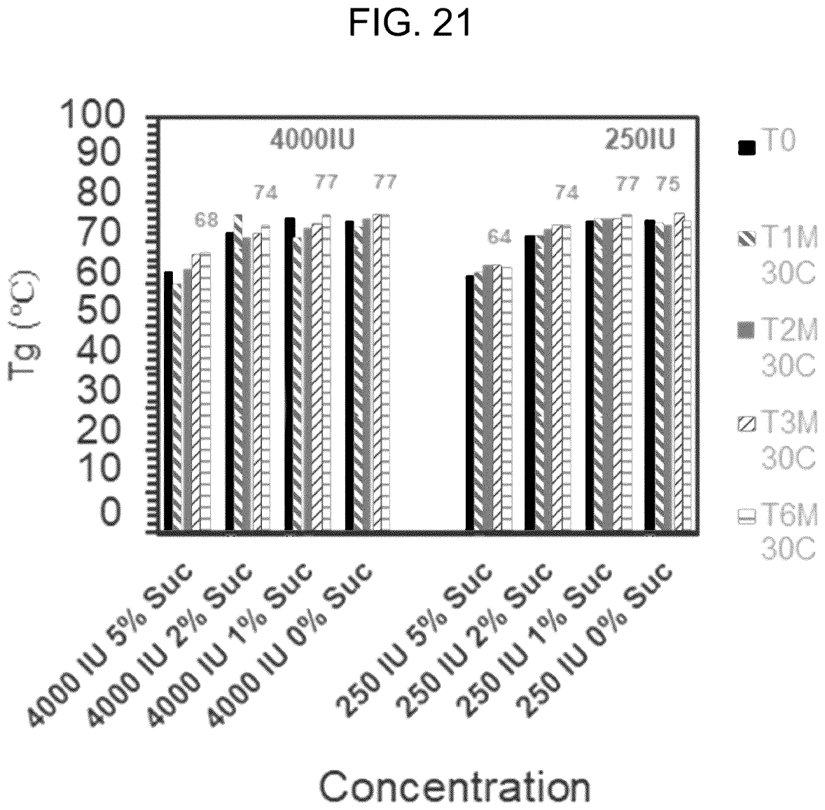

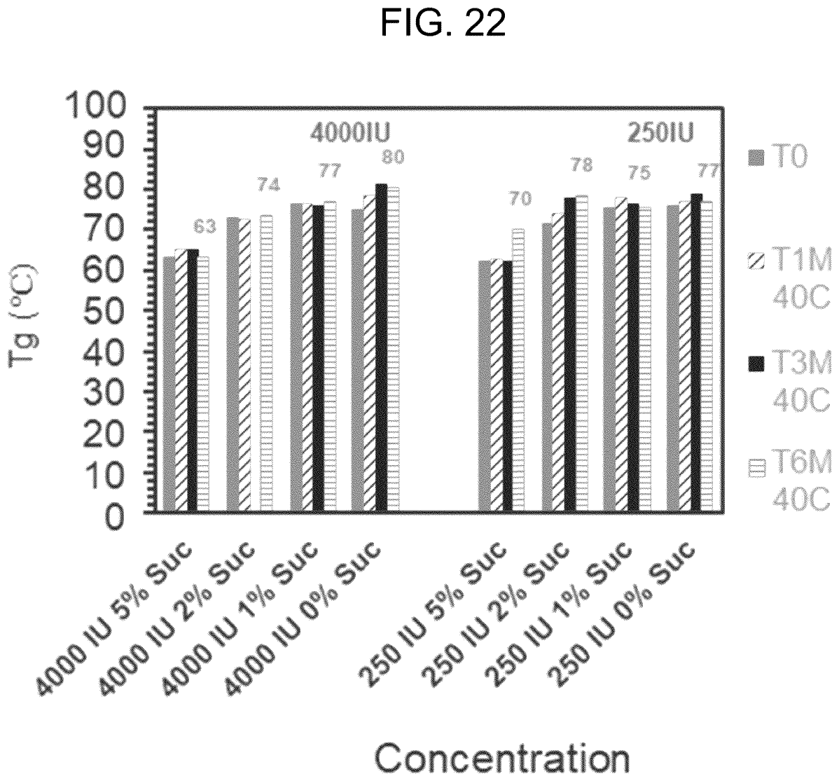

The glass transition temperature (Tg) of compositions of efanesoctocog alfa DP at 250 IU or 4000 IU were tested at 5° C., 30° C., and 40° C. Tg was measured by modulated differential scanning calorimetry (DSC). DP compositions containing 0, 1, 2, and 5% w/v sucrose were tested. Samples were tested at TO, 1 month, 2 months, 3 months, and 6 months. Results are show in in FIG. 20 (5° C.), FIG. 21 (30° C.), and FIG. 22 (40° C.). Generally, Tg was observed to correlate with sucrose concentration, with the higher sucrose concentration showing lower Tg. No significant changes in Tg were observed at 5° C., 30° C. and 40° C. through 6 months for all compositions. No major changes in Tg were observed at the different DP concentrations.

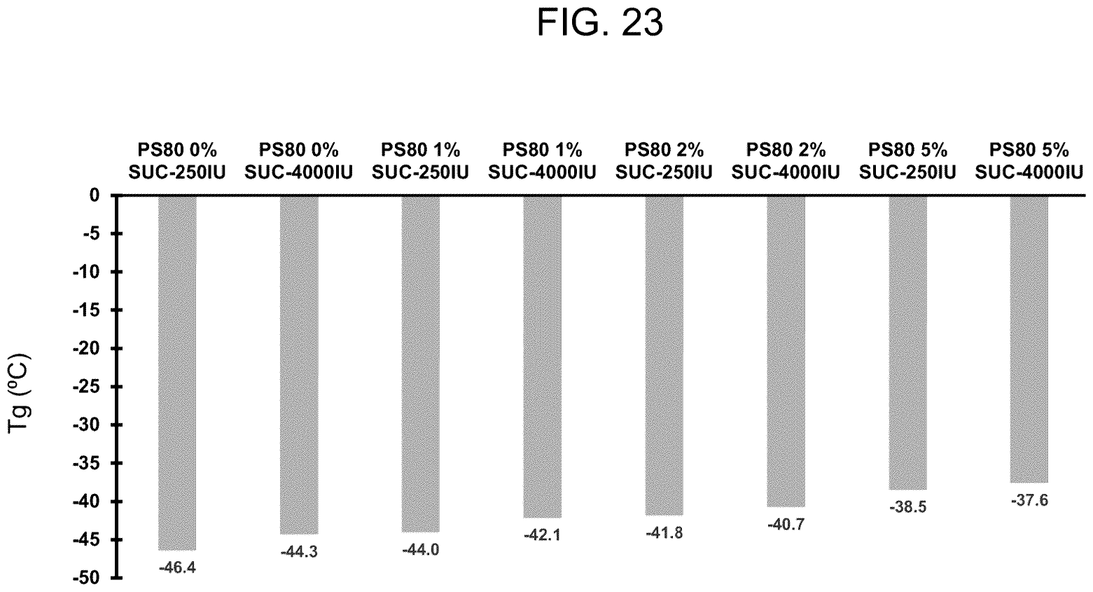

The glass transition temperature (Tg) of efanesoctocog alfa drug product (DP) containing 0, 1, 2, and 5% w/v sucrose was measured by differential scanning calorimetry (DSC). DP samples were tested at two different DP concentrations (4000 IU and 250 IU). Results are shown in FIG. 23. The DSC protocol for Tg determination includes:
(i) equilibration at 15° C.,
(ii) modulate at 1° C. every 60 seconds,
(iii) isothermal for 5 minutes
(iv) ramp at 3° C./min to 130° C.

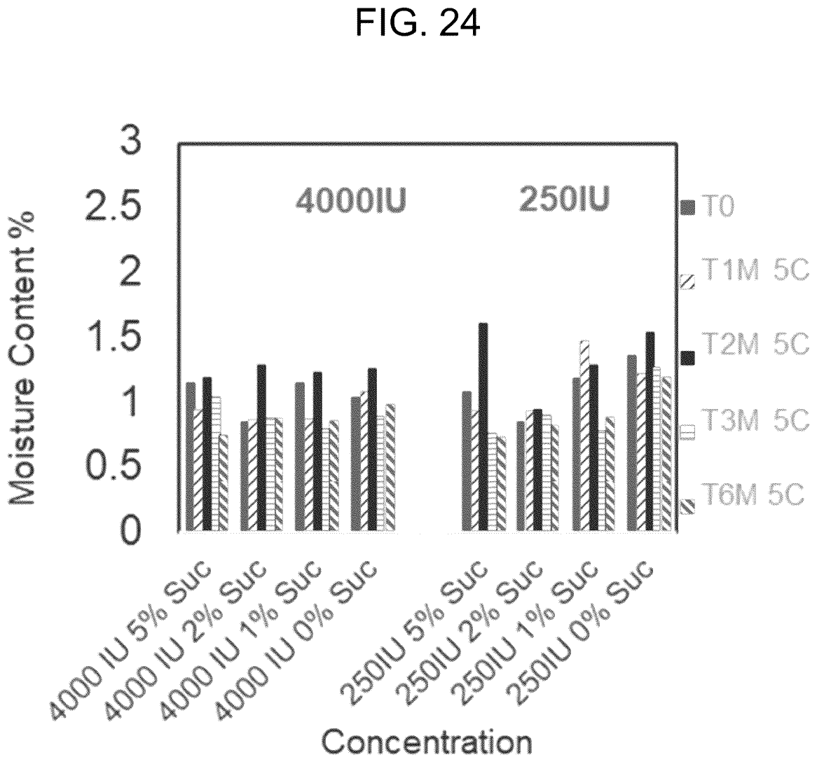

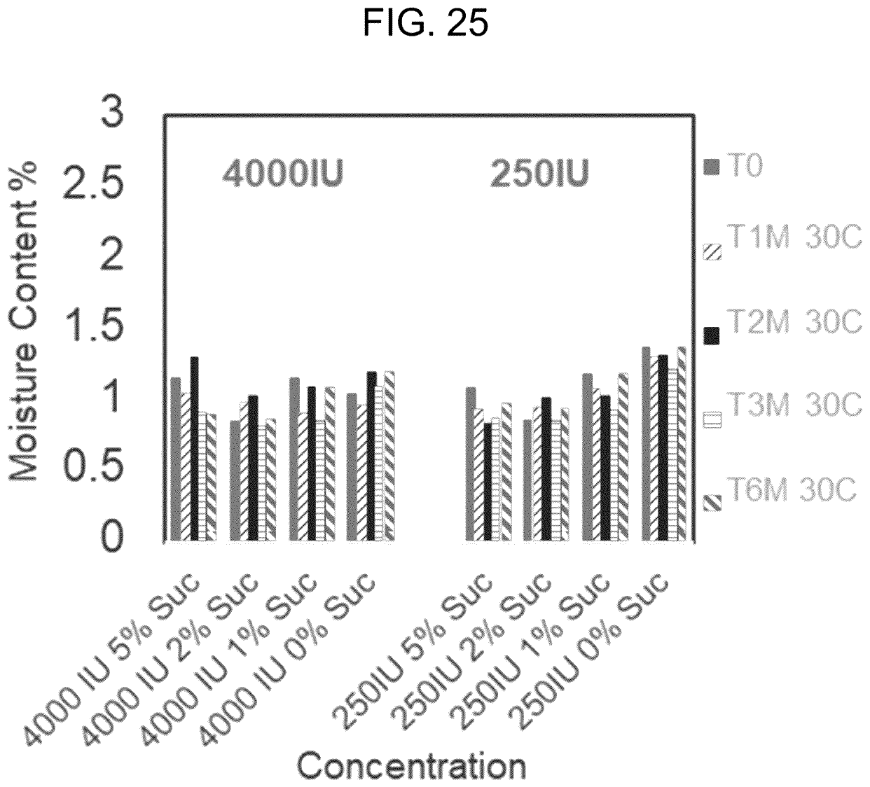

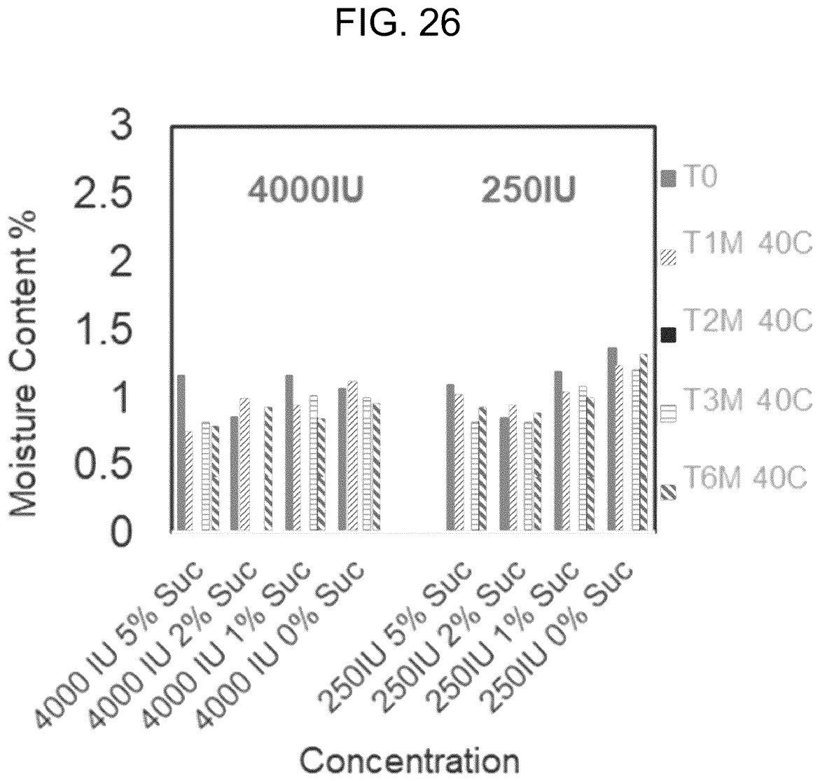

Residual moisture content of compositions of efanesoctocog alfa lyophilized drug product (Lyo DP) at 250 IU or 4000 IU were tested at 5° C., 30° C., and 40° C. Lyo DP compositions containing 0, 1, 2, and 5% w/v sucrose were tested. Samples were tested at TO, 1 month, 2 months, 3 months, and 6 months. Results are shown in FIG. 24 (5° C.), FIG. 25 (30° C.), and FIG. 26 (40° C.). No significant change in residual moisture content was observed at 5° C., 30° C. and 40° C. through 6 months for all compositions. All residual moisture values fell below the spec of 3%.

Upon visual inspection, concentration of sucrose had no effect on cake appearance. over 6 months at 2-8° C., 30° C. and 40° C. (data not shown).

Based on the results of these experiments, sucrose concentrations of 1% to 2% (w/v) appear to be the acceptable concentrations for pharmaceutical compositions of efanesoctocog alfa.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

### SEQUENCES

TABLE 19
Exemplary Chimeric protein Sequences
A-FVIII(ELNN)-Fc: SEQ ID NO: 1

| | |
|---|---|
| ATRRYYLGAV ELSWDYMQSD LGELPVDARF PPRVPKSFPF NTSVVYKKTL FVEFTDHLEN | 60 |
| IAKPRPPWMG LLGPTIQAEV YDTVVITLKN MASHPVSLHA VGVSYWKASE GAEYDDQTSQ | 120 |
| REKEDDKVFP GGSHTYVWQV LKENGPMASD PLCLTYSYLS HVDLVKDLNS GLIGALLVCR | 180 |
| EGSLAKEKTQ TLHKFILLFA VEDEGKSWHS ETKNSLMQDR DAASARAWPK MHTVNGYVNR | 240 |
| SLPGLIGCHR KSVYWHVIGM GTTPEVHSIF LEGHTFLVRN HRQASLEISP ITFLTAQTLL | 300 |
| MDLGQFLLFC HISSHQHDGM EAYVKVDSCP EEPQLRMKNN EEAEDYDDDL TDSEMDVVRF | 360 |
| DDDNSPSFIQ IRSVAKKHPK TWVHYIAAEE EDWDYAPLVL APDDRSYKSQ YLNNGPQRIG | 420 |
| RKYKKVREMA YTDETFKTRE AIQHESGILG PLLYGEVGDT LLIIFKNQAS RPYNIYPHGI | 480 |
| TDVRPLYSRR LPKGVKHLKD FPILPGEIFK YKWTVTVEDG PTKSDPRCLT RYYSSFVNME | 540 |
| RDLASGLIGP LLICYKESVD QRGNQIMSDK RNVILESVED ENRSWYLTEN IQRFLPNPAG | 600 |
| VQLEDPEFQA SNIMHSINGY VEDSLQLSVC LHEVAYWYIL SIGAQTDFLS VFFSGYTFKH | 660 |
| KMVYEDTLTL FPFSGETVEM SMENPGLWIL GCHNSDERNR GMTALLKVSS CDKNTGDYYE | 720 |
| DSYEDISAYL LSKNNAIEPR SFSQNGTSES ATPESGPGSE PATSGSETPG TSESATPESG | 780 |
| PGSEPATSGS ETPGTSESAT PESGPGTSTE PSEGSAPGSP AGSPTSTEEG TSESATPESG | 840 |
| PGSEPATSGS ETPGTSESAT PESGPGSPAG SPTSTEEGSP AGSPTSTEEG TSTEPSEGSA | 900 |
| PGTSESATPE SGPGTSESAT PESGPGTSES ATPESGPGSE PATSGSETPG SEPATSGSET | 960 |
| PGSPAGSPTS TEEGTSTEPS EGSAPGTSTE PSEGSAPGSE PATSGSETPG TSESATPESG | 1020 |
| PGTSTEPSEG SAPASSEITR TTLQSDQEEI DYDDTISVEM KKEDEDIYDE DENQSPRSFQ | 1080 |
| KKTRHYFIAA VERLWDYGMS SSPHVLRNRA QSGSVPQFKK VVFQEFTDGS FTQPLYRGEL | 1140 |
| NEHLGLLGPY IRAEVEDNIM VTFRNQASRP YSFYSSLISY EEDQRQGAEP RKNFVKPNET | 1200 |
| KTYFWKVQHH MAPTKDEFDC KAWAYFSDVD LEKDVHSGLI GPLLVCHTNT LNPAHGROVT | 1260 |
| VQEFALFFTI FDETKSWYFT ENMERNCRAP CNIQMEDPTF KENYRFHAIN GYIMDTLPGL | 1320 |
| VMAQDQRIRW YLLSMGSNEN IHSIHFSGHV FTVRKKEEYK MALYNLYPGV FETVEMLPSK | 1380 |
| AGIWRVECLI GEHLHAGMST LFLVYSNKCQ TPLGMASGHI RDFQITASGQ YGQWAPKLAR | 1440 |
| LHYSGSINAW STKEPFSWIK VDLLAPMIIH GIKTOGARQK FSSLYISQFI IMYSLDGKKW | 1500 |
| QTYRGNSTGT LMVFFGNVDS SGIKHNIFNP PIIARYIRLH PTHYSIRSTL RMELMGCDLN | 1560 |
| SCSMPLGMES KAISDAQITA SSYFTNMFAT WSPSKARLHL QGRSNAWRPQ VNNPKEWLQV | 1620 |
| DFQKTMKVTG VTTQGVKSLL TSMYVKEFLI SSSQDGHQWT LFFQNGKVKV FQGNQDSFTP | 1680 |
| VVNSLDPPLL TRYLRIHPQS WVHQIALRME VLGCEAQDLY DKTHTCPPCP APELLGGPSV | 1740 |
| FLFPPKPKDT LMISRTPEVT CVVVDVSHED PEVKENWYVD GVEVHNAKTK PREEQYNSTY | 1800 |
| RVVSVLTVLH QDWLNGKEYK CKVSNKALPA PIEKTISKAK GQPREPQVYT LPPSRDELTK | 1860 |
| NQVSLTCLVK GFYPSDIAVE WESNGQPENN YKTTPPVLDS DGSFFLYSKL TVDKSRWQQG | 1920 |
| NVFSCSVMHE ALHNHYTQKS LSLSPG | |

VWF(D′D3)- ELNN-a2 Linker-Fc: SEQ ID NO: 2

| | |
|---|---|
| SLSCRPPMVK LVCPADNLRA EGLECTKTCQ NYDLECMSMG CVSGCLCPPG MVRHENRCVA | 60 |
| LERCPCFHQG KEYAPGETVK IGCNTCVCRD RKWNCTDHVC DATCSTIGMA HYLTEDGLKY | 120 |
| LFPGECQYVL VODYCGSNPG TFRILVGNKG CSHPSVKCKK RVTILVEGGE IELFDGEVNV | 180 |
| KRPMKDETHF EVVESGRYII LLLGKALSVV WDRHLSISVV LKQTYQEKVC GLCGNEDGIQ | 240 |
| NNDLTSSNLQ VEEDPVDFGN SWKVSSQCAD TRKVPLDSSP ATCHNNIMKQ TMVDSSCRIL | 300 |
| TSDVFQDCNK LVDPEPYLDV CIYDTCSCES IGDCAAFCDT IAAYAHVCAQ HGKVVTWRTA | 360 |
| TLCPQSCEER NLRENGYEAE WRYNSCAPAC QVTCQHPEPL ACPVQCVEGC HAHCPPGKIL | 420 |
| DELLQTCVDP EDCPVCEVAG RRFASGKKVT LNPSDPEHCQ ICHCDVVNLT CEACQEPGTS | 480 |
| ESATPESGPG SEPATSGSET PGTSESATPE SGPGSEPATS GSETPGTSES ATPESGPGTS | 540 |
| TEPSEGSAPG SPAGSPTSTE EGTSESATPE SGPGSEPATS GSETPGTSES ATPESGPGSP | 600 |
| AGSPTSTEEG SPAGSPTSTE EGASSDKNTG DYYEDSYEDI SAYLLSKNNA IEPRSFSDKT | 660 |
| HTCPPCPAPE LLGGPSVFLF PPKPKDTLMI SRTPEVTCVV VDVSHEDPEV KENWYVDGVE | 720 |
| VHNAKTKPRE EQYNSTYRVV SVLTVLHQDW LNGKEYKCKV SNKALPAPIE KTISKAKGQP | 780 |
| REPQVYTLPP SRDELTKNQV SLTCLVKGFY PSDIAVEWES NGQPENNYKT TPPVLDSDGS | 840 |
| FFLYSKLTVD KSRWQQGNVF SCSVMHEALH NHYTQKSLSL SPG | 883 |

Amino acid sequence of FVIII protein (312A) -
includes signal peptide: SEQ ID NO: 3

| | |
|---|---|
| 1 | MQIELSTCFF LCLLRFCESA TRRYYLGAVE LSWDYMQSDL GELPVDAREP |
| 51 | PRVPKSFPFN TSVVYKKTLF VEFTDHLFNI AKPRPPWMGL LGPTIQAEVY |
| 101 | DTVVITLKNM ASHPVSLHAV GVSYWKASEG AEYDDQTSQR EKEDDKVFPG |
| 151 | GSHTYVWQVL KENGPMASDP LCLTYSYLSH VDLVKDLNSG LIGALLVCRE |
| 201 | GSLAKEKTQT LHKFILLFAV FDEGKSWHSE TKNSLMQDRD AASARAWPKM |
| 251 | HTVNGYVNRS LPGLIGCHRK SVYWHVIGMG TTPEVHSIFL EGHTFLVRNH |
| 301 | ROASLEISPI TELTAQTLLM DLGQFLLFCH ISSHQHDGME AYVKVDSCPE |
| 351 | EPQLRMKNNE EAEDYDDDLT DSEMDVVRED DDNSPSFIQI RSVAKKHPKT |
| 401 | WVHYIAAEEE DWDYAPLVLA PDDRSYKSQY LNNGPORIGR KYKKVREMAY |
| 451 | TDETFKTREA IQHESGILGP LLYGEVGDTL LIIFKNQASR PYNIYPHGIT |
| 501 | DVRPLYSRRL PKGVKHLKDF PILPGEIFKY KWTVTVEDGP TKSDPRCLTR |
| 551 | YYSSFVNMER DLASGLIGPL LICYKESVDQ RGNQIMSDKR NVILESVEDE |
| 601 | NRSWYLTENI QRFLPNPAGV QLEDPEFQAS NIMHSINGYV FDSLQLSVCL |
| 651 | HEVAYWYILS IGAQTDFLSV FFSGYTFKHK MVYEDTLTLF PESGETVEMS |
| 701 | MENPGLWILG CHNSDERNRG MTALLKVSSC DKNTGDYYED SYEDISAYLL |
| 751 | SKNNAIEPRS FSQNGTSESA TPESGPGSEP ATSGSETPGT SESATPESGP |
| 801 | GSEPATSGSE TPGTSESATP ESGPGTSTEP SEGSAPGSPA GSPTSTEEGT |
| 851 | SESATPESGP GSEPATSGSE TPGTSESATP ESGPGSPAGS PTSTEEGSPA |
| 901 | GSPTSTEEGT STEPSEGSAP GTSESATPES GPGTSESATP ESGPGTSESA |
| 951 | TPESGPGSEP ATSGSETPGS EPATSGSETP GSPAGSPTST EEGTSTEPSE |
| 1001 | GSAPGTSTEP SEGSAPGSEP ATSGSETPGT SESATPESGP GTSTEPSEGS |
| 1051 | APASSEITRT TLOSDQEEID YDDTISVEMK KEDEDIYDED ENQSPRSFQK |
| 1101 | KTRHYFIAAV ERLWDYGMSS SPHVLRNRAQ SGSVPQFKKV VFQEFTDGSE |
| 1151 | TOPLYRGELN EHLGLLGPYI RAEVEDNIMV TERNQASRPY SFYSSLISYE |
| 1201 | EDQROGAEPR KNFVKPNETK TYFWKVQHHM APTKDEFDCK AWAYFSDVDL |
| 1251 | EKDVHSGLIG PLLVCHTNTL NPAHGROVTV QEFALFFTIF DETKSWYFTE |
| 1301 | NMERNCRAPC NIQMEDPTEK ENYRFHAING YIMDTLPGLV MAQDQRIRWY |
| 1351 | LLSMGSNENI HSIHFSGHVF TVRKKEEYKM ALYNLYPGVF ETVEMLPSKA |
| 1401 | GIWRVECLIG EHLHAGMSTL FLVYSNKCQT PLGMASGHIR DFQITASGQY |
| 1451 | GQWAPKLARL HYSGSINAWS TKEPFSWIKV DLLAPMIIHG IKTQGARQKE |
| 1501 | SSLYISQFII MYSLDGKKWQ TYRGNSTGTL MVFFGNVDSS GIKHNIFNPP |
| 1551 | IIARYIRLHP THYSIRSTLR MELMGCDLNS CSMPLGMESK AISDAQITAS |
| 1601 | SYFTNMFATW SPSKARLHLQ GRSNAWRPQV NNPKEWLQVD FQKTMKVTGV |
| 1651 | TTOGVKSLLT SMYVKEFLIS SSQDGHQWTL FFQNGKVKVF QGNQDSFTPV |
| 1701 | VNSLDPPLLT RYLRIHPQSW VHOIALRMEV LGCEAQDLYD KTHTCPPCPA |
| 1751 | PELLGGPSVF LFPPKPKDTL MISRTPEVTC VVVDVSHEDP EVKENWYVDG |
| 1801 | VEVHNAKTKP REEQYNSTYR VVSVLTVLHQ DWLNGKEYKC KVSNKALPAP |
| 1851 | IEKTISKAKG QPREPQVYTL PPSRDELTKN QVSLTCLVKG FYPSDIAVEW |
| 1901 | ESNGQPENNY KTTPPVLDSD GSFFLYSKLT VDKSRWQQGN VESCSVMHEA |
| 1951 | LHNHYTQKSL SLSPG* |

Nucleotide sequence encoding FVIII
312A: SEQ ID NO: 4

| | |
|---|---|
| 1 | ATGCAAATAG AGCTCTCCAC CTGCTTCTTT CTGTGCCTTT TGCGATTCTG |
| 51 | CTTTAGTGCC ACCAGAAGAT ACTACCTGGG TGCAGTGGAA CTGTCATGGG |
| 101 | ACTATATGCA AAGTGATCTC GGTGAGCTGC CTGTGGACGC AAGATTTCCT |
| 151 | CCTAGAGTGC CAAAATCTTT TCCATTCAAC ACCTCAGTCG TGTACAAAAA |
| 201 | GACTCTGTTT GTAGAATTCA CGGATCACCT TTTCAACATC GCTAAGCCAA |
| 251 | GGCCACCCTG GATGGGTCTG CTAGGTCCTA CCATCCAGGC TGAGGTTTAT |
| 301 | GATACAGTGG TCATTACACT TAAGAACATG GCTTCCCATC CTGTCAGTCT |
| 351 | TCATGCTGTT GGTGTATCCT ACTGGAAAGC TTCTGAGGGA GCTGAATATG |
| 401 | ATGATCAGAC CAGTCAAAGG GAGAAAGAAG ATGATAAAGT CTTCCCTGGT |
| 451 | GGAAGCCATA CATATGTCTG GCAGGTCCTG AAAGAGAATG GTCCAATGGC |
| 501 | CTCTGACCCA CTGTGCCTTA CCTACTCATA TCTTTCTCAT GTGGACCTGG |
| 551 | TAAAAGACTT GAATTCAGGC CTCATTGGAG CCCTACTAGT ATGTAGAGAA |
| 601 | GGGAGTCTGG CCAAGGAAAA GACACAGACC TTGCACAAAT TTATACTACT |
| 651 | TTTTGCTGTA TTTGATGAAG GGAAAAGTTG GCACTCAGAA ACAAAGAACT |
| 701 | CCTTGATGCA GGATAGGGAT GCTGCATCTG CTCGGGCCTG GCCTAAAATG |
| 751 | CACACAGTCA ATGGTTATGT AAACAGGTCT CTGCCAGGTC TGATTGGATG |
| 801 | CCACAGGAAA TCAGTCTATT GGCATGTGAT TGGAATGGGC ACCACTCCTG |
| 851 | AAGTGCACTC AATATTCCTC GAAGGTCACA CATTTCTTGT GAGGAACCAT |
| 901 | CGCCAGGCGT CCTTGGAAAT CTCGCCAATA ACTTTCCTTA CTGCTCAAAC |
| 951 | ACTCTTGATG GACCTTGGAC AGTTTCTACT GTTTTGTCAT ATCTCTTCCC |
| 1001 | ACCAACATGA TGGCATGGAA GCTTATGTCA AAGTAGACAG CTGTCCAGAG |
| 1051 | GAACCCCAAC TACGAATGAA AAATAATGAA GAAGCGGAAG ACTATGATGA |
| 1101 | TGATCTTACT GATTCTGAAA TGGATGTGGT CAGGTTTGAT GATGACAACT |
| 1151 | CTCCTTCCTT TATCCAAATT CGCTCAGTTG CCAAGAAGCA TCCTAAAACT |
| 1201 | TGGGTACATT ACATTGCTGC TGAAGAGGAG GACTGGGACT ATGCTCCCTT |
| 1251 | AGTCCTCGCC CCCGATGACA GAAGTTATAA AAGTCAATAT TTGAACAATG |
| 1301 | GCCCTCAGCG GATTGGTAGG AAGTACAAAA AAGTCCGATT TATGGCATAC |
| 1351 | ACAGATGAAA CCTTTAAGAC TCGTGAAGCT ATTCAGCATG AATCAGGAAT |
| 1401 | CTTGGGACCT TTACTTTATG GGGAAGTTGG AGACACACTG TTGATTATAT |
| 1451 | TTAAGAATCA AGCAAGCAGA CCATATAACA TCTACCCTCA CGGAATCACT |
| 1501 | GATGTCCGTC CTTTGTATTC AAGGAGATTA CCAAAAGGTG TAAAACATTT |
| 1551 | GAAGGATTTT CCAATTCTGC CAGGAGAAAT ATTCAAATAT AAATGGACAG |
| 1601 | TGACTGTAGA AGATGGGCCA ACTAAATCAG ATCCTCGGTG CCTGACCCGC |
| 1651 | TATTACTCTA GTTTCGTTAA TATGGAGAGA GATCTAGCTT CAGGACTCAT |
| 1701 | TGGCCCTCTC CTCATCTGCT ACAAAGAATC TGTAGATCAA AGAGGAAACC |
| 1751 | AGATAATGTC AGACAAGAGG AATGTCATCC TGTTTTCTGT ATTTGATGAG |
| 1801 | AACCGAAGCT GGTACCTCAC AGAGAATATA CAACGCTTTC TCCCCAATCC |
| 1851 | AGCTGGAGTG CAGCTTGAGG ATCCAGAGTT CCAAGCCTCC AACATCATGC |
| 1901 | ACAGCATCAA TGGCTATGTT TTTGATAGTT TGCAGTTGTC AGTTTGTTTG |
| 1951 | CATGAGGTGG CATACTGGTA CATTCTAAGC ATTGGAGCAC AGACTGACTT |
| 2001 | CCTTTCTGTC TTCTTCTCTG GATATACCTT CAAACACAAA ATGGTCTATG |
| 2051 | AAGACACACT CACCCTATTC CCATTCTCAG GAGAAACTGT CTTCATGTCG |
| 2101 | ATGGAAAACC CAGGTCTATG GATTCTGGGG TGCCACAACT CAGACTTTCG |
| 2151 | GAACAGAGGC ATGACCGCCT TACTGAAGGT TTCTAGTTGT GACAAGAACA |
| 2201 | CTGGTGATTA TTACGAGGAC AGTTATGAAG ATATTTCAGC ATACTTGCTG |
| 2251 | AGTAAAAACA ATGCCATTGA ACCAAGAAGC TTCTCTCAAA ACGGTACCTC |
| 2301 | AGAGTCTGCT ACCCCCGAGT CAGGGCCAGG ATCAGAGCCA GCCACCTCCG |
| 2351 | GGTCTGAGAC ACCCGGGACT TCCGAGAGTG CCACCCCTGA GTCCGGACCC |
| 2401 | GGGTCCGAGC CCGCCACTTC CGGCTCCGAA ACTCCCGGCA CAAGCGAGAG |
| 2451 | CGCTACCCCA GAGTCAGGAC CAGGAACATC TACAGAGCCC TCTGAAGGCT |
| 2501 | CCGCTCCAGG GTCCCCAGCC GGCAGTCCCA CTAGCACCGA GGAGGGAACC |
| 2551 | TCTGAAAGCG CCACACCCGA ATCAGGGCCA GGGTCTGAGC CTGCTACCAG |
| 2601 | CGGCAGCGAG ACACCAGGCA CCTCTGAGTC CGCCACACCA GAGTCCGGAC |
| 2651 | CCGGATCTCC CGCTGGGAGC CCCACCTCCA CTGAGGAGGG ATCTCCTGCT |
| 2701 | GGCTCTCCAA CATCTACTGA GGAAGGAACC TCAACCGAGC CATCCGAGGG |
| 2751 | ATCAGCTCCC GGCACCTCAG AGTCGGCAAC CCCGGAGTCT GGACCCGGAA |
| 2801 | CTTCCGAAAG TGCCACACCA GAGTCCGGTC CCGGGACTTC AGAATCAGCA |
| 2851 | ACACCCGAGT CCGGCCCTGG GTCTGAACCC GCCACAAGTG GTAGTGAGAC |
| 2901 | ACCAGGATCA GAACCTGCTA CCTCAGGGTC AGAGACACCC GGATCTCCGG |
| 2951 | CAGGCTCACC AACCTCCACT GAGGAGGGCA CCAGCACAGA ACCAAGCGAG |
| 3001 | GGCTCCGCAC CCGGAACAAG CACTGAACCC AGTGAGGGTT CAGCACCCGG |
| 3051 | CTCTGAGCCG GCCACAAGTG GCAGTGAGAC ACCCGGCACT TCAGAGAGTG |
| 3101 | CCACCCCCGA GAGTGGCCCA GGCACTAGTA CCGAGCCCTC TGAAGGCAGT |
| 3151 | GCGCCAGCCT CGAGCGAAAT AACTCGTACT ACTCTTCAGT CAGATCAAGA |
| 3201 | GGAAATTGAC TATGATGATA CCATATCAGT TGAAATGAAG AAGGAAGATT |
| 3251 | TTGACATTTA TGATGAGGAT GAAAATCAGA GCCCCCGCAG CTTTCAAAAG |
| 3301 | AAAACACGAC ACTATTTTAT TGCTGCAGTG GAGAGGCTCT GGGATTATGG |
| 3351 | GATGAGTAGC TCCCCACATG TTCTAAGAAA CAGGGCTCAG AGTGGCAGTG |
| 3401 | TCCCTCAGTT CAAGAAAGTT GTTTTCCAGG AATTTACTGA TGGCTCCTTT |
| 3451 | ACTCAGCCCT TATACCGTGG AGAACTAAAT GAACATTTGG GACTCCTGGG |
| 3501 | GCCATATATA AGAGCAGAAG TTGAAGATAA TATCATGGTA ACTTTCAGAA |
| 3551 | ATCAGGCCTC TCGTCCCTAT TCCTTCTATT CTAGCCTTAT TTCTTATGAG |
| 3601 | GAAGATCAGA GGCAAGGAGC AGAACCTAGA AAAAACTTTG TCAAGCCTAA |
| 3651 | TGAAACCAAA ACTTACTTTT GGAAAGTGCA ACATCATATG GCACCCACTA |
| 3701 | AAGATGAGTT TGACTGCAAA GCCTGGGCTT ATTTCTCTGA TGTTGACCTG |
| 3751 | GAAAAAGATG TGCACTCAGG CCTGATTGGA CCCCTTCTGG TCTGCCACAC |
| 3801 | TAACACACTG AACCCTGCTC ATGGGAGACA AGTGACAGTA CAGGAATTTG |
| 3851 | CTCTGTTTTT CACCATCTTT GATGAGACCA AAAGCTGGTA CTTCACTGAA |
| 3901 | AATATGGAAA GAAACTGCAG GGCTCCCTGC AATATCCAGA TGGAAGATCC |
| 3951 | CACTTTTAAA GAGAATTATC GCTTCCATGC AATCAATGGC TACATAATGG |
| 4001 | ATACACTACC TGGCTTAGTA ATGGCTCAGG ATCAAAGGAT TCGATGGTAT |
| 4051 | CTGCTCAGCA TGGGCAGCAA TGAAAACATC CATTCTATTC ATTTCAGTGG |
| 4101 | ACATGTGTTC ACTGTACGAA AAAAAGAGGA GTATAAAATG GCACTGTACA |
| 4151 | ATCTCTATCC AGGTGTTTTT GAGACAGTGG AAATGTTACC ATCCAAAGCT |
| 4201 | GGAATTTGGC GGGTGGAATG CCTTATTGGC GAGCATCTAC ATGCTGGGAT |
| 4251 | GAGCACACTT TTTCTGGTGT ACAGCAATAA GTGTCAGACT CCCCTGGGAA |
| 4301 | TGGCTTCTGG ACACATTAGA GATTTTCAGA TTACAGCTTC AGGACAATAT |
| 4351 | GGACAGTGGG CCCCAAAGCT GGCCAGACTT CATTATTCCG GATCAATCAA |
| 4401 | TGCCTGGAGC ACCAAGGAGC CCTTTTCTTG GATCAAGGTG GATCTGTTGG |
| 4451 | CACCAATGAT TATTCACGGC ATCAAGACCC AGGGTGCCCG TCAGAAGTTC |
| 4501 | TCCAGCCTCT ACATCTCTCA GTTTATCATC ATGTATAGTC TTGATGGGAA |
| 4551 | GAAGTGGCAG ACTTATCGAG GAAATTCCAC TGGAACCTTA ATGGTCTTCT |
| 4601 | TTGGCAATGT GGATTCATCT GGGATAAAAC ACAATATTTT TAACCCTCCA |
| 4651 | ATTATTGCTC GATACATCCG TTTGCACCCA ACTCATTATA GCATTCGCAG |
| 4701 | CACTCTTCGC ATGGAGTTGA TGGGCTGTGA TTTAAATAGT TGCAGCATGC |
| 4751 | CATTGGGAAT GGAGAGTAAA GCAATATCAG ATGCACAGAT TACTGCTTCA |
| 4801 | TCCTACTTTA CCAATATGTT TGCCACCTGG TCTCCTTCAA AAGCTCGACT |
| 4851 | TCACCTCCAA GGGAGGAGTA ATGCCTGGAG ACCTCAGGTG AATAATCCAA |
| 4901 | AAGAGTGGCT GCAAGTGGAC TTCCAGAAGA CAATGAAAGT CACAGGAGTA |
| 4951 | ACTACTCAGG GAGTAAAATC TCTGCTTACC AGCATGTATG TGAAGGAGTT |
| 5001 | CCTCATCTCC AGCAGTCAAG ATGGCCATCA GTGGACTCTC TTTTTTCAGA |
| 5051 | ATGGCAAAGT AAAGGTTTTT CAGGGAAATC AAGACTCCTT CACACCTGTG |
| 5101 | GTGAACTCTC TAGACCCACC GTTACTGACT CGCTACCTTC GAATTCACCC |
| 5151 | CCAGAGTTGG GTGCACCAGA TTGCCCTGAG GATGGAGGTT CTGGGCTGCG |
| 5201 | AGGCACAGGA CCTCTACGAC AAAACTCACA CATGCCCACC GTGCCCAGCT |
| 5251 | CCAGAACTCC TGGGCGGACC GTCAGTCTTC CTCTTCCCCC CAAAACCCAA |
| 5301 | GGACACCCTC ATGATCTCCC GGACCCCTGA GGTCACATGC GTGGTGGTGG |
| 5351 | ACGTGAGCCA CGAAGACCCT GAGGTCAAGT TCAACTGGTA TGTGGACGGC |
| 5401 | GTGGAAGTGC ATAATGCCAA GACAAAGCCG CGGGAGGAGC AGTACAACAG |
| 5451 | CACGTACCGT GTGGTCAGCG TCCTCACCGT CCTGCACCAA GACTGGCTGA |
| 5501 | ATGGCAAGGA GTACAAGTGC AAGGTCTCCA ACAAAGCCCT CCCAGCCCCC |
| 5551 | ATCGAGAAAA CCATCTCCAA AGCCAAAGGG CAGCCCCGAG AACCACAGGT |
| 5601 | GTACACCCTG CCCCCATCCC GGGATGAGCT GACCAAGAAC CAAGTTAGCC |
| 5651 | TGACCTGCCT GGTCAAAGGC TTCTATCCCA GCGACATCGC CGTGGAGTGG |
| 5701 | GAGAGCAATG GGCAGCCGGA GAACAACTAC AAGACCACGC CTCCCGTGTT |
| 5751 | GGACTCCGAC GGCTCCTTCT TCCTCTACTC CAAGCTCACC GTGGACAAGA |
| 5801 | GCAGGTGGCA GCAGGGGAAC GTCTTCTCAT GCTCCGTGAT GCATGAGGCT |
| 5851 | CTGCACAACC ACTACACGCA GAAGAGCCTC TCCCTGTCTC CGGGTTGA |

Amino acid sequence of VWF059A protein -
includes signal peptide and D1D2 region:
SEQ ID NO: 5

| | |
|---|---|
| 1 | MIPARFAGVL LALALILPGT LCAEGTRGRS STARCSLEGS DEVNTEDGSM |
| 51 | YSFAGYCSYL LAGGCQKRSF SIIGDFQNGK RVSLSVYLGE FFDIHLFVNG |
| 101 | TVTQGDQRVS MPYASKGLYL ETEAGYYKLS GEAYGFVARI DGSGNFQVLL |
| 151 | SDRYFNKTCG LCGNFNIFAE DDFMTQEGTL TSDPYDFANS WALSSGEQWC |
| 201 | ERASPPSSSC NISSGEMQKG LWEQCOLLKS TSVFARCHPL VDPEPEVALC |
| 251 | EKTLCECAGG LECACPALLE YARTCAQEGM VLYGWTDHSA CSPVCPAGME |
| 301 | YRQCVSPCAR TCQSLHINEM COERCVDGCS CPEGOLLDEG LCVESTECPC |
| 351 | VHSGKRYPPG TSLSRDCNTC ICRNSQWICS NEECPGECLV TGOSHFKSED |
| 401 | NRYFTESGIC QYLLARDCOD HSFSIVIETV QCADDRDAVC TRSVTVRLPG |
| 451 | LHNSLVKLKH GAGVAMDGQD IQLPLLKGDL RIQHTVTASV RLSYGEDLQM |
| 501 | DWDGRGRLLV KLSPVYAGKT CGLCGNYNGN QGDDELTPSG LAEPRVEDFG |
| 551 | NAWKLHGDCQ DLQKQHSDPC ALNPRMTRES EEACAVLTSP TFEACHRAVS |
| 601 | PLPYLRNCRY DVCSCSDGRE CLCGALASYA AACAGRGVRV AWREPGRCEL |
| 651 | NCPKGQVYLQ CGTPCNLTCR SLSYPDEECN EACLEGCFCP PGLYMDERGD |
| 701 | CVPKAQCPCY YDGEIFQPED IFSDHHTMCY CEDGEMHCTM SGVPGSLLPD |
| 751 | AVLSSPLSHR SKRSLSCRPP MVKLVCPADN LRAEGLECTK TCQNYDLECM |
| 801 | SMGCVSGCLC PPGMVRHENR CVALERCPCF HQGKEYAPGE TVKIGCNTCV |
| 851 | CRDRKWNCTD HVCDATCSTI GMAHYLTEDG LKYLFPGECQ YVLVODYCGS |
| 901 | NPGTFRILVG NKGCSHPSVK CKKRVTILVE GGEIELEDGE VNVKRPMKDE |
| 951 | THFEVVESGR YIILLLGKAL SVVWDRHLSI SVVLKQTYQE KVCGLCGNED |
| 1001 | GIQNNDLTSS NLQVEEDPVD FGNSWKVSSQ CADTRKVPLD SSPATCHNNI |
| 1051 | MKQTMVDSSC RILTSDVFQD CNKLVDPEPY LDVCIYDTCS CESIGDCAAF |
| 1101 | CDTIAAYAHV CAQHGKVVTW RTATLCPQSC EERNLRENGY EAEWRYNSCA |
| 1151 | PACQVTCQHP EPLACPVQCV EGCHAHCPPG KILDELLOTC VDPEDCPVCE |
| 1201 | VAGRRFASGK KVTLNPSDPE HCQICHCDVV NLTCEACQEP GTSESATPES |
| 1251 | GPGSEPATSG SETPGTSESA TPESGPGSEP ATSGSETPGT SESATPESGP |
| 1301 | GTSTEPSEGS APGSPAGSPT STEEGTSESA TPESGPGSEP ATSGSETPGT |
| 1351 | SESATPESGP GSPAGSPTST EEGSPAGSPT STEEGASSDK NTGDYYEDSY |
| 1401 | EDISAYLLSK NNAIEPRSES DKTHTCPPCP APELLGGPSV FLFPPKPKDT |
| 1451 | LMISRTPEVT CVVVDVSHED PEVKENWYVD GVEVHNAKTK PREEQYNSTY |
| 1501 | RVVSVLTVLH QDWLNGKEYK CKVSNKALPA PIEKTISKAK GQPREPQVYT |
| 1551 | LPPSRDELTK NOVSLTCLVK GFYPSDIAVE WESNGOPENN YKTTPPVLDS |
| 1601 | DGSFFLYSKL TVDKSRWOQG NVFSCSVMHE ALHNHYTOKS LSLSPG* |

Nucleotide sequence encoding VWF059A protein:
SEQ ID NO: 6

| | |
|---|---|
| 1 | ATGATTCCTG CCAGATTTGC CGGGGTGCTG CTTGCTCTGG CCCTCATTTT |
| 51 | GCCAGGGACC CTTTGTGCAG AAGGAACTCG CGGCAGGTCA TCCACGGCCC |
| 101 | GATGCAGCCT TTTCGGAAGT GACTTCGTCA ACACCTTTGA TGGGAGCATG |
| 151 | TACAGCTTTG CGGGATACTG CAGTTACCTC CTGGCAGGGG GCTGCCAGAA |
| 201 | ACGCTCCTTC TCGATTATTG GGGACTTCCA GAATGGCAAG AGAGTGAGCC |
| 251 | TCTCCGTGTA TCTTGGGGAA TTTTTTGACA TCCATTTGTT TGTCAATGGT |
| 301 | ACCGTGACAC AGGGGGACCA AAGAGTCTCC ATGCCCTATG CCTCCAAAGG |
| 351 | GCTGTATCTA GAAACTGAGG CTGGGTACTA CAAGCTGTCC GGTGAGGCCT |
| 401 | ATGGCTTTGT GGCCAGGATC GATGGCAGCG GCAACTTTCA AGTCCTGCTG |
| 451 | TCAGACAGAT ACTTCAACAA GACCTGCGGG CTGTGTGGCA ACTTTAACAT |
| 501 | CTTTGCTGAA GATGACTTTA TGACCCAAGA AGGGACCTTG ACCTCGGACC |
| 551 | CTTATGACTT TGCCAACTCA TGGGCTCTGA GCAGTGGAGA ACAGTGGTGT |
| 601 | GAACGGGCAT CTCCTCCCAG CAGCTCATGC AACATCTCCT CTGGGGAAAT |
| 651 | GCAGAAGGGC CTGTGGGAGC AGTGCCAGCT TCTGAAGAGC ACCTCGGTGT |
| 701 | TTGCCCGCTG CCACCCTCTG GTGGACCCCG AGCCTTTTGT GGCCCTGTGT |
| 751 | GAGAAGACTT TGTGTGAGTG TGCTGGGGGG CTGGAGTGCG CCTGCCCTGC |
| 801 | CCTCCTGGAG TACGCCCGGA CCTGTGCCCA GGAGGGAATG GTGCTGTACG |
| 851 | GCTGGACCGA CCACAGCGCG TGCAGCCCAG TGTGCCCTGC TGGTATGGAG |
| 901 | TATAGGCAGT GTGTGTCCCC TTGCGCCAGG ACCTGCCAGA GCCTGCACAT |
| 951 | CAATGAAATG TGTCAGGAGC GATGCGTGGA TGGCTGCAGC TGCCCTGAGG |
| 1001 | GACAGCTCCT GGATGAAGGC CTCTGCGTGG AGAGCACCGA GTGTCCCTGC |
| 1051 | GTGCATTCCG GAAAGCGCTA CCCTCCCGGC ACCTCCCTCT CTCGAGACTG |
| 1101 | CAACACCTGC ATTTGCCGAA ACAGCCAGTG GATCTGCAGC AATGAAGAAT |
| 1151 | GTCCAGGGGA GTGCCTTGTC ACTGGTCAAT CCCACTTCAA GAGCTTTGAC |
| 1201 | AACAGATACT TCACCTTCAG TGGGATCTGC CAGTACCTGC TGGCCCGGGA |
| 1251 | TTGCCAGGAC CACTCCTTCT CCATTGTCAT TGAGACTGTC CAGTGTGCTG |
| 1301 | ATGACCGCGA CGCTGTGTGC ACCCGCTCCG TCACCGTCCG GCTGCCTGGC |
| 1351 | CTGCACAACA GCCTTGTGAA ACTGAAGCAT GGGGCAGGAG TTGCCATGGA |
| 1401 | TGGCCAGGAC ATCCAGCTCC CCCTCCTGAA AGGTGACCTC CGCATCCAGC |
| 1451 | ATACAGTGAC GGCCTCCGTG CGCCTCAGCT ACGGGGAGGA CCTGCAGATG |
| 1501 | GACTGGGATG GCCGCGGGAG GCTGCTGGTG AAGCTGTCCC CCGTCTATGC |
| 1551 | CGGGAAGACC TGCGGCCTGT GTGGGAATTA CAATGGCAAC CAGGGCGACG |
| 1601 | ACTTCCTTAC CCCCTCTGGG CTGGCGGAGC CCCGGGTGGA GGACTTCGGG |
| 1651 | AACGCCTGGA AGCTGCACGG GGACTGCCAG GACCTGCAGA AGCAGCACAG |
| 1701 | CGATCCCTGC GCCCTCAACC CGCGCATGAC CAGGTTCTCC GAGGAGGCGT |
| 1751 | GCGCGGTCCT GACGTCCCCC ACATTCGAGG CCTGCCATCG TGCCGTCAGC |
| 1801 | CCGCTGCCCT ACCTGCGGAA CTGCCGCTAC GACGTGTGCT CCTGCTCGGA |
| 1851 | CGGCCGCGAG TGCCTGTGCG GCGCCCTGGC CAGCTATGCC GCGGCCTGCG |
| 1901 | CGGGGAGAGG CGTGCGCGTC GCGTGGCGCG AGCCAGGCCG CTGTGAGCTG |
| 1951 | AACTGCCCGA AAGGCCAGGT GTACCTGCAG TGCGGGACCC CCTGCAACCT |
| 2001 | GACCTGCCGC TCTCTCTCTT ACCCGGATGA GGAATGCAAT GAGGCCTGCC |
| 2051 | TGGAGGGCTG CTTCTGCCCC CCAGGGCTCT ACATGGATGA GAGGGGGGAC |
| 2101 | TGCGTGCCCA AGGCCCAGTG CCCCTGTTAC TATGACGGTG AGATCTTCCA |
| 2151 | GCCAGAAGAC ATCTTCTCAG ACCATCACAC CATGTGCTAC TGTGAGGATG |
| 2201 | GCTTCATGCA CTGTACCATG AGTGGAGTCC CCGGAAGCTT GCTGCCTGAC |
| 2251 | GCTGTCCTCA GCAGTCCCCT GTCTCATCGC AGCAAAAGGA GCCTATCCTG |
| 2301 | TCGGCCCCCC ATGGTCAAGC TGGTGTGTCC CGCTGACAAC CTGCGGGCTG |
| 2351 | AAGGGCTCGA GTGTACCAAA ACGTGCCAGA ACTATGACCT GGAGTGCATG |
| 2401 | AGCATGGGCT GTGTCTCTGG CTGCCTCTGC CCCCCGGGCA TGGTCCGGCA |
| 2451 | TGAGAACAGA TGTGTGGCCC TGGAAAGGTG TCCCTGCTTC CATCAGGGCA |
| 2501 | AGGAGTATGC CCCTGGAGAA ACAGTGAAGA TTGGCTGCAA CACTTGTGTC |
| 2551 | TGTCGGGACC GGAAGTGGAA CTGCACAGAC CATGTGTGTG ATGCCACGTG |
| 2601 | CTCCACGATC GGCATGGCCC ACTACCTCAC CTTCGACGGG CTCAAATACC |
| 2651 | TGTTCCCCGG GGAGTGCCAG TACGTTCTGG TGCAGGATTA CTGCGGCAGT |
| 2701 | AACCCTGGGA CCTTTCGGAT CCTAGTGGGG AATAAGGGAT GCAGCCACCC |
| 2751 | CTCAGTGAAA TGCAAGAAAC GGGTCACCAT CCTGGTGGAG GGAGGAGAGA |
| 2801 | TTGAGCTGTT TGACGGGGAG GTGAATGTGA AGAGGCCCAT GAAGGATGAG |
| 2851 | ACTCACTTTG AGGTGGTGGA GTCTGGCCGG TACATCATTC TGCTGCTGGG |
| 2901 | CAAAGCCCTC TCCGTGGTCT GGGACCGCCA CCTGAGCATC TCCGTGGTCC |
| 2951 | TGAAGCAGAC ATACCAGGAG AAAGTGTGTG GCCTGTGTGG GAATTTTGAT |
| 3001 | GGCATCCAGA ACAATGACCT CACCAGCAGC AACCTCCAAG TGGAGGAAGA |
| 3051 | CCCTGTGGAC TTTGGGAACT CCTGGAAAGT GAGCTCGCAG TGTGCTGACA |
| 3101 | CCAGAAAAGT GCCTCTGGAC TCATCCCCTG CCACCTGCCA TAACAACATC |
| 3151 | ATGAAGCAGA CGATGGTGGA TTCCTCCTGT AGAATCCTTA CCAGTGACGT |
| 3201 | CTTCCAGGAC TGCAACAAGC TGGTGGACCC CGAGCCATAT CTGGATGTCT |
| 3251 | GCATTTACGA CACCTGCTCC TGTGAGTCCA TTGGGGACTG CGCCGCATTC |
| 3301 | TGCGACACCA TTGCTGCCTA TGCCCACGTG TGTGCCCAGC ATGGCAAGGT |
| 3351 | GGTGACCTGG AGGACGGCCA CATTGTGCCC CCAGAGCTGC GAGGAGAGGA |
| 3401 | ATCTCCGGGA GAACGGGTAT GAGGCTGAGT GGCGCTATAA CAGCTGTGCA |
| 3451 | CCTGCCTGTC AAGTCACGTG TCAGCACCCT GAGCCACTGG CCTGCCCTGT |
| 3501 | GCAGTGTGTG GAGGGCTGCC ATGCCCACTG CCCTCCAGGG AAAATCCTGG |
| 3551 | ATGAGCTTTT GCAGACCTGC GTTGACCCTG AAGACTGTCC AGTGTGTGAG |
| 3601 | GTGGCTGGCC GGCGTTTTGC CTCAGGAAAG AAAGTCACCT TGAATCCCAG |
| 3651 | TGACCCTGAG CACTGCCAGA TTTGCCACTG TGATGTTGTC AACCTCACCT |
| 3701 | GTGAAGCCTG CCAGGAGCCG GGTACATCAG AGAGCGCCAC CCCTGAAAGT |
| 3751 | GGTCCCGGGA GCGAGCCAGC CACATCTGGG TCGGAAACGC CAGGCACATC |
| 3801 | CGAGTCTGCA ACTCCCGAGT CCGGACCTGG CTCCGAGCCT GCCACTAGCG |
| 3851 | GCTCCGAGAC TCCGGGAACT TCCGAGAGCG CTACACCAGA AAGCGGACCC |
| 3901 | GGAACCAGTA CCGAACCTAG CGAGGGCTCT GCTCCGGGCA GCCCAGCCGG |
| 3951 | CTCTCCTACA TCCACGGAGG AGGGCACTTC CGAATCCGCC ACCCCGGAGT |
| 4001 | CAGGGCCAGG ATCTGAACCC GCTACCTCAG GCAGTGAGAC GCCAGGAACG |
| 4051 | AGCGAGTCCG CTACACCGGA GAGTGGGCCA GGGAGCCCTG CTGGATCTCC |
| 4101 | TACGTCCACT GAGGAAGGGT CACCAGCGGG CTCGCCCACC AGCACTGAAG |
| 4151 | AAGGTGCCTC GTCTGACAAG AACACTGGTG ATTATTACGA GGACAGTTAT |
| 4201 | GAAGATATTT CAGCATACTT GCTGAGTAAA AACAATGCCA TTGAACCAAG |
| 4251 | AAGCTTCTCT GACAAAACTC ACACATGCCC ACCGTGCCCA GCTCCAGAAC |
| 4301 | TCCTGGGCGG ACCGTCAGTC TTCCTCTTCC CCCCAAAACC CAAGGACACC |
| 4351 | CTCATGATCT CCCGGACCCC TGAGGTCACA TGCGTGGTGG TGGACGTGAG |
| 4401 | CCACGAAGAC CCTGAGGTCA AGTTCAACTG GTATGTGGAC GGCGTGGAAG |
| 4451 | TGCATAATGC CAAGACAAAG CCGCGGGAGG AGCAGTACAA CAGCACGTAC |
| 4501 | CGTGTGGTCA GCGTCCTCAC CGTCCTGCAC CAAGACTGGC TGAATGGCAA |
| 4551 | GGAGTACAAG TGCAAGGTCT CCAACAAAGC CCTCCCAGCC CCCATCGAGA |
| 4601 | AAACCATCTC CAAAGCCAAA GGGCAGCCCC GAGAACCACA GGTGTACACC |
| 4651 | CTGCCCCCAT CCCGGGATGA GCTGACCAAG AACCAAGTTA GCCTGACCTG |
| 4701 | CCTGGTCAAA GGCTTCTATC CCAGCGACAT CGCCGTGGAG TGGGAGAGCA |
| 4751 | ATGGGCAGCC GGAGAACAAC TACAAGACCA CGCCTCCCGT GTTGGACTCC |
| 4801 | GACGGCTCCT TCTTCCTCTA CTCCAAGCTC ACCGTGGACA AGAGCAGGTG |
| 4851 | GCAGCAGGGG AACGTCTTCT CATGCTCCGT GATGCATGAG GCTCTGCACA |
| 4901 | ACCACTACAC GCAGAAGAGC CTCTCCCTGT CTCCGGGTTG A |

FVIII(ELNN)-Fc: SEQ ID NO: 7

| | |
|---|---|
| TRRYYLGAVE LSWDYMQSDL GELPVDARFP PRVPKSFPEN TSVVYKKTLF VEFTDHLENI | 60 |
| AKPRPPWMGL LGPTIQAEVY DTVVITLKNM ASHPVSLHAV GVSYWKASEG AEYDDQTSQR | 120 |
| EKEDDKVFPG GSHTYVWQVL KENGPMASDP LCLTYSYLSH VDLVKDLNSG LIGALLVCRE | 180 |
| GSLAKEKTQT LHKFILLFAV FDEGKSWHSE TKNSLMQDRD AASARAWPKM HTVNGYVNRS | 240 |
| LPGLIGCHRK SVYWHVIGMG TTPEVHSIFL EGHTFLVRNH RQASLEISPI TELTAQTLLM | 300 |
| DLGQFLLFCH ISSHQHDGME AYVKVDSCPE EPQLRMKNNE EAEDYDDDLT DSEMDVVRED | 360 |
| DDNSPSFIQI RSVAKKHPKT WVHYIAAEEE DWDYAPLVLA PDDRSYKSQY LNNGPQRIGR | 420 |
| KYKKVREMAY TDETEKTREA IQHESGILGP LLYGEVGDTL LIIFKNQASR PYNIYPHGIT | 480 |
| DVRPLYSRRL PKGVKHLKDF PILPGEIFKY KWTVTVEDGP TKSDPRCLTR YYSSFVNMER | 540 |
| DLASGLIGPL LICYKESVDQ RGNQIMSDKR NVILESVEDE NRSWYLTENI QRFLPNPAGV | 600 |
| QLEDPEFQAS NIMHSINGYV FDSLQLSVCL HEVAYWYILS IGAQTDFLSV FFSGYTFKHK | 660 |
| MVYEDTLTLF PFSGETVEMS MENPGLWILG CHNSDERNRG MTALLKVSSC DKNTGDYYED | 720 |
| SYEDISAYLL SKNNAIEPRS FSQNGTSESA TPESGPGSEP ATSGSETPGT SESATPESGP | 780 |
| GSEPATSGSE TPGTSESATP ESGPGTSTEP SEGSAPGSPA GSPTSTEEGT SESATPESGP | 840 |
| GSEPATSGSE TPGTSESATP ESGPGSPAGS PTSTEEGSPA GSPTSTEEGT STEPSEGSAP | 900 |
| GTSESATPES GPGTSESATP ESGPGTSESA TPESGPGSEP ATSGSETPGS EPATSGSETP | 960 |
| GSPAGSPTST EEGTSTEPSE GSAPGTSTEP SEGSAPGSEP ATSGSETPGT SESATPESGP | 1020 |
| GTSTEPSEGS APASSEITRT TLQSDQEEID YDDTISVEMK KEDEDIYDED ENQSPRSFQK | 1080 |
| KTRHYFIAAV ERLWDYGMSS SPHVLRNRAQ SGSVPQFKKV VFQEFTDGSF TOPLYRGELN | 1140 |
| EHLGLLGPYI RAEVEDNIMV TERNQASRPY SFYSSLISYE EDQRQGAEPR KNFVKPNETK | 1200 |
| TYFWKVQHHM APTKDEFDCK AWAYFSDVDL EKDVHSGLIG PLLVCHTNTL NPAHGRQVTV | 1260 |
| QEFALFFTIF DETKSWYFTE NMERNCRAPC NIQMEDPTEK ENYRFHAING YIMDTLPGLV | 1320 |
| MAQDQRIRWY LLSMGSNENI HSIHFSGHVF TVRKKEEYKM ALYNLYPGVF ETVEMLPSKA | 1380 |
| GIWRVECLIG EHLHAGMSTL FLVYSNKCQT PLGMASGHIR DFQITASGQY GQWAPKLARL | 1440 |
| HYSGSINAWS TKEPFSWIKV DLLAPMIIHG IKTQGARQKF SSLYISQFII MYSLDGKKWQ | 1500 |
| TYRGNSTGTL MVFFGNVDSS GIKHNIFNPP IIARYIRLHP THYSIRSTLR MELMGCDLNS | 1560 |
| CSMPLGMESK AISDAQITAS SYFTNMFATW SPSKARLHLQ GRSNAWRPQV NNPKEWLQVD | 1620 |
| FQKTMKVTGV TTQGVKSLLT SMYVKEFLIS SSQDGHQWTL FFQNGKVKVF QGNQDSFTPV | 1680 |
| VNSLDPPLLT RYLRIHPQSW VHQIALRMEV LGCEAQDLYD KTHTCPPCPA PELLGGPSVF | 1740 |
| LFPPKPKDTL MISRTPEVTC VVVDVSHEDP EVKENWYVDG VEVHNAKTKP REEQYNSTYR | 1800 |
| VVSVLTVLHQ DWLNGKEYKC KVSNKALPAP IEKTISKAKG QPREPQVYTL PPSRDELTKN | 1860 |
| QVSLTCLVKG FYPSDIAVEW ESNGQPENNY KTTPPVLDSD GSFFLYSKLT VDKSRWQQGN | 1920 |
| VFSCSVMHEA LHNHYTQKSL SLSPG | 1945 |

TABLE 20
Additional chimeric protein sequences

| Description/ | | |
|---|---|---|
| SEQ ID NO. | Sequence | |
| Full length | ATRRYYLGAV ELSWDYMQSD LGELPVDARF PPRVPKSFPF NTSVVYKKTL FVEFTDHLEN | 60 |
| FVIII | IAKPRPPWMG LLGPTIQAEV YDTVVITLKN MASHPVSLHA VGVSYWKASE GAEYDDQTSQ | 120 |
| SEQ ID NO: 8 | REKEDDKVFP GGSHTYVWQV LKENGPMASD PLCLTYSYLS HVDLVKDLNS GLIGALLVCR | 180 |
| | EGSLAKEKTQ TLHKFILLFA VEDEGKSWHS ETKNSLMQDR DAASARAWPK MHTVNGYVNR | 240 |
| | SLPGLIGCHR KSVYWHVIGM GTTPEVHSIF LEGHTELVRN HRQASLEISP ITELTAQTLL | 300 |
| | MDLGQFLLFC HISSHQHDGM EAYVKVDSCP EEPQLRMKNN EEAEDYDDDL TDSEMDVVRE | 360 |
| | DDDNSPSFIQ IRSVAKKHPK TWVHYIAAEE EDWDYAPLVL APDDRSYKSQ YLNNGPQRIG | 420 |
| | RKYKKVREMA YTDETEKTRE AIQHESGILG PLLYGEVGDT LLIIFKNQAS RPYNIYPHGI | 480 |
| | TDVRPLYSRR LPKGVKHLKD FPILPGEIFK YKWTVTVEDG PTKSDPRCLT RYYSSFVNME | 540 |
| | RDLASGLIGP LLICYKESVD QRGNQIMSDK RNVILESVED ENRSWYLTEN IQRFLPNPAG | 600 |
| | VQLEDPEFQA SNIMHSINGY VEDSLQLSVC LHEVAYWYIL SIGAQTDELS VFFSGYTFKH | 660 |
| | KMVYEDTLTL FPFSGETVEM SMENPGLWIL GCHNSDERNR GMTALLKVSS CDKNTGDYYE | 720 |
| | DSYEDISAYL LSKNNAIEPR SFSQNSRHPS TRQKQFNATT IPENDIEKTD PWFAHRTPMP | 780 |
| | KIQNVSSSDL LMLLRQSPTP HGLSLSDLQE AKYETFSDDP SPGAIDSNNS LSEMTHFRPQ | 840 |
| | LHHSGDMVFT PESGLQLRLN EKLGTTAATE LKKLDEKVSS TSNNLISTIP SDNLAAGTDN | 900 |
| | TSSLGPPSMP VHYDSQLDTT LFGKKSSPLT ESGGPLSLSE ENNDSKLLES GLMNSQESSW | 960 |
| | GKNVSSTESG RLFKGKRAHG PALLTKDNAL FKVSISLLKT NKTSNNSATN RKTHIDGPSL | 1020 |
| | LIENSPSVWQ NILESDTEFK KVTPLIHDRM LMDKNATALR LNHMSNKTTS SKNMEMVQQK | 1080 |
| | KEGPIPPDAQ NPDMSFFKML FLPESARWIQ RTHGKNSLNS GQGPSPKQLV SLGPEKSVEG | 1140 |
| | QNFLSEKNKV VVGKGEFTKD VGLKEMVFPS SRNLELTNLD NLHENNTHNQ EKKIQEEIEK | 1200 |
| | KETLIQENVV LPQIHTVTGT KNEMKNLELL STRQNVEGSY DGAYAPVLQD FRSLNDSTNR | 1260 |
| | TKKHTAHFSK KGEEENLEGL GNQTKQIVEK YACTTRISPN TSQQNFVTQR SKRALKQFRL | 1320 |
| | PLEETELEKR IIVDDTSTQW SKNMKHLTPS TLTQIDYNEK EKGAITQSPL SDCLTRSHSI | 1380 |
| | PQANRSPLPI AKVSSFPSIR PIYLTRVLFQ DNSSHLPAAS YRKKDSGVQE SSHFLQGAKK | 1440 |
| | NNLSLAILTL EMTGDQREVG SLGTSATNSV TYKKVENTVL PKPDLPKTSG KVELLPKVHI | 1500 |
| | YQKDLFPTET SNGSPGHLDL VEGSLLQGTE GAIKWNEANR PGKVPFLRVA TESSAKTPSK | 1560 |
| | LLDPLAWDNH YGTQIPKEEW KSQEKSPEKT AFKKKDTILS LNACESNHAI AAINEGQNKP | 1620 |
| | EIEVTWAKQG RTERLCSQNP PVLKRHQREI TRTTLQSDQE EIDYDDTISV EMKKEDEDIY | 1680 |
| | DEDENQSPRS FQKKTRHYFI AAVERLWDYG MSSSPHVLRN RAQSGSVPQF KKVVFQEFTD | 1740 |
| | GSFTQPLYRG ELNEHLGLLG PYIRAEVEDN IMVTERNQAS RPYSFYSSLI SYEEDQRQGA | 1800 |
| | EPRKNFVKPN ETKTYFWKVQ HHMAPTKDEF DCKAWAYFSD VDLEKDVHSG LIGPLLVCHT | 1860 |
| | NTLNPAHGRQ VTVQEFALFF TIFDETKSWY FTENMERNCR APCNIQMEDP TEKENYRFHA | 1920 |
| | INGYIMDTLP GLVMAQDQRI RWYLLSMGSN ENIHSIHFSG HVFTVRKKEE YKMALYNLYP | 1980 |
| | GVFETVEMLP SKAGIWRVEC LIGEHLHAGM STLFLVYSNK CQTPLGMASG HIRDFQITAS | 2040 |
| | GQYGQWAPKL ARLHYSGSIN AWSTKEPESW IKVDLLAPMI IHGIKTQGAR QKESSLYISQ | 2100 |
| | FIIMYSLDGK KWQTYRGNST GTLMVFFGNV DSSGIKHNIF NPPIIARYIR LHPTHYSIRS | 2160 |
| | TLRMELMGCD LNSCSMPLGM ESKAISDAQI TASSYFTNME ATWSPSKARL HLQGRSNAWR | 2220 |
| | PQVNNPKEWL QVDFQKTMKV TGVTTQGVKS LLTSMYVKEF LISSSQDGHQ WTLFFQNGKV | 2280 |
| | KVFQGNQDSF TPVVNSLDPP LLTRYLRIHP QSWVHQIALR MEVLGCEAQD LY | 2332 |
| AE288 | GTSESATPES GPGSEPATSG SETPGTSESA TPESGPGSEP ATSGSETPGT SESATPESGP | 60 |
| SEQ ID NO: 9 | GTSTEPSEGS APGSPAGSPT STEEGTSESA TPESGPGSEP ATSGSETPGT SESATPESGP | 120 |
| | GSPAGSPTST EEGSPAGSPT STEEGTSTEP SEGSAPGTSE SATPESGPGT SESATPESGP | 180 |
| | GTSESATPES GPGSEPATSG SETPGSEPAT SGSETPGSPA GSPTSTEEGT STEPSEGSAP | 240 |
| | GTSTEPSEGS APGSEPATSG SETPGTSESA TPESGPGTST EPSEGSAP | 288 |
| pSYN VWF059 | TSTEEGASIS DKNTGDYYED SYEDISAYLL SKNNAIEPRS FSDKTH | |
| SEQ ID NO: 10 | | |
| pSYN VWF059A | TSTEEGASSD KNTGDYYEDS YEDISAYLLS KNNAIEPRSF SDKTH | |
| SEQ ID NO: 11 | | |
| pSYN FVIII 312 | ATRRYYLGAV ELSWDYMQSD LGELPVDARF PPRVPKSFPF NTSVVYKKTL FVEFTDHLEN | 60 |
| SEQ ID NO: 12 | IAKPRPPWMG LLGPTIQAEV YDTVVITLKN MASHPVSLHA VGVSYWKASE GAEYDDQTSQ | 120 |
| | REKEDDKVFP GGSHTYVWQV LKENGPMASD PLCLTYSYLS HVDLVKDLNS GLIGALLVCR | 180 |
| | EGSLAKEKTQ TLHKFILLFA VEDEGKSWHS ETKNSLMQDR DAASARAWPK MHTVNGYVNR | 240 |
| | SLPGLIGCHR KSVYWHVIGM GTTPEVHSIF LEGHTFLVRN HRQASLEISP ITFLTAQTLL | 300 |
| | MDLGQFLLFC HISSHQHDGM EAYVKVDSCP EEPQLRMKNN EEAEDYDDDL TDSEMDVVRE | 360 |
| | DDDNSPSFIQ IRSVAKKHPK TWVHYIAAEE EDWDYAPLVL APDDRSYKSQ YLNNGPQRIG | 420 |
| | RKYKKVRFMA YTDETEKTRE AIQHESGILG PLLYGEVGDT LLIIFKNQAS RPYNIYPHGI | 480 |
| | TDVRPLYSRR LPKGVKHLKD FPILPGEIFK YKWTVTVEDG PTKSDPRCLT RYYSSFVNME | 540 |
| | RDLASGLIGP LLICYKESVD QRGNQIMSDK RNVILESVED ENRSWYLTEN IQRFLPNPAG | 600 |
| | VQLEDPEFQA SNIMHSINGY VFDSLQLSVC LHEVAYWYIL SIGAQTDELS VFFSGYTFKH | 660 |
| | KMVYEDTLTL FPFSGETVEM SMENPGLWIL GCHNSDERNR GMTALLKVSS CDKNTGDYYE | 720 |
| | DSYEDISAYL LSKNNAIEPR SFSQNGTSES ATPESGPGSE PATSGSETPG TSESATPESG | 780 |
| | PGSEPATSGS ETPGTSESAT PESGPGTSTE PSEGSAPGSP AGSPTSTEEG TSESATPESG | 840 |
| | PGSEPATSGS ETPGTSESAT PESGPGSPAG SPTSTEEGSP AGSPTSTEEG TSTEPSEGSA | 900 |
| | PGTSESATPE SGPGTSESAT PESGPGTSES ATPESGPGSE PATSGSETPG SEPATSGSET | 960 |
| | PGSPAGSPTS TEEGTSTEPS EGSAPGTSTE PSEGSAPGSE PATSGSETPG TSESATPESG | 1020 |
| | PGTSTEPSEG SAPASSEITR TTLQSDQEEI DYDDTISVEM KKEDEDIYDE DENQSPRSFQ | 1080 |
| | KKTRHYFIAA VERLWDYGMS SSPHVLRNRA QSGSVPQFKK VVFQEFTDGS FTQPLYRGEL | 1140 |
| | NEHLGLLGPY IRAEVEDNIM VTERNQASRP YSFYSSLISY EEDQRQGAEP RKNFVKPNET | 1200 |
| | KTYFWKVQHH MAPTKDEFDC KAWAYESDVD LEKDVHSGLI GPLLVCHTNT LNPAHGRQVT | 1260 |
| | VQEFALFFTI FDETKSWYFT ENMERNCRAP CNIQMEDPTF KENYRFHAIN GYIMDTLPGL | 1320 |
| | VMAQDQRIRW YLLSMGSNEN IHSIHFSGHV FTVRKKEEYK MALYNLYPGV FETVEMLPSK | 1380 |
| | AGIWRVECLI GEHLHAGMST LFLVYSNKCQ TPLGMASGHI RDFQITASGQ YGQWAPKLAR | 1440 |
| | LHYSGSINAW STKEPFSWIK VDLLAPMIIH GIKTQGARQK FSSLYISQFI IMYSLDGKKW | 1500 |
| | QTYRGNSTGT LMVFFGNVDS SGIKHNIFNP PIIARYIRLH PTHYSIRSTL RMELMGCDLN | 1560 |
| | SCSMPLGMES KAISDAQITA SSYFTNMFAT WSPSKARLHL QGRSNAWRPQ VNNPKEWLQV | 1620 |
| | DFQKTMKVTG VTTQGVKSLL TSMYVKEFLI SSSQDGHQWT LFFQNGKVKV FQGNQDSFTP | 1680 |
| | VVNSLDPPLL TRYLRIHPQS WVHQIALRME VLGCEAQDLY DKTHTCPPCP APELLGGPSV | 1740 |
| | FLFPPKPKDT LMISRTPEVT CVVVDVSHED PEVKENWYVD GVEVHNAKTK PREEQYNSTY | 1800 |
| | RVVSVLTVLH QDWLNGKEYK CKVSNKALPA PIEKTISKAK GQPREPQVYT LPPSRDELTK | 1860 |
| | NQVSLTCLVK GFYPSDIAVE WESNGQPENN YKTTPPVLDS DGSFFLYSKL TVDKSRWQQG | 1920 |
| | NVFSCSVMHE ALHNHYTQKS LSLSPGK | |
| ELNN_AE288_2 | GSPAGSPTST EEGTSESATP ESGPGSEPAT SGSETPGTSE SATPESGPGT STEPSEGSAP | 60 |
| SEQ ID NO: 13 | GTSTEPSEGS APGTSTEPSE GSAPGTSTEP SEGSAPGTST EPSEGSAPGT STEPSEGSAP | 120 |
| | GSPAGSPTST EEGTSTEPSE GSAPGTSESA TPESGPGSEP ATSGSETPGT SESATPESGP | 180 |
| | GSEPATSGSE TPGTSESATP ESGPGTSTEP SEGSAPGTSE SATPESGPGS PAGSPTSTEE | 240 |
| | GSPAGSPTST EEGSPAGSPT STEEGTSESA TPESGPGTST EPSEGSAP | 288 |
| ELNN AE144_5A | TSESATPESG PGSEPATSGS ETPGTSESAT PESGPGSEPA TSGSETPGTS ESATPESGPG | 60 |
| SEQ ID NO: 14 | SPAGSPTSTE EGSPAGSPTS TEEGPESGPG SEPATSGSET PGTSESATPE SGPG | 120 |
| | TSTEPSEGSA PGSPAGSPTS TEEGTSESAT | |
| a2 Linker | DKNTGDYYED SYEDISAYLL SKNNAIEPRS FS | 32 |
| of chimeric | | |
| protein | | |
| SEQ ID NO: 15 | | |
| Signal Peptide | MQIELSTCFFLCLLRFCES | |
| of FVIII | | |
| SEQ ID NO: 16 | | |
| FVIII | ATRRYYLGAV ELSWDYMQSD LGELPVDARF PPRVPKSFPF NTSVVYKKTL FVEFTDHLEN | 60 |
| fragment 1 of | IAKPRPPWMG LLGPTIQAEV YDTVVITLKN MASHPVSLHA VGVSYWKASE GAEYDDQTSQ | 120 |
| chimeric | REKEDDKVFP GGSHTYVWQV LKENGPMASD PLCLTYSYLS HVDLVKDLNS GLIGALLVCR | 180 |
| protein | EGSLAKEKTQ TLHKFILLFA VEDEGKSWHS ETKNSLMQDR DAASARAWPK MHTVNGYVNR | 240 |
| SEQ ID NO. 17 | SLPGLIGCHR KSVYWHVIGM GTTPEVHSIF LEGHTFLVRN HRQASLEISP ITFLTAQTLL | 300 |
| | MDLGQFLLFC HISSHQHDGM EAYVKVDSCP EEPQLRMKNN EEAEDYDDDL TDSEMDVVRE | 360 |
| | DDDNSPSFIQ IRSVAKKHPK TWVHYIAAEE EDWDYAPLVL APDDRSYKSQ YLNNGPQRIG | 420 |
| | RKYKKVREMA YTDETFKTRE AIQHESGILG PLLYGEVGDT LLIIFKNQAS RPYNIYPHGI | 480 |
| | TDVRPLYSRR LPKGVKHLKD FPILPGEIFK YKWTVTVEDG PTKSDPRCLT RYYSSFVNME | 540 |
| | RDLASGLIGP LLICYKESVD QRGNQIMSDK RNVILESVED ENRSWYLTEN IQRFLPNPAG | 600 |
| | VQLEDPEFQA SNIMHSINGY VEDSLQLSVC LHEVAYWYIL SIGAQTDELS VFFSGYTEKH | 660 |
| | KMVYEDTLTL FPFSGETVEM SMENPGLWIL GCHNSDERNR GMTALLKVSS CDKNTGDYYE | 720 |
| | DSYEDISAYL LSKNNAIEPR SFSQN | |
| FVIII | EITRTTLQSD QEEIDYDDTI SVEMKKEDED IYDEDENQSP RSFQKKTRHY FIAAVERLWD | 60 |
| fragment 2 | YGMSSSPHVL RNRAQSGSVP QFKKVVFQEF TDGSFTQPLY RGELNEHLGL LGPYIRAEVE | 120 |
| of chimeric | DNIMVTERNQ ASRPYSFYSS LISYEEDQRQ GAEPRKNFVK PNETKTYFWK VQHHMAPTKD | 180 |
| protein | EFDCKAWAYF SDVDLEKDVH SGLIGPLLVC HTNTLNPAHG RQVTVQEFAL FFTIFDETKS | 240 |
| SEQ ID NO. 18 | WYFTENMERN CRAPCNIQME DPTFKENYRF HAINGYIMDT LPGLVMAQDQ RIRWYLLSMG | 300 |
| | SNENIHSIHF SGHVFTVRKK EEYKMALYNL YPGVFETVEM LPSKAGIWRV ECLIGEHLHA | 360 |
| | GMSTLFLVYS NKCQTPLGMA SGHIRDFQIT ASGQYGQWAP KLARLHYSGS INAWSTKEPF | 420 |
| | SWIKVDLLAP MIIHGIKTQG ARQKESSLYI SQFIIMYSLD GKKWQTYRGN STGTLMVFFG | 480 |
| | NVDSSGIKHN IFNPPIIARY IRLHPTHYSI RSTLRMELMG CDLNSCSMPL GMESKAISDA | 540 |
| | QITASSYFTN MFATWSPSKA RLHLQGRSNA WRPQVNNPKE WLQVDFQKTM KVTGVTTQGV | 600 |
| | KSLLTSMYVK EFLISSSQDG HQWTLFFQNG KVKVFQGNQD SFTPVVNSLD PPLLTRYLRI | 660 |
| | HPQSWVHQIA LRMEVLGCEA QDLY | |
| VWF Signal | MIPARFAGVL LALALILPGT LC | |
| Peptide | | |
| SEQ ID NO: 19 | | |
| VWF D1D2 | AEGTRGRSST ARCSLFGSDF VNTEDGSMYS FAGYCSYLLA GGCQKRSFSI IGDFQNGKRV | 60 |
| domain of | SLSVYLGEFF DIHLFVNGTV TQGDQRVSMP YASKGLYLET EAGYYKLSGE AYGFVARIDG | 120 |
| chimeric | SGNFQVLLSD RYFNKTCGLC GNFNIFAEDD FMTQEGTLTS DPYDFANSWA LSSGEQWCER | 180 |
| protein | ASPPSSSCNI SSGEMQKGLW EQCQLLKSTS VFARCHPLVD PEPEVALCEK TLCECAGGLE | 240 |
| SEQ ID NO: 20 | CACPALLEYA RTCAQEGMVL YGWTDHSACS PVCPAGMEYR QCVSPCARTC QSLHINEMCQ | 300 |
| | ERCVDGCSCP EGQLLDEGLC VESTECPCVH SGKRYPPGTS LSRDCNTCIC RNSQWICSNE | 360 |
| | ECPGECLVTG QSHEKSFDNR YFTESGICQY LLARDCQDHS FSIVIETVQC ADDRDAVCTR | 420 |
| | SVTVRLPGLH NSLVKLKHGA GVAMDGQDIQ LPLLKGDLRI QHTVTASVRL SYGEDLQMDW | 480 |
| | DGRGRLLVKL SPVYAGKTCG LCGNYNGNQG DDFLTPSGLA EPRVEDEGNA WKLHGDCQDL | 540 |
| | QKQHSDPCAL NPRMTRESEE ACAVLTSPTF EACHRAVSPL PYLRNCRYDV CSCSDGRECL | 600 |
| | CGALASYAAA CAGRGVRVAW REPGRCELNC PKGQVYLQCG TPCNLTCRSL SYPDEECNEA | 660 |
| | CLEGCFCPPG LYMDERGDCV PKAQCPCYYD GEIFQPEDIF SDHHTMCYCE DGFMHCTMSG | 720 |
| | VPGSLLPDAV LSSPLSHRSK R | |
| VWF D′ domain | SLSCRPPMVK LVCPADNLRA EGLECTKTCQ NYDLECMSMG CVSGCLCPPG MVRHENRCVA | 60 |
| of chimeric | LERCPCFHQG KEYAPGETVK IGCNTCVCRD RKWNCTDHVC DAT | 103 |
| protein | | |
| SEQ ID NO: 21 | | |
| VWF D3 | CSTIGMAHYL TFDGLKYLFP GECQYVLVQD YCGSNPGTER ILVGNKGCSH PSVKCKKRVT | 60 |
| domain of | ILVEGGEIEL FDGEVNVKRP MKDETHFEVV ESGRYIILLL GKALSVVWDR HLSISVVLKQ | 120 |
| chimeric | TYQEKVCGLC GNEDGIQNND LTSSNLQVEE DPVDFGNSWK VSSQCADTRK VPLDSSPATC | 180 |
| protein | HNNIMKQTMV DSSCRILTSD VFQDCNKLVD PEPYLDVCIY DTCSCESIGD CAAFCDTIAA | 240 |
| SEQ ID NO: 22 | YAHVCAQHGK VVTWRTATLC PQSCEERNLR ENGYEAEWRY NSCAPACQVT CQHPEPLACP | 300 |
| | VQCVEGCHAH CPPGKILDEL LQTCVDPEDC PVCEVAGRRF ASGKKVTLNP SDPEHCQICH | 360 |
| | CDVVNLTCEA CQEP | 374 |
| Fc region | DKTHTCPPCP APELLGGPSV FLFPPKPKDT LMISRTPEVT CVVVDVSHED PEVKENWYVD | 60 |
| SEQ ID NO: 23 | GVEVHNAKTK PREEQYNSTY RVVSVLTVLH QDWLNGKEYK CKVSNKALPA PIEKTISKAK | 120 |
| | GQPREPQVYT LPPSRDELTK NQVSLTCLVK GFYPSDIAVE WESNGQPENN YKTTPPVLDS | 180 |
| | DGSFFLYSKL TVDKSRWQQG NVFSCSVMHE ALHNHYTQKS LSLSPG | |
| ELNN AE288_3 | GTSESATPES GPGSEPATSG SETPGTSESA TPESGPGSEP ATSGSETPGT SESATPESGP | 60 |
| SEQ ID NO: 24 | GTSTEPSEGS APGSPAGSPT STEEGTSESA TPESGPGSEP ATSGSETPGT SESATPESGP | 120 |
| | GSPAGSPTST EEGSPAGSPT STEEGTSTEP SEGSAPGTSE SATPESGPGT SESATPESGP | 180 |
| | GTSESATPES GPGSEPATSG SETPGSEPAT SGSETPGSPA GSPTSTEEGT STEPSEGSAP | 240 |
| | GTSTEPSEGS APGSEPATSG SETPGTSESA TPESGPGTST EPSEGSAPAS S | 291 |

What is claimed is:

1. A pharmaceutical composition comprising:
(a) a chimeric protein comprising
a first polypeptide chain which comprises a Factor VIII (“FVIII”) protein and a first immunoglobulin (“Ig”) constant region or a portion thereof, and
a second polypeptide chain which comprises a von Willebrand Factor (“VWF”) protein and a second Ig constant region or a portion thereof;
(b) sucrose;
(c) histidine;
(d) arginine;
(e) calcium chloride; and
(f) a polysorbate.

2. The pharmaceutical composition of claim 1, wherein the pharmaceutical composition comprises about 5% (w/v) to about 7.5% (w/v) sucrose.

3. The pharmaceutical composition of claim 1, wherein the pharmaceutical composition comprises about 1% (w/v) to about 4% (w/v) sucrose.

4. The pharmaceutical composition of any one of claims 1 to 3, wherein the pharmaceutical composition comprises about 5 mM to about 15 mM histidine.

5. The pharmaceutical composition of any one of claims 1-4, wherein the pharmaceutical composition comprises about 200 mM to about 300 mM arginine.

6. The pharmaceutical composition of any one of claims 1-5, wherein the pharmaceutical composition comprises about 2.5 mM to about 10 mM calcium chloride.

7. The pharmaceutical composition of any one of claims 1-6, wherein the polysorbate is polysorbate 20.

8. The pharmaceutical composition of any one of claims 1-7, wherein the polysorbate is polysorbate 80.

9. The pharmaceutical composition of any one of claims 1-8, wherein the pharmaceutical composition comprises about 0.008% (w/v) to about 0.1% (w/v) polysorbate 20 or polysorbate 80.

10. A pharmaceutical composition comprising:
(a) a chimeric protein comprising
a first polypeptide chain which comprises a Factor VIII (“FVIII”) protein and a first immunoglobulin (“Ig”) constant region or a portion thereof, and
a second polypeptide chain which comprises a von Willebrand Factor (“VWF”) protein and a second Ig constant region or a portion thereof;
(b) about 1% (w/v) to about 4% (w/v) sucrose;
(c) about 5 mM to about 15 mM histidine;
(d) about 200 mM to about 300 mM arginine;
(e) about 2.5 mM to about 10 mM calcium chloride; and
(f) about 0.008% (w/v) to about 0.1% (w/v) polysorbate 20 or polysorbate 80.

11. The pharmaceutical composition of claim 10, wherein the polysorbate is polysorbate 20.

12. The pharmaceutical composition of claim 10 or claim 11, wherein the polysorbate is polysorbate 80.

13. The pharmaceutical composition of any one of claims 10-12, wherein the pharmaceutical composition comprises:
(a) about 2% (w/v) sucrose;
(b) about 10 mM histidine;
(c) about 250 mM arginine;
(d) about 5 mM calcium chloride; and
(e) about 0.05% polysorbate 20 or polysorbate 80.

14. The pharmaceutical composition of any one of claims 10-13, wherein the pharmaceutical composition comprises:
(a) about 2% (w/v) sucrose;
(b) about 10 mM L-histidine;
(c) about 250 mM L-arginine-HCl;
(d) about 5 mM calcium chloride; and
(e) about 0.05% polysorbate 80.

15. The pharmaceutical composition of any one of claims 10-12, wherein the pharmaceutical composition comprises:
(a) 10 mg/mL to 40 mg/mL sucrose;
(b) 1.5 mg/mL to 2.0 mg/mL L-histidine;
(c) 40 mg/mL to 70 mg/mL L-arginine-HCl;
(d) 0.4 mg/mL to 0.9 mg/mL calcium chloride; and
(e) 0.4 mg/ml to 0.7 mg/mL polysorbate 80.

16. The pharmaceutical composition of claim 15, wherein the pharmaceutical composition comprises:
(a) 22.45 mg/ml sucrose;
(b) 1.74 mg/ml L-histidine;
(c) 59.11 mg/ml L-arginine-HCl;
(d) 0.82 mg/ml calcium chloride dihydrate; and
(e) 0.56 mg/ml polysorbate 80.

17. The pharmaceutical composition of claim 15, wherein the pharmaceutical composition comprises:
(a) 22.45 mg/ml sucrose;
(b) 1.74 mg/ml L-histidine;
(c) 59.11 mg/ml L-arginine-HCl;
(d) 0.62 mg/ml calcium chloride; and
(e) 0.56 mg/ml polysorbate 80.

18. The pharmaceutical composition of any one of claims 15-17, wherein the pharmaceutical composition comprises:
(a) 10 mg/mL to 40 mg/mL sucrose;
(b) 1.5 mg/mL to 2.0 mg/mL L-histidine;
(c) 40 mg/mL to 70 mg/mL L-arginine-HCl;
(d) 0.5 mg/mL to 0.9 mg/mL calcium chloride dihydrate; and
(e) 0.4 mg/mL to 0.7 mg/mL polysorbate 80.

19. The pharmaceutical composition of any one of claims 15-18, wherein the pharmaceutical composition comprises:
(a) about 20 mg/mL sucrose;
(b) about 1.6 mg/mL L-histidine;
(c) about 52.7 mg/mL L-arginine-HCl;
(d) about 0.7 mg/mL calcium chloride dihydrate; and
(e) about 0.5 mg/mL polysorbate 80.

20. The pharmaceutical composition of any one of claims 15-19, wherein the pharmaceutical composition comprises:
(a) about 20 mg/mL sucrose;
(b) about 1.6 mg/mL L-histidine;
(c) about 52.7 mg/mL L-arginine-HCl;
(d) about 0.6 mg/mL calcium chloride; and
(e) about 0.5 mg/mL polysorbate 80.

21. The pharmaceutical composition of any one of claims 15-20, wherein the pharmaceutical composition comprises:
(a) about 20 mg/mL sucrose;
(b) about 1.6 mg/mL L-histidine;
(c) about 43.6 mg/mL L-arginine;
(d) about 0.7 mg/mL calcium chloride dihydrate; and
(e) about 0.5 mg/mL polysorbate 80.

22. The pharmaceutical composition of any one of claims 15-21, wherein the pharmaceutical composition comprises:
(a) about 20 mg/mL sucrose;
(b) about 1.6 mg/mL L-histidine;
(c) about 43.6 mg/mL L-arginine;
(d) about 0.6 mg/mL calcium chloride; and
(e) about 0.5 mg/mL polysorbate 80.

23. The pharmaceutical composition of any one of claims 15-22, wherein the pharmaceutical composition comprises:
(a) 10 mg/mL to 40 mg/mL sucrose;
(b) 1.5 mg/mL to 2.0 mg/mL L-histidine;
(c) 50 mg/mL to 70 mg/mL L-arginine-HCl;
(d) 0.7 mg/mL to 0.9 mg/mL calcium chloride dihydrate; and
(e) 0.4 mg/mL to 0.7 mg/mL polysorbate 80.

24. The pharmaceutical composition of claim 15, wherein the pharmaceutical composition comprises:
(a) 10 mg/mL to 40 mg/mL sucrose;
(b) 1.5 mg/mL to 2.0 mg/mL L-histidine;
(c) 50 mg/mL to 70 mg/mL L-arginine-HCl;
(d) 0.4 mg/mL to 0.8 mg/mL calcium chloride; and
(e) 0.4 mg/mL to 0.7 mg/mL polysorbate 80.

25. The pharmaceutical composition of claim 15, wherein the pharmaceutical composition comprises:
(a) 10 mg/mL to 40 mg/mL sucrose;
(b) 1.5 mg/ml to 2.0 mg/mL L-histidine;
(c) 40 mg/mL to 60 mg/mL L-arginine;
(d) 0.7 mg/mL to 0.9 mg/mL calcium chloride; and
(e) 0.4 mg/mL to 0.7 mg/mL polysorbate 80.

26. The pharmaceutical composition of claim 15, wherein the pharmaceutical composition comprises:
(a) 10 mg/mL to 40 mg/mL sucrose;
(b) 1.5 mg/ml to 2.0 mg/mL L-histidine;
(c) 40 mg/mL to 60 mg/mL L-arginine;
(d) 0.4 mg/mL to 0.7 mg/mL calcium chloride dihydrate; and
(e) 0.4 mg/ml to 0.7 mg/mL polysorbate 80.

27. The pharmaceutical composition of claim 15, wherein the pharmaceutical composition comprises:
(a) 22.45 mg/ml sucrose;
(b) 1.74 mg/ml L-histidine;
(c) 59.11 mg/ml L-arginine-HCl;
(d) 0.82 mg/ml calcium chloride dihydrate; and
(e) 0.56 mg/ml polysorbate 80.

28. The pharmaceutical composition of claim 15, wherein the pharmaceutical composition comprises:
(a) 22.45 mg/ml sucrose;
(b) 1.74 mg/ml L-histidine;
(c) 48.88 mg/ml L-arginine;
(d) 0.82 mg/ml calcium chloride dihydrate; and
(e) 0.56 mg/ml polysorbate 80.

29. The pharmaceutical composition of claim 15, wherein the pharmaceutical composition comprises:
(a) 22.45 mg/ml sucrose;
(b) 1.74 mg/ml L-histidine;
(c) 59.11 mg/ml L-arginine-HCl;
(d) 0.62 mg/ml calcium chloride; and
(e) 0.56 mg/ml polysorbate 80.

30. The pharmaceutical composition of claim 15, wherein the pharmaceutical composition comprises:
(a) 22.45 mg/ml sucrose;
(b) 1.74 mg/ml L-histidine;
(c) 48.88 mg/ml L-arginine;
(d) 0.62 mg/ml calcium chloride; and
(e) 0.56 mg/ml polysorbate 80.

31. The pharmaceutical composition of any one of claims 1 to 30, wherein the pharmaceutical composition has a pH of about 6.5 to about 7.5.

32. The pharmaceutical composition of any one of claims 1 to 31, wherein the pharmaceutical composition has a pH of about 7.0.

33. The pharmaceutical composition of any one of claims 1 to 31, wherein the pharmaceutical composition has a pH of about 6.8.

34. The pharmaceutical composition of any one of claims 1 to 33, wherein the pharmaceutical composition comprises less than 8.8 mg/mL sodium chloride (NaCl).

35. The pharmaceutical composition of any one of claims 1 to 34, wherein the pharmaceutical composition does not comprise NaCl.

36. The pharmaceutical composition of any one of claims 1 to 13, wherein the histidine is L-histidine.

37. The pharmaceutical composition of any one of claims 1 to 13, wherein the arginine is L-arginine.

38. The pharmaceutical composition of any one of claims 1 to 13, which comprises arginine-HCl.

39. The pharmaceutical composition of any one of claims 1 to 13, which comprises L-arginine-HCl.

40. The pharmaceutical composition of any one of claims 1 to 15, which comprises calcium chloride dihydrate.

41. The pharmaceutical composition of any one of claims 1 to 40, wherein the pharmaceutical composition has a chimeric protein concentration of about 0.8 to about 1.2 mg/mL.

42. The pharmaceutical composition of any one of claims 1 to 41, wherein the pharmaceutical composition comprises 75 IU/mL to 2,000 IU/mL of the chimeric protein.

43. The pharmaceutical composition of any one of claims 1 to 42, wherein the pharmaceutical composition has an osmolality about 525 to about 725 mOsm/kg.

44. The pharmaceutical composition of any one of claims 1 to 43, wherein the pharmaceutical composition comprises has an osmolality about 600 to about 650 mOsm/kg.

45. The pharmaceutical composition of any one of claims 1 to 44, wherein the pharmaceutical composition comprises has a turbidity of less than about 7 Nephelometric Turbidity Units (NTU).

46. The pharmaceutical composition of any one of claims 1 to 45, wherein the first polypeptide chain comprises the amino acid sequence set forth as SEQ ID NO: 1 and the second polypeptide chain comprises the amino acid sequence set forth as SEQ ID NO: 2, wherein the first polypeptide chain and the second polypeptide chain are covalently linked by two disulfide bonds between Fc domains in the first and second polypeptide chains.

47. The pharmaceutical composition of any one of claims 1 to 46, wherein the chimeric protein is efanesoctocog alfa.

48. The pharmaceutical composition of any one of claims 1 to 41 or 43 to 47, which comprises about 250 IU, 500 IU, 1000 IU, 2000 IU, 3000 IU, or 4,000 IU of the chimeric protein.

49. A method of treating hemophilia A in a subject in need thereof, comprising administering to the subject an effective amount of the pharmaceutical composition of any one of claims 1 to 48.

50. The method of claim 49, wherein the pharmaceutical composition is self-administered.

51. The method of claim 49 or 50, wherein the pharmaceutical composition is administered intravenously.

52. The method of any one of claims 49 to 51, wherein the pharmaceutical composition is administered intravenously at a dose of 20 IU/kg to 70 IU/kg.

53. The method of any one of claims 49 to 52, wherein the pharmaceutical composition is administered intravenously at a dose of 50 IU/kg.

54. The method any one of claims 49 to 53, wherein the pharmaceutical composition is administered intravenously once every 7-10 days.

55. The method of claim any one of claims 49 to 54, wherein the pharmaceutical composition is administered intravenously once weekly.

56. A pharmaceutical kit comprising:
(i) a first container comprising a lyophilized pharmaceutical composition comprising
(a) a chimeric protein comprising
a first polypeptide chain which comprises a Factor VIII (“FVIII”) protein or a portion thereof and a first immunoglobulin (“Ig”) constant region or a portion thereof, and
a second polypeptide chain which comprises a von Willebrand Factor (“VWF”) protein and a second Ig constant region or a portion thereof;
(b) about 30 mg to about 135 mg sucrose;
(c) about 2.5 mg to about 7.5 mg histidine;
(d) about 140 mg to about 200 mg arginine;
(e) about 1.5 mg to about 5 mg calcium chloride; and
(f) about 1 mg to about 5 mg polysorbate 20 or polysorbate 80, and
(ii) a second container comprising sterile water.

57. The pharmaceutical kit of claim 56, which does not comprise NaCl.

58. The pharmaceutical kit of claim 56 or 57, wherein the histidine is L-histidine.

59. The pharmaceutical kit any one of claims 56 to 58, wherein the arginine is L-arginine.

60. The pharmaceutical kit of claim any one of claims 56 to 59, which comprises arginine-HCl.

61. The pharmaceutical kit of claim any one of claims 56 to 60, which comprises L-arginine-HCl.

62. The pharmaceutical kit of claim any one of claims 56 to 61, which comprises calcium chloride dihydrate.

63. The pharmaceutical kit of claim any one of claims 56 to 61, wherein the lyophilized pharmaceutical composition comprises:
(a) about 67.3 mg sucrose;
(b) about 5.2 mg L-histidine;
(c) about 177.3 mg L-arginine-HCl;
(d) about 2.5 mg calcium chloride; and
(e) about 1.7 mg polysorbate 20 or polysorbate 80.

64. The pharmaceutical kit of any one of claims 56 to 61, wherein the lyophilized pharmaceutical composition comprises:
(a) about 67.3 mg sucrose;
(b) about 5.2 mg L-histidine;
(c) about 146.6 mg L-arginine-HCl;
(d) about 2.5 mg calcium chloride; and
(e) about 1.7 mg polysorbate 20 or polysorbate 80.

65. The pharmaceutical kit of any one of claims 56 to 64, wherein the lyophilized pharmaceutical composition has a moisture content of less than 2%.

66. The pharmaceutical kit of any one of claims 56 to 65, wherein the lyophilized pharmaceutical composition has a moisture content of less than 1.8%.

67. The pharmaceutical kit of any one of claims 56 to 66, wherein the lyophilized pharmaceutical composition has a moisture content of less than 1.6%.

68. The pharmaceutical kit of any one of claims 56 to 67, wherein the lyophilized pharmaceutical composition is in a lyophilized cake.

69. The pharmaceutical kit of claim 68, wherein the lyophilized cake is white.

70. The pharmaceutical kit of claim 68 or 69, wherein the lyophilized cake is less than Y4 in the European Pharmacopoeia color scale.

71. The pharmaceutical kit of claim any one of claims 56 to 70, wherein the first container comprises 100 IU to 10,000 IU of the chimeric protein.

72. The pharmaceutical kit of any one of claims 56 to 71, wherein the first container comprises 250 IU, 500 IU, 1000 IU, 2000 IU, 3000 IU, or 4,000 IU of the chimeric protein.

73. The pharmaceutical kit of any one of claims 56 to 72, further comprising instructions for combining the lyophilized pharmaceutical composition and the sterile water.

74. The pharmaceutical kit of any one of claims 56 to 73, wherein when the lyophilized pharmaceutical composition and the sterile water are combined, then the lyophilized pharmaceutical composition is reconstituted within 7 to 12 seconds.

75. The pharmaceutical kit of any one of claims 56 to 74, wherein when the lyophilized pharmaceutical composition and the sterile water are combined, then the osmolality of the resulting solution is about 525 to about 725 mOsm/kg.

76. The pharmaceutical kit of any one of claims 56 to 75, wherein when the lyophilized pharmaceutical composition and the sterile water are combined, then the osmolality of the resulting solution is about 600 to about 650 mOsm/kg.

77. The pharmaceutical kit of claim any one of claims 56 to 76, wherein when the lyophilized pharmaceutical composition and the sterile water are combined, then the pH of the resulting solution is about 6.5 to about 7.5.

78. The pharmaceutical kit of any one of claims 56 to 77, wherein when the lyophilized pharmaceutical composition and the sterile water are combined, then the pH of the resulting solution is about 7.0.

79. The pharmaceutical kit of any one of claims 56 to 78, wherein when the lyophilized pharmaceutical composition and the sterile water are combined, then the protein concentration of the resulting solution is about 0.8 to about 1.2 mg/mL.

80. The pharmaceutical kit of claim 79, wherein less than 3% of the protein is aggregated.

81. The pharmaceutical kit of any one of claims 56 to 80, wherein when the lyophilized pharmaceutical composition and the sterile water are combined, then the turbidity of the resulting solution is less than about 7 Nephelometric Turbidity Units (NTU).

82. The pharmaceutical kit of any one of claims 56 to 81, wherein when the lyophilized pharmaceutical composition and the sterile water are combined, then the resulting solution comprises:
(a) 10 mg/mL to 40 mg/mL sucrose;
(b) 1.5 mg/mL to 2.0 mg/mL L-histidine;
(c) 40 mg/mL to 70 mg/mL L-arginine-HCl;
(d) 0.5 mg/mL to 0.9 mg/mL calcium chloride; and
(e) 0.4 mg/mL to 0.7 mg/mL polysorbate 80.

83. The pharmaceutical kit of any one of claims 56 to 81, wherein when the lyophilized pharmaceutical composition and the sterile water are combined, then the resulting solution comprises:
(a) 10 mg/mL to 40 mg/mL sucrose;
(b) 1.5 mg/mL to 2.0 mg/mL L-histidine;
(c) 40 mg/mL to 60 mg/mL L-arginine;
(d) 0.5 mg/mL to 0.9 mg/mL calcium chloride; and
(e) 0.4 mg/mL to 0.7 mg/mL polysorbate 80.

84. The pharmaceutical kit of any one of claims 56 to 81, wherein when the lyophilized pharmaceutical composition and the sterile water are combined, then the resulting solution comprises:
(a) 22.45 mg/ml sucrose;
(b) 1.74 mg/ml L-histidine;
(c) 59.11 mg/ml L-arginine-HCl;
(d) 0.62 mg/ml calcium chloride; and
(e) 0.56 mg/ml polysorbate 80.

85. The pharmaceutical kit of any one of claims 56 to 81, wherein when the lyophilized pharmaceutical composition and the sterile water are combined, then the resulting solution comprises:
(a) 22.45 mg/ml sucrose;
(b) 1.74 mg/ml L-histidine;
(c) 48.88 mg/ml L-arginine;
(d) 0.62 mg/ml calcium chloride; and
(e) 0.56 mg/ml polysorbate 80.

86. The pharmaceutical kit of any one of claims 56 to 85, wherein the second container comprises about 2 mL to about 5 mL of the sterile water.

87. The pharmaceutical kit of any one of claims 56 to 86, wherein the second container comprises about 3 mL of the sterile water.

88. The pharmaceutical kit of any one of claims 56 to 86, wherein the second container comprises about 3.3 mL of the sterile water.

89. The pharmaceutical kit of any one of claims 56 to 88, wherein the first container is a glass vial comprising a rubber stopper.

90. The pharmaceutical kit of claim any one of claims 56 to 89, wherein the second container is a syringe body.

91. The pharmaceutical kit of claim 90, wherein the sterile water is in the syringe body.

92. The pharmaceutical kit of claim 90 or 91, wherein the syringe body is associated with a plunger.

93. The pharmaceutical kit of any one of claims 89 to 92, further comprising an adaptor to connect the glass vial to the syringe body.

94. The pharmaceutical kit of any one of claims 89 to 93, further comprising infusion tubing associated with a needle to be connected to the syringe body, suitable for intravenous infusion.

95. The pharmaceutical kit of any one of claims 56 to 94, wherein the first polypeptide chain comprises the amino acid sequence set forth as SEQ ID NO: 1 and the second polypeptide chain comprises the amino acid sequence set forth as SEQ ID NO: 2, wherein the first polypeptide chain and the second polypeptide chain are covalently linked by two disulfide bonds between Fc domains in the first and second polypeptide chains.

96. A method of treating hemophilia A in a subject in need thereof, comprising combining the lyophilized pharmaceutical composition and the sterile water of the kit of any one of claims 56 to 95, and administering to the subject an effective amount of the resulting combination.

97. The method of claim 96, wherein the subject combines the lyophilized pharmaceutical composition and the sterile water of the kit.

98. The method of claim 96 or 97, wherein the combination is self-administered by the subject.

99. The pharmaceutical composition of any one of claims 1-9, wherein the pharmaceutical composition comprises:
(a) about 5% (w/v) to about 7.5% (w/v) sucrose;
(b) about 5 mM to about 15 mM histidine;
(c) about 200 mM to about 300 mM arginine;
(d) about 2.5 mM to about 10 mM calcium chloride; and
(e) about 0.008% (w/v) to about 0.1% (w/v) polysorbate 20 or polysorbate 80.

100. The pharmaceutical composition of any one of claim 1-9 or 99, wherein the pharmaceutical composition comprises:
(a) about 5% (w/v) sucrose;
(b) about 10 mM histidine;
(c) about 250 mM arginine;
(d) about 5 mM calcium chloride; and
(e) about 0.05% polysorbate 20 or polysorbate 80.

101. The pharmaceutical composition of any one of claim 1-9 or 99-100, wherein the pharmaceutical composition comprises:
(a) about 5% (w/v) sucrose;
(b) about 10 mM L-histidine;
(c) about 250 mM L-arginine-HCl;
(d) about 5 mM calcium chloride; and
(e) about 0.05% polysorbate 80.

102. The pharmaceutical composition of claim 1, wherein the pharmaceutical composition comprises:
(a) 45 mg/mL to 60 mg/mL sucrose;
(b) 1.5 mg/mL to 2.0 mg/mL L-histidine;
(c) 40 mg/mL to 70 mg/mL L-arginine-HCl;
(d) 0.4 mg/mL to 0.9 mg/mL calcium chloride; and
(e) 0.4 mg/mL to 0.7 mg/mL polysorbate 80.

103. The pharmaceutical composition of claim 102, wherein the pharmaceutical composition comprises:
(a) 56.12 mg/ml sucrose;
(b) 1.74 mg/ml L-histidine;
(c) 59.11 mg/ml L-arginine-HCl;
(d) 0.82 mg/ml calcium chloride dihydrate; and
(e) 0.56 mg/ml polysorbate 80.

104. The pharmaceutical composition of any one of claims 102-103, wherein the pharmaceutical composition comprises:
(a) 56.12 mg/ml sucrose;
(b) 1.74 mg/ml L-histidine;
(c) 59.11 mg/ml L-arginine-HCl;
(d) 0.62 mg/ml calcium chloride; and
(e) 0.56 mg/ml polysorbate 80.

105. The pharmaceutical composition of any one of claims 102-104, wherein the pharmaceutical composition comprises:
(a) 45 mg/mL to 60 mg/mL sucrose;
(b) 1.5 mg/mL to 2.0 mg/mL L-histidine;
(c) 40 mg/mL to 70 mg/mL L-arginine-HCl;
(d) 0.5 mg/mL to 0.9 mg/mL calcium chloride dihydrate; and
(e) 0.4 mg/ml to 0.7 mg/mL polysorbate 80.

106. The pharmaceutical composition of any one of claims 102-105, wherein the pharmaceutical composition comprises:
(a) about 50 mg/mL sucrose;
(b) about 1.6 mg/mL L-histidine;
(c) about 52.7 mg/mL L-arginine-HCl;
(d) about 0.7 mg/mL calcium chloride dihydrate; and
(e) about 0.5 mg/mL polysorbate 80.

107. The pharmaceutical composition of any one of claims 102-106, wherein the pharmaceutical composition comprises:
(a) about 50 mg/mL sucrose;
(b) about 1.6 mg/mL L-histidine;
(c) about 52.7 mg/mL L-arginine-HCl;
(d) about 0.6 mg/mL calcium chloride; and
(e) about 0.5 mg/mL polysorbate 80.

108. The pharmaceutical composition of any one of claims 102-107, wherein the pharmaceutical composition comprises:
(a) about 50 mg/mL sucrose;
(b) about 1.6 mg/mL L-histidine;
(c) about 43.6 mg/mL L-arginine;
(d) about 0.7 mg/mL calcium chloride dihydrate; and
(e) about 0.5 mg/mL polysorbate 80.

109. The pharmaceutical composition of any one of claims 102-108, wherein the pharmaceutical composition comprises:
(a) about 50 mg/mL sucrose;
(b) about 1.6 mg/mL L-histidine;
(c) about 43.6 mg/mL L-arginine;
(d) about 0.6 mg/mL calcium chloride; and
(e) about 0.5 mg/mL polysorbate 80.

110. The pharmaceutical composition of any one of claims 102-109, wherein the pharmaceutical composition comprises:
(a) 45 mg/mL to 60 mg/mL sucrose;
(b) 1.5 mg/mL to 2.0 mg/mL L-histidine;
(c) 50 mg/mL to 70 mg/mL L-arginine-HCl;
(d) 0.7 mg/mL to 0.9 mg/mL calcium chloride dihydrate; and
(e) 0.4 mg/mL to 0.7 mg/mL polysorbate 80.

111. The pharmaceutical composition of any one of claims 102-110, wherein the pharmaceutical composition comprises:
(a) 45 mg/mL to 60 mg/mL sucrose;
(b) 1.5 mg/mL to 2.0 mg/mL L-histidine;
(c) 50 mg/mL to 70 mg/mL L-arginine-HCl;
(d) 0.4 mg/mL to 0.8 mg/mL calcium chloride; and
(e) 0.4 mg/mL to 0.7 mg/mL polysorbate 80.

112. The pharmaceutical composition of any one of claims 102-111, wherein the pharmaceutical composition comprises:
(a) 45 mg/ml to 60 mg/mL sucrose;
(b) 1.5 mg/mL to 2.0 mg/mL L-histidine;
(c) 40 mg/mL to 60 mg/mL L-arginine;
(d) 0.7 mg/mL to 0.9 mg/mL calcium chloride; and
(e) 0.4 mg/mL to 0.7 mg/mL polysorbate 80.

113. The pharmaceutical composition of any one of claims 102-112, wherein the pharmaceutical composition comprises:
(a) 45 mg/mL to 60 mg/mL sucrose;
(b) 1.5 mg/ml to 2.0 mg/mL L-histidine;
(c) 40 mg/mL to 60 mg/mL L-arginine;
(d) 0.4 mg/mL to 0.7 mg/mL calcium chloride dihydrate; and
(e) 0.4 mg/mL to 0.7 mg/mL polysorbate 80.

114. The pharmaceutical composition of any one of claims 102-113, wherein the pharmaceutical composition comprises:
(a) 56.12 mg/ml sucrose;
(b) 1.74 mg/ml L-histidine;
(c) 59.11 mg/ml L-arginine-HCl;
(d) 0.82 mg/ml calcium chloride dihydrate; and
(e) 0.56 mg/ml polysorbate 80.

115. The pharmaceutical composition of any one of claims 102-114, wherein the pharmaceutical composition comprises:
(a) 56.12 mg/ml sucrose;
(b) 1.74 mg/ml L-histidine;
(c) 48.88 mg/ml L-arginine;
(d) 0.82 mg/ml calcium chloride dihydrate; and
(e) 0.56 mg/ml polysorbate 80.

116. The pharmaceutical composition of any one of claims 102-115, wherein the pharmaceutical composition comprises:
(a) 56.12 mg/ml sucrose;
(b) 1.74 mg/ml L-histidine;
(c) 59.11 mg/ml L-arginine-HCl;
(d) 0.62 mg/ml calcium chloride; and
(e) 0.56 mg/ml polysorbate 80.

117. The pharmaceutical composition of any one of claims 102-116, wherein the pharmaceutical composition comprises:
(a) 56.12 mg/ml sucrose;
(b) 1.74 mg/ml L-histidine;
(c) 48.88 mg/ml L-arginine;
(d) 0.62 mg/ml calcium chloride; and
(e) 0.56 mg/ml polysorbate 80.

118. The pharmaceutical composition of any one of claims 99-117, wherein the pharmaceutical composition has a pH of about 6.5 to about 7.5.

119. The pharmaceutical composition of claim 118, wherein the pharmaceutical composition has a pH of about 7.0.

120. The pharmaceutical composition of claim 118, wherein the pharmaceutical composition has a pH of about 6.8.

121. The pharmaceutical composition of any one of claims 99-120, wherein the pharmaceutical composition comprises less than 8.8 mg/mL sodium chloride (NaCl).

122. The pharmaceutical composition any one of claims 99-121, which does not comprise NaCl.

123. The pharmaceutical composition of any one of claims 1-11, wherein the histidine is L-histidine.

124. The pharmaceutical composition of any one of claims 99-100, wherein the arginine is L-arginine.

125. The pharmaceutical composition of any one of claims 99-100, which comprises arginine-HCl.

126. The pharmaceutical composition of any one of claims 99-100, which comprises L-arginine-HCl.

127. The pharmaceutical composition of any one of claims 99-102, which comprises calcium chloride dihydrate.

128. The pharmaceutical composition of any one of claims 99-127, wherein the pharmaceutical composition has a chimeric protein concentration of about 0.8 to about 1.2 mg/mL.

129. The pharmaceutical composition of any one of claims 99-127, wherein the pharmaceutical composition comprises 75 IU/mL to 2,000 IU/mL of the chimeric protein.

130. The pharmaceutical composition of any one of claims 99-129, wherein the pharmaceutical composition has an osmolality about 525 to about 725 mOsm/kg.

131. The pharmaceutical composition of claim 130, wherein the pharmaceutical composition comprises has an osmolality about 600 to about 650 mOsm/kg.

132. The pharmaceutical composition of any one of claims 99-131, wherein the pharmaceutical composition comprises has a turbidity of less than about 7 Nephelometric Turbidity Units.

133. The pharmaceutical composition of any one of claims 99-132, wherein the first polypeptide chain comprises the amino acid sequence set forth as SEQ ID NO: 1 and the second polypeptide chain comprises the amino acid sequence set forth as SEQ ID NO: 2, wherein the first polypeptide chain and the second polypeptide chain are covalently linked by two disulfide bonds between Fc domains in the first and second polypeptide chains.

134. The pharmaceutical composition of any one of claims 99-133, wherein the chimeric protein is efanesoctocog alfa.

135. The pharmaceutical composition of any one of claim 99-127 or 130-134, which comprises about 250 IU, 500 IU, 1000 IU, 2000 IU, 3000 IU, or 4,000 IU of the chimeric protein.

136. A method of treating hemophilia A in a subject in need thereof, comprising administering to the subject an effective amount of the pharmaceutical composition of any one of claims 99 to 135.

137. The method of claim 136, wherein the pharmaceutical composition is self-administered.

138. The method of claim 136, wherein the pharmaceutical composition is administered intravenously.

139. The method of claim 137, wherein the pharmaceutical composition is administered intravenously.

140. The method of any one of claims 136-139, wherein the pharmaceutical composition is administered intravenously at a dose of 20 IU/kg to 70 IU/kg.

141. The method of any one of claims 136-139, wherein the pharmaceutical composition is administered intravenously at a dose of 50 IU/kg.

142. The method of any one of claims 136-140, wherein the pharmaceutical composition is administered intravenously once every 7-10 days.

143. The method of any one of claims 136-140, wherein the pharmaceutical composition is administered intravenously once weekly.

144. A pharmaceutical kit comprising:
(i) a first container comprising a lyophilized pharmaceutical composition comprising
(a) a chimeric protein comprising
a first polypeptide chain which comprises a Factor VIII (“FVIII”) protein or a portion thereof and a first immunoglobulin (“Ig”) constant region or a portion thereof, and
a second polypeptide chain which comprises a von Willebrand Factor (“VWF”) protein and a second Ig constant region or a portion thereof;
(b) sucrose;
(c) histidine;
(d) arginine;
(e) calcium chloride; and
(f) polysorbate 20 or polysorbate 80, and
(ii) a second container comprising sterile water.

145. The pharmaceutical kit of claim 144, wherein the lyophilized pharmaceutical composition comprises:
(a) about 160 mg to about 200 mg sucrose;
(b) about 2.5 mg to about 7.5 mg histidine;
(c) about 140 mg to about 200 mg arginine;
(d) about 1.5 mg to about 5 mg calcium chloride; and
(e) about 1 mg to about 5 mg polysorbate 20 or polysorbate 80.

146. The pharmaceutical kit of any one of claims 144-145, which does not comprise NaCl.

147. The pharmaceutical kit of any one of claims 144-146, wherein the histidine is L-histidine.

148. The pharmaceutical kit of any one of claims 144-147, wherein the arginine is L-arginine.

149. The pharmaceutical kit of any one of claims 144-147, which comprises arginine-HCl.

150. The pharmaceutical kit of any one of claims 144-147, which comprises L-arginine-HCl.

151. The pharmaceutical kit of any one of claims 144-150, which comprises calcium chloride dihydrate.

152. The pharmaceutical kit of any one of claims 144-151, wherein the lyophilized pharmaceutical composition comprises:
(a) about 168.3 mg sucrose;
(b) about 5.2 mg L-histidine;
(c) about 177.3 mg L-arginine-HCl;
(d) about 2.5 mg calcium chloride; and
(e) about 1.7 mg polysorbate 20 or polysorbate 80.

153. The pharmaceutical kit of any one of claims 144-151, wherein the lyophilized pharmaceutical composition comprises:
(a) about 168.3 mg sucrose;
(b) about 5.2 mg L-histidine;
(c) about 146.6 mg L-arginine-HCl;
(d) about 2.5 mg calcium chloride; and
(e) about 1.7 mg polysorbate 20 or polysorbate 80.

154. The pharmaceutical kit of any one of claims 144-153, wherein the lyophilized pharmaceutical composition has a moisture content of less than 2%.

155. The pharmaceutical kit of any one of claims 144-154, wherein the lyophilized pharmaceutical composition has a moisture content of less than 1.8%.

156. The pharmaceutical kit of any one of claims 144-155, wherein the lyophilized pharmaceutical composition has a moisture content of less than 1.6%.

157. The pharmaceutical kit of any one of claims 144-156, wherein the lyophilized pharmaceutical composition is in a lyophilized cake.

158. The pharmaceutical kit of claim 157, wherein the lyophilized cake is white.

159. The pharmaceutical kit of claim 158, wherein the lyophilized cake is less than Y4 in the European Pharmacopoeia color scale.

160. The pharmaceutical kit of any one of claims 144-159, wherein the first container comprises 100 IU to 10,000 IU of the chimeric protein.

161. The pharmaceutical kit of any one of claims 144-160, wherein the first container comprises 250 IU, 500 IU, 1000 IU, 2000 IU, 3000 IU, or 4,000 IU of the chimeric protein.

162. The pharmaceutical kit of any one of claims 144-161, further comprising instructions for combining the lyophilized pharmaceutical composition and the sterile water.

163. The pharmaceutical kit of any one of claims 144-162, wherein when the lyophilized pharmaceutical composition and the sterile water are combined, then the lyophilized pharmaceutical composition is reconstituted within 7 to 12 seconds.

164. The pharmaceutical kit of any one of claims 144-163, wherein when the lyophilized pharmaceutical composition and the sterile water are combined, then the osmolality of the resulting solution is about 525 to about 725 mOsm/kg.

165. The pharmaceutical kit of any one of claims 144-164, wherein when the lyophilized pharmaceutical composition and the sterile water are combined, then the osmolality of the resulting solution is about 600 to about 650 mOsm/kg.

166. The pharmaceutical kit of any one of claims 144-165, wherein when the lyophilized pharmaceutical composition and the sterile water are combined, then the pH of the resulting solution is about 6.5 to about 7.5.

167. The pharmaceutical kit of any one of claims 144-166, wherein when the lyophilized pharmaceutical composition and the sterile water are combined, then the pH of the resulting solution is about 7.0.

168. The pharmaceutical kit of any one of claims 144-167, wherein when the lyophilized pharmaceutical composition and the sterile water are combined, then the protein concentration of the resulting solution is about 0.8 to about 1.2 mg/mL.

169. The pharmaceutical kit of claim 168, wherein less than 3% of the protein is aggregated.

170. The pharmaceutical kit of any one of claims 144-169, wherein when the lyophilized pharmaceutical composition and the sterile water are combined, then the turbidity of the resulting solution is less than about 7 Nephelometric Turbidity Units.

171. The pharmaceutical kit of claim 144, wherein when the lyophilized pharmaceutical composition and the sterile water are combined, then the resulting solution comprises:
(a) 45 mg/mL to 60 mg/mL sucrose;
(b) 1.5 mg/mL to 2.0 mg/mL L-histidine;
(c) 40 mg/mL to 70 mg/mL L-arginine-HCl;
(d) 0.5 mg/mL to 0.9 mg/mL calcium chloride; and
(e) 0.4 mg/mL to 0.7 mg/mL polysorbate 80.

172. The pharmaceutical kit of claim 171, wherein when the lyophilized pharmaceutical composition and the sterile water are combined, then the resulting solution comprises:
(a) 45 mg/mL to 60 mg/mL sucrose;
(b) 1.5 mg/mL to 2.0 mg/mL L-histidine;
(c) 40 mg/mL to 60 mg/mL L-arginine;
(d) 0.5 mg/mL to 0.9 mg/mL calcium chloride; and
(e) 0.4 mg/mL to 0.7 mg/mL polysorbate 80.

173. The pharmaceutical kit of any one of claim 144 or 171-172, wherein when the lyophilized pharmaceutical composition and the sterile water are combined, then the resulting solution comprises:
(a) 56.12 mg/ml sucrose;
(b) 1.74 mg/ml L-histidine;
(c) 59.11 mg/ml L-arginine-HCl;
(d) 0.62 mg/ml calcium chloride; and
(e) 0.56 mg/ml polysorbate 80.

174. The pharmaceutical kit of any one of claims 144 or 171-173, wherein when the lyophilized pharmaceutical composition and the sterile water are combined, then the resulting solution comprises:
(a) 56.12 mg/ml sucrose;
(b) 1.74 mg/ml L-histidine;
(c) 48.88 mg/ml L-arginine;
(d) 0.62 mg/ml calcium chloride; and
(e) 0.56 mg/ml polysorbate 80.

175. The pharmaceutical kit of any one of claims 144-174, wherein the second container comprises about 2 mL to about 5 mL of the sterile water.

176. The pharmaceutical kit of any one of claims 144-175, wherein the second container comprises about 3 mL of the sterile water.

177. The pharmaceutical kit of any one of claims 144-175, wherein the second container comprises about 3.3 mL of the sterile water.

178. The pharmaceutical kit of any one of claims 144-177, wherein the first container is a glass vial comprising a rubber stopper.

179. The pharmaceutical kit of any one of claims 144-178, wherein the second container is a syringe body.

180. The pharmaceutical kit of claim 179, wherein the sterile water is in the syringe body.

181. The pharmaceutical kit of claim 179 or 180, wherein the syringe body is associated with a plunger.

182. The pharmaceutical kit of any one of claims 179-181, further comprising an adaptor to connect the glass vial to the syringe body.

183. The pharmaceutical kit of any one of claims 179-182, further comprising infusion tubing associated with a needle to be connected to the syringe body, suitable for intravenous infusion.

184. The pharmaceutical kit of any one of claims 144 to 183, wherein the first polypeptide chain comprises the amino acid sequence set forth as SEQ ID NO: 1 and the second polypeptide chain comprises the amino acid sequence set forth as SEQ ID NO: 2, wherein the first polypeptide chain and the second polypeptide chain are covalently linked by two disulfide bonds between Fc domains in the first and second polypeptide chains.

185. A method of treating hemophilia A in a subject in need thereof, comprising combining the lyophilized pharmaceutical composition and the sterile water of the kit of any one of claims 144 to 184, and administering to the subject an effective amount of the resulting combination.

186. The method of claim 185, wherein the subject combines the lyophilized pharmaceutical composition and the sterile water of the kit.

187. The method of claim 185 or 186, wherein the combination is self-administered by the subject.

188. The pharmaceutical composition of any one of claims 1 to 34, wherein the pharmaceutical composition does not comprise NaOH.

189. The pharmaceutical composition of any one of claims 1 to 34, wherein the pharmaceutical composition does not comprise sodium ions.

190. The pharmaceutical kit of claim 56, which does not comprise NaOH.

191. The pharmaceutical kit of claim 56, which does not comprise sodium ions.

192. The pharmaceutical composition any one of claims 99-121, which does not comprise NaOH.

193. The pharmaceutical composition any one of claims 99-121, which does not comprise sodium ions.

194. The pharmaceutical kit of any one of claims 144-145, which does not comprise NaOH.

195. The pharmaceutical kit of any one of claims 144-145, which does not comprise sodium ions.


SEQUENCE LISTING (24 sequences)

SEQ ID NO: 1 - protein, 1946 aa, Artificial Sequence
  Synthesized
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 883 aa, Artificial Sequence
  Synthesized
SLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSDKNTGDYYEDSYEDISAYLLSKNNAIEPRSFSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 1965 aa, Artificial Sequence
  Synthesized
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - DNA, 5898 bp, Artificial Sequence
  Synthesized
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggaacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccagcctcgagcgaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctacgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaagtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaagttagcctgacctgcctggtcaaaggcttctatcccagcgacatcgccgtggagtgggagagcaatgggcagccggagaacaactacaagaccacgcctcccgtgttggactccgacggctccttcttcctctactccaagctcaccgtggacaagagcaggtggcagcaggggaacgtcttctcatgctccgtgatgcatgaggctctgcacaaccactacacgcagaagagcctctccctgtctccgggttga

SEQ ID NO: 5 - protein, 1646 aa, Artificial Sequence
  Synthesized
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSDKNTGDYYEDSYEDISAYLLSKNNAIEPRSFSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - DNA, 4941 bp, Artificial Sequence
  Synthesized
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcactggtcaatcccacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacatccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgccgcattctgcgacaccattgctgcctatgcccacgtgtgtgcccagcatggcaaggtggtgacctggaggacggccacattgtgcccccagagctgcgaggagaggaatctccgggagaacgggtatgaggctgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggtacatcagagagcgccacccctgaaagtggtcccgggagcgagccagccacatctgggtcggaaacgccaggcacatccgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgtctgacaagaacactggtgattattacgaggacagttatgaagatatttcagcatacttgctgagtaaaaacaatgccattgaaccaagaagcttctctgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaagtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaagttagcctgacctgcctggtcaaaggcttctatcccagcgacatcgccgtggagtgggagagcaatgggcagccggagaacaactacaagaccacgcctcccgtgttggactccgacggctccttcttcctctactccaagctcaccgtggacaagagcaggtggcagcaggggaacgtcttctcatgctccgtgatgcatgaggctctgcacaaccactacacgcagaagagcctctccctgtctccgggttga

SEQ ID NO: 7 - protein, 1945 aa, Artificial Sequence
  Synthesized
TRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 9 - protein, 288 aa, Artificial Sequence
  Synthesized
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 10 - protein, 46 aa, Artificial Sequence
  Synthesized
TSTEEGASISDKNTGDYYEDSYEDISAYLLSKNNAIEPRSFSDKTH

SEQ ID NO: 11 - protein, 45 aa, Artificial Sequence
  Synthesized
TSTEEGASSDKNTGDYYEDSYEDISAYLLSKNNAIEPRSFSDKTH

SEQ ID NO: 12 - protein, 1947 aa, Artificial Sequence
  Synthesized
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 288 aa, Artificial Sequence
  Synthesized
GSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 14 - protein, 144 aa, Artificial Sequence
  Synthesized
TSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEG

SEQ ID NO: 15 - protein, 32 aa, Artificial Sequence
  Synthesized
DKNTGDYYEDSYEDISAYLLSKNNAIEPRSFS

SEQ ID NO: 16 - protein, 19 aa, Homo sapiens
MQIELSTCFFLCLLRFCFS

SEQ ID NO: 17 - protein, 745 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQN

SEQ ID NO: 18 - protein, 684 aa, Homo sapiens
EITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 19 - protein, 22 aa, Homo sapiens
MIPARFAGVLLALALILPGTLC

SEQ ID NO: 20 - protein, 741 aa, Homo sapiens
AEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKR

SEQ ID NO: 21 - protein, 103 aa, Artificial Sequence
  Synthesized
SLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDAT

SEQ ID NO: 22 - protein, 374 aa, Artificial Sequence
  Synthesized
CSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEP

SEQ ID NO: 23 - protein, 226 aa, Artificial Sequence
  Synthesized
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 291 aa, Artificial Sequence
  Synthesized
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASS